(19) United States
(12) Patent Application Publication
(10) Pub. No.: US 2019/0054022 A1
(43) Pub. Date: Feb. 21, 2019

(54) FORMULATIONS OF SITE-SPECIFIC, MICROPARTICULATE COMPOSITIONS AND THEIR USE TO IMPROVE OUTCOME AFTER ANEURSYMAL SUBARACHNOID HEMORRHAGE

(71) Applicant: Edge Therapeutics, Inc., Berkeley Heights, NJ (US)

(72) Inventor: R. Loch Macdonald, Chatham Township, NJ (US)

(21) Appl. No.: 15/480,151

(22) Filed: Apr. 5, 2017

Related U.S. Application Data
(60) Provisional application No. 62/319,723, filed on Apr. 7, 2016.

(51) Int. Cl. A61K 9/16; A61K 31/4422; A61K 9/00; A61P 25/00

(52) U.S. Cl. CPC A61K 9/1647; A61K 31/4422; A61P 25/00; A61K 9/0085; A61K 9/1694; A61K 9/1652

(57) ABSTRACT

The described invention provides site-specific sustained release microparticulate formulations containing a therapeutic amount of an L-type voltage gated calcium channel inhibitor, a PLGA polymer comprising from 25% to 50% glycolide, and a hyaluronic acid. A therapeutic amount of the formulation is effective to reduce signs or symptoms of delayed cerebral ischemia comprising one or more of a cortical spreading ischemia, a cortical spreading depolarization, a plurality of microthromboemboli, or an angiographic vasospasm after brain injury in a mammal, while reducing the risk of systemic hypotension, cardiac dysfunction, anoxia, and intracranial hypertension.

### CROSS-REFERENCE TO RELATED APPLICATION

This application claims the benefit of priority to U.S. Provisional Application No. 62/319,723 (filed Apr. 7, 2016), entitled “Formulations of Site-Specific Microparticulate Compositions and Their Use to Improve Outcome after Aneurysmal Subarachnoid Hemorrhage,” the content of which is incorporated by reference herein in its entirety.

### FIELD OF INVENTION

The described invention relates to pharmaceutical formulations and therapeutic methods of use.

### BACKGROUND OF THE INVENTION

### 1. Central Nervous System

The central nervous system is a bilateral and essentially symmetrical structure with seven main parts: the spinal cord, medulla oblongata, pons, cerebellum, midbrain, diencephalon, and the cerebral hemispheres. FIG. 1 shows a lateral view of the human brain from Stedman's Medical Dictionary, 27^{th} Edition, plate 7 at A7 (2000).

The spinal cord, the most caudal part of the central nervous system, receives and processes sensory information from the skin, joints, and muscles of the limbs and trunk and controls movement of the limbs and the trunk. It is subdivided into cervical, thoracic, lumbar and sacral regions. The spinal cord continues rostrally as the brainstem, which consists of the medulla, pons, and midbrain. The brainstem receives sensory information from the skin and muscles of the head and provides the motor control for the muscles of the head. It also conveys information from the spinal cord to the brain and from the brain to the spinal cord, and regulates levels of arousal and awareness through the reticular formation. The brainstem contains several collections of cell bodies, the cranial nerve nuclei. Some of these receive information from the skin and muscles of the head; others control motor output to muscles of the face, neck and eyes. Still others are specialized for information from the special senses: hearing, balance and taste. (Kandel, E. et al., Principles of Neural Science, 4^{th} Ed., p. 8, 2000).

The medulla oblongata, which lies directly rostral to the spinal cord, includes several centers responsible for vital autonomic functions, such as digestion, breathing and the control of heart rate (Kandel, E. et al., Principles of Neural Science, 4th Ed., p. 8, 2000).

The pons, which lies rostral to the medulla, conveys information about movement from the cerebral hemispheres to the cerebellum (Kandel, E. et al., Principles of Neural Science, 4th Ed., p. 8, 2000).

The cerebellum lies behind the pons and is connected to the brain stem by several major fiber tracts called peduncles. The cerebellum modulates the force and range of movement, and is involved in the learning of motor skills. It also contributes to learning and cognition (Kandel, E. et al., Principles of Neural Science, 4th Ed., p. 8, 2000).

The midbrain, which lies rostral to the pons, controls many sensory and motor functions, including eye movements and the coordination of visual and auditory reflexes (Kandel, E. et al., Principles of Neural Science, 4th Ed., p. 8, 2000).

The diencephalon lies rostral to the midbrain and contains two structures. One, the thalamus, processes most of the information reaching the cerebral cortex from the rest of the central nervous system and is involved in other functions including motor control, autonomic function and cognition. The other, the hypothalamus, regulates autonomic, endocrine, and visceral function (Kandel, E. et al., Principles of Neural Science, 4^{th} Ed., p. 8, 2000).

The cerebral hemispheres consist of a heavily wrinkled outer layer, the cerebral cortex, and deep-lying gray-matter structures—the basal ganglia, which participate in regulating motor performance; the hippocampus, which is involved with aspects of learning and memory storage; and the amygdaloid nuclei, which coordinate the autonomic and endocrine responses of emotional states (Kandel, E. et al., Principles of Neural Science, 4^{th} Ed., p. 8, 2000).

The cerebral cortex is divided into four lobes: the frontal lobe, parietal lobe, temporal lobe and occipital lobe. The surfaces of the cerebral hemispheres contain many grooves or furrows, known as fissures and sulci. The portions of brain lying between these grooves are called convolutions or gyri. The lateral cerebral fissure (fissure of Sylvius) separates the temporal from the frontal lobe. The central sulcus (Rolandic sulcus) separates the frontal from the parietal lobe (Kandel, E. et al., Principles of Neural Science, 4^{th} Ed., p. 8, 2000).

1.1. Meninges of the Brain, Spinal Cord and their Spaces

The meninges, three distinct connective tissue membranes that enclose and protect the brain and spinal cord, are named (from outer to inner layer) the dura mater, the arachnoid, and the pia mater. FIG. 2 shows an illustrative sagittal view of the human brain (J. G. Chusid, Correlative Neuroanatomy & Functional Neurology, 18^{th} Ed., p. 46, 1982). The meninges are associated with three spaces or potential spaces: the epidural potential space, subdural potential space and the subarachnoid space. FIG. 3 is a drawing of a cross section of the three meningeal layers that cover the brain and the sub-arachnoid space (SAS) between the outer cellular layer of the arachnoid and pia mater. (Haines, D. E., Anatomical Record 230: 3-21, 1991). FIG. 4 is a schematic drawing depicting the meninges and their spaces surrounding the spinal cord. (Kulkarni, N. V., “Clinical anatomy for students: problem solving approach,” Jaypee Brothers Medical Publishers (P) Ltd., New Delhi, p. 348-349 (2006)).

The epidural space is a physiological space in the spinal cord; it is not normally present in the brain, but it can develop in response to arterial bleeding, resulting in accumulation of blood between the skull and the dura mater (extradural hemorrhage or epidural hematoma). (Schuenke, M. et al., “Thieme Atlas of Anatomy: Head and Neuroanatomy,” Georg Thieme Verlag, Germany, p. 191 (2007); Stedman's Medical Dictionary, Lippincott, Williams & Wilkins, 27^{th} Ed. (2000)). In the spinal cord, the epidural space refers to the space between the dura mater and the lining of the vertebral canal. The spinal epidural space contains loose areolar tissue, internal vertebral venous plexus, roots of spinal nerves, spinal branches of regional arteries, recurrent meningeal branches of spinal nerves and semi fluid fat. Anesthetic agents are commonly administered in the epidural space for pain management associated with surgical procedures to numb the spinal nerves that traverse the space. (Kulkarni, N. V., “Clinical anatomy for students: problem solving approach,” Jaypee Brothers Medical Publishers (P) Ltd., New Delhi, p. 348-349 (2006)).

The subdural space refers to the potential space that extends from the dura mater to the arachnoid. It can develop as a result of extravasation of blood from bridging veins that artificially open the subdural space between the meningeal layer of the dura mater and the upper layer of the arachnoid membrane (subdural hematoma or subdural hemorrhage). (Schuenke, M. et al., “Thieme Atlas of Anatomy: Head and Neuroanatomy,” Georg Thieme Verlag, Germany, p. 191 (2007); Stedman's Medical Dictionary, Lippincott, Williams & Wilkins, 27^{th} Ed. (2000)).

The subarachnoid space (SAS) or subarachnoid cavity refers to the physiologically normal space that lies between the arachnoid and pia mater. It is filled with cerebrospinal fluid (CSF) and is traversed by blood vessels. (See section titled 1.1.3. “Subarachnoid Cavity” and “Subarachnoid Cisternae”). Spontaneous bleeding into the subarachnoid space (subarachnoid hemorrhage) is usually as a result of arterial bleeding from an aneurysm, although it can occur due to trauma as well. (See section 3 below titled “Subarachnoid hemorrhage”). The subarachnoid space in the spinal cord is of uniform size up to the lower end of the spinal cord beyond which it expands. (Kulkarni, N. V., “Clinical anatomy for students: problem solving approach,” Jaypee Brothers Medical Publishers (P) Ltd., New Delhi, p. 348-349 (2006)).

1.1.1. Dura Mater

The dura mater sends inward four processes that divide the cavity of the skull into a series of freely communicating compartments and further provides for the protection of the different parts of the brain. The dura mater is a dense fibrous structure that covers the brain and spinal cord. It has an inner meningeal and an outer periosteal or endosteal layer. The dural layers over the brain generally are fused, except where they separate to provide space for the venous sinuses and where the inner layer forms septa between brain portions. The outer layer attaches firmly to the inner surface of the cranial bones and sends vascular and fibrous extensions into the bone itself. Around the margin of the foramen magnum (the large opening in the base of the skull forming the passage from the cranial cavity to the spinal cavity) it is closely adherent to the bone, and is continuous with the spinal dura mater.

The cranial dura mater consists of fibroblasts, abundant extracellular collagen and a few elastic fibers arranged in flattened laminae which are imperfectly separated by lacunar spaces and blood vessels into two layers: an inner (meningeal) layer and an outer (endosteal) layer, closely connected together, except in certain situations, where they separate to form sinuses for the passages of venous blood or form septae between portions of the brain. The outer surface of the dura mater is rough and fibrillated (composed of fibers), and adheres closely to the inner surfaces of the bones, the adhesions being most marked opposite the cranial sutures (the immovable joints between the bones of the skull or cranium). The endosteal layer is the internal periosteum for the cranial bones, and contains the blood vessels for their supply. The meningeal layer is lined on its inner surface by a layer of unique elongated, flattened fibroblasts that have been called dural border cells. There is no collagen in this layer and the cells are not connected by cell junctions. They are frequently separated by extracellular spaces filled with amorphous nonfilamentous material. The meningeal layer further comprises two lamellas: the compact lamella and the loose lamella; the former generally contains tight fibrous tissue and few blood vessels, but the latter contains some blood vessels.

The processes of the cranial dura mater, which project into the cavity of the skull, are formed by reduplications of the inner (or meningeal) layer of the membrane. These processes include: (1) the falx cerebri, (2) the tentorium cerebelli, (3) the falx cerebelli, and (4) the diaphragma sellae.

The falx cerebri is a strong, arched process with a sickle-like form which descends vertically in the longitudinal fissure between the cerebral hemispheres. It is narrow in front, where it is attached to the ethmoid bone (the bone at the base of the cranium and the root of the nose) at the crista galli (the triangular midline process of the ethmoid bone); and broad behind, where it is connected with the upper surface of the tentorium cerebelli (an arched fold of dura mater that covers the upper surface of the cerebellum). Its upper margin is convex, and attached to the inner surface of the skull in the middle line, as far back as the internal occipital protuberance; it contains the superior sagittal sinus. Its lower margin is free and concave, and contains the inferior sagittal sinus.

The tentorium cerebelli is an arched lamina, elevated in the middle, and inclining downward toward the circumference. It covers the superior surface of the cerebellum, and supports the occipital lobes of the brain. Its anterior border is free and concave, and bounds a large oval opening (the incisura tentorii) for the transmission of the cerebral peduncles (the massive bundle of corticofugal nerve fibers passing longitudinally over the ventral surface of the midbrain on each side of the midline) as well as ascending sensory and autonomic fibers and other fiber tracts. The tentorium cerebelli is attached behind, by its convex border, to the transverse ridges upon the inner surface of the occipital bone, and there encloses the transverse sinuses; and, in front, to the superior angle of the petrous part of the temporal bone on either side, enclosing the superior petrosal sinuses. At the apex of the petrous part of the temporal bone, the free and attached borders meet, and, crossing one another, are continued forward to be fixed to the anterior and posterior clinoid processes respectively. The posterior border of the falx cerebri is attached to the middle line of its upper surface. The straight sinus is placed at the junction of the falx cerebri and the tentorium cerebelli.

The falx cerebelli is a small triangular process of dura mater that separates the two cerebellar hemispheres. Its base is attached, above, to the under and back part of the tentorium; and its posterior margin is attached to the lower division of the vertical crest on the inner surface of the occipital bone. As it descends, it sometimes divides into two smaller folds, which are lost on the sides of the foramen magnum.

The diaphragma sellae is a small circular horizontal fold, which roofs in the sella turcica (a saddlelike prominence on the upper surface of the sphenoid bone of the skull, situated in the middle cranial fossa and dividing it into two halves) and almost completely covers the pituitary gland (hypophysis); a central opening of variable size transmits the infundibulum (a funnel-shaped extension of the hypothalamus connecting the pituitary gland to the base of the brain).

The arteries of the dura mater are numerous. The meningeal branches of the anterior and posterior ethmoidal arteries and of the internal carotid artery, and a branch from the middle meningeal artery supply the dura of the anterior cranial fossa. The middle and accessory meningeal arteries of the internal maxillary artery; a branch from the ascending pharyngeal artery, which enters the skull through the foramen lacerum; branches from the internal carotid artery, and a recurrent branch from the lacrimal artery supply the dura of the middle cranial fossa. Meningeal branches from the occipital artery, one entering the skull through the jugular foramen, and another through the mastoid foramen; the posterior meningeal artery from the vertebral artery; occasional meningeal branches from the ascending pharyngeal artery, entering the skull through the jugular foramen and hypoglossal canal; and a branch from the middle meningeal artery supply the dura of the posterior cranial fossa.

The veins returning the blood from the cranial dura mater anastomose with the diploic veins or end in the various sinuses. Many of the meningeal veins do not open directly into the sinuses, but open indirectly through a series of ampullae, termed venous lacunae. These are found on either side of the superior sagittal sinus, especially near its middle portion, and are often invaginated by arachnoid granulations; they also exist near the transverse and straight sinuses. They communicate with the underlying cerebral veins, and also with the diploic and emissary veins.

The nerves of the cranial dura mater are filaments derived from the trigeminal, glossopharyngeal, vagal, second and third spinal, sphenopalatine, otic, and superior cervical ganglia and supply unmyelinated and myelinated sensory and autonomic fibers.

1.1.2. Arachnoid

The middle meningeal layer, the arachnoid, is a delicate avascular membrane lying between the pia mater and the dura mater. It is separated from the overlying dura mater by a layer of dural border cells that are loosely adherent and are a natural cleavage plane between the dura and arachnoid. This is the layer in which subdural bleeding occurs. It has been termed the subdural space but there is no true subdural space. The arachnoid is separated from the underlying pia mater by the subarachnoid space, which contains cerebrospinal fluid.

The arachnoid consists of an outer cell layer of low cuboidal mesothelium. There is a space of variable thickness filled with cerebrospinal fluid and traversed by trabeculae and membranes consisting of collagen fibrils and cells resembling fibroblasts. The inner layer and the trabeculæ are covered by a somewhat low type of cuboidal mesothelium, which in places are flattened to a pavement type and blends on the inner deep layer with the cells of the pia mater. The arachnoid further contains a plexus of nerves derived from the motor root of the trigeminal, the facial, and the accessory cranial nerves.

The cranial part (arachnoidea encephali) of the arachnoid invests the brain loosely, and does not dip into the sulci (depressions or fissures in the surface of the brain) between the gyri (upraised folds or elevations in the surface of the brain), nor into the fissures, with the exception of the longitudinal fissure and several other larger sulci and fissures. On the upper surface of the brain, the arachnoid is thin and transparent; at the base it is thicker. It is slightly opaque toward the central part of the brain, where it extends across between the two temporal lobes in front of the pons so as to leave a considerable space between the pons and the brain.

The arachnoid surrounds the cranial and spinal nerves, and encloses them in loose sheaths as far as their points of exit from the skull.

1.1.3. Subarachnoid Cavity

The subarachnoid cavity or subarachnoid space, which is the space between the outer cellular layer of the arachnoid and the pia mater, is occupied by tissue consisting of trabeculae of delicate connective tissue and intercommunicating channels in which the cerebrospinal fluid is contained. This cavity is small on the surface of the hemispheres of the brain; on the summit of each gyms, the pia mater and the arachnoid are in close contact, but triangular spaces are left in the sulci between the gyri, in which the subarachnoid trabecular tissue is found, because the pia mater dips into the sulci, whereas the arachnoid bridges across them from gyrus to gyrus. At certain parts of the base of the brain, the arachnoid is separated from the pia mater by wide intervals, which communicate freely with each other and are named subarachnoid cisternae; the subarachnoid tissue in these cisternae is less abundant.

### Subarachnoid Cisternae (Cisternae Subarachnoidales)

The cisterna cerebellomedullaris (cisterna magna) is triangular on sagittal section, and results from the arachnoid bridging over the space between the medulla oblongata and the under surfaces of the hemispheres of the cerebellum; it is continuous with the subarachnoid cavity of the spinal cord at the level of the foramen magnum.

The cisterna pontis is a considerable space on the ventral aspect of the pons. It contains the basilar artery, and is continuous caudal to the pons with the subarachnoid cavity of the spinal cord, and with the cisterna cerebellomedullaris; in front of the pons, it is continuous with the cisterna interpeduncularis.

The cisterna interpeduncularis (cisterna basalis) or the basal cistern is a wide cavity where the arachnoid extends across between the two temporal lobes. It encloses the cerebral peduncles and the structures contained in the interpeduncular fossa, and contains part of the arterial circle of Willis. In front, the cisterna interpeduncularis extends forward across the optic chiasma, forming the cisterna chiasmatis, and further on to the upper surface of the corpus callosum. The arachnoid stretches across from one cerebral hemisphere to the other immediately beneath the free border of the falx cerebri, and thus leaves a space in which the anterior cerebral arteries are contained. The cisterna fossae cerebri lateralis is formed in front of either temporal lobe by the arachnoid bridging across the lateral fissure. This cavity contains the middle cerebral artery. The cisterna venae magnae cerebri occupies the interval between the splenium of the corpus callosum and the superior surface of the cerebellum; it extends between the layers of the tela chorioidea of the third ventricle and contains the great cerebral vein.

The subarachnoid cavity communicates with the general ventricular cavity of the brain by three openings; one, the foramen of Majendie, is in the middle line at the inferior part of the roof of the fourth ventricle; the other two (the foramina of Luschka) are at the extremities of the lateral recesses of that ventricle, behind the upper roots of the glossopharyngeal nerves.

The arachnoid villi are tufted prolongations of pia-arachnoid that protrude through the meningeal layer of the dura mater and have a thin limiting membrane. Tufted prolongations of pia-arachnoid composed of numerous arachnoid villi that penetrate dural venous sinuses and effect transfer of cerebrospinal fluid to the venous system are called arachnoid granulations.

An arachnoidal villus represents an invasion of the dura by the arachnoid membrane, whereby arachnoid mesothelial cells come to lie directly beneath the vascular endothelium of the great dural sinuses. Each villus consists of the following parts: (1) in the interior is a core of subarachnoid tissue, continuous with the meshwork of the general subarachnoid tissue through a narrow pedicle, by which the villus is attached to the arachnoid; (2) around this tissue is a layer of arachnoid membrane, limiting and enclosing the subarachnoid tissue; (3) outside this is the thinned wall of the lacuna, which is separated from the arachnoid by a potential space, which corresponds to and is continuous with the potential subdural space; and (4) if the villus projects into the sagittal sinus, it will be covered by the greatly thinned wall of the sinus, which may consist merely of endothelium. Fluid injected into the subarachnoid cavity will find its way into these villi. Such fluid passes from the villi into the venous sinuses into which they project.

1.1.4. Pia Mater

The pia mater is a thin connective tissue membrane that is applied to the surface of the brain and spinal cord. Blood vessels supplying the brain travel through the pia into the brain. The pia mater, which is continuous with the ependyma at the foramen of Majendie and the two foramina of Luschka, is perforated by all the blood vessels as they enter or leave the nervous system, and therefore is considered to be an incomplete membrane. In perivascular spaces, the pia apparently enters as a mesothelial lining of the outer surface of the space; a variable distance from the exterior, these cells become unrecognizable and are apparently lacking, replaced by neuroglia elements. The inner walls of the perivascular spaces likewise seem to be covered for a certain distance by the mesothelial cells, reflected with the vessels from the arachnoid covering of these vascular channels as they traverse the subarachnoid spaces.

The cranial pia mater (pia mater encephali; pia of the brain) invests the entire surface of the brain, dips between the cerebral gyri and cerebellar laminae, and is invaginated to form the tela chorioidea of the third ventricle, and the choroid plexuses of the lateral and third ventricles. As it passes over the roof of the fourth ventricle, it forms the tela chorioidea and the choroid plexuses of the fourth ventricle. On the cerebellum the membrane is more delicate; the vessels from its deep surface are shorter, and its relations to the cortex are not so intimate.

The pia mater forms sheaths for the cranial nerves.

### 2. Circulation of the Brain

FIGS. 5, 6, 7 and 8 show schematic illustrations of the brain's blood vessels. Each cerebral hemisphere is supplied by an internal carotid artery, which arises from a common carotid artery beneath the angle of the jaw, enters the cranium through the carotid foramen, traverses the cavernous sinus, penetrates the dura (giving off the ophthalmic artery) and divides into the anterior and middle cerebral arteries. The large surface branches of the anterior cerebral artery supply the cortex and white matter of the inferior frontal lobe, the medial surface of the frontal and parietal lobes and the anterior corpus callosum. Smaller penetrating branches supply the deeper cerebrum and diencephalon, including limbic structures, the head of the caudate, and the anterior limb of the internal capsule. The large surface branches of the middle cerebral artery supply most of the cortex and white matter of the hemisphere's convexity, including the frontal, parietal, temporal and occipital lobes, and the insula. Smaller penetrating branches supply the deep white matter and diencephalic structures such as the posterior limb of the internal capsule, the putamen, the outer globus pallidus, and the body of the caudate. After the internal carotid artery emerges from the cavernous sinus, it also gives off the anterior choroidal artery, which supplies the anterior hippocampus and, at a caudal level, the posterior limb of the internal capsule. Each vertebral artery arises from a subclavian artery, enters the cranium through the foramen magnum, and gives off an anterior spinal artery and a posterior inferior cerebellar artery. The vertebral arteries join at the junction of the pons and the medulla to form the basilar artery, which at the level of the pons gives off the anterior inferior cerebellar artery and the internal auditory artery, and, at the midbrain, the superior cerebellar artery. The basilar artery then divides into the two posterior cerebral arteries. The large surface branches of the posterior cerebral arteries supply the inferior temporal and medial occipital lobes and the posterior corpus callosum; the smaller penetrating branches of these arteries supply diencephalic structures, including the thalamus and the subthalamic nuclei, as well as part of the midbrain (see Principles of Neural Sciences, 2d Ed., Eric R. Kandel and James H. Schwartz, Elsevier Science Publishing Co., Inc., New York, pp. 854-56 (1985)).

Interconnections between blood vessels (anastomoses) protect the brain when part of its vascular supply is compromised. At the circle of Willis, the two anterior cerebral arteries are connected by the anterior communicating artery and the posterior cerebral arteries are connected to the internal carotid arteries by the posterior communicating arteries. Other important anastomoses include connections between the ophthalmic artery and branches of the external carotid artery through the orbit, and connections at the brain surface between branches of the middle, anterior, and posterior cerebral arteries (Principles of Neural Sciences, 2d Ed., Eric R. Kandel and James H. Schwartz, Elsevier Science Publishing Co., Inc., New York, pp. 854¬56 (1985)).

The circle of Willis at the base of the brain is the principal arterial anastomotic trunk of the brain. Blood reaches it mainly via the vertebral and internal carotid arteries (See FIG. 5); anastomoses occur between arterial branches of the circle of Willis over the cerebral hemispheres and via extracranial arteries that penetrate the skull through various foramina.

The circle of Willis is formed by anastamoses between the internal carotid, basilar, anterior cerebral, anterior communicating, posterior cerebral, and posterior communicating arteries. The internal carotid artery terminates in the anterior cerebral and middle cerebral arteries. Near its termination, the internal carotid artery gives rise to the posterior communicating artery, which joins caudally with the posterior cerebral artery. The anterior cerebral arteries connect via the anterior communicating artery.

2.1. Cerebral Arteries

The blood supply to the cerebral cortex mainly is via cortical branches of the anterior cerebral, middle cerebral, and posterior cerebral arteries, which reach the cortex in the pia mater. FIG. 6 shows an illustrative view of the arterial supply of the cerebral cortex where 1 is the orbitofrontal artery; 2 is the prerolandic artery; 3 is the rolandic artery; 4 is the anterior parietal artery; 5 is the posterior parietal artery; 6 is the angular artery; 7 is the posterior temporal artery; 8 is the anterior temporal artery; 9 is the orbital artery; 10 is the frontopolar artery; 11 is the callosomarginal artery; 12 is the posterior internal frontal artery; and 13 is the pericallosal artery (Correlative Neuroanatomy & Functional Neurology, 18^{th} Ed., p. 50, 1982).

The lateral surface of each cerebral hemisphere is supplied mainly by the middle cerebral artery. The medial and inferior surfaces of the cerebral hemispheres are supplied by the anterior cerebral and posterior cerebral arteries.

The middle cerebral artery, a terminal branch of the internal carotid artery, enters the lateral cerebral fissure and divides into cortical branches that supply the adjacent frontal, temporal, parietal and occipital lobes. Small penetrating arteries, the lenticulostriate arteries, arise from the basal portion of the middle cerebral artery to supply the internal capsule and adjacent structures.

The anterior cerebral artery extends medially from its origin from the internal carotid artery into the longitudinal cerebral fissure to the genu of the corpus callosum, where it turns posteriorly close to the corpus callosum. It gives branches to the medial frontal and parietal lobes and to the adjacent cortex along the medial surface of these lobes.

The posterior cerebral artery arises from the basilar artery at its rostral end usually at the level of the midbrain, curves dorsally around the cerebral peduncle, and sends branches to the medial and inferior surfaces of the temporal lobe and to the medial occipital lobe. Branches include the calcarine artery and perforating branches to the posterior thalamus and subthalamus.

The basilar artery is formed by the junction of the vertebral arteries. It supplies the upper brain stem via short paramedian, short circumferential, and long circumferential branches.

The midbrain is supplied by the basilar, posterior cerebral, and superior cerebellar arteries. The pons is supplied by the basilar, anterior cerebellar, inferior cerebellar, and superior cerebellar arteries. The medulla oblongata is supplied by the vertebral, anterior spinal, posterior spinal, posterior inferior cerebellar, and basilar arteries. The cerebellum is supplied by the cerebellar arteries (superior cerebellar, anterior inferior cerebellar, and posterior inferior cerebellar arteries).

The choroid plexuses of the third and lateral ventricles are supplied by branches of the internal carotid and posterior cerebral arteries. The choroid plexus of the fourth ventricle is supplied by the posterior inferior cerebellar arteries.

Venous drainage from the brain chiefly is into the dural sinuses, vascular channels lying within the tough structure of the dura. The dural sinuses contain no valves and, for the most part, are triangular in shape. The superior longitudinal sinus is in the falx cerebri.

The human brain constitutes only about 2% of the total weight of the body, but it receives about 15% of cardiac output, and its oxygen consumption is approximately 20% of that for the total body. These values indicate the high metabolic rate and oxygen requirement of the brain that are compensated by a correspondingly high rate of blood flow per unit brain weight. Cerebral circulation is supplied by the internal carotid arteries and the vertebral arteries. The total blood flow to the brain is about 750-1000 mL/min; of this amount about 350 mL flows through each internal carotid artery and about 100-200 mL flows through the vertebral basilar system. The venous outflow is drained by the internal jugular veins and the vertebral veins.

The term “stroke” or “cerebrovascular accident” as used herein refers to the neurological symptoms and signs, usually focal and acute that result from diseases involving blood vessels. Strokes are either occlusive (due to closure of a blood vessel) or hemorrhagic (due to bleeding from a vessel). The term “ischemia” as used herein refers to a lack of blood supply and oxygen that occurs when reduced perfusion pressure distal to an abnormal narrowing (stenosis) of a blood vessel is not compensated by autoregulatory dilation of the resistance vessels. When ischemia is sufficiently severe and prolonged, neurons and other cellular elements die; this condition is referred to as “infarction.”

Hemorrhage may occur at the brain surface (extraparenchymal), for example from the rupture of congenital aneurysms at the circle of Willis, causing subarachnoid hemorrhage (SAH). Hemorrhage also may be intraparenchymal, for example from rupture of vessels damaged by long-standing hypertension, and may cause a blood clot (intracerebral hematoma) within the cerebral hemispheres, in the brain stem, or in the cerebellum. Hemorrhage may be accompanied by ischemia or infarction. The mass effect of an intracerebral hematoma may compromise the blood supply of adjacent brain tissue; or SAH may cause reactive vasospasm of cerebral surface vessels, leading to further ischemic brain damage. Infarcted tissue may also become secondarily hemorrhagic. Aneurysms occasionally can rupture into the brain, causing an intracerebral hematoma, and into the cerebral ventricles, causing intraventricular hemorrhage.

Although most occlusive strokes are due to atherosclerosis and thrombosis, and most hemorrhagic strokes are associated with hypertension or aneurysms, strokes of either type may occur at any age from many causes, including, without limitation, cardiac disease, trauma, infection, neoplasm, blood dyscrasia, vascular malformation, immunological disorder, and exogenous toxins.

2.2. Vasoconstriction and Vasodilation

The term “vasoconstriction” as used herein refers to the narrowing of blood vessels resulting from contracting of the muscular wall of the vessels. When blood vessels constrict, the flow of blood is restricted or slowed. The term “vasodilation”, which is the opposite of vasoconstriction as used herein, refers to the widening of blood vessels. The terms “vasoconstrictors,” “vasopressors,” or “pressors” as used herein refer to factors causing vasoconstriction. Vasoconstriction usually results in an increase of blood pressure and may be slight or severe. Vasoconstriction may result from disease, medication, or psychological conditions. Medications that cause vasoconstriction include, but are not limited to, catecholamines, antihistamines, decongestants, methylphenidate, cough and cold combinations, pseudoephedrine, and caffeine.

A vasodilator is a drug or chemical that relaxes the smooth muscle in blood vessels causing them to dilate. Dilation of arterial blood vessels (mainly arterioles) leads to a decrease in blood pressure. The relaxation of smooth muscle relies on removing the stimulus for contraction, which depends predominately on intracellular calcium ion concentrations and phosphorylation of myosin light chain (MLC). Thus, vasodilation predominantly works either 1) by lowering intracellular calcium concentration, or 2) by dephosphorylation of MLC, which includes the stimulation of myosin light chain phosphatase and the induction of calcium symporters and antiporters (which pump calcium ions out of the intracellular compartment). The re-uptake of ions into the sarcoplasmic reticulum of smooth muscle via exchangers and expulsion of ions across the plasma membrane also helps to accomplish vasodilation. The specific mechanisms to accomplish these effects vary from vasodilator to vasodilator and may be grouped as endogenous and exogenous. The term “endogenous” as used herein refers to proceeding from within or derived internally; or resulting from conditions within the organism rather than externally caused. The term “exogenous” as used herein refers to originating from outside; derived externally; or externally caused rather than resulting from conditions within the organism.

Vasodilation directly affects the relationship between mean arterial pressure and cardiac output and total peripheral resistance (TPR). Cardiac output may be computed by multiplying the heart rate (in beats/minute) and the stroke volume (the volume of blood ejected during systole). TPR depends on several factors, including, but not limited to, the length of the vessel, the viscosity of blood (determined by hematocrit), and the diameter of the blood vessel. Blood vessel diameter is the most important variable in determining resistance. An increase in either cardiac output or TPR cause a rise in the mean arterial pressure. Vasodilators work to decrease TPR and blood pressure through relaxation of smooth muscle cells in the tunica media layer of large arteries and smaller arterioles.

Vasodilation occurs in superficial blood vessels of warm-blooded animals when their ambient environment is hot; this process diverts the flow of heated blood to the skin of the animal, where heat may be more easily released into the atmosphere. Vasoconstriction is the opposite physiological process. Vasodilation and vasoconstriction are modulated naturally by local paracrine agents produced by endothelial cells (e.g., bradykinin, adenosine, nitric oxide, endothelins), as well as by an organism's autonomic nervous system and adrenal glands, both of which secrete catecholamines, such as norepinephrine and epinephrine, respectively.

Vasodilators are used to treat conditions such as hypertension, where the patient has an abnormally high blood pressure, as well as angina and congestive heart failure, where maintaining a lower blood pressure reduces the patient's risk of developing other cardiac problems.

Cerebral Ventricles

Cerebral ventricles, which are chambers in the brain that contain cerebrospinal fluid, include two lateral ventricles, one third ventricle, and one fourth ventricle. The lateral ventricles are in the cerebral hemispheres. They drain via the foramen of Monroe into the third ventricle, which is located between the two diencephalic structures of the brain. The third ventricle leads, by way of the aqueduct of Sylvius, to the fourth ventricle. The fourth ventricle is in the posterior fossa between the brainstem and the cerebellum. The cerebrospinal fluid drains out of the fourth ventricle through the foramenae of Luschka and Magendie to the basal cisterns. The cerebrospinal fluid then percolates through subarachnoid cisterns and drains out via arachnoid villi into the venous system.

FIG. 9 is a diagram of the ventricular system of the brain. The system is a series of cavities (ventricles) within the brain and is continuous with both the subarachnoid space and central canal of the spinal cord. There are four cerebral ventricles: the right and left lateral ventricles, and the midline third and fourth ventricles. The two lateral ventricles are located within the cerebrum and each connects to the third ventricle through an interventricular foramen of Monroe. The third ventricle is located in the diencephalon and is connected to the fourth ventricle by the cerebral aqueduct of Sylvius. The fourth ventricle is located in the hind brain and it is continuous with the central canal of the spinal cord, at least embryologically. Three foramina connect the fourth ventricle to the subarachnoid space: the median aperture or foramen of Magendie, and left and right lateral apertures (foramena) of Luschka.

2.4. CSF Flow in the Brain

FIG. 10 shows an illustrative view of CSF flow from the ventricles to the subarachnoid space. Cerebrospinal fluid (CSF) is a clear bodily fluid that occupies the ventricular system, subarachnoid space of the brain, and central canal of the spinal cord. CSF is produced by modified ependymal cells of the choroid plexus found throughout the ventricular system; it is also formed around blood vessels and ventricular walls, presumably from the extracellular space of the brain. CSF flows from the lateral ventricles via interventricular foramina into the third ventricle. CSF then flows into the fourth ventricle through the cerebral aqueduct. CSF flows out in the subarachnoid space via the median aperture and left and right lateral apertures. Finally, the CSF is reabsorbed into the dural venous sinuses through arachnoid granulations and arachnoid villi. Arachnoid granulations consist of collections of villi. The villi are visible herniations of the arachnoid membrane through the dura and into the lumen of the superior sagittal sinus and other venous structures. The granulations appear to function as valves that allow one-way flow of CSF from the subarachnoid spaces into venous blood. All constituents of CSF leave with the fluid, including small molecules, proteins, microorganisms, and red blood cells.

CSF is produced at a rate of approximately 0.3-0.37 mL/minute or 20 mL/hour or 500 mL/day. The volume of the CSF space is about 150 mL and the CSF turns over 3.7 times a day.

The choroid plexus uses capillary filtration and epithelial secretory mechanisms to maintain the chemical stability of the CSF. While the capillaries that traverse the choroid plexus are freely permeable to plasma solutes, a barrier exists at the level of the epithelial cells that make up the choroid plexus, which is responsible for carrier-mediated active transport. CSF and extracellular fluids of the brain are in a steady state and blood plasma and CSF are in osmotic equilibrium under normal physiological conditions.

2.5. Blood Brain Barrier

The blood brain barrier (BBB) prevents entry of blood-borne substances into the brain and maintains a stable environment for neurons to function effectively. It results from specialized properties of brain microvessel endothelial cells, the principal anatomic site of the BBB, their intercellular junctions, and a relative lack of vesicular transport, which makes such cells different from those of general capillaries. Endothelial cells of blood-brain barrier vessels also are not fenestrated; instead they are interconnected by complex arrays of tight junctions, which block diffusion across the vessel wall.

### 3. Subarachnoid Hemorrhage

The term “subarachnoid hemorrhage” (also referred to as “SAH”) refers to bleeding into the subarachnoid space. SAH may occur spontaneously, usually from a cerebral aneurysm, or may result from trauma. A cerebral aneurysm is a weakness in the wall of an artery of the brain that results in circumscribed dilation of the artery, such that the wall(s) of the blood vessel expand outward. Cerebral aneurysms tend to be located in the circle of Willis and its branches. Where SAH is caused by a rupture of an intracranial aneurysm, i.e., aneurysmal SAH (“aSAH”), bleeding is seen in the subarachnoid space, and less commonly in the intraventricular and intracerebral spaces. Bleeding due to SAH may result in brain damage, brain shift, decreased cerebral perfusion and hydrocephalus. Symptoms include an intense headache with a rapid onset (sometimes referred to as a “thunderclap headache”), vomiting and an altered level of consciousness. Diagnosis generally is made with computed tomography (CT scanning), or occasionally by lumbar puncture. FIG. 11A shows a flow diagram for prognosis following SAH and FIG. 11B shows a flow diagram of pathways proposed to be involved in delayed complications after SAH.

SAH is a medical emergency and may lead to death or severe disability even if recognized and treated at an early stage. About 35% of all SAH cases are fatal, with 10-15% of patients dying before arriving at a hospital. SAH is considered a form of stroke, and causes between 1% and 7% of all strokes. Aneurysmal SAH constitutes on an average about 85% of all cases of spontaneous SAH. While most cases of SAH are due to bleeding from small aneurysms, larger aneurysms (which are rarer) are more likely to rupture. No aneurysm is detected from the first angiogram in 15% of cases of spontaneous SAH. Non-aneurysmal perimesencephalic hemorrhage, in which the blood is limited to the area of the prepontine, interpeduncular and adjacent subarachnoid cisterns, causes 67% of the SAH cases in which no aneurysm is detected. The remaining 33% of cases are due to vasculitic damage to arteries, other disorders affecting the vessels, disorders of the spinal cord blood vessels, bleeding into various tumors, and a number of other causes. Most traumatic SAHs occur near a skull fracture or intracerebral contusion.

In the United States, it is estimated that the incidence of SAH from a ruptured intracranial aneurysm is 1 case per 10,000 persons per year, yielding approximately 35,000 new cases of SAH each year. These ruptured aneurysms have a 30-day mortality rate of about 35%. About 15% of patients die before reaching hospital and an additional 20% or so die within 30 days of the hemorrhage. (Nieuwkamp D J et al., “Changes in case fatality of aneurysmal subarachnoid hemorrhage over time, according to age, sex, and region: a meta-analysis,” Lancet Neurol., 8:635-642 (2009)). An estimated 30% of survivors will have moderate-to-severe disability. The morbidity is substantial in those who survive, with 75% suffering permanent neurological or neurocognitive impairment. (Al-Khindi T. et al., “Cognitive and functional outcome after aneurysmal subarachnoid hemorrhage,” Stroke, 41:e519-e536, (2010)). Thus, only about 20% of all patients survive and resume their previous lifestyle by 3 to 6 months after aneurysmal SAH. The burden of aneurysmal SAH is disproportionately high compared to ischemic stroke because of the high likelihood of permanent disability and the relative youth of those affected (51 years of age for aSAH compared to 75-years old for ischemic stroke). (Taylor, T. N. et al., “Lifetime cost of stroke in the United States,” Stroke, 27:1459-1466 (1996)). FIG. 12 shows time trends in outcome of SAH in seven population-based studies of SAH, which shows 50% decrease in mortality over 20 years.

A systematic review of the incidence of SAH revealed that the overall incidence of SAH is on average 9.1 per 100,000 annually. Studies from Japan and Finland show higher rates in those countries (22.7 per 100,000 and 19.7 per 100,000, respectively), for reasons that are not entirely understood. South and Central America, in contrast, have a rate of 4.2 per 100,000 on average. The group of people at risk for SAH is younger than the population usually affected by stroke, but the risk still increases with age. Young people are much less likely than middle-aged people (risk ratio 0.1, or 10%) to suffer a SAH. The risk continues to rise with age and is 60% higher in the very elderly (over 85) than in those between 45 and 55. Risk of SAH is about 25% higher in women above 55, possibly reflecting the hormonal changes that result from the menopause (de Rooij, N. K. et al., “Incidence of subarachnoid hemorrhage: a systematic review with emphasis on region, age, gender and time trends,” Journal of Neurology, Neurosurgery, and Psychiatry, 2007, 78(12): 1365-1372; Feigin, V. L. et al., “Risk factors for subarachnoid hemorrhage an updated systematic review of epidemiological studies,” Stroke, 2005, 36(12): 2773-2780).

Symptoms of SAH

The classic symptom of SAH is thunderclap headache (a headache described as the “worst ever” or an “explosion in the head,” developing over seconds to minutes) although it is a symptom in only about a third of all SAH patients. Approximately 10% of patients who seek medical care with this symptom have an underlying SAH. Patients also may present with vomiting, and 1 in 14 have seizures. Neck stiffness and other signs of meningism may be present, as may confusion, decreased level of consciousness or coma. Intraocular hemorrhage may occur in response to the raised pressure inside the head (intracranial pressure). Subhyaloid (the hyaloid membrane envelopes the vitreous body of the eye) and vitreous hemorrhage may be visible on fundoscopy. This is known as Terson syndrome (occurring in 3-13% of cases), and is more common in more severe SAH. In a patient with thunderclap headache, none of the aforementioned signs are helpful in confirming or ruling out hemorrhage, although seizures are more common if the bleeding is the result of a ruptured aneurysm as opposed to other causes. Oculomotor nerve abnormalities (affected eye movement downward and outward, inability to lift the eyelid on the same side but normal pupillary reflexes) may indicate bleeding from an aneurysm arising near the posterior communicating artery. Isolated dilation of a pupil may also reflect brain herniation as a result of increased intracranial pressure.

The body releases large amounts of adrenaline and similar hormones as a result of the bleeding, which leads to a sudden increase in the blood pressure. The heart comes under substantial strain, and neurogenic pulmonary edema, stunned myocardium, cardiac arrhythmias, electrocardiographic changes (with occasional giant inverted “cerebral” T waves), tsako tsubo cardiomyopathy and cardiac arrest (3%) may rapidly occur after the onset of hemorrhage.

SAH also may occur in people who have suffered a head injury. Symptoms may include headache, decreased level of consciousness or hemiparesis. SAH is regarded as a severe complication of head injury, especially if it is associated with lower Glasgow Coma Scale levels.

Diagnosis of SAH

The initial steps for evaluating a person with a suspected SAH are the steps of obtaining a medical history and performing a physical examination. Since only 10-25% of patients admitted to a hospital with a thunderclap headache are suffering from a SAH, other possible causes usually are considered simultaneously, such as meningitis, migraine and cerebral venous sinus thrombosis. Intracerebral hemorrhage, which is twice as common as SAH, occasionally is misdiagnosed as SAH.

A diagnosis of SAH cannot be made on clinical grounds alone. Generally, medical imaging [usually computed tomography (CT) scan, which has a high sensitivity (>95% correct identification especially on the first day after the onset of bleeding)] of the brain is required to confirm or exclude bleeding. Magnetic resonance imaging (MRI) may be more sensitive after several days when compared to CT scan. In people with normal CT or MRI scans, lumbar puncture, in which CSF is removed with a needle from the lumbar sac, shows evidence of hemorrhage in 3% of the group in whom the CT was found to be normal; lumbar puncture is therefore regarded as mandatory if imaging is negative. The CSF sample is examined for xanthochromia, the yellow appearance of centrifuged fluid, or by using spectrophotometry for bilirubin, a breakdown product of hemoglobin in the CSF.

After an SAH is confirmed, its origin needs to be determined. CT angiography (“CTA”) (visualizing blood vessels with radiocontrast on a CT scan) to identify aneurysms is generally the first step, although the more invasive catheter angiography (injecting radiopaque contrast through a catheter advanced to the brain arteries) is the gold standard test but has a higher risk of complications. The latter is useful if there are plans to obliterate the source of bleeding, such as an aneurysm, at the same time.

Classification of SAH

Several grading scales available for SAH have been derived by retrospectively matching characteristics of patients with their outcomes.

The Glasgow Coma Scale (GCS) has been used ubiquitously in the clinical assessment of post-traumatic unconsciousness; it assesses 15 points covering three components: eye (E), verbal (V) and motor (M) response to external stimuli. (Teasdale G. et al., “Assessment of coma and impaired consciousness,” Lancet, 2(7872): 81084 (1974); Teasdale, G. et al., “Assessment and prognosis of coma after head injury,” Acta Neurochir., 34: 45-55 (1976)). Table 1 shows the categorization of the Glasgow Coma Scale.

TABLE 1
Categorization of Glasgow Coma Scale

| COMPONENTS | POINTS OF ASSESSMENT |
|---|---|
| E—Eye Opening | C. Not assessable |
| | 4. Spontaneous |
| | 3. To speech |
| | 2. To pain |
| | 1. None |
| V—Verbal | T. Not assessable |
| Response | 5. Oriented conversation |
| | 4. Confused speech |
| | 3. Inappropriate words |
| | 2. Incomprehensible sounds |
| | 1. None |
| M—Motor | 6. Obeys simple commands |
| Response | 5. Localizes pain |
| | 4. Withdraws (normal flexion) |
| | 3. Stereotyped flexion |
| | 2. Stereotyped extension |
| | 1. None |

The Glasgow Outcome Scale (GOS) and its extended form (eGOS or GOSE) are global scales measuring functional outcome of patient status. The five categories of the Glasgow outcome scale were extended to eight categories in the extended Glasgow Outcome Scale. (Jennett, B. and Bond, M., “Assessment of outcome after severe brain damage,” Lancet, 1: 480-484 (1975); Teasdale, G. M. et al., “Analyzing outcome of treatment of severe head injury: A review and update on advancing the use of the Glasgow Outcome Scale,” Journal of Neurotrauma, 15: 587-597 (1998); Wilson, J. T. L. et al., “Structured interviews for the Glasgow Outcome Scale and the Extended Glasgow Outcome Scale,” Journal of Neurotrauma, 15(8): 573-585 (1997); Wilson, J. T. et al., “Observer variation in the assessment of outcome in traumatic brain injury: experience from a multicenter, international randomized clinical trial,” Neurosurgery, 61(1): 123-128 (2007)). Tables 2 and 3 show the categorization scheme used in the Glasgow Outcome Scale (GOS) and in the extended Glasgow Outcome Scale (eGOS or GOSE), respectively.

TABLE 2
Categorization of the Glasgow Outcome Scale

| SCORE | CATEGORY | SYMBOL |
|---|---|---|
| 1 | DEAD | D |
| 2 | VEGETATIVE STATE | VS |
| | Unable to interact with environment; | |
| | unresponsive | |
| 3 | SEVERE DISABILTY | SD− |
| | Able to follow commands/unable to live | |
| | independently | |
| 4 | MODERATE DISABILITY | MD |
| | Able to live independently; unable to | |
| | return to work or school | |
| 5 | GOOD RECOVERY | GR |
| | Able to return to school | |

TABLE 3
Categorization of the Extended Glasgow Outcome Scale

| SCORE | CATEGORY | SYMBOL |
|---|---|---|
| 1 | Death | D |
| 2 | Vegetative State | VS |
| 3 | Lower severe disability | SD− |
| 4 | Upper severe disability | SD+ |
| 5 | Lower moderate disability | MD− |
| 6 | Upper moderate disability | MD+ |
| 7 | Lower good recovery | GR− |
| 8 | Upper good recovery | GR+ |

A scale of severity was described by Hunt and Hess in 1968 (“Hunt and Hess scale”) and categorizes the clinical condition of the patient (Hunt W E, Hess R M: Surgical risk as related to time of intervention in the repair of intracranial aneurysms. Journal of Neurosurgery 28:14-20, 1968). The Fisher Grade classifies the appearance of SAH on CT scan (Fisher C M, Kistler J P, Davis J M: Relation of cerebral vasospasm to subarachnoid hemorrhage visualized by computerized tomographic scanning. Neurosurgery 6:1-9, 1980). The Fisher scale has been modified by Claassen and coworkers (“Claassen scale”), reflecting the additive risk from SAH size and accompanying intraventricular hemorrhage and then again by Frontera, et al. (modified Fisher scale, Claassen J, Bernardini G L, Kreiter K, Bates J, Du Y E, Copeland D, et al: Effect of cisternal and ventricular blood on risk of delayed cerebral ischemia after subarachnoid hemorrhage: the Fisher scale revisited. Stroke 32:2012-2020, 2001; Frontera J A, Claassen J, Schmidt J M, Wartenberg K E, Temes R, Connolly E S, Jr., et al: Prediction of symptomatic vasospasm after subarachnoid hemorrhage: the modified Fisher scale. Neurosurgery 59:21-27, 2006). The World Federation of Neurological Surgeons classification uses GCS and focal neurological deficit to gauge severity of symptoms (Drake C G, Hunt W E, Sano K, Kassell N, Teasdale G, Pertuiset B, et al: Report of World Federation of Neurological Surgeons committee on a universal subarachnoid hemorrhage grading scale. jns 68:985-986, 1988). A comprehensive classification scheme has been suggested by Ogilvy and Carter to predict outcome and gauge therapy. The Ogilvy system has five grades, assigning one point for the presence or absence of each of five factors: (1) age greater than 50; (2) Hunt and Hess grade 4 or 5; (3) Fisher scale 3 or 4; (4) aneurysm size greater than 10 mm; and (5) posterior circulation aneurysm 25 mm or more.

The Barthel index, frequently used in stroke evaluation, is an objective functional scale that measures a patient's independence in activities of daily living (ADL), including feeding, bathing, grooming, dressing, bowel and bladder control, wheelchair management and ascending and descending stairs. (Granger C. V. et al., “Measurement of outcome of care for stroke patients,” Stroke, 6:34-41 (1975)). The Montreal Cognitive Assessment (MoCA) test is a screening tool for mild cognitive dysfunction. (Nasreddine Z. S. et al., “The Montreal Cognitive Assessment (MoCA): A brief screening tool for mild cognitive impairment,” J. Am. Geriatr. Soc., 53: 695-699 (2005)). The modified Rankin scale is a 7-point scale (0 is the best and 6 is the worst score) that assesses patient condition based on their or their care-givers' response to simple questions about their daily functioning (van Swieten, J. C. et al., “Interobserver agreement for the assessment of handicap in stroke patients,” Stroke 19:604-607 (1988)). The National Institutes of Health Stroke Scale (NIHSS) is a 15-item neurological examination stroke scale that is used to evaluate the severity of neurological deficit after a stroke, such as an ischemic stroke or delayed cerebral ischemia (DCI) (Lyden P, Brott T, Tilley B, Welch K M, Mascha E J, Levine S, et al: Improved reliability of the NIH Stroke Scale using video training. NINDS TPA Stroke Study Group. Stroke 25:2220-2226, 1994). It assesses level of consciousness, language, neglect, visual field loss, extraocular movement, motor strength, ataxia, dysarthria and sensory loss.

Prognosis of SAH

Early Morbidity and Mortality

The mortality rate for SAH is between 30% and 40%. Of those who survive initial hospitalization, treatment and complications, at least 25% have significant restrictions in their lifestyle, and less than 20% have no residual symptoms whatsoever. Delay in diagnosis of minor SAH without coma (or mistaking the sudden headache for migraine or some other less serious illness) contributes to poor outcome. Risk factors for poor outcome include higher age, poorer neurological grade, more blood and larger aneurysm on the initial CT scan, location of an aneurysm in the posterior circulation, systolic hypertension and a previous diagnosis of heart attack, hypertension, liver disease or a previous SAH. During the hospital stay, occurrence of delayed ischemia resulting from angiographic vasospasm, cortical spreading ischemia and microthrombosis, development of intracerebral hematoma or intraventricular hemorrhage (bleeding into the ventricles of the brain) and presence of fever on the eighth day of admission also worsen the prognosis.

Angiographic vasospasm was suggested to cause death after aneurysmal SAH in up to 35% of patients in the 1970s and in less than 10% currently. However, outcome overall is still poor, and current rescue therapies, such as hemodynamic therapy and endovascular balloon or pharmacological angioplasty, are associated with substantial morbidity and are expensive and labor intensive (Durrant J C, Hinson H E: Rescue therapy for refractory vasospasm after subarachnoid hemorrhage. Curr Neurol Neurosci Rep 15:521, 2015; Abruzzo T, Moran C, Blackham K A, Eskey C J, Lev R, Meyers P, et al: Invasive interventional management of post-hemorrhagic cerebral vasospasm in patients with aneurysmal subarachnoid hemorrhage. J. Neurointerv. Surg 4:169-177, 201).

Among patients with aneurysmal SAH, the incremental cost for symptomatic vasospasm, which is roughly the same as DCI, was $39,971 in the United States in 2006. (Chou C H et al., “Costs of vasospasm in patients with aneurysmal subarachnoid hemorrhage,” Neurosurgery, 67:345-352 (2010)).

SAH that does not show an aneurysm by complete catheter angiography may be referred to as “angiogram-negative SAH.” This carries a better prognosis than SAH from an aneurysm; however, it still is associated with a risk of ischemia, rebleeding and hydrocephalus. Perimesencephalic SAH (bleeding around the mesencephalon part of the brain) is a subgroup of angiogram-negative SAH. It has a very low rate of rebleeding or delayed ischemia and the prognosis of this subtype is better.

Long-Term Outcomes

Symptoms, such as fatigue, mood disturbances, depression, executive dysfunction and related neurocognitive symptoms, are common in people who have suffered SAH. Even in those who have made a good neurological recovery, anxiety, depression, posttraumatic stress disorder and cognitive impairment are common. Over 60% report frequent headaches. Aneurysmal SAH may lead to damage of the hypothalamus and the pituitary gland, two areas of the brain that play a central role in hormonal regulation and production. Studies indicate that at least 25% of people with a previous SAH may develop deficiencies in one or more of the hypothalamic-pituitary hormones, such as growth hormone, prolactin or thyroid-stimulating hormone.

### 4. Secondary Complications of SAH

Patients who survive SAH also are at risk of secondary complications. Among these complications are, most notably, aneurysmal re-bleeding, angiographic cerebral vasospasm and delayed cerebral ischemia (DCI). (Macdonald R L et al., “Preventing vasospasm improves outcome after aneurysmal subarachnoid hemorrhage: rationale and design of CONSCIOUS-2 and CONSCIOUS-3 trials,” Neurocrit. Care, 13:416-424 (2010); Macdonald R L et al., “Factors associated with the development of vasospasm after planned surgical treatment of aneurysmal subarachnoid hemorrhage,” J. Neurosurg. 99:644-652 (2003); Macdonald R L: Delayed neurological deterioration after subarachnoid haemorrhage. Nat. Rev. Neurol 10:44-58, 2014).

4.1. Delayed Cerebral Ischemia (DCI)

Delayed cerebral ischemia occurs in 30% of patients with aSAH and causes death or permanent disability in half of these patients. (Dorsch N W C, and King M T, “A review of cerebral vasospasm in aneurysmal subarachnoid hemorrhage. Part 1: Incidence and effects,” Journal of Clinical Neuroscience, 1:19-26 (1994)). The risk of DCI is not easily predicted; the most important factor is the volume of SAH seen on admission cranial computed tomography (C T). (Harrod C G et al., “Prediction of cerebral vasospasm in patients presenting with aneurysmal subarachnoid hemorrhage: a review,” Neurosurgery, 56:633-654 (2005); Reilly C et al., “Clot volume and clearance rate as independent predictors of vasospasm after aneurysmal subarachnoid hemorrhage,” J. Neurosurg. 101:255-261 (2004)).

DCI is delayed neurological deterioration due to ischemia, associated with the occurrence of focal neurological impairment (such as hemiparesis, aphasia, apraxia, hemianopia, or neglect), and/or a decrease in the Glasgow coma scale (either the total score or one of its individual components [eye, motor on either side, verbal]). (Frontera J A et al., “Defining vasospasm after subarachnoid hemorrhage: what is the most clinically relevant definition?” Stroke, 40:1963-1968 (2009); Kassell N F et al., “The International Cooperative Study on the Timing of Aneurysm Surgery. Part 1: Overall management results,” J. Neurosurg., 73:18-36 (1990); Vergouwen M D et al., “Effect of statin treatment on vasospasm, delayed cerebral ischemia, and functional outcome in patients with aneurysmal subarachnoid hemorrhage: a systematic review and meta-analysis update,” Stroke, 41:e47-e52 (2010)). This may or may not last for at least one hour, is not apparent immediately after aneurysm occlusion and cannot be attributed to other causes by means of clinical assessment, CT or MRI scanning of the brain and appropriate laboratory studies. DCI and development of delayed cerebral infarction are among the most important causes of poor outcome after SAH.

Cerebral infarction may be a consequence of DCI; infarction due to DCI is defined as the presence of an area of brain cell death resulting from insufficiency of arterial or venous blood supply to the brain. It is detected by CT or MRI scan of the brain within 6 weeks after SAH, or on the latest CT or MRI scan made before death within 6 weeks, or proven at autopsy, not present on the CT or MRI scan between 24 and 48 hours after early aneurysm occlusion, and not attributable to other causes such as surgical clipping or endovascular treatment. Hypodensities on CT imaging resulting from ventricular catheter or intraparenchymal hematoma generally are not regarded as evidence of cerebral infarction from DCI.

Angiographic vasospasm is one process that contributes to DCI. Other processes that may contribute to DCI are cortical spreading ischemia and formation of microthromboemboli. Cortical spreading ischemia, which was described in animal models of SAH as a novel mechanism that may cause DCI, has been detected in humans with SAH and angiographic vasospasm.

4.2. Vasospasm

DCI is usually associated with angiographic cerebral vasospasm. The term “angiographic cerebral vasospasm” refers to the narrowing of the large capacitance arteries at the base of the brain (i.e., cerebral arteries) following hemorrhage into the subarachnoid space, leads to reduced perfusion of distal brain regions, and can be detected by either CT angiography [CTA], MR angiography [MRA] or catheter angiography [CA]). It is the most common cause of focal ischemia after SAH; it adversely affects outcome in patients with SAH as it accounts for up to 23% of SAH-related disability and death. Of all types of ischemic stroke, angiographic vasospasm is unique in that it is, to some degree, preventable and treatable (see Macdonald, R. L. and Weir. B. In Cerebral Vasospasm. Academic Press, Burlington, Mass., USA (2001)).

Generally, angiographic vasospasm of the cerebral arteries begins 3 days after SAH, is maximal 7 to 8 days later and resolves by 14 days. (Weir B. et al., “Time course of vasospasm in man,” J. Neurosurg., 48:173-178 (1978)). About 67% of patients with SAH develop vasospasm, 33% develop DCI and 15% of SAH patients die or sustain permanent disability from DCI.

While angiographic vasospasm is a consequence of SAH, it also can occur after any condition that deposits blood in the subarachnoid space. Vasospasm results in decreased cerebral blood flow and increased cerebral vascular resistance. Without being limited by theory, it generally is believed that vasospasm is caused by local injury to vessels, such as that which results from traumatic head injury, aneurysmal subarachnoid hemorrhage and other causes of SAH. Cerebral vasospasm is a naturally occurring vasoconstriction that also may be triggered by the presence of blood in the CSF, a common occurrence after rupture of an aneurysm or following traumatic head injury. Cerebral vasospasm ultimately can lead to brain cell damage, in the form of cerebral ischemia and infarction, due to interrupted blood supply. Potential manifestation of symptoms from vasospasm occurs only in those patients who survive past the first few days.

The incidence of vasospasm is less than the incidence of SAH (since only some patients with SAH develop vasospasm). The incidence of vasospasm will depend on the type of patient a given hospital receives and the methods by which vasospasm is diagnosed.

The unqualified term “vasospasm” is usually used with reference to angiographically determined arterial narrowing as defined above. “Clinical vasospasm” most often is used synonymously with delayed cerebral ischemia (DCI). When used in another fashion, for instance, vasospasm based on increased middle cerebral artery transcranial Doppler velocities, this should be specified (Vergouwen, M. D. et al., “Definition of delayed cerebral ischemia after aneurysmal subarachnoid hemorrhage as an outcome event in clinical trials and observational studies: proposal of a multidisciplinary research group,” Stroke 41:2391-2395 (2010)).

Some degree of angiographic narrowing will occur in at least two-thirds of patients having angiography between 4 and 12 days after SAH. The numbers of patients developing neurological deterioration from angiographic vasospasm varies with the diligence with which the patient is monitored and the efficacy of prophylaxis, but it has been estimated at about one-third. Of hospitalized SAH patients, about 5% die from vasospasm. When compared to post-SAH patients of intermediate grade, post-SAH patients in very good condition are less likely to develop vasospasm as they have small volume SAH, while post-SAH patients in very poor condition are more likely to die earlier from the initial episode. The presence of thick, widespread subarachnoid clot which can be visualized on the computerized tomographic (CT) scan done in close proximity to the bleeding episode is a key prognostic factor. The chance of vasospasm and consequently DCI is decreased by factors decreasing the duration of exposure to clot. Conversely, the incidence of vasospasm and DCI is increased by the use of antifibrinolytic drugs which prolong the exposure of arteries to clot and possibly cause ischemia by other mechanisms. Poor admission clinical grade is associated with DCI, presumably because they both indicate larger volumes of SAH. A definite relationship between age, hypertension or sex and DCI has not been established. It is possible that smokers are more prone to vasospasm and DCI. Factors unrelated to the development of vasospasm include season, geography, contrast material and diabetes.

Patients who develop vasospasm do worse than those who do not. If neurosurgical clipping or endovascular coiling of the ruptured aneurysm is performed earlier (within the first day or so) the outcome tends to be better than if treatment is delayed. When operations were preferentially performed during the peak period for vasospasm, outcomes were generally worse. Vasospasm does not result from early surgery or coiling; early surgery or coiling permits more vigorous treatment should vasospasm develop. If a thick clot is present, an attempt at careful removal of the clot is sometimes made. The amount of residual clot postoperatively is a prognostic factor for DCI. Open operation exposes the patient to retractor pressure, venous sacrifice, temporary clipping ischemia and arterial injury. Studies have shown post operative decrease in cerebral blood flow, regional cerebral metabolic rate of oxygen and oxygen extraction ratio. Vasospasm and DCI may be more common in patients who undergo neurosurgical clipping of a ruptured aneurysm as compared to endovascular coiling.

Independent variables, such as admission neurologic grade, increasing age, and massive intracranial or intraventricular hemorrhage, are more closely linked to outcome than angiographic vasospasm. Infarction from delayed ischemia is strongly linked to poor outcome. Since vasospasm is a graded process, it is expected that only the extreme cases will result in infarction in the absence of systemic hypotension, cardiac dysfunction, anoxia and intracranial hypertension. Preexisting hypertension and advanced age also strongly influence the vulnerability of the brain to ischemia. The etiological relationship between vasospasm and infarction in fatal cases is not in dispute.

There is evidence that vasospasm may be reduced by clot removal either surgically or pharmacologically. There also are data suggesting that DCI may be lessened by pharmacologically induced hypertension and hypervolemia as well as by calcium antagonists. Vasospasm also may be abolished by mechanical or transiently by pharmacologic angioplasty.

Incidence of Vasospasm

The incidence of angiographic vasospasm depends on the time interval after the SAH. The peak incidence occurs 6-8 days after SAH (range, 3-14 days). In addition to the time after the SAH, other principal factors that affect the prevalence of vasospasm are the volume, density, temporal persistence and distribution of subarachnoid blood.

Prognostic Factors for Vasospasm

Prognostic factors for angiographic vasospasm include: the amount of subarachnoid blood on CT scan; hypertension; anatomical and systemic factors; clinical grade; and whether the patient is receiving antifibrinolytics.

Diagnosis of Vasospasm

The diagnosis of angiographic vasospasm rests on comparison of blood vessel imaging studies. The diagnosis of delayed cerebral ischemia (DCI) is primarily clinical. Angiographic vasospasm can be asymptomatic; however, when the cerebral blood flow is below ischemic threshold, symptoms become apparent, and this is called DCI. Symptoms typically develop subacutely and may fluctuate. Symptoms may include excess sleepiness, lethargy, stupor, hemiparesis or hemiplegia, abulia, language disturbances, visual field deficits, gaze impairment and cranial nerve palsies. Although some symptoms are localized, they are not diagnostic of any specific pathological process; therefore alternative diagnoses, such as rebleeding, hydrocephalus and seizures should be excluded promptly using radiographic, clinical and laboratory assessments. Cerebral angiography is the gold standard for visualizing and studying cerebral arteries; CT angiography and CT perfusion are increasingly widely used. Transcranial Doppler ultrasonography is also utilized.

The pathophysiology of angiographic vasospasm may involve structural changes and biochemical alterations within the vascular endothelium and smooth muscle cells. The presence of blood in the subarachnoid space initiates these changes. In addition, hypovolemia and an impaired cerebral autoregulatory function may concurrently interfere with cerebral perfusion and contribute to DCI due to angiographic vasospasm. The cumulative effects of these processes can lead to reduction in cerebral blood flow so severe as to cause cerebral ischemia leading to infarction. Additionally, a period of severe constriction could lead to morphologic changes in the walls of the cerebral arteries, which may cause them to remain narrowed without the continued presence of vasoactive substances. The area of the brain supplied by the affected artery then would experience ischemia (meaning a restriction in blood supply).

Other Complications

Hydrocephalus (a condition marked by an excessive accumulation of CSF resulting in dilation of the cerebral ventricles and raised intracranial pressure) may complicate SAH in both the short- and long-term, and may be detected on CT scanning. If the level of consciousness is decreased, surgical drainage of the excess fluid (for instance with a ventricular drain or shunt) is occasionally necessary.

Fluctuations in blood pressure and electrolyte disturbances, as well as pneumonia and cardiac decompensation, occur in about 50% of hospitalized patients with SAH, and may worsen prognosis. They are managed symptomatically.

Seizures occur in about a tenth of all cases of SAH.

### 5. Voltage-Gated Ion Channels

Voltage-gated ion channels are a class of integral membrane proteins that allow the passage of selected inorganic ions across the cell membrane by opening and closing in response to changes in transmembrane voltage. (Sands, Z. et al., “Voltage-gated ion channels,” Current Biology, 15(2): R44-R47 (2005)). These types of ion channels are especially critical in neurons, but are common in many types of cells. They have an important role in excitable neuronal and muscle tissues as they allow a rapid and coordinated depolarization in response to triggering voltage change. Positioned along the axon and at the synapse, voltage-gated ion channels directionally propagate electrical signals.

Structure

Voltage-gated potassium, sodium and calcium ion channels are thought to have similar overall architectures. (Sands, Z. et al., “Voltage-gated ion channels,” Current Biology, 15(2): R44-R47 (2005)). Voltage-gated ion channels generally are composed of several subunits arranged such that there is a central pore through which ions can travel down their electrochemical gradients. The channels tend to be quite ion-specific, although similarly sized and charged ions may also travel through them to some extent.

Mechanism

Crystallographic structural studies of a potassium channel, assuming that this structure remains intact in the corresponding plasma membrane, suggest that when a potential difference is introduced over the membrane, the associated electromagnetic field induces a conformational change in the potassium channel. The conformational change distorts the shape of the channel proteins sufficiently such that the channel, or cavity, opens to admit ion influx or efflux to occur across the membrane, down its electrochemical gradient. This subsequently generates an electrical current sufficient to depolarize the cell membrane.

Voltage-gated sodium channels and calcium channels are made up of a single polypeptide with four homologous domains. Each domain contains 6 membrane spanning alpha helices. The voltage sensing helix, S4, has multiple positive charges such that a high positive charge outside the cell repels the helix and induces a conformational change such that ions may flow through the channel. Potassium channels function in a similar way, with the exception that they are composed of four separate polypeptide chains, each comprising one domain. The voltage-sensitive protein domain of these channels (the “voltage sensor”) generally contains a region composed of S3b and S4 helices, known as the “paddle” due to its shape, which appears to be a conserved sequence.

5.1. Voltage-Dependent Calcium Channels

Voltage-dependent calcium channels (VDCC) are a group of voltage-gated ion channels that control calcium entry into cells in response to membrane potential changes. (Van Petegem F. et al., Biochemical Society Transactions, 34(5): 887-893 (2006)). Voltage-dependent calcium channels are found in excitable cells (e.g., muscle, glial cells, neurons, etc.). At physiologic or resting membrane potential, VDCCs are normally closed. They are activated (i.e., opened) at depolarized membrane potentials. Activation of particular VDCCs allows Ca²⁺ entry into the cell; muscular contraction, excitation of neurons, upregulation of gene expression, or release of hormones or neurotransmitters results, depending upon the cell type. (Catterall W. A. et al., “International Union of Pharmacology. XLVIII. Nomenclature and structure-function relationships of voltage-gated calcium channels,” Pharmacol. Rev., 57(4): 411-25 (2005); Yamakage M. et al, “Calcium channels—basic aspects of their structure, function and gene encoding; anesthetic action on the channels—a review,” Can. J. Anaesth., 49(2): 151-64 (2002)).

Voltage-dependent calcium channels are formed as a complex of several different subunits: α₁, α₂δ, β₁₋₄, and γ. The α subunit forms the ion conducting pore while the associated subunits have several functions including modulation of gating. (Dolphin A. C. “A short history of voltage-gated calcium channels,” Br. J. Pharmacol., 147 (Suppl 1): S56-62 (2006))

α1 Subunit

The α₁ subunit pore (about 190 kDa in molecular mass) is the primary subunit necessary for channel functioning in the VDCC, and consists of the characteristic four homologous I-IV domains containing six transmembrane α-helices each. The α subunit forms the Ca²⁺ selective pore, which contains voltage-sensing machinery and the drug/toxin-binding sites. Ten a subunits that have been identified in humans. (Dolphin A. C. “A short history of voltage-gated calcium channels,” Br. J. Pharmacol., 147 (Suppl 1): S56-62 (2006)).

α2δ Subunit

The α₂δ gene encodes two subunits, α₂ and δ. They are linked to each other via a disulfide bond and have a combined molecular weight of 170 kDa. The α₂ is the extracellular glycosylated subunit that interacts the most with the al subunit. The δ subunit has a single transmembrane region with a short intracellular portion, which serves to anchor the protein in the plasma membrane. There are 4 α₂δ genes: CACNA2D1 (CACNA2D1), (CACNA2D2), (CACNA2D3), and (CACNA2D4). Co-expression of the α₂δ enhances the level of expression of the al subunit and causes an increase in current amplitude, faster activation and inactivation kinetics and a hyperpolarizing shift in the voltage dependence of inactivation. Some of these effects are observed in the absence of the beta subunit, whereas, in other cases, the co-expression of beta is required. The α₂δ-1 and α₂δ-2 subunits are binding sites for at least two anticonvulsant drugs, gabapentin and pregabalin, that also find use in treating chronic neuropathic pain. (Dolphin A. C. “A short history of voltage-gated calcium channels,” Br. J. Pharmacol., 147 (Suppl 1): S56-62 (2006))

β Subunit

The intracellular β subunit (55 kDa) is an intracellular membrane-associated guanylate kinase (MAGUK)-like protein containing a guanylate kinase (GK) domain and an SH3 (src homology 3) domain. The guanylate kinase domain of the β subunit binds to the alpha subunit I-II cytoplasmic loop and regulates HVGCC activity. There are four known isoforms of the β subunit: CACNB1, CACNB2, CACNB3, and CACNB4. (Dolphin A. C. “A short history of voltage-gated calcium channels,” Br. J. Pharmacol., 147 (Suppl 1): S56-62 (2006))

Without being limited by theory, it is postulated the cytosolic β subunit has a major role in stabilizing the final a subunit conformation and delivering it to the cell membrane by its ability to mask an endoplasmic reticulum retention signal in the a subunit. The endoplasmic retention brake is contained in the I-II loop of the a subunit that becomes masked when the β subunit binds. Therefore the β subunit functions initially to regulate the current density by controlling the amount of a subunit expressed at the cell membrane.

In addition to this potential trafficking role, the β subunit has the added important functions of regulating activation and inactivation kinetics, and hyperpolarizing the voltage-dependence for activation of the a subunit pore, so that more current passes for smaller depolarizations. The β subunit acts as an important modulator of channel electrophysiological properties. The interaction between a highly conserved 18-amino acid region on the al subunit intracellular linker between domains I and II (the Alpha Interaction Domain, AIDBP) and a region on the GK domain of the β subunit (Alpha Interaction Domain Binding Pocket) is responsible for the regulatory effects exerted by the β subunit. Additionally, the SH3 domain of the β subunit also gives added regulatory effects on channel function, indicating that the β subunit may have multiple regulatory interactions with the al subunit pore. The α interaction domain sequence does not appear to contain an endoplasmic reticulum retention signal; this may be located in other regions of the I-II α1 subunit linker.

γ Subunit

The γ1 subunit is known to be associated with skeletal muscle VDCC complexes, but the evidence is inconclusive regarding other subtypes of calcium channel. The γ1 subunit glycoprotein (33 kDa) is composed of four transmembrane spanning helices. The γ1 subunit does not affect trafficking, and, for the most part, is not required to regulate the channel complex. However, γ2, γ3, γ4 and γ8 also are associated with a-amino-3-hydroxy-S-methyl-4-isoxazolepropionic acid (AMPA) glutamate receptors, non-NMDA-type ionotropic transmembrane receptors for glutamate that mediate fast synaptic transmissions in the central nervous system (CNS). An NMDA-type receptor is a receptor to which NMDA (N-methyl-D-aspartate) binds specifically. There are 8 genes for gamma subunits: γ1 (CACNG1), γ2 (CACNG2), γ3 (CACNG3), γ4 (CACNG4), (CACNG5), (CACNG6), (CACNG7), and (CACNG8). (Chu P. J. et al., “Calcium channel gamma subunits provide insights into the evolution of this gene family,” Gene, 280 (1-2): 37-48 (2002)).

Voltage dependent calcium channels vary greatly in structure and form. Calcium channels are classified as L-, N-, P/Q, T- and R-type according to their pharmacological and electrophysiological properties. These channel subtypes have distinct physiological functions. Molecular cloning has clarified the al subunit sequence of each channel. The al subunit has a specific role in eliciting activity in an individual channel. Nonetheless, selective antagonists for these channel subtypes are required for defining specific channels involved in each activity. The neural N-type channels are blocked by w-conotoxin GVIA; the R-type channels are resistant to other antagonists and toxins, are blocked by SNX-482, and may be involved in processes in the brain; the closely related P/Q-type channels are blocked by some Ω-agatoxins. The dihydropyridine-sensitive L-type channels are responsible for excitation-contraction coupling of skeletal, smooth, and cardiac muscle and for hormone secretion in endocrine cells and also are antagonized by phenylalkylamines and benzothiazepines.

5.2. Types of Voltage-Dependent Calcium Channels

L-Type Calcium Channels

L-type voltage-gated calcium channels are opened when a smooth muscle cell is depolarized. This depolarization may be brought about by stretching of the cell, by an agonist-binding its G protein-coupled receptor (GPCR), or by autonomic nervous system stimulation. Opening of the L-type calcium channel causes influx of extracellular Ca²⁺, which then binds calmodulin. The activated calmodulin molecule activates myosin light-chain kinase (MLCK), which phosphorylates the myosin in thick filaments. Phosphorylated myosin is able to form cross bridges with actin thin filaments, and the smooth muscle fiber (i.e., cell) contracts via the sliding filament mechanism. (Yamakage M. et al, “Calcium channels—basic aspects of their structure, function and gene encoding; anesthetic action on the channels—a review,” Can. J. Anaesth., 49(2): 151-64 (2002))

L-type calcium channels also are enriched in the t-tubules of striated muscle cells, such as, skeletal and cardiac myofibers. As in smooth muscle, L-type calcium channels open when these cells are depolarized. In skeletal muscle, since the L-type calcium channel and the calcium-release channel (ryanodine receptor, or RYR) are mechanically gated to each other with the latter located in the sarcoplasmic reticulum (SR), the opening of the L-type calcium channel causes the opening of the RYR. In cardiac muscle, opening of the L-type calcium channel permits influx of calcium into the cell. The calcium binds to the calcium release channels (RYRs) in the SR, opening them (referred to as “calcium-induced calcium release” or “CICR”). Ca²⁺ is released from the SR and is able to bind to troponin C on the actin filaments regardless of how the RYRs are opened, either through mechanical-gating or CICR. The muscles then contract through the sliding filament mechanism, causing shortening of sarcomeres and muscle contraction.

R-Type Voltage Dependent Calcium Channels

R-type voltage dependent calcium channels (VDCC) are involved in regulating calcium flow. The R-type VDCCs may play a role in decreased cerebral blood flow observed following SAH. Without being limited by theory, R-type voltage-dependent Ca²⁺ channels that may be located within small diameter cerebral arteries may regulate global and local cerebral blood flow, since the concentration of intracellular free calcium ions determines the contractile state of vascular smooth muscle. (Yamakage M. et al, “Calcium channels—basic aspects of their structure, function and gene encoding; anesthetic action on the channels—a review,” Can. J. Anaesth., 49(2): 151-64 (2002)).

R-type voltage dependent calcium channel inhibitors are calcium entry blocking drugs whose main pharmacological effect is to prevent or slow the entry of calcium into cells via R-type voltage-gated calcium channels. The gene Caᵥ2.3 encodes the principal pore-forming unit of R-type voltage-dependent calcium channels being expressed in neurons.

N-Type Calcium Channels

N-type (‘N’ for “Neural-Type”) calcium channels are found primarily at presynaptic terminals and are involved in neurotransmitter release. Strong depolarization by an action potential causes these channels to open and allow influx of Ca²⁺, initiating vesicle fusion and release of stored neurotransmitter. N-type channels are blocked by w-conotoxin. (Yamakage M. et al, “Calcium channels—basic aspects of their structure, function and gene encoding; anesthetic action on the channels—a review,” Can. J. Anaesth., 49(2): 151-64 (2002)).

P/Q-Type Calcium Channels

P-type (‘P’ for cerebellar Purkinje cells) calcium channels play a similar role to the N-type calcium channel in neurotransmitter release at the presynaptic terminal, and in neuronal integration in many neuronal types. They also are found in Purkinje fibers in the electrical conduction system of the heart (Winds, R., et al., J. Physiol. (Lond.) 305: 171-95 (1980); Llinds, R. et al., Proc. Natl. Acad. Sci. U.S.A. 86 (5): 1689-93 (1989)). Q-type calcium channel antagonists appear to be present in cerebellar granule cells. They have a high threshold of activation and relatively slow kinetics. (Yamakage M. et al, “Calcium channels—basic aspects of their structure, function and gene encoding; anesthetic action on the channels—a review,” Can. J. Anaesth., 49(2): 151-64 (2002)).

T-Type Calcium Channels

T-type (‘T’ for transient) calcium channel antagonists are low voltage-activated. They most often are found in neurons and cells that have pacemaker activity and in osteocytes. Mibefradil shows some selectivity for T-type over other types of VDCC. (Yamakage M. et al, “Calcium channels—basic aspects of their structure, function and gene encoding; anesthetic action on the channels—a review,” Can. J. Anaesth., 49(2): 151-64 (2002)).

5.3. Antagonists and Inhibitors of Calcium Channels

Calcium channel antagonists are a class of drugs and natural substances having effects on many excitable cells of the body, such as the muscle of the heart, smooth muscles of the vessels or neuron cells. The primary action of many calcium channel antagonists is to decrease blood pressure, via L-type calcium channel blockade. (Survase, S. et al., “Actions of calcium channel blockers on vascular proteoglycan synthesis: relationship to atherosclerosis,” Vasc. Health Risk Manag., 1(3): 199-208 (2005)).

Calcium channel antagonists act upon voltage-dependent calcium channels (VDCCs) in muscle cells of the heart and blood vessels. By blocking the calcium channel they prevent increases of calcium concentrations in the cells when stimulated, which subsequently leads to less muscle contraction. In the heart, a decrease in calcium available for each beat results in a decrease in cardiac contractility. In blood vessels, a decrease in calcium results in less contraction of the vascular smooth muscle and therefore an increase in blood vessel diameter. The resultant vasodilation decreases total peripheral resistance, while a decrease in cardiac contractility decreases cardiac output. Since blood pressure is in part determined by cardiac output and peripheral resistance, blood pressure drops.

Calcium channel antagonists do not decrease the responsiveness of the heart to input from the sympathetic nervous system. Since blood pressure regulation is carried out by the sympathetic nervous system (via the baroreceptor reflex), calcium channel antagonists allow blood pressure to be maintained more effectively than do β-blockers. However, because calcium channel antagonists result in a decrease in blood pressure, the baroreceptor reflex often initiates a reflexive increase in sympathetic activity leading to increased heart rate and contractility. The decrease in blood pressure also likely reflects a direct effect of antagonism of VDCC in vascular smooth muscle, leading to vasodilation. A β-blocker may be combined with a calcium channel antagonist to minimize these effects.

Calcium channel antagonists may decrease the force of myocardial contraction, an effect that depends on the chemical class of antagonist. This is known as the “negative inotropic effect” of calcium channel antagonists. (Bryant, B. et al., “Pharmacology for health professionals,” 3rd Ed., Elsevier Australia (2010)). Most calcium channel antagonists are not the preferred choice of treatment in individuals with cardiomyopathy due to their negative inotropic effects. (Lehne, R., “Pharmacology for nursing care,” 7th Ed., St. Louis, Mo., Saunders Elsevier., p. 505 (2010)).

Some calcium channel antagonists exhibit a negative dromotropic effect in that they slow the conduction of electrical activity within the heart by blocking the calcium channel during the plateau phase of the action potential of the heart. This effect is known as a “negative dromotropic effect”. Some calcium channel antagonists can also cause a lowering of the heart rate and may cause heart block (which is known as the “negative chronotropic effect” of calcium channel antagonists). The negative chronotropic effects of calcium channel antagonists make them a commonly used class of agents for control of the heart rate in individuals with atrial fibrillation or flutter. (See for example, Murphy C. E. et al., “Calcium channel blockers and cardiac surgery,” J. Card. Surg., 2(2): 299-325 (1987)).

The antagonists for L, N, and P/Q-types of calcium channels are utilized in distinguishing channel subtypes. For the R-type calcium channel subtype, for example, w-agatoxin IIIA shows blocking activity, even though its selectivity is rather low. This peptide binds to all of the high voltage-activated channels including L, N, and P/Q subtypes (J. Biol. Chem., 275, 21309 (2000)). A putative R-type (or class αlE) selective blocker, SNX-482, a toxin from the tarantula Hysterocrates gigas, is a 41 amino acid residue peptide with 3 disulfide linkages (1-4, 2-5 and 3-6 arrangement) (Biochemistry, 37, 15353 (1998), Peptides 1998, 748 (1999)). This peptide blocks the class E calcium channel (IC₅₀=15 nM to 30 nM) and R-type calcium current in the neurohypophysial nerve endings at 40 nM concentration. R-type (class E) calcium channel blocking activity is highly selective; no effect is observed on K⁺ and Na⁺ currents, and L, P/Q and T-type calcium currents. N-type calcium current is blocked only weakly 30-50% at 300 nM to 500 nM. Regionally, different sensitivity of R-type current to SNX-482 is observed; no significant effect on R-type current occurs in preparations of the neuronal cell body, retinal ganglion cells and hippocampal pyramidal cells. Using SNX-482, three α E-calcium subunits with distinct pharmacological properties are recognized in cerebellar R-type calcium channels (J. Neurosci., 20, 171 (2000)). Similarly, it has been shown that secretion of oxytocin, but not vasopressin, is regulated by R-type calcium current in neurohypophysial terminals (J. Neurosci., 19, 9235 (1999)).

Dihydropyridine calcium channel antagonists often are used to reduce systemic vascular resistance and arterial pressure, but are not used to treat angina (with the exception of amlodipine, which carries an indication to treat chronic stable angina as well as vasospastic angina) since the vasodilation and hypotension can lead to reflex tachycardia. This calcium channel antagonist class is easily identified by the suffix “-dipine.”

Phenylalkylamine calcium channel antagonists are relatively selective for myocardium. They reduce myocardial oxygen demand and reverse coronary vasospasm. They have minimal vasodilatory effects compared with dihydropyridines. Their action is intracellular.

Benzothiazepine calcium channel antagonists are an intermediate class between phenylalkylamine and dihydropyridines in their selectivity for vascular calcium channels. Benzothiazepines are able to reduce arterial pressure without producing the same degree of reflex cardiac stimulation caused by dihydropyridines due to their cardiac depressant and vasodilator actions.

L-type VDCC inhibitors are calcium entry blocking drugs whose main pharmacological effect is to prevent or slow entry of calcium into cells via L-type voltage-gated calcium channels. Examples of such L-type calcium channel inhibitors include, but are not limited to: dihydropyridine L-type antagonists such as nisoldipine, AHF (such as 4aR,9aS)-(+)-4a-Amino-1,2,3,4,4a,9a-hexahydro-4a14-fluorene, HC1), isradipine (such as 4-(4-Benzofurazanyl)-1,-4-dihydro-2,6-dimethyl-3,5-pyridinedicarboxylic acid methyl 1-methylethyl ester), calciseptine (such as isolated from (Dendroaspis polylepis ploylepis), H-Arg-Ile-Cys-Tyr-Ile-His-Lys-Ala-Ser-Leu-Pro-Arg-Ala-Thr-Lys-Thr-Cys-Val-Glu-Asn-Thr-Cys-Tyr-Lys-Met-Phe-Ile-Arg-Thr-Gln-Arg-Glu- Tyr-Ile-Ser-Glu-Arg-Gly-Cys-Gly-Cys-Pro-Thr-Ala-Met-Trp-Pro-Tyr-G1-n-Thr-Glu-Cys-Cys-Lys-Gly-Asp-Arg-Cys-Asn-Lys-OH [SEQ ID NO: 1], Calcicludine (such as isolated from Dendroaspis angusticeps (eastern green mamba)), (H-Trp-Gln-Pro-Pro-Trp-Tyr-Cys-Lys-Glu-Pro-Val-Arg-Ile-Gly-Ser-Cys-Lys-Lys-Gln-Phe-Ser-Ser-Phe-Tyr-Phe-Lys-Trp-Thr-Ala-Lys-Lys-Cys-Leu-Pro-Phe-Leu-Phe-Ser-Gly-Cys-Gly-Gly-Asn-Ala-Asn-Arg-Phe-Gln-Thr-Ile-Gly-Glu-Cys-Arg- Lys-Lys-Cys-Leu-Gly-Lys-OH [SEQ ID NO: 2], Cilnidipine (such as also FRP-8653, a dihydropyridine-type inhibitor), Dilantizem (such as (2S,3S)-(+)-cis-3-Acetoxy-5-(2-dimethylaminoethyl)-2,3-dihydro-2-(4-methoxyphenyl)-1,5-benzothiazepine-4(5H)-one hydrochloride), diltiazem (such as benzothiazepine-4(5H)-one, 3-(acetyloxy)-5-[2-(dimethylamino)ethyl]-2,3-dihydro-2-(4-methoxyphenyl)-(+)-cis-monohydrochloride), Felodipine (such as 4-(2,3-Dichlorophenyl)-1,4-dihydro-2,6-dimethyl-3,5-pyridinecarboxylic acid ethyl methyl ester), FS-2 (such as an isolate from Dendroaspis polylepis polylepis venom), FTX-3.3 (such as an isolate from Agelenopsis aperta), Neomycin sulfate (such as C₂₃H₄₆N₆O₁₃. 3H₂SO₄), Nicardipine (such as 1,4-Dihydro-2,6-dimethyl-4-(3-nitrophenylmethyl-2-[methyl(phenylmethylamino]-3,5-pyridinedicarboxylic acid ethyl ester hydrochloride, also YC-93, Nifedipine (such as 1,4-Dihydro-2,6-dimethyl-4-(2-nitrophenyl)-3,5-pyridinedicarboxylic acid dimethyl ester), Nimodipine (such as 4-Dihydro-2,6-dimethyl-4-(3-nitrophenyl)-3,5-pyridinedicarboxylic acid 2-methoxyethyl 1-methylethyl ester) or (Isopropyl 2-methoxyethyl 1,4-dihydro-2,6-dimethyl-4-(m-nitrophenyl)-3,5-pyridinedicarboxylate), Nitrendipine (such as 1,4-Dihydro-2,6-dimethyl-4-(3-nitrophenyl)-3,5-pyridinedicarboxylic acid ethyl methyl ester), S-Petasin (such as (3 S,4aR,5R,6R)-[2,3,4,4a,5,6,7,8-Octahydro-3-(2-propenyl)-4a,5-dimethyl-2-o-xo-6-naphthyl]Z-3′-methylthio-1′-propenoate), Phloretin (such as 2′,4′,6′-Trihydroxy-3-(4-hydroxyphenyl)propiophenone, also 3-(4-Hydroxyphenyl)-1-(2,4,6-trihydroxyphenyl)-1-propanone, also b-(4-Hydroxyphenyl)-2,4,6-trihydroxypropiophenone), Protopine (such as C₂₀H₁₉NO₅C1), SKF-96365 (such as 1-[b-[3-(4-Methoxyphenyl)propoxyl-4-methoxyphenethyl]-1H-imidazole, HC1), Tetrandine (such as 6,6′,7,12-Tetramethoxy-2,2′-dimethylberbaman), (+/−)-Methoxyverapamil or (+)-Verapamil (such as 54N-(3,4-Dimethoxyphenylethyl)methylamino]-2-(3,4-dimethoxyphenyl)-2-iso-propylvaleronitrile hydrochloride), and (R)-(+)-Bay K8644 (such as R-(+)-1,4-Dihydro-2,6-dimethyl-5-nitro-4-442-(trifluoromethyl)phenyl]-3-pyridinecarboxylic acid methyl ester). The foregoing examples may be specific to L-type voltage-gated calcium channels or may inhibit a broader range of voltage-gated calcium channels, e.g. N, P/Q, R, and T-type.

### 6. Endothelins

Endothelins are small vasoconstricting peptides (21 amino acids) produced in vivo primarily in the endothelium that increase blood pressure and vascular tone, and play an important role in vascular homeostasis. This family of peptides includes endothelin-1 (ET-1), endothelin-2 (ET-2) and endothelin-3 (ET-3). ET-1 is secreted mostly by vascular endothelial cells. The predominant ET-1 isoform is expressed in vasculature and is the most potent vasoconstrictor. ET-1 also has inotropic, chemotactic and mitogenic properties. It stimulates the sympathetic nervous system, and influences salt and water homeostasis through its effects on the renin-angiotensin-aldosterone system (RAAS), vasopressin and atrial natriuretic peptide. Endothelins are among the strongest vasoconstrictors known and have been implicated in vascular diseases of several organ systems, including the heart, general circulation and brain.

There are two key endothelin receptor types, ETA and ETB. ETA and ETB have distinct pharmacological characteristics. The ETA-receptor affinity is much higher for ET-1 than for ET-3. ETA receptors are located in the vascular smooth muscle cells, but not in endothelial cells. The binding of endothelin to ETA increases vasoconstriction and the retention of sodium, leading to increased blood pressure. ETB receptors primarily are located on the endothelial cells that line the interior of the blood vessels. There may be ETB receptors on smooth muscle cells which mediate contraction. Endothelin binding to ETB receptors lowers blood pressure by increasing natriuresis and diuresis, and releasing nitric oxide. ET-1 and ET-3 activate the ETB receptor equally, which in turn leads to vasodilation via production of NO and prostaglandins. Endothelin-1 (ET-1) also has been demonstrated to cause vascular smooth muscle constriction via ETA receptor stimulation and to induce nitric oxide (NO) production in endothelial cells via ETB receptors. Some ETB receptors are located in vascular smooth muscle, where they may mediate vasoconstriction. A number of endothelin receptors are regulated by various factors. Angiotensin II and phorbol esters down-regulate endothelin receptors whereas ischemia and cyclosporin increase the number of endothelin receptors. (Reviewed in Aapitov, A. V. et al., “Role of endothelin in cardiovascular disease,” Journal of Renin-Angiotensin-Aldosterone System, 3(1): 1-15 (2002)).

A number of peptide and nonpeptide ET antagonists have been studied. ETA receptor antagonists may include, but are not limited to, A-127722 (non-peptide), ABT-627 (non-peptide), BMS 182874 (non-peptide), BQ-123 (peptide), BQ-153 (peptide), BQ-162 (peptide), BQ-485 (peptide), BQ-518 (peptide), BQ-610 (peptide), EMD-122946 (non-peptide), FR 139317 (peptide), IPI-725 (peptide), L-744453 (non-peptide), LU 127043 (non-peptide), LU 135252 (non-peptide), PABSA (non-peptide), PD 147953 (peptide), PD 151242 (peptide), PD 155080 (non-peptide), PD 156707 (non-peptide), RO 611790 (non-peptide), SB-247083 (non-peptide), clazosentan (non-peptide), atrasentan (non-peptide), sitaxsentan sodium (non-peptide), TA-0201 (non-peptide), TBC 11251 (non-peptide), TTA-386 (peptide), WS-7338B (peptide), ZD-1611 (non-peptide), and aspirin (non-peptide). ETA/B receptor antagonists may include, but are not limited to, A-182086 (non-peptide), CGS 27830 (non-peptide), CP 170687 (non-peptide), J-104132 (non-peptide), L-751281 (non-peptide), L-754142 (non-peptide), LU 224332 (non-peptide), LU 302872 (non-peptide), PD 142893 (peptide), PD 145065 (peptide), PD 160672 (non-peptide), RO-470203 (bosentan, non-peptide), RO 462005 (non-peptide), RO 470203 (non-peptide), SB 209670 (non-peptide), SB 217242 (non-peptide), and TAK-044 (peptide). ETB receptor antagonists may include, but are not limited to, A-192621 (non-peptide), A-308165 (non-peptide), BQ-788 (peptide), BQ-017 (peptide), IRL 1038 (peptide), IRL 2500 (peptide), PD-161721 (non-peptide), RES 701-1 (peptide), and RO 468443 (peptide). (Aapitov, A. V. et al., “Role of endothelin in cardiovascular disease,” Journal of Renin-Angiotensin-Aldosterone System, 3(1): 1-15 (2002)).

ET-1 is translated initially to a 212 amino-acid peptide (pre-proendothelin-1). It is further converted to proendothelin-1 after removal of the secretory sequence. Proendothelin-1 then is cleaved by furin to generate the biologically-inactive precursor big endothelin-1. Mature ET-1 is formed upon cleavage of big endothelin-1 by one of several endothelin-converting enzymes (ECEs). There are two splice variants of ECE-1; these are ECE-1a and ECE-1b. Each has functionally distinct roles and tissue distribution. ECE-1a is expressed in the Golgi network of endothelin-producing cells and cleaves big endothelin-1 to form ET-1. ECE-1b is localized at the plasma membrane and cleaves extracellular big endothelin-1. Both ECE-1a and ECE-1b are inhibited by metalloprotease inhibitor phosphoramidon. ECEs also are located on a-actin filaments in smooth muscle cells. ECE inhibition by phosphoramidon completely blocks vasoconstriction to big endothelin-1. ECE inhibitors may include, but are not limited to, B-90063 (non-peptide), CGS 26393 (non-peptide), CGS 26303 (non-peptide), CGS 35066 (non peptide), phosphoramidon (peptide), PP-36 (peptide), SM-19712 (non-peptide), and TMC-66 (non-peptide). (Aapitov, A. V. et al., “Role of endothelin in cardiovascular disease,” Journal of Renin-Angiotensin-Aldosterone System, 3(1): 1-15 (2002)).

In a healthy individual, a delicate balance between vasoconstriction and vasodilation is maintained by endothelin and other vasoconstrictors on the one hand and nitric oxide, prostacyclin and other vasodilators on the other. Endothelin antagonists may have a role in the treatment of cardiac, vascular and renal diseases associated with regional or systemic vasoconstriction and cell proliferation, such as essential hypertension, pulmonary hypertension, chronic heart failure, chronic renal failure and SAH.

### 7. Transient Receptor Potential Channels

The transient receptor potential (TRP) channel family is a member of the calcium channel group. These channels include transient receptor potential protein and homologues thereof, the vanilloid receptor subtype I, stretch-inhibitable non-selective cation channel, olfactory, mechanosensitive channel, insulin-like growth factor I-regulated calcium channel, and vitamin D-responsive apical, epithelial calcium channel (ECaC). (see for example, Montell C. et al., “Molecular characterization of the Drosophila trp locus: a putative integral membrane protein required for phototransduction, Neuron, 2(4):1313-1323 (1989); Caterina et al., “The capsaicin receptor: a heat-activated ion channel in the pain pathway,” Nature, 389 (6653): 816-824 (1997); Suzuki et al., “Cloning of a stretch-inhibitable nonselective cation channel,” J. Biol. Chem. 274: 6330-6335 (1999); Kiselyov et al., “Functional interaction between InsP3 receptors and store-operated Htrp3 channels,” Nature 396 (6710): 478-482 (1998); Hoenderop et al., “Molecular identification of the apical Ca2+ channel in 1, 25-dihydroxyvitamin D3-responsive epithelia,” J. Biol. Chem. 274(13): 8375-8378 (1999); and Chen et al., “Polycystin-L is a calcium-regulated cation channel permeable to calcium ions,” Nature, 401(6751): 383-386 (1999)). Each of these molecules is at least 700 amino acids in length, and shares certain conserved structural features. Predominant among these structural features are six transmembrane domains, with an additional hydrophobic loop present between the fifth and sixth transmembrane domains. It is believed that this loop is integral to the activity of the pore of the channel formed upon membrane insertion. TRP channel proteins also include one or more ankyrin domains and frequently display a proline-rich region at the N-terminus.

Based on amino acid homology, the TRP superfamily can be further subdivided into sub-families. In mammals, these include TRPC (canonical), TRPV (vanilloid), TRPM (melastanin), TRPP (polycystin), TRPML (mucolipin), and TRPA (ankyrin) groups. The TRPC (canonical) subfamily includes 7 TRP channels (TRPC1-7); the TRPM (melastanin) subfamily includes eight different channels (TRPM1-8); the TRPV (vanilloid) subfamily includes six members (TRPV1-6); the TRPA (ankyrin) subfamily includes one member (TRPA1) and the TRPP (polycystin) and TRPML (mucolipin) subfamilies each include three mammalian members. In addition, the TRPN (No mechanopotential) found in hearing assisting sensory neurons have been identified in Drosophila and zebrafish. (Nilius, B. et al., “Transient receptor potential cation channels in disease,” Physiol. Rev. 87: 165-217 (2007)).

Transient receptor potential (TRP) cation channels are present in vascular smooth muscle and are involved in the smooth muscle depolarizing response to stimuli such as membrane stretch. Uridine triphosphate (UTP) invokes membrane depolarization and constriction of vascular smooth muscle by activating a cation current that exhibits inward rectification, is not rapidly desensitized and is blocked by gadolinium ions (Gd³⁺). Canonical transient receptor potential (TRPC) proteins form Ca²⁺ permeable, non-selective cation channels in a variety of mammalian tissues. Suppression of one member of this family of channels, TRPC6, has been reported to prevent an a-adenoreceptor-activated cation current in cultured rabbit portal vein myocytes. However, suppression of TRPC6 channels in cerebral vascular smooth muscle does not attenuate the UTP-induced membrane depolarization and vasoconstriction. In contrast, TRPC3, unlike TRPC6, has been found to mediate the agonist induced depolarization, as observed in rat cerebral artery, following UTP activation of the P2Y receptor. Thus, TRPC3 channels in vascular smooth muscle mediate agonist-induced depolarization which contributes to vasoconstriction in resistance-sized cerebral arteries.

The TRP1 channel family comprises a large group of channels mediating an array of signal and sensory transduction pathways. The proteins of the mammalian TRPC subfamily are the products of at least 7 genes coding for cation channels that appear to be activated in response to phospholipase C (PLC)-coupled receptors. The putative ion channel subunits TRPC3, TRPC6, and TRPC7 comprise a structurally related subgroup of the family of mammalian TRPC channels. The ion channels formed by these proteins appear to be activated downstream of phospholipase C (PLC). PLC-dependent activation of TRPC6 and TRPC7 has been shown to involve diacylglycerol and is independent of G proteins or inositol 1,4,5-triphosphate (IP₃).

TRPC channels are widely expressed among cell types and may play important roles in receptor-mediated Ca²⁺ signaling. The TRPC3 channel is known to be a Ca²⁺-conducting channel activated in response to PLC-coupled receptors. TRPC3 channels have been shown to interact directly with intracellular inositol IP₃ receptors (InsP₃Rs), i.e., channel activation is mediated through coupling to InsP₃Rs.

Agents useful for increasing arterial blood flow, inhibiting vasoconstriction or inducing vasodilation are agents that inhibit TRP channels. These inhibitors embrace compounds that are TRP channel antagonists. Such inhibitors are referred to as activity inhibitors or TRP channel activity inhibitors. As used herein, the term “activity inhibitor” refers to an agent that interferes with or prevents the activity of a TRP channel. An activity inhibitor may interfere with the ability of the TRP channel to bind an agonist such as UTP. An activity inhibitor may be an agent that competes with a naturally occurring activator of TRP channel for interaction with the activation binding site on the TRP channel Alternatively, an activity inhibitor may bind to the TRP channel at a site distinct from the activation binding site, but in doing so, it may, for example, cause a conformational change in the TRP channel, which is transduced to the activation binding site, thereby precluding binding of the natural activator. Alternatively, an activity inhibitor may interfere with a component upstream or downstream of the TRP channel but which interferes with the activity of the TRP channel. This latter type of activity inhibitor is referred to as a functional antagonist. Non-limiting examples of a TRP channel inhibitor that is an activity inhibitor are gadolinium chloride, lanthanum chloride, SKF 96365 and LOE-908.

### 8. Regression Analyses for Selection of Eligible Subjects

The severity of the SAH, as measured semiquantitatively by clot thickness on CT scan, is the most important predictor of the risk for developing DCI and infarction. Since DCI is a well-documented risk factor for poor outcome, it follows that clinical grade at presentation alone cannot adequately predict patients at risk for DCI and poor outcome, and that the volume of the initial hemorrhage must be taken into account when making a judgment about which patients to treat.

DCI is a well-documented risk factor for poor outcome. Clinical grade at presentation alone cannot adequately predict patients at risk for DCI and poor outcome; the volume of the initial hemorrhage must be taken into account when making a judgment about which patients to treat. The severity of the SAH, as measured semiquantitatively by clot thickness on CT scan, is the most important predictor of the risk for developing DCI and infarction.

A systematic review and meta analysis of twenty one randomized, double-blind, placebo-controlled trials that studied the efficacy of pharmaceutical preventive strategies in SAH patients, including 7788 patients, and had both cerebral infarction and clinical outcome as outcome events showed that there is an association between infarction, a principle component of the diagnosis of DCI, and outcome. (Asano T et al., “Effects of a hydroxyl radical scavenger on delayed ischemic neurological deficits following aneurysmal subarachnoid hemorrhage: results of a multicenter, placebo-controlled double-blind trial,” J. Neurosurg., 84:792-803 (1996); Chou S H et al., “A randomized, double-blind, placebo-controlled pilot study of simvastatin in aneurysmal subarachnoid hemorrhage,” Stroke, 39:2891-2893 (2008); Fisher C M et al., “Cerebral vasospasm with ruptured saccular aneurysm—the clinical manifestations,” Neurosurgery, 1:245-248 (1977); Gomis P et al., “Randomized, double-blind, placebo-controlled, pilot trial of high-dose methylprednisolone in aneurysmal subarachnoid hemorrhage,” J. Neurosurg., 112:681-688 (2010); Haley E C, Jr. et al., “A randomized, double-blind, vehicle-controlled trial of tirilazad mesylate in patients with aneurysmal subarachnoid hemorrhage: a cooperative study in North America,” J. Neurosurg., 86:467-474 (1997); Haley E C, Jr. et al., “A randomized controlled trial of high-dose intravenous nicardipine in aneurysmal subarachnoid hemorrhage. A report of the Cooperative Aneurysm Study,” J. Neurosurg., 78:537-547 (1993); Hop J W et al., “Randomized pilot trial of postoperative aspirin in subarachnoid hemorrhage,” Neurology, 54:872-878 (2000); Kassell N F et al., “Randomized, double-blind, vehicle-controlled trial of tirilazad mesylate in patients with aneurysmal subarachnoid hemorrhage: a cooperative study in Europe, Australia, and New Zealand,” J. Neurosurg., 84:221-228 (1996); Lanzino G, and Kassell N F, “Double-blind, randomized, vehicle-controlled study of high-dose tirilazad mesylate in women with aneurysmal subarachnoid hemorrhage. Part II. A cooperative study in North America,” J. Neurosurg., 90:1018-1024 (1999); Lanzino G et al., “Double-blind, randomized, vehicle-controlled study of high-dose tirilazad mesylate in women with aneurysmal subarachnoid hemorrhage. Part I. A cooperative study in Europe, Australia, New Zealand, and South Africa,” J. Neurosurg., 90:1011-1017 (1999); Macdonald R L et al., “Clazosentan to overcome neurological ischemia and infarction occurring after subarachnoid hemorrhage (CONSCIOUS-1): randomized, double-blind, placebo-controlled phase 2 dose-finding trial,” Stroke, 39:3015-3021 (2008); Ohman J, and Heiskanen O, “Effect of nimodipine on the outcome of patients after aneurysmal subarachnoid hemorrhage and surgery,” J. Neurosurg. 69:683-686 (1988); Pickard J D et al., “Effect of oral nimodipine on cerebral infarction and outcome after subarachnoid haemorrhage: British aneurysm nimodipine trial,” BMJ, 298:636-642 (1989); Saito I et al., “Neuroprotective effect of an antioxidant, ebselen, in patients with delayed neurological deficits after aneurysmal subarachnoid hemorrhage,” Neurosurgery, 42:269-277 (1998); Shaw M D, et al. “Efficacy and safety of the endothelin, receptor antagonist TAK-044 in treating subarachnoid hemorrhage: a report by the Steering Committee on behalf of the UK/Netherlands/Eire TAK-044 Subarachnoid Haemorrhage Study Group,” J. Neurosurg., 93:992-997 (2000); Springborg J B et al., “Erythropoietin in patients with aneurysmal subarachnoid haemorrhage: a double blind randomised clinical trial,” Acta Neurochir. (Wien) 149:1089-1101 (2007); Tseng M Y, et al., “Interaction of Neuroprotective and Hematopoietic Effects of Acute Erythropoietin Therapy with Age, Sepsis, and Statins Following Aneurysmal Subarachnoid Hemorrhage,” Presented at the XIV World Congress of Neurological Surgery of the World Federation of Neurosurgical Societies, Boston, Mass., Aug. 30-Sep. 4, 2009 (Abstract); van den Bergh W M et al., “Randomized controlled trial of acetylsalicylic acid in aneurysmal subarachnoid hemorrhage: the MASH Study,” Stroke 37:2326-2330 (2006); Westermaier T et al., “Prophylactic intravenous magnesium sulfate for treatment of aneurysmal subarachnoid hemorrhage: a randomized, placebo-controlled, clinical study,” Crit. Care Med. 38:1284-1290 (2010); Etminan, N. et al., “Effect of pharmaceutical treatment on vasospasm, delayed cerebral ischemia, and clinical outcome in patients with aneurysmal subarachnoid hemorrhage: a systematic review and meta-analysis,” J. Cereb. Blood Flow Metab. 31:1443-1451 (2011)). Pharmaceutical treatments decreased the incidence of both cerebral infarction (Relative Risk (“RR”) 0.83; 95% CI ranging from 0.74-0.94) and of poor functional outcome (Relative Risk (“RR”) 0.91; 95% CI ranging from 0.85-0.98). (Vergouwen, M. D. et al., “Lower incidence of cerebral infarction correlates with improved functional outcome after aneurysmal subarachnoid hemorrhage,” J. Cereb. Blood Flow Metab., 31:1545-1553 (2011)). Since the mechanism of action of most of the drugs used is to either reverse angiographic vasospasm or protect the brain, these data suggest that the association between cerebral infarction and functional outcome implies causality.

Logistic regression analysis performed with randomized clinical trial data with 3,567 patients between 1991 and 1997 to assess the relationships and interactions between admission neurological grade assessed on the WFNS, subarachnoid clot thickness, DCI and clinical outcome (Haley E C, Jr., Kassell N F, Apperson-Hansen C, Maile M H, Alves W M: A randomized, double-blind, vehicle-controlled trial of tirilazad mesylate in patients with aneurysmal subarachnoid hemorrhage: a cooperative study in North America. Journal of Neurosurgery 86:467-474, 1997; Kassell N F, Haley E C, Jr, Apperson-Hansen C, Alves W M: Randomized, double-blind, vehicle-controlled trial of tirilazad mesylate in patients with aneurysmal subarachnoid hemorrhage: a cooperative study in Europe, Australia, and New Zealand. Journal of Neurosurgery 84:221-228, 1996; Lanzino G, Kassell N F: Double-blind, randomized, vehicle-controlled study of high-dose tirilazad mesylate in women with aneurysmal subarachnoid hemorrhage. Part II. A cooperative study in North America. J. Neurosurg 90:1018-1024, 1999; Lanzino G, Kassell N F, Dorsch N W, Pasqualin A, Brandt L, Schmiedek P, et al: Double-blind, randomized, vehicle-controlled study of high-dose tirilazad mesylate in women with aneurysmal subarachnoid hemorrhage. Part I. A cooperative study in Europe, Australia, New Zealand, and South Africa. J. Neurosurg 90:1011-1017, 1999) showed that both neurological grade and subarachnoid clot thickness predict subsequent development of DCI. Clinical outcome was the dependent variable and was assessed 3 months after SAH on the Glasgow Outcome Scale (GOS). (Jennett B, and Bond M, “Assessment of outcome after severe brain damage. A practical scale. Lancet 1:480-484, 1975). Independent variables assessed included World Federation of Neurosurgical Surgeons (WFNS) grade, age and subarachnoid clot thickness, factors found to be associated with outcome. (Rosengart A J, et al. “Prognostic factors for outcome in patients with aneurysmal subarachnoid hemorrhage,” Stroke 38:2315-2321 (2007)). The other variables that affected outcome and were present on admission were intraventricular hemorrhage, intracerebral hemorrhage and history of hypertension. Both subarachnoid clot volume and WFNS grade were important. If DCI is the dependent variable, then the variables significantly associated with it are age, showing an inverted U-shaped relationship with a peak incidence among patients 40 to 59-years-old. (Macdonald R L et al., “Factors associated with the development of vasospasm after planned surgical treatment of aneurysmal subarachnoid hemorrhage,” J. Neurosurg. 99:644-652 (2003)). Other significant variables were history of hypertension, WFNS grade, subarachnoid clot thickness, aneurysm size and intraventricular hemorrhage.

The Co-operative Study on Timing of Aneurysm Surgery collected data from 68 centers across Europe, North America, Australia, Japan and South Africa. (Kassell N F et al., “The International Cooperative Study on the Timing of Aneurysm Surgery. Part 1: Overall management results,” J. Neurosurg., 73:18-36 (1990)). 3521 patients were enrolled within 3 days of an SAH. At admission 75% of patients had a good neurological grade, defined as having normal speech at admission. Logistic regression analysis showed that that the extent of SAH as assessed by clot thickness on the admission CT scan, is an independent risk factor for development of DCI and infarction. The study found that patients who had a normal CT scan had a low risk of developing DCI, and the risk increased progressively with increasing amounts of blood on CT, with patients having thick focal blood being at the highest risk. The study also showed that development of DCI could not be predicted by the presence of focal motor signs, cranial nerve palsies, language defects, impaired responsiveness, nuchal rigidity or severity of headache at admission. Based on the results of this study, the predictive power of CT for DCI exceeds that of clinical neurological examination.

Hijdra, et al., reported on 176 patients admitted within 72 hours of SAH, who were prospectively studied to assess the predictive value of clinical and radiological features for DCI, rebleeding and outcome. (Hijdra A et al., “Prediction of delayed cerebral ischemia, rebleeding, and outcome after aneurysmal subarachnoid hemorrhage,” Stroke 19:1250-1256 (1988)). At baseline, 49% of patients were Hunt and Hess grades 1-2, and 51% were Hunt and Hess 3-5. Hunt and Hess grades 1-2 would be roughly equivalent to WFNS grade 1 and Hunt and Hess 3-5 to WFNS grades 2-5. 24% of the patients with admission Hunt and Hess grades 1-2 developed DCI and 51% of them died or were vegetative or had severe disability (poor or unfavorable GOS) at 3 months. Stepwise logistic regression analysis showed that death, vegetative state or severe disability was best predicted by the amount of subarachnoid blood on CT scan within 72 hours of rupture (p=0.0001) and admission Glasgow coma score (GCS, p=0.0030). Blood on CT was a stronger predictor than GCS. The analysis also showed that amount of SAH on CT was the most important predictor of DCI, followed by amount of intraventricular hemorrhage, and that the predictive power of these two factors could not be improved further by taking into account the patient's initial neurological condition.

Öhman, et al., prospectively studied 265 good grade patients with aneurysmal SAH to examine which radiological and clinical factors forecast the development of cerebral infarct as a consequence of DCI. (Ohman J et al., “Risk factors for cerebral infarction in good-grade patients after aneurysmal subarachnoid hemorrhage and surgery: a prospective study,” J. Neurosurg. 74:14-20 (1991)). Of these, 104 patients were randomized to receive nimodipine, 109 placebo and 52 received no treatment. The 161 patients who received either placebo or no treatment were analyzed together. At admission 31% of patients were Hunt and Hess grade 1, 44% were grade 2, and 25% were grade 3. Baseline CT showed that 21% of patient had no or small amount of blood on CT, 18% had thin layers of blood, 42% had thick layers of blood and 18% had severe bleeding. Patients were followed up at 1-3 years post-hemorrhage at which time CT scans were performed and evaluated for presence or absence of infarction and GOS was assessed at the same time. Logistic regression analysis showed that, in order of importance, the following factor were strongly predictive of infarction: severe bleeding on admission CT, history of hypertension and thick layers of blood in the basal cisterns on admission CT. Post-operative angiograms were done on 213 patients. 78 patients had moderate or severe vasospasm and 65% of them had infarction on follow up CT scans. Clinical grade at admission had no significant effect on cerebral infarction. There was an apparent trend for grade 3 patients to have more infarcts but the differences between neurological grades did not reach significance.

Woertgen and colleagues studied 292 patients with aneurysmal SAH (“aSAH”) between 1995 and 2000 with the aim of comparing clinical scales and CT findings to predict DCI. (Woertgen C et al., “Comparison of the Claassen and Fisher CT classification scale to predict ischemia after aneurysmatic SAH?” Zentralbl Neurochir 64:104-108 (2003)). DCI was defined as new cerebral infarction on CT. Correlations between admission Hunt and Hess grade, Fisher grade 39 and Claassen grades 23 with cerebral infarction on CT were analyzed. The outcome at 3 months, based on the GOS, was also analyzed, with unfavorable outcome defined as death, vegetative or severe disability and favorable outcome defined as moderate disability or good recovery. The odds ratio (meaning the ratio of the odds of developing an infarct in one grade to the odds of developing an infarct in the control group) for infarction was calculated at each level of the grading scales. The control group was the grade with the lowest risk of infarction, that is, Hunt and Hess grade 0. In terms of the impact of infarction on outcome at 3 months, 63% of patients (183/292) had favorable outcome and 37% had unfavorable outcome. Of those that had favorable outcome, only 9% had an infarct on CT, whereas of those that had unfavorable outcome, 62% had an infarct on CT (p<0.0001). According to this data, both clinical grade and clot thickness are independently related to risk of infarction, and infarction is associated with poor outcome.

Data from the Cooperative Study on Timing of Aneurysm Surgery was analyzed to assess the prognostic value of various neurological signs and CT parameters for predicting survival and degree of recovery. (Adams H P, Jr. et al., “Usefulness of computed tomography in predicting outcome after aneurysmal subarachnoid hemorrhage: a preliminary report of the Cooperative Aneurysm Study,” Neurology 35:1263-1267 (1985)). Baseline CT was graded as normal or having SAH, intraventricular hemorrhage, intracerebral hemorrhage, subdural blood, hydrocephalus, edema, aneurysm or infarct. If SAH was present, clot thickness was graded as diffuse, local thick or local thin. Outcome was assessed by a blinded assessor, at 6 months, using the GOS. The prognostic value of each parameter was evaluated individually. Logistic regression analysis was then used to determine whether CT factors predicted outcome regardless of level of consciousness at admission. 1778 patients were eligible for evaluation. 44 patients were excluded because CT was not done within the prescribed time frame. The remaining 1734 patients were evaluated. Mortality was higher among patients who had blood on CT compared to those who did not (5% versus 27%). Mortality was greater in patients that had diffuse or local thick blood, compared to those who had local thin blood (33% versus 32% versus 10% respectively). Mortality was greater in patients with local thin blood than those with no blood (10% versus 6%). Among 124 alert patients with no blood on CT, mortality was 2.4% at 6 months and good recovery was 93%. Among 684 alert patients with blood on CT, mortality was 12% and good recovery 73%.

### 9. Drug Delivery to Target Sites in the Brain

The limited permeability of the brain capillary endothelial wall, constituting the blood brain barrier (BBB), poses challenges to the development of methods of drug delivery to target sites in the brain. Such challenges can be overcome by bypassing the BBB and administering a drug locally into the brain near the site of action. Alternatively, the drug can be administered into the subarachnoid space of the spine, i.e., spinal (intrathecal) drug administration, such that the drug is carried from the site of delivery in the spine to the site of action in the brain via the CSF. However, such localized intracranial or spinal administrations are invasive and are associated with a risk of CNS infections, which increases if more injections have to be given or if a catheter has to be left in place to repeat the injection. Furthermore, most drugs delivered directly into the CSF are rapidly cleared, exhibiting very short half-lives, thus requiring frequent invasive administrations to maintain therapeutic levels at target sites of the action. This limits the practical applicability of localized drug delivery to the CNS.

In order to overcome such shortcomings, strategies have been developed to circumvent the BBB. These include, for example, osmotic disruption of the BBB, infusion pumps delivering drugs to the CSF, intravenous injection of surface coated nanoparticles, coupling of drugs to a carrier undergoing receptor-mediated transcytosis through the BBB, implantation of tissue or cells, and gene therapy (reviewed in Tamargo, R. J. et al., “Drug delivery to the central nervous system: a review,” Neurosurg., Quarterly 2: 259-279 (1992)). Carriers can affect drug level, location, longevity and antigenicity. (Reviewed in Langer, R., “New methods of drug delivery,” Science, 249: 1527-1533 (1990); and Langer, R., “Drug delivery and targeting,” Nature, 392 (Supp.): 5-10 (1998)). For example, a drug may be chemically modified to selectively alter such properties as biodistribution, pharmacokinetics, solubility, or antigenicity; or a drug can be complexed to agents that enables it to cross a normally impermeable barrier, for example, by rendering the drug more lipophilic or coupling it to a molecule that has a specific transport mechanism. (Bodor, N and Simpkins, Science 221 65 (1983); Kumagai et al, J Biol Chem. 262, 15214 (1987), Jacob et al, J Med. Chem. 33, 733 (1990)).

9.1. Controlled Release Polymeric Drug Delivery Systems

Biodegradable polymeric drug delivery systems that control the release rate of the contained drug in a predetermined manner can overcome practical limitations to targeted brain delivery. A drug can be attached to soluble macromolecules, such as proteins, polysaccharides, or synthetic polymers via degradable linkages. For example, in animals, antitumor agents such as doxorubicin coupled to N-(2-hydroxypropyl) methacrylamide copolymers showed radically altered pharmacokinetics resulting in reduced toxicity. The half-life of the drug in plasma and the drug levels in the tumor were increased while the concentrations in the periphery decreased. (Kopecek and Duncan, J Controlled Release 6, 315 (1987)). Polymers, such as polyethylene glycol (PEG) can be attached to drugs to either lengthen their lifetime or alter their immunogenicity; drug longevity and immunogenicity also may be affected by biological approaches, including protein engineering and altering glycosylation patterns.

Controlled release systems have been developed both for localized delivery to target sites in the brain, as well as for localized delivery to sites in the spinal cord. (Reviewed in Fournier, E. et al., “Biocompatibility of implantable synthetic polymeric drug carriers: focus on brain compatibility,” Biomaterials, 24(19): 331-3331 (2003); Lagarce, F. et al., “Sustained release formulations for spinal drug delivery,” J. Drug Del. Sci. Tech., 14(5): 331-343 (2004)).

Controlled release systems deliver a drug at a predetermined rate for a definite time period. (Reviewed in Langer, R., “New methods of drug delivery,” Science, 249: 1527-1533 (1990); and Langer, R., “Drug delivery and targeting,” Nature, 392 (Supp.): 5-10 (1998)). Generally, release rates are determined by the design of the system, and are nearly independent of environmental conditions, such as pH. These systems also can deliver drugs for long time periods (days or years). Controlled release systems provide advantages over conventional drug therapies. For example, after ingestion or injection of standard dosage forms, the blood level of the drug rises, peaks and then declines. Since each drug has a therapeutic range above which it is toxic and below which it is ineffective, oscillating drug levels may cause alternating periods of ineffectiveness and toxicity. A controlled release preparation maintains the drug in the desired therapeutic range by a single administration. Other potential advantages of controlled release systems include: (i) localized delivery of the drug to a particular body compartment, thereby lowering the systemic drug level; (ii) preservation of medications that are rapidly destroyed by the body; (iii) reduced need for follow-up care; (iv) increased comfort; and (v) improved compliance. (Langer, R., “New methods of drug delivery,” Science, 249: at 1528).

Optimal control is afforded if the drug is placed in a polymeric material or pump. Polymeric materials generally release drugs by the following mechanisms: (i) diffusion; (ii) chemical reaction, or (iii) solvent activation. The most common release mechanism is diffusion. In this approach, the drug is physically entrapped inside a solid polymer that can then be injected or implanted in the body. The drug then migrates from its initial position in the polymeric system to the polymer's outer surface and then to the body. There are two types of diffusion-controlled systems: reservoirs, in which a drug core is surrounded by a polymer film, which produce near-constant release rates, and matrices, where the drug is uniformly distributed through the polymer system. Drugs also can be released by chemical mechanisms, such as degradation of the polymer, or cleavage of the drug from a polymer backbone. Exposure to a solvent also can activate drug release; for example, the drug may be locked into place by polymer chains, and, upon exposure to environmental fluid, the outer polymer regions begin to swell, allowing the drug to move outward, or water may permeate a drug-polymer system as a result of osmotic pressure, causing pores to form and bringing about drug release. Such solvent-controlled systems have release rates independent of pH. Some polymer systems can be externally activated to release more drug when needed. Release rates from polymer systems can be controlled by the nature of the polymeric material (for example, crystallinity or pore structure for diffusion-controlled systems; the lability of the bonds or the hydrophobicity of the monomers for chemically controlled systems) and the design of the system (for example, thickness and shape). (Langer, R., “New methods of drug delivery,” Science, 249: at 1529).

Polyesters such as lactic acid-glycolic acid copolymers display bulk (homogeneous) erosion, resulting in significant degradation in the matrix interior. To maximize control over release, it is often desirable for a system to degrade only from its surface. For surface-eroding systems, the drug release rate is proportional to the polymer erosion rate, which eliminates the possibility of dose dumping, improving safety; release rates can be controlled by changes in system thickness and total drug content, facilitating device design. Achieving surface erosion requires that the degradation rate on the polymer matrix surface be much faster than the rate of water penetration into the matrix bulk. Theoretically, the polymer should be hydrophobic but should have water-labile linkages connecting monomers. For example, it was proposed that, because of the lability of anhydride linkages, polyanhydrides would be a promising class of polymers. By varying the monomer ratios in polyanhydride copolymers, surface-eroding polymers lasting from 1 week to several years were designed, synthesized and used to deliver nitrosoureas locally to the brain. ((Langer, R., “New methods of drug delivery,” Science, 249: at 1531 citing. Rosen et al, Biomaterials 4, 131 (1983); Leong et al, J. Biomed. Mater. Res. 19, 941 (1985); Domb et al, Macromolecules 22, 3200 (1989); Leong et al, J. Biomed. Mater. Res. 20, 51 (1986), Brem et al, Selective Cancer Ther. 5, 55 (1989); Tamargo et al, J. Biomed. Mater. Res. 23, 253 (1989)).

Several different surface-eroding polyorthoester systems have been synthesized. Additives are placed inside the polymer matrix, which causes the surface to degrade at a different rate than the rest of the matrix. Such a degradation pattern can occur because these polymers erode at very different rates, depending on pH, and the additives maintain the matrix bulk at a pH different from that of the surface. By varying the type and amount of additive, release rates can be controlled. ((Langer, R., “New methods of drug delivery,” Science, 249: at 1531 citing. Heller, et al, in Biodegradable Polymers as Drug Delivery Systems, M. Chasin and R. Langer, Eds (Dekker, New York, 1990), pp. 121-161)).

Polymeric materials used in controlled release drug delivery systems described for delivery to the CNS include poly (α-hydroxyacids), acrylic, polyanhydrides and other polymers, such as polycaprolactone, ethylcellulose, polystyrene, etc. A wide range of delivery systems suitable for delivery to the brain and spinal cord have been developed. These include: macroscopic implants, microcapsules, gels and nanogels, microparticles/microspheres, nanoparticles, and composite hydrogel systems. The different types of systems exhibit differences in pharmokinetic and pharmacodynamic profiles of drugs by affecting different physical and chemical processes involved in drug release, such as water penetration, drug dissolution, and degradation of matrix and drug diffusion. (Reviewed in Siepmann, J. et al., “Local controlled drug delivery to the brain: mathematical modeling of the underlying mass transport mechanisms,” International Journal of Pharmaceutics, 314: 101-119 (2006).

### 10. Current Treatment Options

Many drugs have been studied but have failed to improve outcome after serious brain diseases such as ischemic stroke, traumatic brain injury, SAH and malignant brain tumors [van der Worp H B, Howells D W, Sena E S, et al. Can animal models of disease reliably inform human studies? PLoS Med 2010; 7:e1000245.]. The reasons for this may be multifactorial, but include the inability to target drugs to their site of action in order to administer efficacious doses while avoiding off-target effects. For some drugs, adverse effects may limit the dose that can be administered systemically to achieve therapeutic concentrations in the brain or CSF. Strategies that have been used to circumvent this and that have had limited success include blood brain barrier (BBB) opening and use of transporters [Begley D J. Delivery of therapeutic agents to the central nervous system: the problems and the possibilities. Pharmacol Ther 2004; 104:29-45; Gabathuler R. Approaches to transport therapeutic drugs across the blood-brain barrier to treat brain diseases. Neurobiol Dis 2010; 37:48-57.]. Another is to deliver drugs directly into the brain or subarachnoid space by injection, surgical implantation or endonasal route [Begley D J. Delivery of therapeutic agents to the central nervous system: the problems and the possibilities. Pharmacol Ther 2004; 104:29-45.]. Only one such formulation has been developed to date [Brem H, Piantadosi S, Burger P C, et al. Placebo-controlled trial of safety and efficacy of intraoperative controlled delivery by biodegradable polymers of chemotherapy for recurrent gliomas. The Polymer-brain Tumor Treatment Group. Lancet 1995; 345:1008-12.]. Limitations of subarachnoid drug delivery are that injection into the CSF may not produce adequate drug concentrations in the brain. Another is that most diseases require sustained drug concentrations for some time and there is limited ability to access the brain directly without invasive procedures that carry some risk.

An ideal disease for which to develop intracranial drug delivery would be a serious, self-limited, subarachnoid based disease. One example is aneurysmal SAH. While the pathophysiology of brain injury after ischemic stroke and traumatic brain injury may require delivering drug to diffuse areas of brain parenchyma, SAH is unique in that much of the brain injury may be secondary to DCI which is mediated by processes in the confined subarachnoid space. Furthermore, this disease is transient, the subarachnoid space is frequently accessed as part of the routine care of these patients and enteral nimodipine has shown some efficacy in the disease. The enteral dose of nimodipine, however, is limited by systemic hypotension, since the L-type calcium channels upon which it acts are distributed throughout the arterial circulation [Cribbs L L. Vascular smooth muscle calcium channels: could “T” be a target? Circ Res 2001; 89:560-2.].

Outcome from SAH remains poor with 35% of patients dying and many having permanent neurological and neurocognitive morbidity (Al-Khindi T, Macdonald R L, Schweizer T A. Cognitive and functional outcome after aneurysmal subarachnoid hemorrhage. Stroke 2010; 41:e519-e536; Nieuwkamp D J, Setz L E, Algra A, et al. Changes in case fatality of aneurysmal subarachnoid haemorrhage over time, according to age, sex, and region: a meta-analysis. Lancet Neurol 2009; 8:635-42.). Furthermore, an important cause of morbidity after SAH is DCI which does not occur until days after SAH (Suarez J I, Tarr R W, Selman W R. Aneurysmal subarachnoid hemorrhage. N Engl J Med 2006; 354:387-96; Lovelock C E, Cordonnier C, Naka H, et al. Antithrombotic drug use, cerebral microbleeds, and intracerebral hemorrhage: a systematic review of published and unpublished studies. Stroke 2010; 41:1222-8.). Delayed cerebral ischemia is hypothesized to be due to a combination of angiographic vasospasm, microcirculatory dysfunction, cortical spreading ischemia, microthromboembolism and delayed effects of early brain injury (Macdonald R L. Delayed neurological deterioration after subarachnoid haemorrhage. Nat Rev Neurol 2014; 10:44-58.). Despite testing of over 50 drugs, no pharmacologic treatment for improving outcome after SAH has been developed since nimodipine was approved for use in the United States in 1988 (Weyer G W, Jahromi B S, Aihara Y, et al. Expression and function of inwardly rectifying potassium channels after experimental subarachnoid hemorrhage. J Cereb Blood Flow Metab 2006; 26:382-91; Dorhout Mees S M, Rinkel G J, Feigin V L, et al. Calcium antagonists for aneurysmal subarachnoid haemorrhage. Cochrane Database Syst Rev 2007; CD000277.). More effective treatment regimens are still required.

Nimodipine was noted to be a somewhat cerebral-artery selective vasodilator that was studied in patients with SAH in order to reduce angiographic vasospasm. In the clinical trials, however, a minimal or no decrease in angiographic vasospasm was observed in patients treated with nimodipine. Despite this and unlike other vasodilator drugs (Macdonald R L, Kassell N F, Mayer S, et al. Clazosentan to overcome neurological ischemia and infarction occurring after subarachnoid hemorrhage (CONSCIOUS-1): randomized, double-blind, placebo-controlled phase 2 dose-finding trial. Stroke 2008; 39:3015-21; Etminan N, Vergouwen M D, Ilodigwe D, Macdonald R L. Effect of pharmaceutical treatment on vasospasm, delayed cerebral ischemia, and clinical outcome in patients with aneurysmal subarachnoid hemorrhage: a systematic review and meta-analysis. J Cereb Blood Flow Metab 2011; 31:1443-51.), it improved clinical outcome. In addition to being a potent vasodilator even of arteries with angiographic vasospasm (Barth M, Capelle H H, Weidauer S, et al. Effect of nicardipine prolonged-release implants on cerebral vasospasm and clinical outcome after severe aneurysmal subarachnoid hemorrhage: a prospective, randomized, double-blind phase IIa study. Stroke 2007; 38:330-6; Pierot L, Aggour M, Moret J. Vasospasm after aneurysmal subarachnoid hemorrhage: recent advances in endovascular management. Curr Opin Crit Care 2010), it was later noted that nimodipine administered intravenously inhibited other delayed effects of SAH that may contribute to DCI, such as cortical spreading ischemia (Dreier J P, Windmuller O, Petzold G, et al. Ischemia triggered by red blood cell products in the subarachnoid space is inhibited by nimodipine administration or moderate volume expansion/hemodilution in rats. Neurosurgery 2002; 51:1457-65), and that it had fibrinolytic activity that could reduce microthromboemboli (Vergouwen M D, Vermeulen M, de Haan R J, Levi M, Roos Y B. Dihydropyridine calcium antagonists increase fibrinolytic activity: a systematic review. J Cereb Blood Flow Metab 2007; 27:1293-308.). Other vasodilators may not have these pleiotrophic effects (Sabri M, Ai J, Macdonald R L. Dissociation of vasospasm and secondary effects of experimental subarachnoid hemorrhage by clazosentan. Stroke 2011; 42:1454-60.). One hypothesis is that nimodipine improved outcome after SAH because it reduced these complications of SAH but that in the doses administered the effects were not measurable in clinical trials.

There have been prior reports of sustained-release formulations of dihydropyridines and other drugs administered into the subarachnoid space for SAH (Barth M, Capelle H H, Weidauer S, et al. Effect of nicardipine prolonged-release implants on cerebral vasospasm and clinical outcome after severe aneurysmal subarachnoid hemorrhage: a prospective, randomized, double-blind phase IIa study. Stroke 2007; 38:330-6; Bege N, Renette T, Endres T, et al. In situ forming nimodipine depot system based on microparticles for the treatment of posthemorrhagic cerebral vasospasm. Eur J Pharm Biopharm 2013; Omeis I, Jayson N A, Murali R, Abrahams J M. Treatment of cerebral vasospasm with biocompatible controlled-release systems for intracranial drug delivery. Neurosurgery 2008; 63:1011-9). The limitations of these formulations include lack of characterization of pharmacokinetics, stability and injectability, use of materials with known or unknown toxicity and limited data on efficacy of the active drug.

10.1. Treatment of SAH

The management of SAH consists of general measures to stabilize the patient, specific measures to prevent rebleeding by obliterating the bleeding source, prevention of vasospasm, and prevention and treatment of complications.

General Measures

The first priority is to stabilize the patient. Those with a depressed level of consciousness may need to be intubated and mechanically ventilated. Blood pressure, pulse, respiratory rate and Glasgow Coma Scale are monitored frequently. Once the diagnosis is confirmed, admission to an intensive care unit may be preferable, especially given that 15% of such patients have a further episode (rebleeding) in the first hours after admission. Nutrition is an early priority, with oral or nasogastric tube feeding being preferable over parenteral routes. Analgesia (pain control) is important in order to permit good blood pressure control but must be balanced against oversedating patient, which impacts mental status and thus interfere with the ability to monitor the level of consciousness. Deep vein thrombosis is prevented with compression stockings, intermittent pneumatic compression of the calves, pharmacologic agents or a combination.

Prevention of Rebleeding

Patients with a large intracerebral hematoma associated with depressed level of consciousness or focal neurological symptoms may be candidates for urgent surgical removal of the hematoma and neurosurgical clipping of the ruptured aneurysm. A catheter or tube may be inserted into the ventricles to treat hydrocephalus. The remainder are stabilized and undergo a transfemoral catheter angiogram or CT angiogram later. After the first 24 hours, rebleeding risk remains about 20% over the subsequent four weeks, leading to the recommendation that the aneurysm should be repaired by clipping or endovascular coiling as soon as possible.

Rebleeding is hard to predict but may happen at any time and carries a dismal prognosis. Interventions to prevent rebleeding, therefore are performed as early as possible. If a cerebral aneurysm is identified on angiography, two measures are available to reduce the risk of further bleeding from the same aneurysm: neurosurgical clipping and endovascular coiling. Clipping requires a craniotomy (opening of the skull) to locate the aneurysm, followed by the placement of a clip or clips across the neck of the aneurysm. Coiling is performed through the large blood vessels: a catheter is inserted into the femoral artery in the groin, and advanced through the aorta to the arteries (both carotid arteries and both vertebral arteries) that supply the brain. When the aneurysm has been located, metallic coils are deployed that lead to formation of a blood clot in the aneurysm and obliteration. The decision as to which treatment is undertaken typically is made by a multidisciplinary team, often including a neurosurgeon and a neuroradiologist.

Aneurysms of the middle cerebral artery and its related vessels are hard to reach and of less optimal configuration for endovascular coiling and tend to be amenable to clipping, while those of the basilar artery and posterior arteries are hard to reach surgically and tend to be more accessible for endovascular management. The main drawback of coiling is the possibility that the aneurysm may recur; this risk is lower in the surgical approach. Patients who have undergone coiling are typically followed up for many years with angiography or other measures to ensure recurrence of aneurysms is identified early.

10.2. Current Treatment Options for Aneurysmal SAH

Changes in management of patients with aneurysmal SAH, including early neurosurgical aneurysm clipping or endovascular coiling, nimodipine and improved intensive care, are believed to account for the reduction in overall mortality due to aneurysmal SAH, and to a reduction in the contribution of angiographic vasospasm and DCI to death and disability after aneurysmal SAH. (Lovelock C E et al., “Antithrombotic Drug Use, Cerebral Microbleeds, and Intracerebral Hemorrhage. A Systematic Review of Published and Unpublished Studies,” Stroke, 41(6): 1222-1228 (2010)).

Rhoney et al. presents a review on the currently available treatment considerations in the management of aneurysmal SAH. (Rhoney, D. H. et al., “Current and future treatment considerations in the management of aneurysmal subarachnoid hemorrhage,” J. Pharm. Pract., 23(5): 408-424 (2010)). Treatment is usually divided into three categories: supportive therapy, prevention of complications and treatment of complications. Initial supportive therapy upon diagnosis of aneurysmal SAH can include, but is not limited to, to ensuring adequate oxygenation, prevention of blood pressure fluctuations, isotonic or hypertonic IV fluids in order to maintain systemic euvolemia, and avoidance of hyperthermia, hyponatremia, hypoglycemia, hyperglycemia, hypotension, hypomagnesemia, hypercarbia and hypoxia. Rebleeding can be reduced by maintaining systolic blood pressure below a threshold value that varies from patient to patient until the aneurysm is secured by endovascular coiling or neurosurgical clipping. Administration of anti-fibrinolytic agents, such as tranexamic acid or amniocaproic acid until the aneurysm is repaired is an option. Medical complications, such as stress related mucosal damage prophylaxis is used either with proton pump inhibitors or histamine type 2 blocking agents in patients at risk for stress ulceration. Venous thrombo-embolism (VTE) prophylaxis is implemented either through a mechanical device or chemically with anticoagulants, such as heparin or enoxaparin. Glycemic control is utilized to maintain a serum glucose range between 80-140 mg/dL.

Nimodipine, a dihydropyridine L-type calcium channel antagonist, has slight selective vasodilatory action on cerebral compared to systemic arteries, and is the only widely used drug for SAH (Towart R, Wehinger E, Meyer H, Kazda S. The effects of nimodipine, its optical isomers and metabolites on isolated vascular smooth muscle. Arzneimittelforschung 1982; 32:338-46.). Clinically, however, the dose that can be administered is limited because L-type calcium channels are located on arteries throughout the brain and body and doses that dilate the cerebral arteries have some dilatory effect on systemic arteries, causing potentially deleterious adverse effects such as hypotension. One theoretical solution has been to deliver drugs directly to the cerebral arteries in the subarachnoid space. Surgically-implanted pellets containing nicardipine, another dihydropyridine, have been suggested to reduce DCI and cerebral infarction and to improve outcome in experimental and clinical studies of aneurysmal SAH (Kasuya H, Onda H, Sasahara A, Takeshita M, Hori T. Application of nicardipine prolonged-release implants: analysis of 97 consecutive patients with acute subarachnoid hemorrhage. Neurosurgery 2005; 56:895-902; Barth M, Capelle H H, Weidauer S, et al. Effect of nicardipine prolonged-release implants on cerebral vasospasm and clinical outcome after severe aneurysmal subarachnoid hemorrhage: a prospective, randomized, double-blind phase IIa study. Stroke 2007; 38:330-6.). However, the pellets can only be implanted during a craniotomy conducted for aneurysm repair and currently at least 50% of aneurysms are repaired endovascularly. In addition, although the pellets are potentially beneficial to surgically-treated patients, they are prepared with dichloromethane, a neurotoxin that may not be optimal for human use (Kasuya H, Onda H, Sasahara A, Takeshita M, Hori T. Application of nicardipine prolonged-release implants: analysis of 97 consecutive patients with acute subarachnoid hemorrhage. Neurosurgery 2005; 56:895-902.). Furthermore, the pellets remain where they are implanted surgically and do not flow throughout the subarachnoid space to exert a diffuse effect on the complications of SAH that lead to DCI and poor outcome.

Nicardipine is a short acting dihydropyridine calcium channel antagonist that is more water soluble than nimodipine. Nicardipine has an onset action of 1 to 5 minutes and duration of action up to 3 hours. High blood pressure associated with SAH can be managed with nicardipine. Alternatively be treated with alpha/beta adrenergic antagonists, such as labetalol. Clevidipine is an alternative dihydropyridine calcium channel antagonist that can lower blood pressure with a quick offset of effect within 5 to 15 minutes. Esmolol is an antihypertensive agent that can be used with in the treatment of hypertension in patients with acute neurological illness. The effect of any antihypertensive agent on cerebral oxygenation is another consideration factor.

10.3. Treatment of Secondary Complications Associated with SAH

Current treatments to prevent or reduce angiographic vasospasm and DCI consist of measures to prevent or minimize secondary brain injury, use of calcium channel antagonists, hemodynamic management and endovascular therapies. Therapy often is initiated prophylactically in patients and may include: (in stage 1) hemodynamic stabilization including maintaining normovolemia, managing blood pressure and orally-administered L-type voltage-gated calcium channel antagonists; and (in stage 2) further hemodynamic manipulation or infusion of vasodilator drugs into vasospastic arteries or dilating them with balloons. However, the aforementioned treatments are expensive, time consuming and only partially effective.

For over 35 years, physicians have been trying to prevent or reduce the incidence of adverse consequences of SAH, including angiographic vasospasm and DCI, and have had limited effect due to side effects of current agents or lack of efficacy. There currently are no FDA approved agents for the prevention of vasospasm or the reduction of delayed ischemic neurologic deficits also known as delayed cerebral ischemia (DCI). Current methods to prevent vasospasm have failed due to lack of efficacy or to safety issues, primarily hypotension and cerebral edema. Currently, the only FDA-approved available agent is nimodipine, which has minimal effect on angiographic vasospasm in clinically-used doses, although it improved outcome in SAH patients.

Voltage-dependent calcium channel antagonists may be effective in preventing and reversing vasospasm to a certain extent, however, prior art treatments administer doses too low to exert a maximal pharmacologic effect. Endothelin-receptor antagonists also may be effective at preventing and reversing angiographic vasospasm to a certain extent, but this reversal or prevention of angiographic vasospasm does not translate into as marked an improvement in outcome as would be anticipated by the reduction in angiographic vasospasm. Without being limited by theory, it is postulated that the systemic delivery of the voltage-dependent calcium channel antagonists may cause side effects that mitigate the beneficial effects on angiographic vasospasm, such as, for example, systemic hypotension and pulmonary vasodilation with pulmonary edema, which prevent the administration of higher systemic doses. Dilation of blood vessels in the lungs also may cause lung edema and lung injury. Without being limited by theory, it is postulated that systemic delivery of the voltage-dependent calcium channel antagonists may limit other effects of SAH that contribute to DCI, including cortical spreading ischemia and microthromboemboli.

Treatment of DCI

Current treatment for DCI that develops after aneurysmal SAH includes oral or intravenous nimodipine in North America and Europe for up to 3 weeks post aneurysmal SAH. Medical management directed at optimizing cerebral blood flow by raising the blood pressure and avoiding factors that adversely affect cerebral blood flow or that increase brain metabolism are believed to be important. If, despite these measures, a patient deteriorates from DCI, rescue therapies are instituted, including induced hypertension, cerebral balloon angioplasty or local administration of calcium channel antagonists or other vasodilators.

Treatment of Vasospasm

Nimodipine, an oral calcium channel antagonist, has been shown in clinical trials to reduce the chance of a poor outcome, however it may not significantly reduce the amount of angiographic vasospasm detected on angiography. Other calcium channel antagonists and magnesium sulfate have been studied, but are not presently recommended. There is no evidence that shows benefit if nimodipine is given intravenously but the studies conducted have included small numbers of patients. In traumatic SAH, the efficacy of oral nimodipine remains in question.

When administered in the doses used clinically for oral or intravenous administration, nimodipine is associated with dose-limiting hypotension in up to 50% of patients. (Radhakrishnan D, and Menon D K, “Haemodynamic effects of intravenous nimodipine following aneurysmal subarachnoid haemorrhage: implications for monitoring,” Anaesthesia, 52:489-491 (1997)). Plasma concentrations exceed those associated with hypotension, yet CSF concentrations are well below therapeutic concentrations. (Allen G. S. et al., “Cerebral arterial spasm—a controlled trial of nimodipine in patients with subarachnoid hemorrhage,” N. Engl. J. Med. 308:619-624 (1983)). Hypotension is deleterious to patients with aneurysmal SAH because it may lower cerebral perfusion pressure and worsen DCI. (Dankbaar J W et al., “Effect of different components of triple-H therapy on cerebral perfusion in patients with aneurysmal subarachnoid haemorrhage: a systematic review,” Crit. Care, 14:R23 (2010); Darby J. M. et al., “Acute cerebral blood flow response to dopamine-induced hypertension after subarachnoid hemorrhage,” J. Neurosurg., 80:857-864 (1994)).

Evidence suggesting that nimodipine can have neuroprotective effects is not conclusive. For example, Aslan et al. found that intravenous administration of nimodipine to patients with severe traumatic brain injury resulted in significantly higher cerebral perfusion pressure (CPP), higher jugular venous oxygen saturation, and higher scores on Glasgow Coma Scale, while lower intracranial pressure, jugular lactate and glucose levels, in treated versus control groups. However, the study was limited to patients who had severe head trauma with a Glasgow Coma Scale ≤8 and patients with traumatic or chronic lung pathology or brain lesion who required surgical intervention were excluded from this study. (Aslan, A. et al., “Nimodipine can improve cerebral metabolism and outcome in patients with severe head trauma,” Pharmacol. Res., 59(2): 120-124 (2008)). Zhao et al. (1) reported that intravenous administration of nimodipine in a cisterna magna SAH rat model is capable of restoring the regional cerebral blood flow that is significantly reduced as a result of SAH; (2) reported the concomitant nimodipine-induced angiographic dilation of major cerebral arteries that were constricted as a result of SAH, and (3) demonstrated that the integrity of the blood brain barrier, which is disrupted as a result of SAH correlating with poor neurologic grade, can be restored with nimodipine administration. (Zhao, W. J. et al., “Nimodipine attenuation of early brain dysfunctions is partially related to its inverting acute vasospasm in a cisterna magna subarachnoid hemorrhage (SAH) model in rats,” Int. J. Neurosci., PMID: 22694164 (2012)). Nimodipine has also been reported to enhance the excitability of hippocampal neurons in a rabbit study. (Disterhot. J. F. et al., “Nimodipine facilitates learning and increases excitability of hippocampal neurons in aging rabbits,” Drugs in Development, 2: 395-403; discussion, p. 405, (1993)).

Dreier et al. reported that intravenous administration of nimodipine to rats can reverse cortical spreading ischemia after SAH triggered by hemoglobin in rats to cortical spreading hyperemia, but conceded that no conclusion could be drawn from their study regarding territorial infarctions after SAH, which likely include other pathogenic cascades. (Dreier, J. P. et al., “Ischemia triggered by red blood cell products in the subarachnoid space is inhibited by nimodipine administration or moderate volume expansion/hemodilution in rats,” Neurosurgery, 51(6): 1457-1465 (2002)).

Hemodynamic manipulation, previously referred to as “triple H” therapy, often is used as a measure to treat angiographic vasospasm and DCI. This entails the use of intravenous fluids and vasoconstrictor drugs to achieve a state of hypertension (high blood pressure), hypervolemia (excess fluid in the circulation) and hemodilution (mild dilution of the blood). Induced hypertension is believed to be the most important component of this treatment although evidence for the use of this approach is inconclusive, and no sufficiently large randomized controlled trials ever have been undertaken to demonstrate its benefits. At present, hypervolemia and hemodilution are generally not recommended.

If angiographic vasospasm or DCI is resistant to medical treatment, angiography may be attempted to identify the sites of angiographic vasospasm and to administer vasodilator medication (drugs that relax the blood vessel wall) directly into the artery (pharmacological angioplasty) and mechanical angioplasty (opening the constricted area with a balloon) may be performed.

Removal of subarachnoid blood clots with recombinant tissue plasminogen activator (r-t-PA) in patents with aneurysmal SAH has been reported to reduce angiographic vasospasm and DCI but with inconclusive results due to the small number of patients treated and the fact that there is only one randomized, blinded trial (Amin-Hanjani, S. et al., “Does intracisternal thrombolysis prevent vasospasm after aneurysmal subarachnoid hemorrhage? A meta-analysis,” Neurosurgery, 54(2): 326-334; discussion 334-335 (2004); Kramer A H, Fletcher J J: Locally-administered intrathecal thrombolytics following aneurysmal subarachnoid hemorrhage: a systematic review and meta-analysis. Neurocrit Care 14: 489-499 (2011)). Hydroxymethylglutaryl coenzyme A reductase inhibitors (statins), such as simvastatin, pravastatin, etc. have also become routine practice at some institutions for the prevention of angiographic vasospasm and DCI following aneurysmal SAH owing to their pleiotropic effects. In experimental models, statins are associated with increase endothelial nitric oxide (NO) production, anti-inflammatory effects by inhibition of adhesion molecules, free radical scavenging and inhibition of platelet aggregation (McGirt, M. J. et al., “Simvastatin increases endothelial nitric oxide synthase and ameliorates cerebral vasospasm resulting from subarachnoid hemorrhage,” Stroke, 33(12): 2950-2956 (2002); McGirt, M. J. et al., “Systemic administration of simvastatin after the onset of experimental subarachnoid hemorrhage attenuates cerebral vasospasm,” Neurosurgery, 58(5): 945-951; discussion 945-951 (2006)). On the other hand, a large, randomized, double-blind clinical trial and meta analysis of the other trials found that simvastatin did not improve outcome after SAH (Kirkpatrick P J, Turner C L, Smith C, Hutchinson P J, Murray G D: Simvastatin in aneurysmal subarachnoid haemorrhage (STASH): a multicentre randomised phase 3 trial. Lancet Neurol, 2014).

Magnesium, acting as an NMDA receptor antagonist and calcium channel blocker leading to smooth muscle relaxation and vessel dilation, has been investigated for the prevention of angiographic vasospasm and DCI (Macdonald, R. L. et al., “Magnesium and experimental vasospasm,” J. Neurosurg., 100(1): 106-110 (2004)). Hypomagnesemia is common following aneurysmal SAH and is associated with poor outcome and development of angiographic vasospasm (van den Bergh, W. M. et al., “Magnesium sulfate in aneurysmal subarachnoid hemorrhage: a randomized controlled trial,” Stroke, 36(5): 1011-1015 (2005)). A randomized clinical trial that included 1204 patients did not find that intravenous magnesium sulphate improved outcome in patients with SAH (Dorhout Mees, S. M. et al., “Magnesium for aneurysmal subarachnoid haemorrhage (MASH-2): a randomised placebo-controlled trial,” Lancet 380:44-49 (2012)). Meta-analysis of the 7 main randomized trials of magnesium in SAH confirmed this so that routine administration of intravenous magnesium to raise serum magnesium concentrations above normal is not recommended.

Clazosentan, a selective endothelin (ET) receptor antagonist, was the subject of investigation in the CONSCIOUS trials. In the CONSCIOUS-1 study, clazosentan significantly reduced the incidence of angiographic vasospasm after aneurysmal SAH (Macdonald, R. L. et al., “Clazosentan to overcome neurological ischemia and infarction occurring after subarachnoid hemorrhage (CONSCIOUS-1): randomized, double-blind, placebo-controlled phase 2 dose-finding trial,” Stroke, 39(11): 3015-3021 (2008). CONSCIOUS-2 was a randomized, double-blind, placebo-controlled, phase 3 study that assigned patients with SAH secured by surgical clipping to clazosentan (5 mg/h, n=768) or placebo (n=389) for up to 14 days. The primary composite endpoint (week 6) included all-cause mortality, vasospasm-related new cerebral infarcts, delayed ischemic neurological deficit due to vasospasm, and rescue therapy for vasospasm. In the all-treated dataset, the primary endpoint was met in 161 (21%) of 764 clazosentan-treated patients and 97 (25%) of 383 placebo-treated patients (relative risk reduction 17%, 95% CI −4 to 33; p=0.10). Poor functional outcome (GOSE score <1=4) occurred in 224 (29%) clazosentan-treated patients and 95 (25%) placebo-treated patients (−18%, −45 to 4; p=0.10). Lung complications, anaemia, and hypotension were more common with clazosentan. Mortality (week 12) was 6% in both groups. Clazosentan at 5 mg/h had no significant effect on mortality and vasospasm-related morbidity or functional outcome. (Macdonald, R. L. et al., “Clazosentan, an endothelin receptor antagonist, in patients with aneurysmal subarachnoid haemorrhage undergoing surgical clipping: a randomised, double-blind, placebo-controlled phase 3 trial (CONSCIOUS-2),” Lancet Neurol. 10:618-625 (2011). CONSCIOUS-3 was a double-blind, placebo-controlled, randomized phase III trial in patients with SAH secured by endovascular coiling and randomized to <1=14 days intravenous clazosentan (5 or 15 mg/h) or placebo (Macdonald, R. L. et al., “Randomized trial of clazosentan in patients with aneurysmal subarachnoid hemorrhage undergoing endovascular coiling,” Stroke 43:1463-1469 (2012)). The primary composite end point was the same as CONSCIOUS-2. CONSCIOUS-3 was halted prematurely following completion of CONSCIOUS-2; 577/1500 of planned patients (38%) were enrolled and 571 were treated (placebo, n=189; clazosentan 5 mg/h, n=194; clazosentan 15 mg/h, n=188). The primary end point occurred in 50/189 of placebo-treated patients (27%), compared with 47/194 patients (24%) treated with clazosentan 5 mg/h (odds ratio [OR], 0.786; 95% CI, 0.479-1.289; P=0.340), and 28/188 patients (15%) treated with clazosentan 15 mg/h (OR, 0.474; 95% CI, 0.275-0.818; P=0.007). Poor outcome (extended Glasgow Outcome Scale score <1=4) occurred in 24% of patients with placebo, 25% of patients with clazosentan 5 mg/h (OR, 0.918; 95% CI, 0.546-1.544; P=0.748), and 28% of patients with clazosentan 15 mg/h (OR, 1.337; 95% CI, 0.802-2.227; P=0.266). Pulmonary complications, anemia, and hypotension were more common in patients who received clazosentan than in those who received placebo. Clazosentan 15 mg/h significantly reduced post aneurysmal SAH vasospasm-related morbidity/all-cause mortality; however, neither dose improved outcome (extended Glasgow Outcome Scale). Clazosentan currently is not approved for use for SAH patients.

Current therapies to prevent or reduce SAH, the incidence of secondary complications after SAH, such as DCI and angiographic vasospasm, are risky, only marginally efficacious, expensive and time-consuming. Thus, there is a large unmet medical need for safe, effective treatments to reduce the need for rescue therapy and improve functional outcome. While conventional therapies have been focusing on treating angiographic vasospasm following SAH, accumulating evidence suggests that there are additional complications derived from SAH, which need to be targeted for treatment interventions to improve prognosis following SAH treatment. The described invention, which provides a sustained-release pharmaceutical formulation comprising materials with brain biocompatibility, methods that allow programming of the polymers for desired release characteristics, stability, commercial scalability and an active pharmacologic agent with an established safety profile, offers such an approach.

### SUMMARY

According to one aspect, the described invention provides a site-specific microparticulate pharmaceutical formulation, comprising: (a) a therapeutic amount of an L-type voltage-gated calcium channel antagonist; (b) a poly(DL-lactide-co-glycolide) (PLGA) polymer comprising from 25% to 50% glycolide; and (c) less than 5% hyaluronic acid, wherein the microparticles of the formulation are characterized by: (i) a particle size from about 20 μm to about 125 μm; (ii) a drug load of from about 50% to about 70%; (iii) sustained release; and at least 99% purity.

According to another aspect, the described invention provides a site-specific microparticulate pharmaceutical formulation for administration into an intracisternal site of administration comprising, (i) from 40 mg to about 1200 mg of an L-type voltage gated calcium channel antagonist; (ii) a poly(DL-lactide-co-glycolide) (PLGA) polymer comprising 50% glycolide; (iii) less than 5% of a hyaluronic acid characterized by a zero shear rate viscosity of 1677 Poise, molecular weight 1.0-2.9 million Da; wherein consistency of the formulation is that of a paste, wherein the microparticles of the formulation are characterized by: (a) a particle size from about 20 μm to about 125 μm; (b) a drug load of from about 50% to about 70%; (c) sustained release; and (d) at least 99% purity.

According to another aspect, the described invention provides a site-specific microparticulate pharmaceutical formulation for administration into an intraventricular, intracisternal or intrathecal site of administration, comprising: (a) from 40 mg to 1200 mg of an L-type voltage gated calcium channel antagonist; (b) a poly(DL-lactide-co-glycolide) (PLGA) polymer comprising 50% glycolide; (c) less than 5% of a hyaluronic acid characterized by a zero shear rate viscosity of 2 Poise, molecular weight of 0.500-0.750 million Da, wherein viscosity of the formulation ranges from about 1.5 Poise to about 3.5 poise, wherein the microparticles of the formulation are characterized by: (i) a particle size from about 20 μm to about 125 μm; (ii) a drug load of from about 50% to about 70%; (iii) sustained release; and (iv) at least 99% purity.

According to one embodiment, the L-type voltage-gated calcium channel antagonist is a dihydropyridine. According to another embodiment, the dihydropyridine is selected from the group consisting of nimodipine, nifedipine, nicardipine, clevidipine, and nisoldipine. According to another embodiment, the formulation comprises about 40 mg to about 1200 mg of nimodipine. According to another embodiment, the nimodipine contains at least 51% Form 1 of nimodipine.

According to one embodiment, the hyaluronic acid is characterized by a zero shear rate viscosity of 2 Poise and a molecular weight from about 0.500 million to about 0.750 million Da. According to another embodiment, the hyaluronic acid is characterized by a zero shear rate viscosity of 1677 Poise and a molecular weight from about 1.0 million to about 2.9 million Da.

According to one embodiment, the formulation is stable after storage for up to 12 months at −25° C. and 5° C.

According to one embodiment, initial burst of release of the therapeutic agent within 24 hours of administration is <25%.

According to one embodiment, the mean particle size is about 70 μm to about 100 μm.

According to one embodiment, the site-specific microparticulate pharmaceutical formulation is prepared by a process comprising: (a) providing the at least 51% pure L-type voltage gated calcium channel antagonist; (b) adding the L-type voltage gated calcium channel antagonist to a PLGA polymer solution containing 50% glycolide and a solvent, thereby creating a mixture of the bioactive agent and the polymer solution; (c) homogenizing the mixture to form a disperse phase comprising the L-type voltage gated calcium channel antagonist and the PLGA solution; (d) mixing the disperse phase with a continuous phase comprising a surfactant dissolved in deionized water, thereby forming an emulsion comprising the L-type voltage gated calcium channel antagonist; (e) forming the particles comprising the L-type voltage gated calcium channel antagonist by precipitating the polymer and extracting the solvent; (f) collecting the microparticles on sieves, lyophilizing and storing the microparticles at −20° C.; (g) sterilizing the microparticles using gamma irradiation; (h) drying the particles; and (i) formulating the microparticles with less than 5% of a hyaluronic acid, wherein viscosity of the formulation ranges from about 1.5 Poise to about 3.5 Poise.

According to one embodiment, the solvent is ethyl acetate, the surfactant is PVA, and the process comprises >91% encapsulation efficiency.

According to one embodiment, the described invention provides a method for treating a delayed complication of a brain injury associated with interruption of a cerebral artery comprising a delayed cerebral ischemia, comprising administering a pharmaceutical composition containing a therapeutic amount of the formulation to a subject in need thereof, wherein the therapeutic amount is effective to reduce incidence of a poor outcome, as measured on the Glasgow outcome score (GOS), extended Glasgow outcome score (GOSE), modified rankin scale (mRS), Montreal cognitive assessment, or a neurocognitive assessment compared to the outcome expected without treatment or in patients treated with preservative free saline solution without toxicity in either brain or systemic tissues.

According to one embodiment, the poor outcome is a score of 1, 2 or 3 on the Glasgow outcome scale (GOS). According to another embodiment, the poor outcome is a score of 1, 2, 3 or 4 on the extended Glasgow outcome scale (GOSE). According to another embodiment, the poor outcome is a score of 1, 2, 3, 4 or 5 on the extended Glasgow outcome scale (GOSE).

According to one embodiment, the formulation is terminally sterilized.

### BRIEF DESCRIPTION OF THE DRAWINGS

FIG. 1 shows an illustrative lateral view of the human brain (Stedman's Medical Dictionary, 27^{th} Edition, plate 7 at A7 (2000)).

FIG. 2 shows an illustrative sagittal view of the human brain (Correlative Neuroanatomy & Functional Neurology, 18^{th} Ed., p. 46 (1982)).

FIG. 3 shows an illustrative view of a cross section of the intact meninges from the inner surface of the skull (upper) to the external surface of the brain (lower). Collagen is present in the periosteal and meningeal dura (large dots, orientation of fibrils not indicated) and in the subarachnoid space (SAS), usually in folds of trabecular cells. The dural border cell layer has no extracellular collagen, few cell junctions, enlarged extracellular spaces (but no basement membrane), and fibroblasts that are distinct from those of the outer portions of the dura. The arachnoid barrier cell layer has essentially no extracellular space, numerous cell junctions, more plump appearing cells, and a comparatively continuous basement membrane on its surface toward the SAS. Note the continuity of cell layers from the arachnoid to the dura (no intervening space), the characteristic appearance of the arachnoid trabeculae, and the relationship of the pia (from Haines D E: On the question of subdural space. Anat Rec 230:3-21, 1991).

FIG. 4 is a schematic drawing depicting the meninges and their spaces surrounding the spinal cord. (Kulkarni, N. V., “Clinical anatomy for students: problem solving approach,” Jaypee Brothers Medical Publishers (P) Ltd., New Delhi, p. 348-349 (2006)). The meninges are associated with three spaces: epidural space, subdural space and subarachnoid space.

FIG. 5 shows an illustrative view of the circle of Willis and principal arteries of the brain (Correlative Neuroanatomy & Functional Neurology, 18^{th} Ed., p. 48 (1982)).

FIG. 6 shows an illustrative view of the arterial supply of the cerebral cortex. 1: orbitofrontal artery; 2: prerolandic artery; 3: rolandic artery; 4: anterior parietal artery; 5: posterior parietal artery; 6: angular artery; 7: posterior temporal artery; 8: anterior temporal artery; 9: orbital artery; 10: frontopolar artery; 11: callosomarginal artery; 12: posterior internal frontal artery; 13: pericallosal artery. (Correlative Neuroanatomy & Functional Neurology, 18^{th} Ed., p. 50 (1982)).

FIG. 7 shows an illustrative view of the cerebral arteries.

FIG. 8 shows an illustrative view of the cerebral arteries. (from Netter F H. The CIBA Collection of Medical Illustrations: Volumes 1, Nervous System. Vol. 1. Part I. CIBA: USA. 1986. pp. 256).

FIG. 9 shows an illustrative view of the cerebral ventricles (page 192, Ross L M, Lamperti E D, Taub E (eds), Schuenke M, Schulte E, Schumacher U. Thieme Atlas of Anatomy. Georg Thieme Verlag: Stuttgart. 2006).

FIG. 10 shows an illustrative view of the CSF flow from the ventricles to the subarachnoid space (page 194, Ross L M, Lamperti E D, Taub E (eds), Schuenke M, Schulte E, Schumacher U. Thieme Atlas of Anatomy. Georg Thieme Verlag: Stuttgart. 2006).

FIG. 11A shows a simple flow diagram for prognosis following SAH.

FIG. 11B shows a flow diagram of pathways proposed to be involved in delayed complications after SAH.

FIG. 12 shows time trends in outcome of subarachnoid hemorrhage in seven population-based studies of SAH, which shows 50% decrease in mortality over 20 years.

FIG. 13 shows cumulative release of nimodipine in vitro from 6 nimodipine-PLGA microparticle formulations under optimized sink conditions. Values are means±standard deviations (n=3 per measurement).

FIG. 14 shows cumulative release of nimodipine in vitro from 3 nimodipine-PLGA formulations under optimized sink conditions before and after irradiation (2.2 kGray). There was no change in release characteristics after irradiation. Values are means±standard deviations (n=3 per measurement).

FIG. 15 shows Scanning electron microscopy (A, D, G, J) and Raman spectroscopy (B, C, E, F, H, I, K, L) of fresh nimodipine (A-C, G-I) microparticles or the same formulations after storage at 30-35° C. for 30 days (D-F, J-L). Formulation 00447-108 showed no change in morphology or spectroscopy (top 2 rows, A-F) whereas 00447-110 showed agglomeration of particles after storage and change in nimodipine from amorphous to form 2 (scale bar=20 μm). B, E, H and K: Red is nimodipine, green is PLGA and blue is epoxy in Raman spectroscopy. C, F, I and L: Red is nimodipine form 1, green is amorphous nimodipine and blue is nimodipine form 2 in Raman spectroscopy.

FIG. 16 shows nimodipine plasma concentrations after subcutaneous injection of nimodipine (n=4) or different nimodipine-PLGA formulations in rats. Values are means±standard deviations (n=2-3 per measurement from groups of 6 rats).

FIG. 17 shows histologic (hematoxylin and eosin) effects of nimodipine microparticles in rats sacrificed 15 (top row) or 29 (bottom row) days after intraventricular injection of 0.9% NaCl, placebo microparticles or nimodipine-PLGA microparticles (0.33, 1 or 2 mg nimodipine). Gliosis, hydrocephalus, pigmented and foamy macrophages, focal mineralization and vacuolation was observed after injection of microparticles that was not dose-related. Increased intracranial hemorrhage was observed 15 days after injection of nimodipine-PLGA microparticles (2 mg) that partly resolved by 29 days. Scale bar=500 μm.

FIG. 18 shows plasma concentration of nimodipine in rats after intraventricular injection of 0.9% NaCl, placebo microparticles or nimodipine-PLGA microparticles (0.33, 1 or 2 mg nimodipine). Values are means±standard deviations (n=7-32 per measurement).

FIG. 19A shows blood pressure in beagles after intracisternal or intraventricular injection of 0.9% NaCl, placebo microparticles or nimodipine-PLGA microparticles (17, 51 or 103 mg nimodipine). There were no significant differences between groups at any time (ANOVA). Values are means±standard deviations (n=5-24 per measurement).

FIG. 19B shows ventriculocranial ratio in beagles after intracisternal or intraventricular injection of 0.9% NaCl, placebo microparticles or nimodipine-PLGA microparticles (17, 51 or 103 mg nimodipine). There were no significant differences between groups at any time (ANOVA). Values are means±standard deviations (n=5-24 per measurement).

FIG. 20 shows histologic (hematoxylin and eosin) effects of nimodipine microparticles in beagles sacrificed 15 (top row) or 29 (bottom row) days after intraventricular injection of 0.9% NaCl, intraventricular nimodipine-PLGA microparticles (51 or 103 mg nimodipine) or intracisternal nimodipine-PLGA microparticles (103 mg nimodipine). Granulomatous foreign body type reaction was noted in the cerebral ventricles and subarachnoid space after injection of any of the microparticle formulations. The presence of the foreign material and inflammatory reaction was incompletely resolved by day 29. Scale bar=500 μm.

FIG. 21A shows plasma concentration of nimodipine in beagles after intraventricular or intracisternal injection of nimodipine-PLGA microparticles (17, 51 or 103 mg nimodipine). Values are means±standard deviations.

FIG. 21B shows cerebrospinal fluid (CSF) concentration of nimodipine in beagles after intraventricular or intracisternal injection of nimodipine-PLGA microparticles (17, 51 or 103 mg nimodipine). Values are means±standard deviations.

FIG. 22 shows behavior assessment of mongrel dogs after subarachnoid hemorrhage (SAH) treated with intracisternal placebo microparticles plus oral nimodipine (5.2 mg/kg daily for 21 days), intracisternal nimodipine-PLGA microparticles (40 mg), intracisternal nimodipine-PLGA microparticles (100 mg), intraventricular nimodipine-PLGA microparticles (100 mg) or intracisternal placebo microparticles. The scores were significantly different between groups at each time only for placebo versus 40 mg intracisternal and placebo versus 100 mg intraventricular on day 2 (ANOVA P=0.005, pairwise comparisons by Holm-Sidak P<0.05). There were significant differences over time within groups in behavior, with significantly worse behavior in the placebo group on day 4 compared to days 6, 7, 11, 12, 14 and 28 (ANOVA, P<0.001, pairwise comparisons by Holm-Sidak, P<0.05), oral nimodipine day 2 significantly worse than days 9, 10, 11, 12, 13, 14 and 28 and day 5 significantly different than days 11, 13 and 14 (ANOVA P<0.001, pairwise comparisons by Holm-Sidak, P<0.05), 40 mg intracisternal day 2 significantly worse than days 7, 10, 11, 12, 13, 14 and 28 (ANOVA P=0.025, pairwise comparisons by Holm-Sidak, P<0.05), 100 mg intracisternal day 2 significantly worse than day 28 (ANOVA P<0.001, pairwise comparisons by Holm-Sidak, P<0.05) and 100 mg intraventricular day 2 significantly worse than all other days (ANOVA P<0.001, pairwise comparisons by Holm-Sidak, P<0.05). Values are means±standard deviations (n=6-8 per group).

FIG. 23A shows mean blood pressure in mongrel dogs after subarachnoid hemorrhage (SAH) treated with intracisternal placebo microparticles without (n=8) or with oral nimodipine (n=8, 5.2 mg/kg daily for 21 days), intracisternal nimodipine-PLGA microparticles (n=8, 40 mg), intracisternal nimodipine-PLGA microparticles (n=6, 100 mg) or intraventricular nimodipine-PLGA microparticles (n=8, 100 mg). There were no significant differences in blood pressure within groups over time or between groups at each time (ANOVA). Values are means±standard deviations.

FIG. 23B shows plasma nimodipine concentrations in mongrel dogs after subarachnoid hemorrhage (SAH) treated with intracisternal placebo microparticles without (n=8) or with oral nimodipine (n=8, 5.2 mg/kg daily for 21 days), intracisternal nimodipine-PLGA microparticles (n=8, 40 mg), intracisternal nimodipine-PLGA microparticles (n=6, 100 mg) or intraventricular nimodipine-PLGA microparticles (n=8, 100 mg). Values are means±standard deviations.

FIG. 23C shows cerebrospinal fluid (CSF) nimodipine concentrations in mongrel dogs after SAH treated with intracisternal placebo microparticles without (n=8) or with oral nimodipine (n=8, 5.2 mg/kg daily for 21 days), intracisternal nimodipine-PLGA microparticles (n=8, 40 mg), intracisternal nimodipine-PLGA microparticles (n=6, 100 mg) or intraventricular nimodipine-PLGA microparticles (n=8, 100 mg). Values are means±standard deviations.

FIG. 24 shows percent change in basilar artery diameter 8 and 15 days after SAH in dogs treated with intracisternal placebo microparticles without (n=7) or with oral nimodipine (n=8, 5.2 mg/kg daily for 21 days), intracisternal nimodipine-PLGA microparticles (n=8, 40 mg), intracisternal nimodipine-PLGA microparticles (n=6, 100 mg) or intraventricular nimodipine-PLGA microparticles (n=8, 100 mg). Significantly less angiographic vasospasm was observed at 8 days after treatment with 100 mg nimodipine-PLGA microparticles intraventricularly or at 15 days after treatment with 40 mg intracisternal or 100 mg intraventricular nimodipine-PLGA microparticles (P<0.05, ANOVA). Values are means±standard deviations.

### DETAILED DESCRIPTION OF THE INVENTION

### Glossary

The term “active” as used herein refers to the ingredient, component or constituent of the composition of the present invention responsible for the intended therapeutic effect. The term “active ingredient” (“AI”, “active pharmaceutical ingredient”, “API”, or “bulk active”) is the substance in a drug that is pharmaceutically active. As used herein, the phrase “additional active ingredient” refers to an agent, other than a compound of the described formulation, that exerts a pharmacological, or any other beneficial activity.

The term “additive effect”, as used herein, refers to a combined effect of two or more chemicals that is equal to the sum of the effect of each agent given alone.

The term “admixture” or “blend” as used herein generally refers to a physical combination of two or more different components.

The term “administer” as used herein means to give or to apply. The term “administering” as used herein includes in vivo administration, as well as administration directly to tissue ex vivo. Generally, compositions may be administered systemically either orally, buccally, parenterally, topically, by inhalation or insufflation (i.e., through the mouth or through the nose), administered rectally in dosage unit formulations containing conventional nontoxic pharmaceutically acceptable carriers, adjuvants, and vehicles as desired, or administered locally by means such as, but not limited to, injection, implantation, grafting, topical application or parenterally.

The term “agent” as used herein refers generally to compounds that are contained in or on the long-acting formulation. Agent may include an antibody or nucleic acid or an excipient or, more generally, any additive in the long-acting formulation. “Agent” includes a single such compound and is also intended to include a plurality of such compounds.

The term “adverse event” (AE), as used herein, refers to any undesirable change from a patient's baseline condition associated with the use of a medical product in a patient. An undesirable change refers to any unfavorable or unintended sign including, but are not limited to, an abnormal laboratory finding, symptom or disease that occurs during the course of a study, whether or not considered related to the study drug, etc. The term “treatment-emergent AE” as used herein refers to any AE temporally associated with the use of a study drug, whether or not considered related to the study drug.

Exemplary adverse events include but are not limited to, any unfavorable and unintended sign including an abnormal laboratory finding, symptom or disease that occurs during the course of the study, whether or not considered related to the study drug; exacerbation of pre-existing disease; increase in frequency or intensity of a pre-existing episodic disease or medical condition; a disease or medical condition detected or diagnosed after study drug administration even though it may have been present prior to the start of the study; continuous persistent disease or symptoms present at baseline that worsen following the start of the study; lack of efficacy in the acute treatment of a life threatening disease; events considered by the investigator to be related to study mandated procedure; abnormal assessments, e.g., electrocardiographic findings if representing a clinically significant finding not present at baseline or worsened during the course of the study; laboratory test abnormalities if representing a clinically significant finding not present at baseline or worsened during the course of the study or that led to dose reduction, interruption or permanent discontinuation of study drug. Adverse events do not include: a medical or surgical procedure, e.g., surgery, endoscopy, tooth extraction, transfusion; pre-existing disease or a medical condition that does not worsen; or situations in which an adverse change did not occur, e.g., hospitalizations for cosmetic elective surgery.

Adverse events are assessed by the investigators as to whether or not there is a reasonable possibility of causal relationship to the study drug and reported as either related or unrelated. The term “adverse drug reactions related to the study drug” can apply to any adverse event (including serious adverse event) that appears to have a reasonable possibility of a causal relationship to the use of the study drug. The term “adverse drug reactions unrelated to the study drug” applies to any adverse event (including serious adverse event) that does not appear to have a reasonable relationship to the use of the study drug.

The intensity of clinical adverse events is graded on a three-point scale: mild, moderate, severe. If the intensity of an adverse event worsens during study drug administration, only the worst intensity is reported. If the adverse event lessens in intensity, no change in the severity is required. A mild adverse event is one noticeable to subject, but that does not influence daily activities, and usually does not require intervention. A moderate adverse event is one that may make the subject uncomfortable, may influence performance of daily activities, and may require intervention. A severe adverse event is one that may cause noticeable discomfort, usually interferes with daily activities, a result of which a subject may not be able to continue in the study, and for which treatment or intervention is usually needed.

The term “agonist” as used herein refers to a chemical substance capable of activating a receptor to induce a full or partial pharmacological response. Receptors can be activated or inactivated by either endogenous or exogenous agonists and antagonists, resulting in stimulating or inhibiting a biological response. A physiological agonist is a substance that creates the same bodily responses, but does not bind to the same receptor. An endogenous agonist for a particular receptor is a compound naturally produced by the body which binds to and activates that receptor. A superagonist is a compound that is capable of producing a greater maximal response than the endogenous agonist for the target receptor, and thus an efficiency greater than 100%. This does not necessarily mean that it is more potent than the endogenous agonist, but is rather a comparison of the maximum possible response that can be produced inside a cell following receptor binding. Full agonists bind and activate a receptor, displaying full efficacy at that receptor. Partial agonists also bind and activate a given receptor, but have only partial efficacy at the receptor relative to a full agonist. An inverse agonist is an agent which binds to the same receptor binding-site as an agonist for that receptor and reverses constitutive activity of receptors. Inverse agonists exert the opposite pharmacological effect of a receptor agonist. An irreversible agonist is a type of agonist that binds permanently to a receptor in such a manner that the receptor is permanently activated. It is distinct from a mere agonist in that the association of an agonist to a receptor is reversible, whereas the binding of an irreversible agonist to a receptor is believed to be irreversible. This causes the compound to produce a brief burst of agonist activity, followed by desensitization and internalization of the receptor, which with long-term treatment produces an effect more like an antagonist. A selective agonist is specific for one certain type of receptor.

The terms “anastomosis” and “anastomoses” are used interchangeably to refer to interconnections between blood vessels. These interconnections protect the brain when part of its vascular supply is compromised. At the circle of Willis, the two anterior cerebral arteries are connected by the anterior communicating artery and the posterior cerebral arteries are connected to the internal carotid arteries by the posterior communicating arteries. Other important anastomoses include connections between the ophthalmic artery and branches of the external carotid artery through the orbit, and connections at the brain surface between branches of the middle, anterior, and posterior cerebral arteries (Principles of Neural Sciences, 2d Ed., Eric R. Kandel and James H. Schwartz, Elsevier Science Publishing Co., Inc., New York, pp. 854-56 (1985)).

The term “angina pectoris” as used herein refers to a severe constricting chest pain, often radiating from the shoulder to the arm.

The term “angiographic vasospasm” as used herein refers to the reduction of vessel size that can be detected on angiographic exams, including, but not limited to, computed tomographic, magnetic resonance or catheter angiography, occurring in approximately 67% of patients following SAH. On the other hand, the term “clinical vasospasm” as used herein refers to the syndrome of confusion and decreased level of consciousness associated with reduced blood flow to the brain parenchyma, occurring in approximately 30% of patients, and is now defined as DCI.

The term “antagonist” as used herein refers to a substance that interferes with the effects of another substance. Functional or physiological antagonism occurs when two substances produce opposite effects on the same physiological function. Chemical antagonism or inactivation is a reaction between two substances to neutralize their effects. Dispositional antagonism is the alteration of the disposition of a substance (its absorption, biotransformation, distribution, or excretion) so that less of the agent reaches the target or its persistence there is reduced. Antagonism at the receptor for a substance entails the blockade of the effect of an antagonist with an appropriate antagonist that competes for the same site.

The term “anti-inflammatory agent” as used herein refers to an agent that prevents or reduces symptoms associated with inflammation.

The term “anti-coagulant” as used herein refers to an agent that prevents formation of a blood clot.

The term “anti-fibrinolytic agent” as used herein refers to an agent used to prevent dissolution of a fibrin clot.

The terms “aseptic manufacturing” and “aseptic processing” as used herein refer to a process by which a final sterile product is realized over several manufacturing process steps. The products/components are sterilized separately and combined later in a sterile environment to produce the final sterile product.

The term “ataxia” as used herein refers to an inability to coordinate muscle activity during voluntary movement.

The term “bioactive agent” as used herein refers to a compound of interest contained in or on a pharmaceutical formulation or dosage form that is used for pharmaceutical or medicinal purposes to provide some form of therapeutic effect or elicit some type of biologic response or activity. “Bioactive agent” includes a single such agent and is also intended to include a plurality of bioactive agents including, for example, combinations of two or more bioactive agents.

The term “bioavailable” as used herein refers to the rate and extent to which an active ingredient is absorbed from a drug product and becomes available at the site of action.

The term “biocompatible” as used herein refers to that which causes no clinically relevant tissue irritation, injury, toxic reaction, or immunological reaction to living tissue.

The term “biodegradable”, as used herein, refers to a material that will erode to soluble species or that will degrade under physiologic conditions to smaller units or chemical species that are, themselves, non-toxic (biocompatible) to the subject and capable of being metabolized, eliminated, or excreted by the subject.

The term “biomimetic” as used herein refers to materials, substances, devices, processes, or systems that imitate or “mimic” natural materials made by living organisms.

The term “blood vessel”, as used herein, refers to a structure, e.g. a tube or a duct conveying or containing blood. Exemplary blood vessels include, but are not limited to, arteries, arterioles, capillaries, veins and venules.

The term “carrier” as used herein describes a material that does not cause significant irritation to an organism and does not abrogate the biological activity and properties of the active compound of the composition of the described invention. Carriers must be of sufficiently high purity and of sufficiently low toxicity to render them suitable for administration to the mammal being treated. The carrier can be inert, or it can possess pharmaceutical benefits, cosmetic benefits or both. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components.

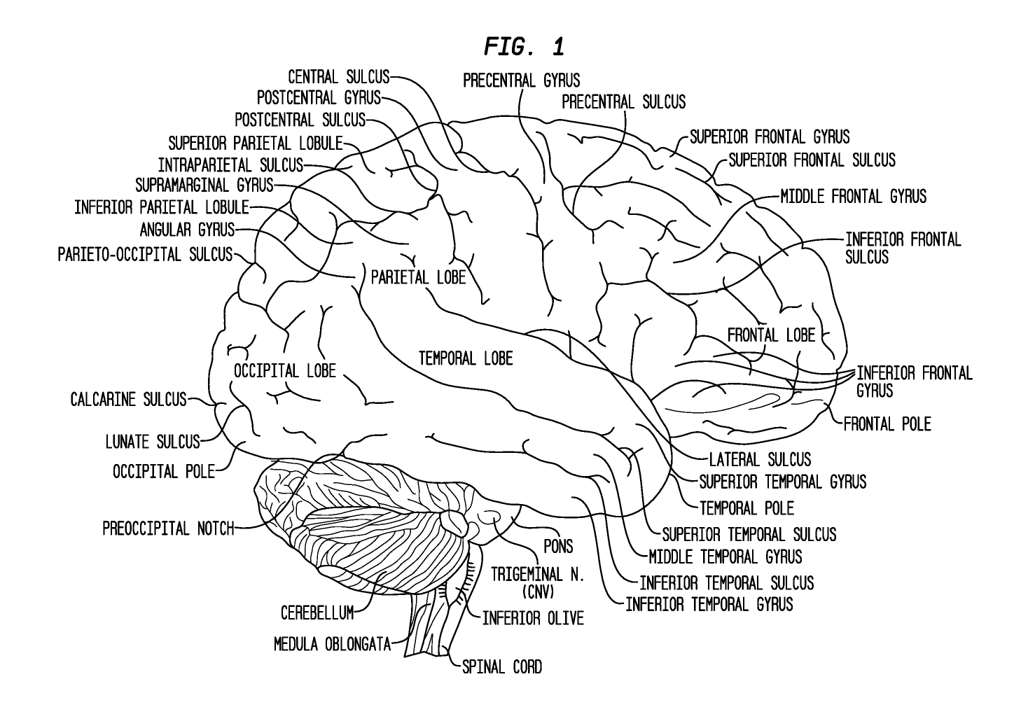

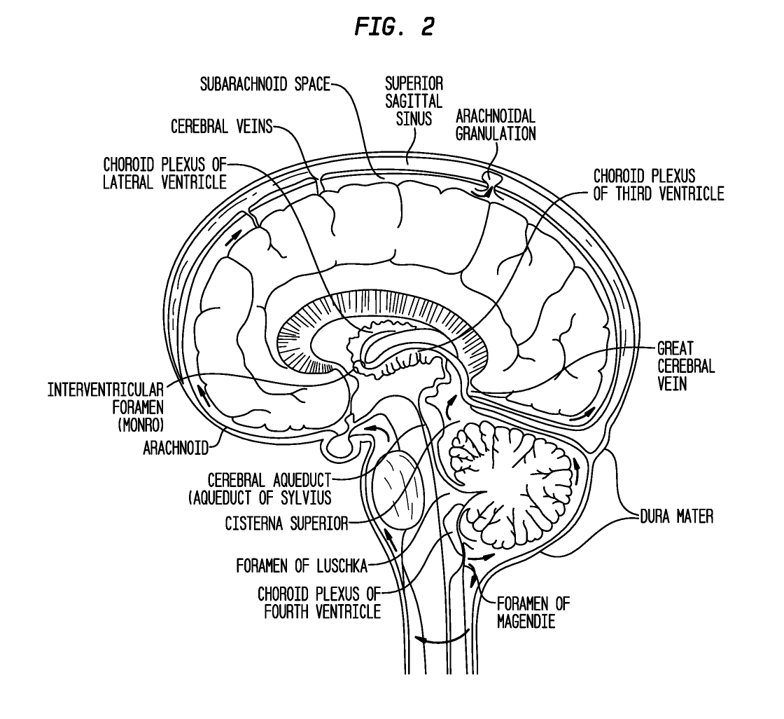

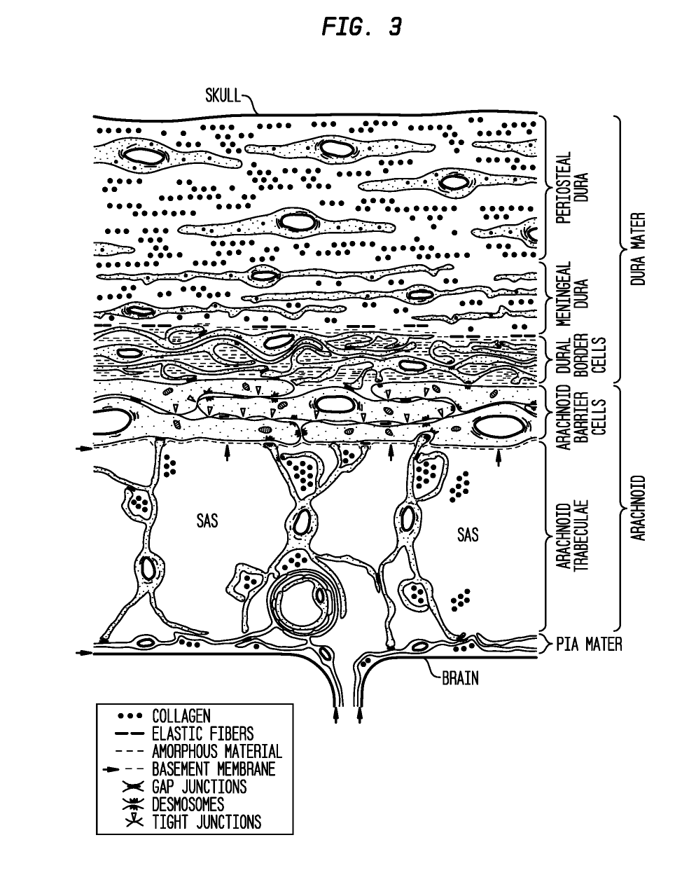

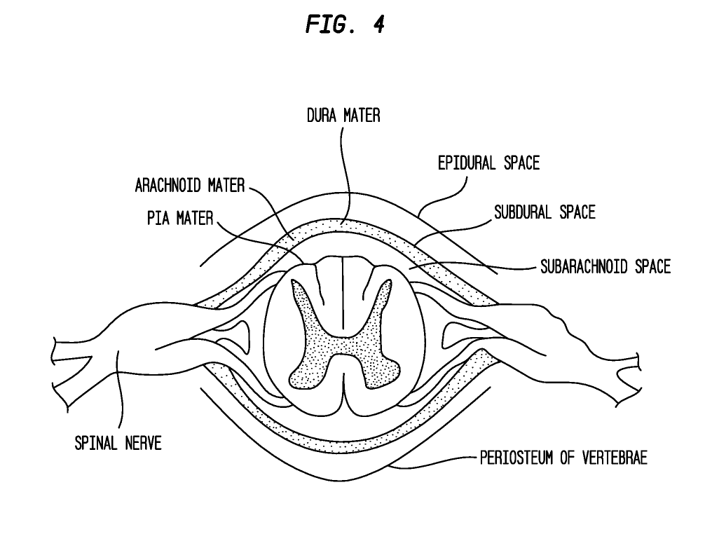

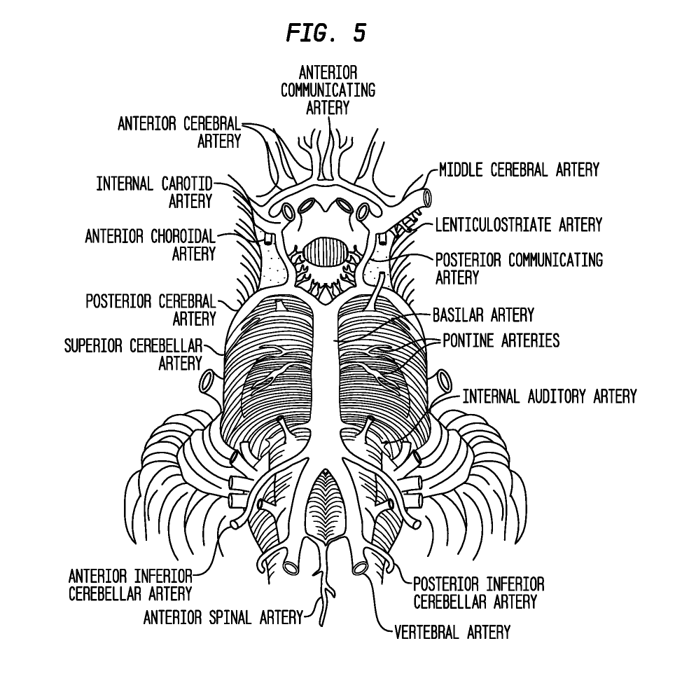

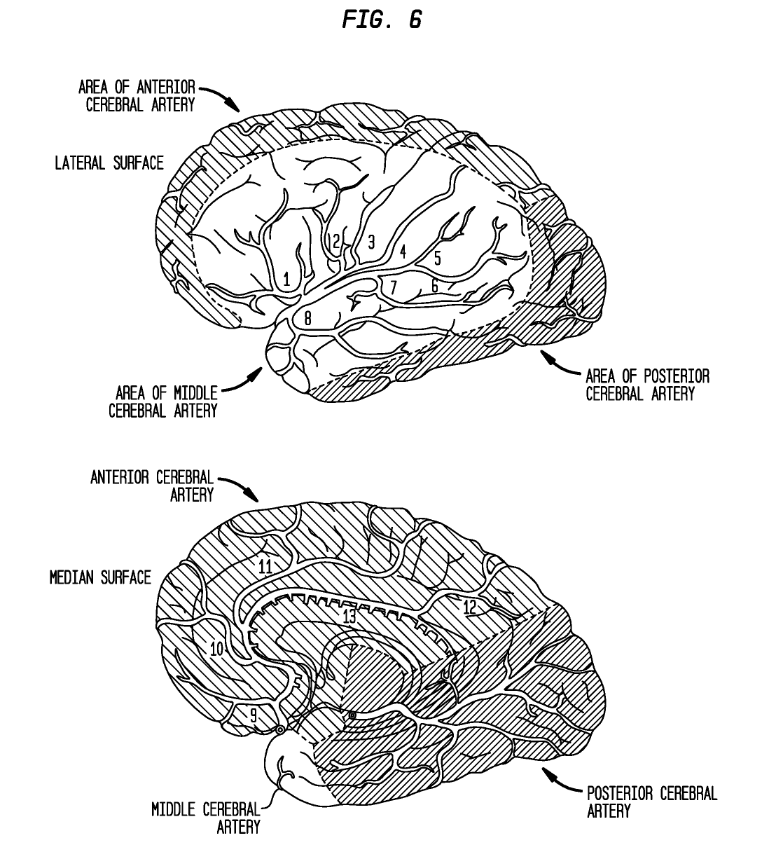

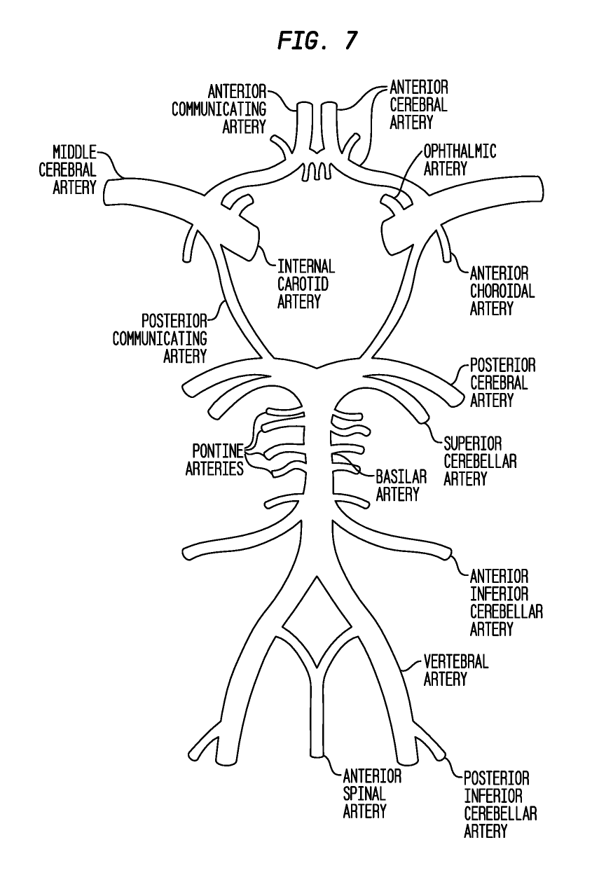

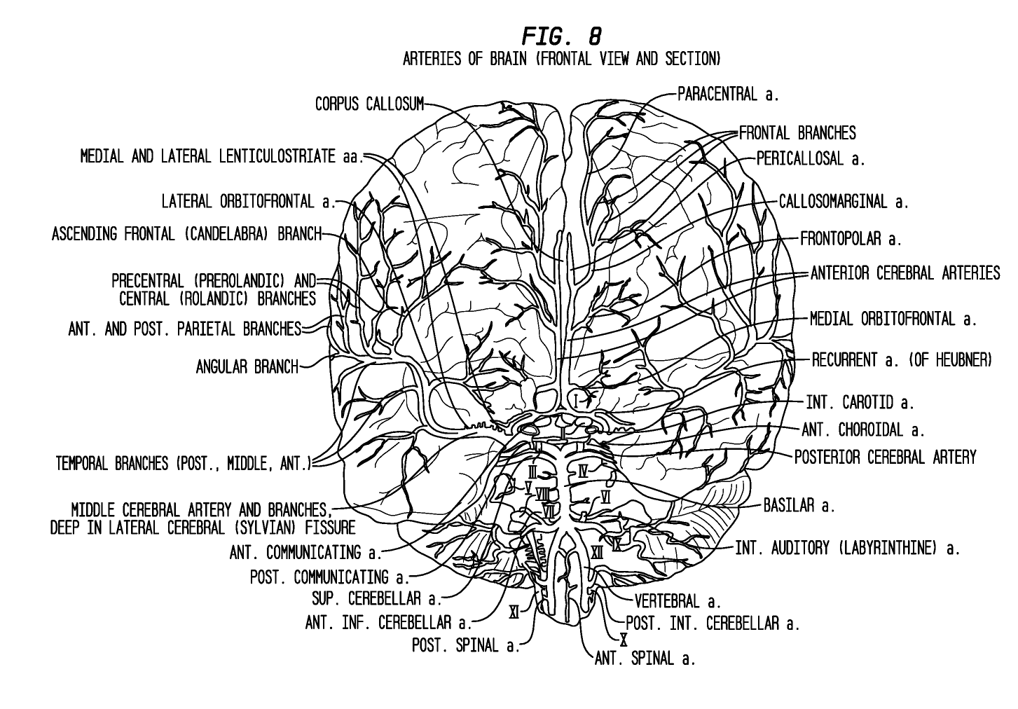

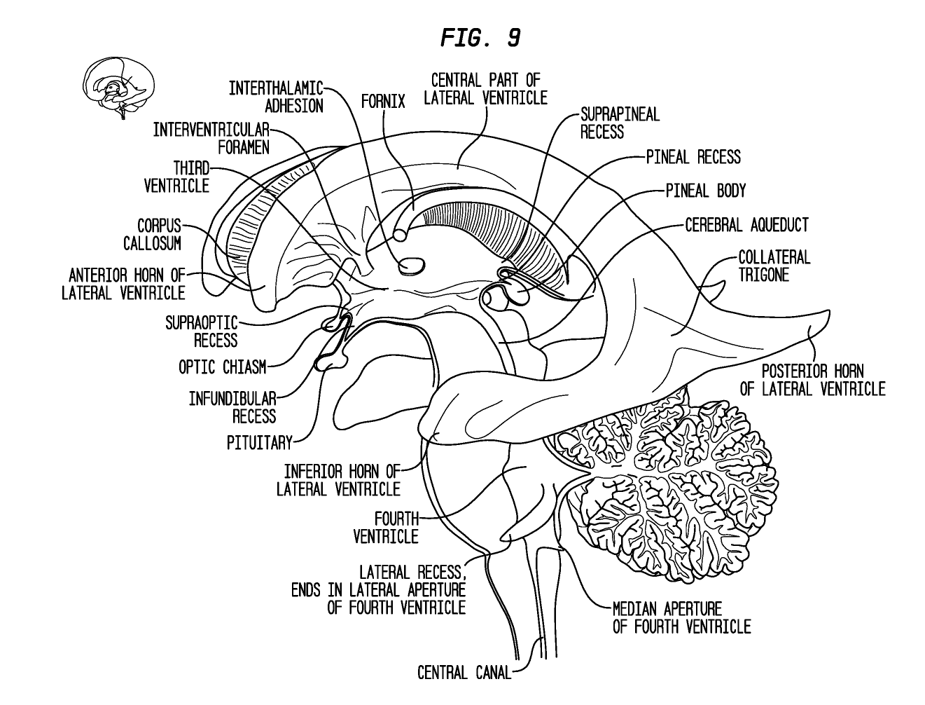

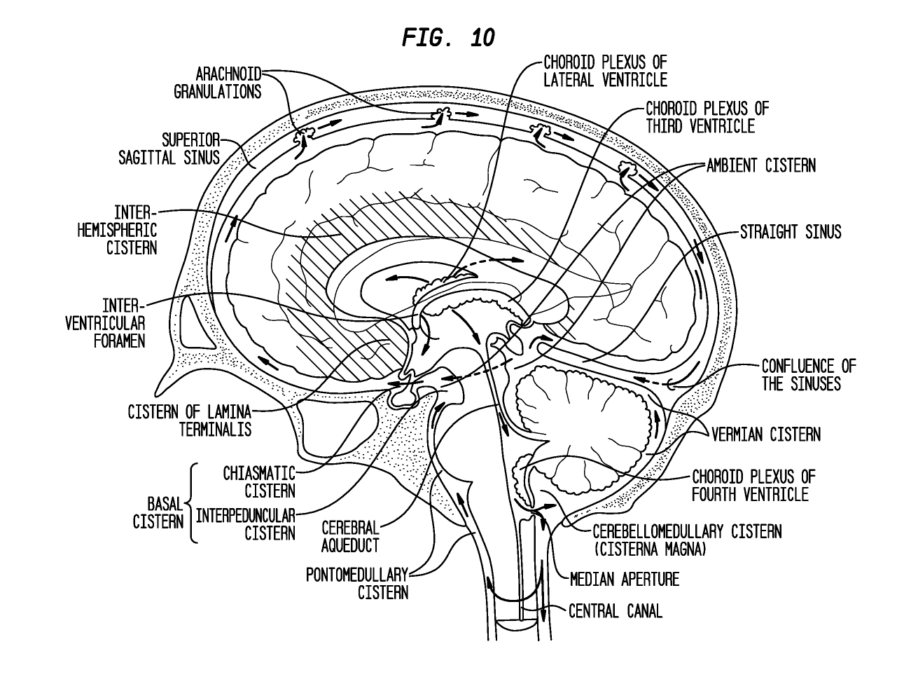

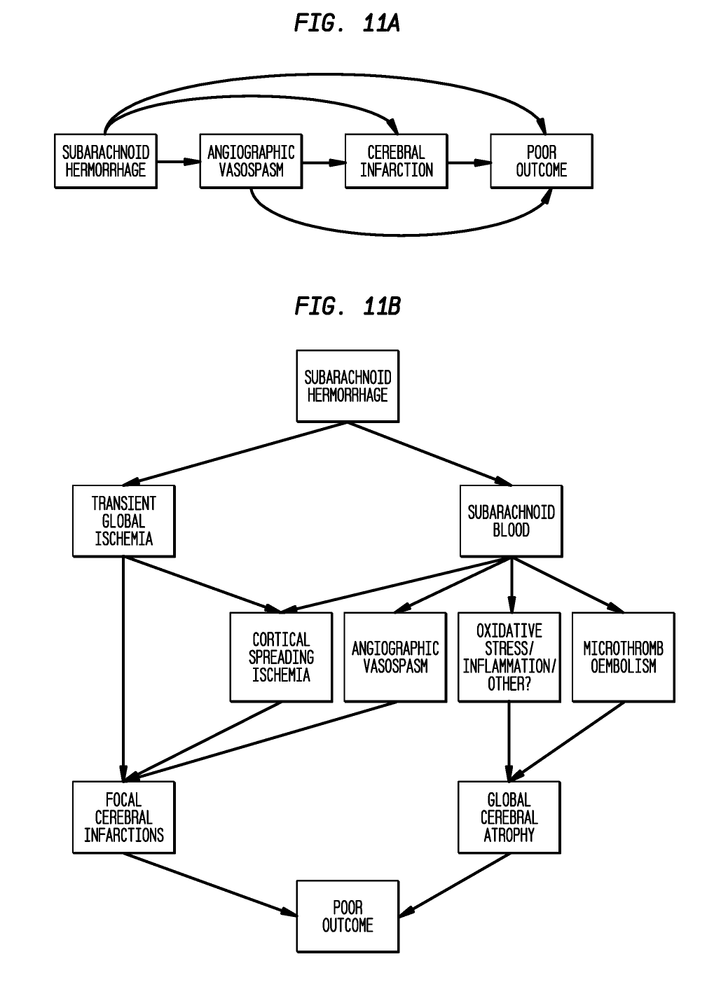

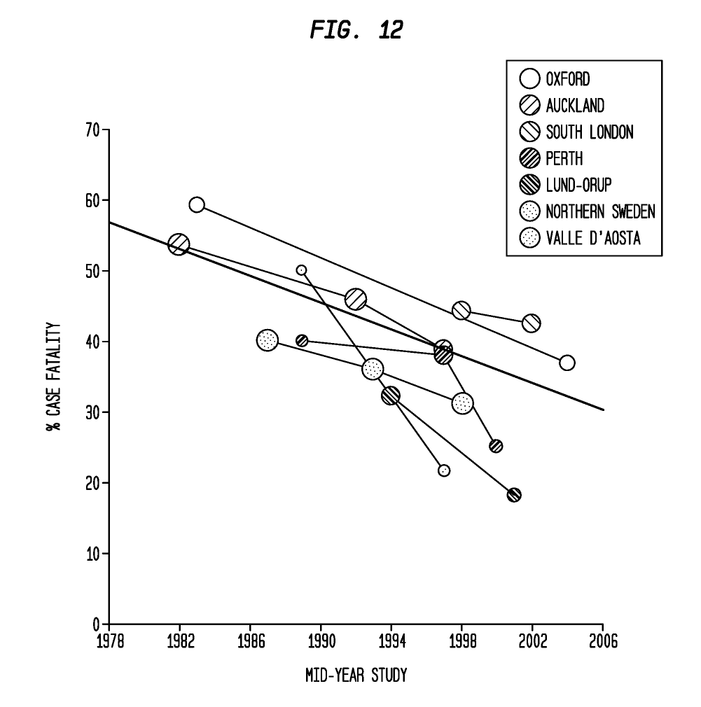

As shown in FIG. 1, the term “cerebral artery” or its numerous grammatical forms refers to the anterior communication artery, middle cerebral artery, internal carotid artery, anterior cerebral artery, ophthalmic artery, anterior choroidal artery, posterior communicating artery, basilar artery and vertebral artery, among others.

The term “cerebral vasospasm” as used herein refers to the delayed occurrence of narrowing of large capacitance arteries at the base of the brain after SAH, often associated with diminished perfusion in the territory distal to the affected vessel. The current preferred term is angiographic vasospasm. Angiographic or cerebral vasospasm begins to occur 3 days after rupture of an aneurysm, most commonly peaks at seven days following the hemorrhage and often resolves within 14 days when the blood has been absorbed by the body.

The term “cohesion” and its other grammatical forms as used herein relates to an attractive force between like molecules.

The term “complication” as used herein refers to a pathological process or event during a disorder that is not an essential part of the disease, although it may result from it or from independent causes. A delayed complication is one that occurs some time after a triggering effect. Complications associated with SAH include, but are not limited to, angiographic vasospasm, microthromboemboli, cortical spreading ischemia, hydrocephalus, DCI, early and delayed cerebral infarction, seizures and increased intracranial pressure.

The term “component” as used herein refers to a constituent part, element or ingredient.

The term “composition” as used herein refers to a material formed of two or more substances.

The term “condition”, as used herein, refers to a variety of health states and is meant to include disorders or diseases caused by any underlying mechanism, disorder, or injury.

The term “consistency” as used herein refers to a degree of thickness or viscosity.

The term “contact” and all its grammatical forms as used herein refers to a state or condition of touching or of immediate or local proximity.

The term “controlled release” as used herein refers to any drug-containing formulation in which the manner and profile of drug release from the formulation are regulated. This refers to immediate as well as non-immediate release formulations, with non-immediate release formulations including, but not limited to, sustained release and delayed release formulations.

The term “cortical spreading depolarization” or “CSD” as used herein refers to a wave of near-complete neuronal depolarization and neuronal swelling in the brain that is ignited when passive cation influx across the cellular membrane exceeds ATP-dependent sodium and calcium pump activity. The cation influx is followed by water influx and shrinkage of the extracellular space by about 70%. If normal ion homoeostasis is not restored through additional recruitment of sodium and calcium pump activity, the cell swelling is maintained—a process then termed “cytotoxic edema,” since it potentially leads to cell death through a protracted intracellular calcium surge and mitochondrial depolarization. CSD induces dilation of resistance vessels in healthy tissue; hence regional cerebral blood flow increases during the neuronal depolarization phase. (Dreier, J. P. et al., Brain 132: 1866-81 (2009).

The term “cortical spreading ischemia” or “CSI,” or “inverse hemodynamic response” refers to a severe microvascular spasm that is coupled to the neuronal depolarization phase. The resulting spreading perfusion deficit prolongs neuronal depolarization [as reflected by a prolonged negative shift of the extracellular direct current (DC) potential] and the intracellular sodium and calcium surge. The hypoperfusion is significant enough to produce a mismatch between neuronal energy demand and supply. (Id.).

The term “delayed cerebral ischemia” or “DCI” as used herein refers to the occurrence of focal neurological impairment (such as hemiparesis, aphasia, apraxia, hemianopia, or neglect), or a decrease in the Glasgow coma scale (either on the total score or on one of its individual components [eye, motor on either side, verbal]). This may or may not last for at least one hour, is not apparent immediately after aneurysm occlusion, and cannot be attributed to other causes by means of clinical assessment, CT or magnetic resonance imaging (MRI) scanning of the brain, and appropriate laboratory studies. Angiographic cerebral vasospasm is a description of a radiological test (either CT angiography [CTA], MR angiography [MRA] MRA or catheter angiography [CA]), and may be a cause of DCI.

The term “delayed release” is used herein in its conventional sense to refer to a drug formulation in which there is a time delay between administration of the formulation and the release of the drug there from. “Delayed release” may or may not involve gradual release of drug over an extended period of time, and thus may or may not be “sustained release.”

The term “diffuse pharmacologic effect”, as used herein, refers to a pharmacologic effect that spreads, disperses or scatters widely over a space or surface.

The term “disease” or “disorder”, as used herein, refers to an impairment of health or a condition of abnormal functioning.

The term “dispersion”, as used herein, refers to a two-phase system, in which one phase is distributed as particles or droplets in the second, or continuous phase. In these systems, the dispersed phase frequently is referred to as the discontinuous or internal phase, and the continuous phase is called the external phase or dispersion medium. For example, in coarse dispersions, the particle size is 0.5 μm. In colloidal dispersions, size of the dispersed particle is in the range of approximately 1 nm to 0.5 μm. A molecular dispersion is a dispersion in which the dispersed phase consists of individual molecules; if the molecules are less than colloidal size, the result is a true solution.

The term “disposed”, as used herein, refers to being placed, arranged or distributed in a particular fashion.

The term “drug” as used herein refers to a therapeutic agent or any substance, other than food, used in the prevention, diagnosis, alleviation, treatment or cure of disease.

The term “effective amount” refers to the amount necessary or sufficient to realize a desired biologic effect.

The term “emulsion” as used herein refers to a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will occur unless a third substance, an emulsifying agent, is incorporated. Thus, a basic emulsion contains at least three components, the two immiscible liquid carriers and the emulsifying agent, as well as the active ingredient. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil.

The term “excipient” is used herein to include any other agent or compound that may be contained in a long-acting formulation that is not the bioactive agent. As such, an excipient should be pharmaceutically or biologically acceptable or relevant (for example, an excipient should generally be non-toxic to the subject). “Excipient” includes a single such compound and is also intended to include a plurality of such compounds.

The term “flowable”, as used herein, refers to that which is capable of movement in, or as if in, a stream by continuous change of relative position.

The term “fluidity” as used herein refers to the reciprocal of viscosity (1/q).

The term “formulation” as used herein refers to a mixture prepared according to a formula, recipe or procedure.

The terms “functional equivalent” or “functionally equivalent” are used interchangeably herein to refer to substances, molecules, polynucleotides, proteins, peptides, or polypeptides having similar or identical effects or use. For example, a substance functionally equivalent to nimodipine may have a biologic activity substantially similar or identical to nimodipine.

The term “granulomatous inflammation” as used herein refers to an inflammation reaction characterized by a predominance of regular to epithelioid macrophages with or without multinucleated giant cells and connective tissue.

The term “hemostatic agent” as used herein refers to an agent that arrests the flow of blood within the vessels.

The term “histamine type-2 blocking agent” as used herein refers to an agent that blocks the action of histamine on parietal cells in the stomach by blocking the histamine 2 receptor, decreasing the production of acid by these cells.

The term “hydrocephalus” as used herein refers to a condition marked by an excessive accumulation of cerebrospinal fluid (CSF) resulting in dilation of the cerebral ventricles, with or without raised intracranial pressure.

The term “hydrogel” as used herein refers to a substance resulting in a solid, semisolid, pseudoplastic, or plastic structure containing a necessary aqueous component to produce a gelatinous or jelly-like mass.

The term “hydrophilic” or “hydrophilic agent” as used herein refers to a material or substance having an affinity for polar substances, such as water.

The term “hypersensitivity reaction” as used herein refers to an exaggerated response of the body to a foreign agent. A hypersensitivity reaction can be delayed or immediate. A delayed hypersensitivity reaction is a cell mediated response that occurs in immune individuals peaking at about 24-48 hours after challenge with the same antigen used in an initial challenge. The interaction of T-helper I lymphocytes (Th-I) with MHC Class II positive antigen presenting cells initiates the delayed hypersensitivity reaction. This interaction induces T-helper 1 lymphocytes and macrophages at the site to secrete cytokines. An immediate hypersensitivity reaction is an exaggerated immune response mediated by antibodies occurring within minutes after exposing a sensitized individual to the approximate antigen.

The term “hypertension” as used herein refers to high systemic blood pressure, a transitory or sustained elevation of systemic blood pressure to a level likely to induce cardiovascular damage or other adverse consequences.

The term “hypotension” as used herein refers to subnormal systemic arterial blood pressure; or a reduced pressure or tension of any kind.

The term “impregnate”, as used herein in its various grammatical forms refers to causing to be infused or permeated throughout; or to fill interstices with a substance.

The phrase “in proximity” as used herein refers to being in the subarachnoid space within less than 10 mm, less than 9.9 mm, less than 9.8 mm, less than 9.7 mm, less than 9.6 mm, less than 9.5 mm, less than 9.4 mm, less than 9.3 mm, less than 9.2 mm, less than 9.1 mm, less than 9.0 mm, less than 8.9 mm, less than 8.8 mm, less than 8.7 mm, less than 8.6 mm, less than 8.5 mm, less than 8.4 mm, less than 8.3 mm, less than 8.2 mm, less than 8.1 mm, less than 8.0 mm, less than 7.9 mm, less than 7.8 mm, less than 7.7 mm, less than 7.6 mm, less than 7.5 mm, less than 7.4 mm, less than 7.3 mm, less than 7.2 mm, less than 7.1 mm, less than 7.0 mm, less than 6.9 mm, less than 6.8 mm, less than 6.7 mm, less than 6.6 mm, less than 6.5 mm, less than 6.4 mm, less than 6.3 mm, less than 6.2 mm, less than 6.1 mm, less than 6.0 mm, less than 5.9 mm, less than 5.8 mm, less than 5.7 mm, less than 5.6 mm, less than 5.5 mm, less than 5.4 mm, less than 5.3 mm, less than 5.2 mm, less than 5.1 mm, less than 5.0 mm, less than 4.9 mm, less than 4.8 mm, less than 4.7 mm, less than 4.6 mm, less than 4.5 mm, less than 4.4 mm, less than 4.3 mm, less than 4.2 mm, less than 4.1 mm, less than 4.0 mm, less than 3.9 mm, less than 3.8 mm, less than 3.7 mm, less than 3.6 mm, less than 3.5 mm, less than 3.4 mm, less than 3.3 mm, less than 3.2 mm, less than 3.1 mm, less than 3.0 mm, less than 2.9 mm, less than 2.8 mm, less than 2.7 mm, less than 2.6 mm, less than 2.5 mm, less than 2.4 mm, less than 2.3 mm, less than 2.2 mm, less than 2.1 mm, less than 2.0 mm, less than 1.9 mm, less than 1.8 mm, less than 1.7 mm, less than 1.6 mm, less than 1.5 mm, less than 1.4 mm, less than 1.3 mm, less than 1.2 mm, less than 1.1 mm, less than 1.0 mm, less than 0.9 mm, less than 0.8 mm, less than 0.7 mm, less than 0.6 mm, less than 0.5 mm, less than 0.4 mm, less than 0.3 mm, less than 0.2 mm, less than 0.1 mm, less than 0.09 mm, less than 0.08 mm, less than 0.07 mm, less than 0.06 mm, less than 0.05 mm, less than 0.04 mm, less than 0.03 mm, less than 0.02 mm, less than 0.01 mm, less than 0.009 mm, less than 0.008 mm, less than 0.007 mm, less than 0.006 mm, less than 0.005 mm, less than 0.004 mm, less than 0.003 mm, less than 0.002 mm less than 0.001 mm from a blood vessel at risk of interruption, including without limitation, that caused by a brain injury.

The term “infarction” as used herein refers to a sudden insufficiency of arterial or venous blood supply due to emboli, thrombi, mechanical factors, or pressure that produces a macroscopic area of necrosis. The term “cerebral infarction” as used herein refers to s loss of brain tissue subsequent to the transient or permanent loss of circulation and/or oxygen delivery to the cerebrum region of the brain. The term “infarct” as used herein refers to an area of necrosis resulting from a sudden insufficiency of arterial or venous blood supply.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98% or at least 99%.

The term “inhibitor” as used herein refers to a second molecule that binds to a first molecule thereby decreasing the first molecule's activity. For example, enzyme inhibitors are molecules that bind to enzymes thereby decreasing enzyme activity. The binding of an inhibitor may stop substrate from entering the active site of the enzyme and/or hinder the enzyme from catalyzing its reaction. Inhibitor binding is either reversible or irreversible. Irreversible inhibitors usually react with the enzyme and change it chemically, for example, by modifying key amino acid residues needed for enzymatic activity. In contrast, reversible inhibitors bind non-covalently and produce different types of inhibition depending on whether these inhibitors bind the enzyme, the enzyme-substrate complex, or both. Enzyme inhibitors often are evaluated by their specificity and potency.

The term “injection”, as used herein, refers to introduction into subcutaneous tissue, or muscular tissue, a vein, an artery or other canals or cavities in the body by force.

The term “injury,” as used herein, refers to damage or harm to a structure or function of the body caused by an outside agent or force, which may be physical or chemical.

The term “interruption” and its various grammatical forms, as used herein, refers to an alteration in the continuity of blood flow through a blood vessel that is caused by dilation or constriction of the blood vessel induced by chemical, mechanical, and/or physical effects.

The terms “intracisternal administration”, “intracisternal site”, and “intracisternal site of administration” are used interchangeably to refer to administration of a substance, for example a drug formulation, into a subarachnoid cistern of the brain.

The terms “intraventricular administration”, “intraventricular site” and “intraventricular site of administration” are used interchangeably to refer to administration of a substance, for example a drug formulation, into a cerebral ventricle.

The terms “intrathecal administration”,“intrathecal site”, or “intrathecal site of administration” are used interchangeably to refer to administration of a substance, for example a drug formulation, into the spinal subarachnoid space.

The term “ischemia” as used herein refers to a lack of blood supply and oxygen that occurs when reduced perfusion pressure distal to an abnormal narrowing (stenosis) of a blood vessel is not compensated by autoregulatory dilation of the resistance vessels.

The term “isolated molecule” as used herein refers to a molecule that is substantially pure and is free of other substances with which it is ordinarily found in nature or in vivo systems to an extent practical and appropriate for its intended use.

The terms “in the body”, “void volume”, “resection pocket”, “excavation”, “injection site”, “deposition site” or “implant site” or “site of delivery” as used herein are meant to include all tissues of the body without limit, and may refer to spaces formed therein from injections, surgical incisions, tumor or tissue removal, tissue injuries, abscess formation, or any other similar cavity, space, or pocket formed thus by action of clinical assessment, treatment or physiologic response to disease or pathology as non-limiting examples thereof.

The term “lipophilic” or “lipophilic agent” as used herein refers to a material or substance preferring or possessing an affinity for a non-polar environment compared to a polar or aqueous environment; an agent that is capable of dissolving, of being dissolved in, or of absorbing lipids.

The phrase “localized administration”, as used herein, refers to administration of a therapeutic agent in a particular location in the body.

The phrase “localized pharmacologic effect”, as used herein, refers to a pharmacologic effect limited to a certain location, i.e. in proximity to a certain location, place, area or site. The phrase “predominantly localized pharmacologic effect”, as used herein, refers to a pharmacologic effect of a drug limited to a certain location by at least 1 to 3 orders of magnitude, which is achieved by a localized administration as compared to a systemic administration.

The term “long-term” release, as used herein, refers to delivery of therapeutic levels of the active ingredient for at least 7 days, and potentially up to about 30 to about 60 days. Terms such as “long-acting”, “sustained-release” or “controlled release” are used generally to describe a formulation, dosage form, device or other type of technologies used, such as, for example, in the art to achieve the prolonged or extended release or bioavailability of a bioactive agent to a subject; it may refer to technologies that provide prolonged or extended release or bioavailability of a bioactive agent to the general systemic circulation or a subject or to local sites of action in a subject including (but not limited to) cells, tissues, organs, joints, regions, and the like. Furthermore, these terms may refer to a technology that is used to prolong or extend the release of a bioactive agent from a formulation or dosage form or they may refer to a technology used to extend or prolong the bioavailability or the pharmacokinetics or the duration of action of a bioactive agent to a subject or they may refer to a technology that is used to extend or prolong the pharmacodynamic effect elicited by a formulation. A “long-acting formulation,” a “sustained release formulation,” or a “controlled release formulation” (and the like) is a pharmaceutical formulation, dosage form, or other technology that is used to provide long-acting release of a bioactive agent to a subject.

Generally, long-acting or sustained release formulations comprise a bioactive agent or agents (including, for example, an antibody or nucleic acid, steroid or nimodipine) that is/are incorporated or associated with a biocompatible polymer in one manner or another. The polymers typically used in the preparation of long-acting formulations include, but are not limited, to biodegradable polymers (such as the polyesters poly(lactide), poly(lactide-co-glycolide), poly(caprolactone), poly(hydroxybutyrates), and the like) and non-degradable polymers (such as ethylenevinyl acetate (EVA), silicone polymers and the like). The agent may be blended homogeneously throughout the polymer or polymer matrix or the agent may be distributed unevenly (or discontinuously or heterogeneously) throughout the polymer or polymer matrix (as in the case of a bioactive agent-loaded core that is surrounded by a polymer-rich coating or polymer wall forming material as in the case of a microcapsule, nanocapsule, a coated or encapsulated implant and the like). The dosage form may be in the physical form of particles, film, a fiber, a filament, a sheet, a thread, a cylindrical implant, a asymmetrically-shaped implant or a fibrous mesh (such as a woven or non-woven material; felt; gauze, sponge, and the like). When in the form of particles, the formulation may be in the form of microparticles, nanoparticles, microspheres, nanospheres, microcapsules or nanocapsules, and particles, in general, and combinations thereof. As such, the long-acting (or sustained-release) formulations of the present invention may include any variety of types or designs that are described, used or practiced in the art.

Long-acting formulations containing bioactive agents can be used to deliver those agents to the systemic circulation or they can be used to achieve local or site-specific delivery to cells, tissues, organs, bones and the like that are located nearby the site of administration. Further, formulations can be used to achieve systemic delivery of the bioactive agent and/or local delivery of the bioactive agent. Formulations can be delivered by injection (through, for example, a needle, a syringe, a trocar, a cannula, and the like) or by implantation. Delivery can be made via any variety of routes of administration commonly used for medical, clinical, surgical purposes including, but not limited to, intravenous, intraarterial, intramuscular, intraperitoneal, subcutaneous, intradermal, infusion and intracatheter delivery (and the like) in addition to delivery to specific locations (such as local delivery) including intraventricular, intracisternal, intrathecal, intracardiac, intraosseous (bone marrow), stereotactic-guided delivery, infusion delivery, CNS delivery, stereo-tactically administered delivery, orthopedic delivery (for example, delivery to joints, into bone, into bone defects and the like), cardiovascular delivery, inter-, intra-, and para-ocular (including intravitreal and scleral and retrobulbar and sub-tenons delivery and the like) delivery, and any delivery to any multitude of other sites, locations, organs, tissues, etc.

The term “maximum feasible dose” (MFD) as used herein refers to the highest dose of a drug possible based on physical properties that limit the dose formulation concentration; limitations on volume that can be administered, availability of compound or a combination thereof.

The term “maximum tolerated dose” (MTD) as used herein in the context of a toxicity study refers to the highest dose of a drug that does not produce unacceptable toxicity.

The term “meningitis” as used herein refers to an inflammation of the meninges of the brain and spinal cord.

The term “microparticle composition” or “microparticulate composition”, as used herein, refers to a composition comprising a microparticle formulation and a pharmaceutically acceptable carrier, where the microparticle formulation comprises a therapeutic agent and a plurality of microparticles.

The term “microthromboembolus” (or plural “microthromboemboli”) as used herein refers to a small fragment of blood clot that causes obstruction or occlusion of a blood vessel.

The term “modulate” as used herein means to regulate, alter, adapt, or adjust to a certain measure or proportion.

The term “muscle relaxant” as used herein refers to an agent that reduces muscle tension or produces skeletal muscle paralysis.

The term “myocardial infarction” refers to a sudden insufficiency of arterial or venous blood supply to the heart due to emboli, thrombi, mechanical factors, or pressure that produces a macroscopic area of necrosis.

The term “onset of a delayed complication”, as used herein, refers to the start or beginning of symptoms associated with the delayed complication.

The term “outcome” as used herein refers to a specific result or effect that can be measured. The term “poor outcome” as used herein refers to a score of 1, 2 or 3 on the Glasgow outcome scale (GOS) or a score of 1, 2, 3, 4 or 5 on the extended Glasgow outcome scale (GOSE). The term “good outcome” as used herein refers to a score of 4 or 5 on the GOS or a score of 6, 7 or 8 on the GOSE.

The term “paste” as used herein refers to a semisoft substance of pliable (meaning flexible, easily bent, deformable) consistency.

The term “parenteral” as used herein refers to introduction into the body by way of an injection (i.e., administration by injection) outside the gastrointestinal tract, including, for example, subcutaneously (i.e., an injection beneath the skin), intramuscularly (i.e., an injection into a muscle); intravenously (i.e., an injection into a vein), intrathecally (i.e., an injection into the subarachnoid space of the spine), intracisternally, intraventricularly, or by infusion techniques. A parenterally administered composition is delivered using a needle, e.g., a surgical needle. The term “surgical needle” as used herein, refers to any needle adapted for delivery of fluid (i.e., those capable of flow) compositions into a selected anatomical structure. Injectable preparations, such as sterile injectable aqueous or oleaginous suspensions, may be formulated according to the known art using suitable dispersing or wetting agents and suspending agents.

The term “paresis” as used herein refers to partial or incomplete paralysis.

The terms “particles”, as used herein, refer to extremely small constituents, e.g., femoparticles (10⁻¹⁵ m), picoparticles (10⁻¹² m), nanoparticles (10⁻⁹ m), microparticles (10⁻⁶ m), milliparticles (10⁻³ m) that may contain in whole or in part at least one therapeutic agent as described herein. The particles may contain therapeutic agent(s) in a core surrounded by a coating. Therapeutic agent(s) also may be dispersed throughout the particles. Therapeutic agent(s) also may be adsorbed into the particles. The particles may be of any order release kinetics, including zero order release, first order release, second order release, delayed release, sustained release, immediate release, etc., and any combination thereof. The particle may include, in addition to therapeutic agent(s), any of those materials routinely used in the art of pharmacy and medicine, including, but not limited to, erodible, nonerodible, biodegradable, or nonbiodegradable material or combinations thereof. The particles may be microcapsules that contain the voltage-gated calcium channel antagonist in a solution or in a semi-solid state. The particles may be of virtually any shape.

The term “pharmaceutically acceptable carrier” as used herein refers to one or more compatible solid or liquid filler, diluent or encapsulating substance which is/are suitable for administration to a human or other vertebrate animal. The components of the pharmaceutical compositions also are capable of being commingled in a manner such that there is no interaction which would substantially impair the desired pharmaceutical efficiency.

The term “pharmaceutical formulation” or “pharmaceutical composition” is used herein to refer to a formulation or a composition that is employed to prevent, reduce in intensity, cure or otherwise treat a target condition or disease.

The term “pharmacologic effect” as used herein, refers to a result or consequence of exposure to an active agent.

The term “placebo” as used herein, refers to an inert substance or an inert compound identical in appearance to material being tested in experimental research, in a clinical trial, etc., which is administered to distinguish between drug action and suggestive effect of the material under study.

The term “positive end expiratory pressure” or “PEEP” as used herein refers to an elevation of transpulmonary pressure at the end of expiration.

The term “prevent” as used herein refers to the keeping, hindering or averting of an event, act or action from happening, occurring, or arising.

The term “prodrug” as used herein means a peptide or derivative which is in an inactive form and which is converted to an active form by biological conversion following administration to a subject.

The term “prognosis” as used herein refers to an expected future cause and outcome of a disease or disorder, based on medical knowledge.

The term “proton pump inhibitor” as used herein refers to a compound that suppresses gastric acid secretion leading to relief of acid related symptoms. (McDonagh, E. M. et al., “From pharmacogenomic knowledge acquisition to clinical applications: the PharmGKB as a clinical pharmacogenomic biomarker resource,” Biomarkers in Medicine (2011) December; 5(6):795-806).

The term “pulsatile release” as used herein refers to any drug-containing formulation in which a burst of the drug is released at one or more predetermined time intervals.

The term “reduce” or “reducing” as used herein refers to a diminution, a decrease, an attenuation, limitation or abatement of the degree, intensity, extent, size, amount, density, number or occurrence of disorder in individuals at risk of developing the disorder.

The term “release” and its various grammatical forms, refers to dissolution of an active drug component and diffusion of the dissolved or solubilized species by a combination of the following processes: (1) hydration of a matrix, (2) diffusion of a solution into the matrix; (3) dissolution of the drug; and (4) diffusion of the dissolved drug out of the matrix.

The term “serious adverse event” as used herein refers to an adverse event that has one or more of the following patient outcomes, or, based on reasonable medical judgment, requires a medical or surgical intervention to prevent one of the following patient outcomes: death, a life-threatening experience, inpatient hospitalization, prolongation of existing hospitalization, a persistent or significant disability or incapacity; a congenital anomaly or birth defect. The term “life-threatening experience” refers to an event in which the subject/patient was at risk of death at the time of the event. It does not refer to an event that hypothetically might have caused death if it were more severe. Important medical events that may not immediately result in death, be life-threatening, or require hospitalization may be considered as a serious adverse event when, based upon appropriate medical judgment, they may jeopardize the patient and may require medical or surgical intervention to prevent one of the outcomes listed in the definitions above. The term “inpatient hospitalization” as used herein refers to an overnight stay in a hospital unit and/or emergency room due to an adverse event. The term “prolongation of existing hospitalization” as used herein refers to at least one overnight stay in the hospital unit and/or emergency room due to the adverse event in addition to the initial inpatient hospitalization. The treatment on an emergency or outpatient basis for an event not fulfilling the definition of seriousness given above and not resulting in hospitalization is not considered a serious adverse event and is reported as an adverse event only. The following reasons for hospitalizations are not considered adverse events or serious adverse events: hospitalizations for cosmetic elective surgery, social and/or convenience reasons; standard monitoring of a pre-existing disease or medical condition that did not worsen, e.g., hospitalization for coronary angiography in a patient with stable angina pectoris; elective treatment of a pre-existing disease or medical condition that did not worsen.

The term “similar” is used interchangeably with the terms analogous, comparable, or resembling, meaning having traits or characteristics in common.

The term “stability” of a pharmaceutical product as used herein refers to the capability of a particular formulation to remain within its physical, chemical, microbiological, therapeutic and toxicological specifications.

The term “statin” as used herein refers to a cholesterol-lowering agent that inhibits the enzyme 3-hydroxy-3-methylglutaryl-coenzyme (HMG-CoA) reductase.

The term “sterile barrier system” as used herein refers to a minimum package that prevents ingress of microorganisms and allows aseptic presentation of a product at the point of use.

The term “subacute inflammation” as used herein refers to a tissue reaction typically seen subsequent to the early inflammatory process characterized by a mixture of neutrophils, lymphocytes, and occasionally macrophages and/or plasma cells.

The term “subarachnoid hemorrhage” or “SAH” is used herein to refer to a condition in which blood collects beneath the arachnoid mater. This area, called the subarachnoid space, normally contains CSF. The accumulation of blood in the subarachnoid space may lead to stroke, seizures, and other complications. Additionally, SAH may cause permanent brain damage and a number of harmful biochemical events in the brain. Causes of SAH include bleeding from a cerebral aneurysm, vascular anomaly, trauma and extension into the subarachnoid space from a primary intracerebral hemorrhage. Symptoms of SAH include, for example, sudden and severe headache, nausea and/or vomiting, symptoms of meningeal irritation (e.g., neck stiffness, low back pain, bilateral leg pain), photophobia and visual changes, and/or loss of consciousness. SAH is often secondary to a head injury or a blood vessel defect known as an aneurysm. In some instances, SAH can induce angiographic vasospasm that may in turn lead to an ischemic stroke or DCI. By definition there is blood in the CSF after SAH. Subjects having a SAH may be identified by a number of symptoms. For example, a subject having a SAH will present with blood in the subarachnoid space. Subjects having a SAH can also be identified by an intracranial pressure that approximates mean arterial pressure, by a fall in cerebral perfusion pressure, or by the sudden transient loss of consciousness (sometimes preceded by a painful headache). In about half of cases, subjects present with a severe headache which may be associated with physical exertion. Other symptoms associated with SAH include nausea, vomiting, memory loss, temporary or prolonged loss of consciousness, hemiparesis and aphasia. Subjects having a SAH also may be identified by the presence of creatine kinase-BB isoenzyme activity in their CSF. This enzyme is enriched in the brain but normally is not present in the CSF. Thus, its presence in the CSF is indicative of “leak” from the brain into the subarachnoid space. Assay of creatine-kinase BB isoenzyme activity in the CSF is described by Coplin et al. (Coplin et al 1999 Arch Neurol 56, 1348-1352), which is incorporated herein by reference. Additionally, a spinal tap or lumbar puncture may be used to demonstrate whether blood is present in the CSF, the presence of blood being diagnostic of an SAH. A cranial CT scan or an MRI also may be used to identify blood in the subarachnoid region. Angiography also may be used to determine not only whether a hemorrhage has occurred, but also the location of the hemorrhage. SAH commonly results from rupture of an intracranial saccular aneurysm or from malformation of the arteriovenous system in the brain. Accordingly, a subject at risk of having a SAH includes a subject having a saccular aneurysm as well as a subject having a malformation of the arteriovenous system. Common sites of saccular aneurysms are the anterior communicating artery, posterior communicating artery, middle cerebral artery, internal carotid artery, top of the basilar artery and the junction of the basilar artery with the superior cerebellar or the anterior inferior cerebellar artery. A subject with a saccular aneurysm may be identified through routine medical imaging techniques, such as CT and MRI. A saccular or cerebral aneurysm forms a mushroom-like or berry-like shape (sometimes referred to as “a dome with a neck” shape). When the SAH is caused by an aneurysm, it is termed an “aneurysmal SAH” (aSAH).

The terms “subject” or “individual” or “patient” are used interchangeably to refer to a member of an animal species of mammalian origin, including humans.

The phrase “a subject having cerebral vasospasm” as used herein refers to one who has symptoms of or has been diagnosed with cerebral vasospasm and/or presents with diagnostic markers consistent with angiographic vasospasm. A “subject at risk of cerebral vasospasm” is one who has one or more predisposing factors to the development of cerebral vasospasm. A predisposing factor includes, but is not limited to, existence of a SAH. A subject who has experienced a recent SAH is at significantly higher risk of developing cerebral vasospasm than a subject who has not had a recent SAH. MR angiography, CT angiography and catheter angiography can be used to diagnose cerebral vasospasm Angiography is a technique in which a contrast agent is introduced into the blood stream in order to view blood flow and/or arteries. A contrast agent is required because blood flow and/or arteries sometimes are only weakly apparent in a regular MR scan, CT scan or radiographic film for catheter angiography. Appropriate contrast agents will vary depending upon the imaging technique used. For example, gadolinium is commonly used as a contrast agent used in MR scans. Other MR appropriate contrast agents are known in the art. Diagnostic markers include, but are not limited to, the presence of blood in the CSF, a recent history of a SAH and/or reduction in the lumen diameter of cerebral arteries observed on a catheter, computed tomographic or magnetic resonance angiogram one to 14 days after an SAH or TBI. Presence of blood in the CSF may be detected using CT scans. However, in some instances where the amount of blood is so small as to not be detected by CT, a lumbar puncture is warranted.

The phrase “a subject having delayed cerebral ischemic” or “DCI” as used herein refers to a subject who presents with diagnostic markers associated with DCI. Diagnostic markers include, but are not limited to, the presence of blood in the CSF and/or a recent history of a SAH and/or development of neurological deterioration 3 to 14 days after SAH when the neurological deterioration is not due to another cause that can be diagnosed, including but not limited to seizures, hydrocephalus, increased intracranial pressure, infection, intracranial hemorrhage or other systemic factors. DCI-associated symptoms include, but are not limited to, paralysis on one side of the body, inability to vocalize the words or to understand spoken or written words, and inability to perform tasks requiring spatial analysis. Such symptoms may develop over a few days, or they may fluctuate in their appearance, or they may present abruptly.

The phrase “a subject having microthromboemboli” as used herein refers to a subject who presents with diagnostic markers associated with microthromboemboli. Such diagnostic markers include, but are not limited to, the presence of blood in the CSF and/or a recent history of a SAH and/or development of neurological deterioration 3 to 14 days after SAH when the neurological deterioration is not due to another cause that can be diagnosed, including, but not limited to, seizures, hydrocephalus, increased intracranial pressure, infection, intracranial hemorrhage or other systemic factors, and embolic signals detected on transcranial Doppler ultrasound of large conducting cerebral arteries. Microthromboemboli-associated symptoms include, but are not limited to, paralysis on one side of the body, inability to vocalize the words or to understand spoken or written words, and inability to perform tasks requiring spatial analysis. Such symptoms may develop over a few days, or they may fluctuate in their appearance, or they may present abruptly.

The phrase “a subject having cortical spreading ischemia” as used herein refers to a subject who presents with diagnostic markers associated with cortical spreading ischemia. Such diagnostic markers include, but are not limited to, the presence of blood in the CSF and/or a recent history of a SAH and/or development of neurological deterioration one to 14 days after SAH when the neurological deterioration is not due to another cause that can be diagnosed, including but not limited to seizures, hydrocephalus, increased intracranial pressure, infection, intracranial hemorrhage or other systemic factors and detection of propagating waves of depolarization with vasoconstriction detected by electrocorticography. Cortical spreading ischemia-associated symptoms include, but are not limited to, paralysis on one side of the body, inability to vocalize the words or to understand spoken or written words, and inability to perform tasks requiring spatial analysis. Such symptoms may develop over a few days, or they may fluctuate in their appearance, or they may present abruptly.

The term “a subject at risk of DCI, microthromboemboli, cortical spreading ischemia, cortical spreading depolarization” (CSD), or angiographic vasospasm” as used herein refers to a subject who has one or more predisposing factors to the development of these conditions. A predisposing factor includes, but is not limited to, existence of a SAH. A subject who has experienced a recent SAH is at significantly higher risk of developing angiographic vasospasm and DCI than a subject who has not had a recent SAH. MR angiography, CT angiography and catheter angiography can be used to diagnose at least one of DCI, microthromboemboli, cortical spreading ischemia or angiographic vasospasm. Angiography is a technique in which a contrast agent is introduced into the blood stream in order to view blood flow and/or arteries. A contrast agent is required because blood flow and/or arteries sometimes are only weakly apparent in a regular MR scan, CT scan or radiographic film for catheter angiography. Appropriate contrast agents will vary depending upon the imaging technique used. For example, gadolinium is commonly used as a contrast agent used in MR scans. Other MR appropriate contrast agents are known in the art.

The phrase “subject in need thereof” as used herein refers to a patient that (i) will be administered a formulation containing at least one therapeutic agent, (ii) is receiving a formulation containing at least one therapeutic agent; or (iii) has received a formulation containing at least one therapeutic agent, unless the context and usage of the phrase indicates otherwise.

The term “suitable for delivery”, as used herein, refers to being apt, appropriate for, designed for, or proper for release only in a subarachnoid space.

The term “substantially pure”, as used herein, refers to a condition of a therapeutic agent such that it has been substantially separated from the substances with which it may be associated in living systems or during synthesis. According to some embodiments, a substantially pure therapeutic agent is at least 70% pure, at least 75% pure, at least 80% pure, at least 85% pure, at least 90% pure, at least 95% pure, at least 96% pure, at least 97% pure, at least 98% pure, or at least 99% pure.

The term “susceptible” as used herein refers to a member of a population at risk.

The term “sustained release” (also referred to as “extended release”) is used herein in its conventional sense to refer to a drug formulation that provides for gradual release of a drug over an extended period of time, and that preferably, although not necessarily, results in substantially constant blood levels of a drug over an extended time period. Alternatively, delayed absorption of a parenterally administered drug form is accomplished by dissolving or suspending the drug in an oil vehicle. Nonlimiting examples of sustained release biodegradable polymers include polyesters, polyester polyethylene glycol copolymers, polyamino-derived biopolymers, polyanhydrides, polyorthoesters, polyphosphazenes, sucrose acetate isobutyrate (SAIB), photopolymerizable biopolymers, protein polymers, collagen, polysaccharides, chitosans, and alginates.

The term “symptom” as used herein refers to a phenomenon that arises from and accompanies a particular disease or disorder and serves as an indication of it.

The term “syndrome,” as used herein, refers to a pattern of symptoms indicative of some disease or condition.

The term “synergistic effect”, as used herein, refers to a combined effect of two chemicals, which is greater than the sum of the effects of each agent given alone.

The phrase “systemic administration”, as used herein, refers to administration of a therapeutic agent with a pharmacologic effect on the entire body. Systemic administration includes enteral administration (e.g. oral) through the gastrointestinal tract and parenteral administration (e.g. intravenous, intramuscular, etc.) outside the gastrointestinal tract.

The terms “terminal sterilization” and “terminally sterilized” as used herein refer to a process whereby a product is sterilized within its sterile barrier system or whereby the final product is sterilized. The terminal sterilization process is considered a manufacturing process step and usually takes place at, or near, the end of the manufacturing process.

The term “therapeutic agent” as used herein refers to a drug, molecule, nucleic acid, protein, composition or other substance that provides a therapeutic effect. The terms “therapeutic agent” and “active agent” are used interchangeably. The term “first therapeutic agent” as used herein includes a calcium channel antagonist, an endothelin antagonist, or a transient receptor potential (TRP) protein antagonist. The term “second therapeutic agent” as used herein may include a hemostatic agent, a proton pump inhibitor, a histamine type-2 blocking agent, an anticoagulant, a vasodilator, a statin, an anti-inflammatory agent, a muscle relaxant, etc.

The terms “therapeutic amount”, “therapeutic effective amount” or an “amount effective” of one or more of the therapeutic agents is an amount that is sufficient to provide the intended benefit of treatment. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen may be planned which does not cause substantial toxicity and yet is effective to treat the particular subject. A therapeutic effective amount of the therapeutic agents that can be employed ranges from generally 0.1 mg/kg body weight and about 50 mg/kg body weight. A therapeutic effective amount for any particular application may vary depending on such factors as the disease or condition being treated, the particular therapeutic agent being administered, the size of the subject, or the severity of the disease or condition. One of ordinary skill in the art may determine empirically the effective amount of a particular inhibitor and/or other therapeutic agent without necessitating undue experimentation. It is preferred generally that a maximum dose be used, that is, the highest safe dose according to some medical judgment. However, dosage levels are based on a variety of factors, including the type of injury, the age, weight, sex, medical condition of the patient, the severity of the condition, the route of administration, and the particular therapeutic agent employed. Thus the dosage regimen may vary widely, but can be determined routinely by a surgeon using standard methods. “Dose” and “dosage” are used interchangeably herein.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED₅₀ which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

The term “therapeutically effective amount” or an “amount effective” of one or more of the active agents is an amount that is sufficient to provide the intended benefit of treatment. An effective amount of the active agents that can be employed ranges from generally 0.1 mg/kg body weight and about 50 mg/kg body weight. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a surgeon using standard methods.

The term “thrombocytopenia” as used herein refers to a condition in which the number of platelets circulating in the blood is below the normal range of platelets.

The term “transient receptor potential (TRP) protein antagonist” as used herein refers to a protein that is structurally distinct from other calcium channel antagonists and that blocks or antagonizes intracellular calcium increases in cells due to receptor-mediated calcium influx. Transient receptor potential (TRP) protein antagonists include, but are not limited to, SK&F 96365 (1-(beta-[3-(4-methoxy-phenyl)propoxy]-4-methoxyphenethyl)-1H-imidazole hydrochloride) and LOE 908 (RS)-(3,4-dihydro-6,7-dimethoxyisoquinoline-1-gamma 1)-2-phenyl-N, N-dit2-(2,3,4-trimethoxyphenypethyl]acetamide).

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “vasoconstriction” as used herein refers to the narrowing of the blood vessels resulting from contracting of the muscular wall of the vessels. When blood vessels constrict, the flow of blood is restricted or slowed. The terms “vasoconstrictors,” “vasopressors,” or “pressors” as used herein refer to factors or agents causing vasoconstriction.

The term “vasodilation” which is the opposite of vasoconstriction as used herein refers to the widening of blood vessels. The term “vasodilators” as used herein refers to factors or agents that cause vasodilation. Examples of vasodilators include for example calcium channel antagonists, endothelin receptor antagonists, TRP protein antagonists, etc.

The term “vasospasm” as used herein refers to a decrease in the internal diameter of a cerebral artery that results from contraction of smooth muscle within the wall of the artery which causes a decrease in blood flow, but generally without an increase in systemic vascular resistance. Vasospasm results in decreased cerebral blood flow and increased cerebral vascular resistance. Without being limited by theory, it generally is believed that vasospasm is caused by local injury to vessels, such as that which results from traumatic head injury, aneurysmal SAH and other causes of SAH. Cerebral vasospasm is a pathologic vasoconstriction that is triggered by blood in the CSF, a common occurrence after rupture of an aneurysm or following traumatic head injury. Cerebral vasospasm ultimately can lead to brain cell damage, in the form of cerebral ischemia and infarction, due to interrupted blood supply. The term “cerebral vasospasm” as used herein further refers to the delayed occurrence of narrowing of large capacitance arteries at the base of the brain after SAH, often associated with diminished perfusion in the territory distal to the affected vessel. Cerebral vasospasm develops beginning 3 days after rupture of an aneurysm, peaks at seven days following the hemorrhage and often resolves within 14 days when the blood has been absorbed by the body. Angiographic vasospasm is a consequence of SAH, but also can occur after any condition that deposits blood in the subarachnoid space. More specifically, the term “angiographic cerebral vasospasm” refers to the narrowing of the large capacitance arteries at the base of the brain (i.e., cerebral arteries) following hemorrhage into the subarachnoid space, and leads to reduced perfusion of distal brain regions.

The term “vehicle” as used herein refers to a substance that facilitates the use of a drug or other material that is mixed with it.

The term “ventriculitis”, as used herein refers to inflammation of the ventricles of the brain.

The term “ventriculocranial ratio” as used herein refers to the ratio of the average size of the cerebral ventricles, where the ventricular width is measured at the level of the foramen of Monroe, and compared to the width of the cranium at the same level.

The term “viscosity”, as used herein refers to the tendency of a fluid to resist flow. Viscosity is a measure of the fluid's resistance to flow. The resistance is caused by intermolecular friction exerted when layers of fluids attempt to slide by one another. Viscosity can be of two types: dynamic (or absolute) viscosity and kinematic viscosity. Absolute viscosity or the coefficient of absolute viscosity is a measure of the internal resistance. Dynamic (or absolute) viscosity is the tangential force per unit area required to move one horizontal plane with respect to the other at unit velocity when maintained a unit distance apart by the fluid. Dynamic viscosity is usually denoted in poise (P) or centipoise (cP), wherein 1 poise=1 g/cm², and 1 cP=0.01 P.

Kinematic viscosity, a measure of the resistive flow of a fluid under the influence of gravity, is the ratio of absolute or dynamic viscosity to density. It is frequently measured using a capillary viscometer; when two fluids of equal volume are placed in identical capillary viscometers and allowed to flow under the influence of gravity; a viscous liquid takes longer than a less viscous fluid to flow through the tube. Kinematic viscosity is usually denoted in Stoke (St) or Centistokes (cSt), wherein 1 St=10⁴ m²/s, and 1 cSt=0.01 St. Exemplary viscosities are shown in the table below.

| Substance | Temperature (° C.) | η* | |
|---|---|---|---|
| Water | 20 | 1.0020 | mPa s |
| Blood | 37 | 3-4 | mPa s |
| Maple syrup | 20 | 2-3 | Pa s |
| Honey | 20 | 10 | Pa s |
| molasses | 20 | 5 | mPa s |
| Mustard | 25 | 70 | Pa s |
| Olive oil | 20 | 84 | mPa s |
| Peanut butter | 20 | 150-250 | Pa s |
| Hyalgan ® | 20 | 2 | Poise |
| Toothpaste | 20 | 700-1000 | Poise |
| Orthovisc ® | 20 | 1670 | Poise |

*Ten poise (P) equals one pascal second (Pa s), making the centipoise (cP) and millipascal second (mPa s) identical.

Most ordinary liquids have viscosities on the order of 1 to 1000 mPa s, while gases have viscosities on the order of 1 to 10 μPa s. Pastes, gels, emulsions, and other complex liquids are more variable. For example, some are so viscous that they seem more like soft solids than like flowing liquids. Molten glass is extremely viscous and approaches infinite viscosity as it solidifies.

In general, increasing the concentration of a dissolved or dispersed substance generally gives rise to increasing viscosity (that is, thickening), as does increasing the molecular weight of a solute. With Newtonian fluids (typically water and solutions containing only low molecular weight material), viscosity of the fluid is independent of shear strain rate and a plot of shear strain rate (for example, the rate of stirring) against shear stress (for example, force, per unit area stirred, required for stirring) is linear and passes through the origin. Generally the viscosity of a simple liquid decreases with increasing temperature (and vice versa). As temperature increases, the average speed of the molecules in a liquid increases and the amount of time they spend in contact with their nearest neighbors decreases. Thus, as temperature increases, the average intermolecular forces decrease. The exact manner in which the two quantities vary is nonlinear and changes abruptly when the liquid changes phase.

With respect to hydrocolloid solutions (meaning a substance that forms a gel in the presence of water), at moderate concentrations above a critical value (C*), these solutions exhibit non-Newtonian behavior, where their viscosity depends on the shear strain rate, typically as opposite, where γ is the shear strain rate, η0 and η∞ are the viscosities at zero and infinite shear strain rate respectively and τ is a shear-dependent time constant that represents the reciprocal of the shear strain rate required to halve the viscosity. At low flow rates, molecules with preferred conformations that are long and thin have effectively large cross-sections due to them tumbling in solution but at high shear strain rate the molecules align with the flow, giving much smaller effective cross-sections and hence much lower viscosities. More compact molecules are not so much affected by their orientation relative to flow and hence their viscosity changes little with shear strain rate.

In dilute solutions, the relationship of linear and substantially linear polymers depends effectively on the hydrodynamic volume (meaning the volume of a polymer coil when it is in solution, which can vary depending on how well the polymer interacts with the solvent and the polymer's molecular weight) of the molecules as they freely rotate. The relationship between viscosity with concentration is generally linear up to viscosity values of about twice that of water. This dependency means that more extended molecules increase the viscosity to greater extents at low concentrations than more compact molecules of similar molecular weight. At higher concentrations (above C*) all the polymer molecules in the solution effectively overlap, interpenetrate and become entangled (that is, their total hydrodynamic volume appears greater than the solution volume) even without being stressed, changing the solution behavior from mainly viscous to mainly elastic with the viscosity (η0 at zero stress) governed by the mobility of the polymer molecules. C* will depend on the shear strain rate as, at high shear strain rate, the molecules take up a less voluminous shape. At higher concentrations the viscosity increases to cause apparently synergic behavior of hydrocolloid mixtures.

The term “zero-shear viscosity” as used herein refers to the maximum plateau value of viscosity in an at-rest condition, i.e., as shear stress or shear rate is reduced.

### Anatomical Terms

When referring to animals that typically have one end with a head and mouth, with the opposite end often having the anus and tail, the head end is referred to as the cranial end, while the tail end is referred to as the caudal end. Within the head itself, rostral refers to the direction toward the end of the nose, and caudal is used to refer to the tail direction. The surface or side of an animal's body that is normally oriented upwards, away from the pull of gravity, is the dorsal side; the opposite side, typically the one closest to the ground when walking on all legs, swimming or flying, is the ventral side. On the limbs or other appendages, a point closer to the main body is “proximal”; a point farther away is “distal”. Three basic reference planes are used in zoological anatomy. A “sagittal” plane divides the body into left and right portions. The “midsagittal” plane is in the midline, i.e. it would pass through midline structures such as the spine, and all other sagittal planes are parallel to it. A “coronal” plane divides the body into dorsal and ventral portions. A “transverse” plane divides the body into cranial and caudal portions. When referring to humans, the body and its parts are always described using the assumption that the body is standing upright. Portions of the body which are closer to the head end are “superior” (corresponding to cranial in animals), while those farther away are “inferior” (corresponding to caudal in animals). Objects near the front of the body are referred to as “anterior” (corresponding to ventral in animals); those near the rear of the body are referred to as “posterior” (corresponding to dorsal in animals). A transverse, axial, or horizontal plane is an X-Y plane, parallel to the ground, which separates the superior/head from the inferior/feet. A coronal or frontal plane is a Y-Z plane, perpendicular to the ground, which separates the anterior from the posterior. A sagittal plane is an X-Z plane, perpendicular to the ground and to the coronal plane, which separates left from right. The midsagittal plane is the specific sagittal plane that is exactly in the middle of the body.

Structures near the midline are called medial and those near the sides of animals are called lateral. Therefore, medial structures are closer to the midsagittal plane, lateral structures are further from the midsagittal plane. Structures in the midline of the body are median. For example, the tip of a human subject's nose is in the median line.

Ipsilateral means on the same side, contralateral means on the other side and bilateral means on both sides. Structures that are close to the center of the body are proximal or central, while ones more distant are distal or peripheral. For example, the hands are at the distal end of the arms, while the shoulders are at the proximal ends.

### Characteristics of the Site-Specific Sustained Release Microparticulate Pharmaceutical Formulation

According to some embodiments, the site-specific sustained release microparticle pharmaceutical formulation comprises a uniform distribution of microparticles from about 20 μm to about 125 μm in particle size. According to some embodiments, the particle size of the site-specific sustained release microparticulate pharmaceutical formulation is about 20 μm. According to some embodiments, the particle size of the site-specific sustained release microparticulate pharmaceutical formulation is about 25 μm. According to some embodiments, the particle size of the site-specific sustained release microparticulate pharmaceutical formulation is about 30 μm. According to some embodiments, the particle size of the site-specific sustained release microparticulate pharmaceutical formulation is about 31 μm. According to some embodiments, the particle size of the site-specific sustained release microparticulate pharmaceutical formulation is about 32 μm. According to some embodiments, the particle size of the site-specific sustained release microparticulate pharmaceutical formulation is about 33 μm. According to some embodiments, the particle size of the site-specific sustained release microparticulate pharmaceutical formulation is about 34 μm. According to some embodiments, the particle size of the site-specific sustained release microparticulate pharmaceutical formulation is about 35 μm. According to some embodiments, the particle size of the site-specific sustained release microparticulate pharmaceutical formulation is about 36 μm. According to some embodiments, the particle size of the site-specific sustained release microparticulate pharmaceutical formulation is about 37 μm. According to some embodiments, the particle size of the site-specific sustained release microparticulate pharmaceutical formulation is about 38 μm. According to some embodiments, the particle size of the site-specific sustained release microparticulate pharmaceutical formulation is about 39 μm. According to some embodiments, the particle size of the site-specific sustained release microparticulate pharmaceutical formulation is about 40 μm. According to some embodiments, the particle size of the site-specific sustained release microparticulate pharmaceutical formulation is about 45 μm. According to some embodiments, the particle size of the site-specific sustained release microparticulate pharmaceutical formulation is about 50 μm. According to some embodiments, the particle size of the site-specific sustained release microparticulate pharmaceutical formulation is about 55 μm. According to some embodiments, the particle size of the site-specific sustained release microparticulate pharmaceutical formulation is about 60 μm. According to some embodiments, the particle size of the site-specific sustained release microparticulate pharmaceutical formulation is about 65 μm. According to some embodiments, the site-specific sustained release microparticulate pharmaceutical formulation is about 70 μm. According to some embodiments, the particle size of the site-specific sustained release microparticulate pharmaceutical formulation is about 75 μm. According to some embodiments, the particle size of the site-specific sustained release microparticulate pharmaceutical formulation is about 80 μm. According to some embodiments, the particle size of the site-specific sustained release microparticulate pharmaceutical formulation is about 85 μm. According to some embodiments, the particle size of the site-specific sustained release microparticulate pharmaceutical formulation is about 90 μm. According to some embodiments, the particle size of the site-specific sustained release microparticulate pharmaceutical formulation is about 95 μm. According to some embodiments, the particle size of the site-specific sustained release microparticulate pharmaceutical formulation is about 100 μm. According to some embodiments, the particle size of the site-specific sustained release microparticulate pharmaceutical formulation is about 110 μm. According to some embodiments, the particle size of the site-specific sustained release microparticulate pharmaceutical formulation is about 120 μm. According to some embodiments, the particle size of the site-specific sustained release microparticulate pharmaceutical formulation is about 125 μm.

Techniques for measuring particle size include, but are not limited to, microscopy, sieving, sedimentation, optical sensing, electrical sensing, laser light scattering and surface area measurement techniques. Non-limiting examples of microscopy techniques include optical microscopy and electron microscopy. Electron microscopy includes, but is not limited to, Transmission Electron Microscopy (TEM) and Scanning Electron Microscopy (SEM). Non-limiting examples sieving include wet, dry, machine, hand, shaking, vibration, ultrasonic and the like. Sedimentation techniques include, but are not limited to, pipette and photosedimentation. Electrical sensing is performed by an apparatus for counting and sizing particles suspended in electrolytes (e.g., a coulter counter). The number and size of particles suspended in an electrolyte is determined by causing by causing the particles to pass through an opening flanked on either side by an electrode immersed in the electrolyte. Changes in electric impedence (i.e., resistance) as particles pass through the opening generate voltage pulses, the amplitude of which is proportional to the volumes of the particles. Laser light scattering techniques include, but are not limited to, laser diffraction particle size analysis and photon correlation spectroscopy. Laser diffraction instruments (e.g., Mastersizer range from Malvern, LS 13320 from Beckman Coulter) and photon correlation spectrometers (e.g., Photocor Complex from Photocor Instruments) are available from commercial vendors.

According to one embodiment, the site-specific sustained release microparticulate pharmaceutical formulation comprises a plurality of particles. According to another embodiment, the site-specific sustained release microparticulate pharmaceutical formulation comprises a plurality of particles comprising at least one therapeutic agent. According to another embodiment, the site-specific sustained release microparticulate pharmaceutical formulation comprising at least one therapeutic agent further comprises a plurality of particles comprising an additional therapeutic agent.

According to one embodiment, the site-specific sustained release microparticulate pharmaceutical formulation comprises a plurality of microparticles of uniform size distribution, and a therapeutic amount of at least one therapeutic agent, wherein the therapeutic agent is dispersed throughout each microparticle, adsorbed onto the microparticles, or in a core surrounded by a coating.

According to some embodiments, the therapeutic agent is provided in the form of a microparticle. According to another embodiment, the therapeutic agent is disposed on or in the microparticle. According to one embodiment, the therapeutic agent is dispersed throughout each microparticle. According to some embodiments, the therapeutic agent is impregnated on the surface of each microparticle. According to another embodiment, the therapeutic agent is contained within a core of the microparticle surrounded by a coating. According to another embodiment, the therapeutic agent is adsorbed into each microparticle.

According to some embodiments, the additional therapeutic agent is provided in the form of a microparticle. According to another embodiment, the additional therapeutic agent is disposed on or in the microparticle. According to one embodiment, the additional therapeutic agent is dispersed throughout each microparticle. According to some embodiments, the additional therapeutic agent is impregnated on the surface of each microparticle. According to another embodiment, the additional therapeutic agent is contained within a core of the microparticle surrounded by a coating. According to another embodiment, the additional therapeutic agent is adsorbed into each microparticle.

According to some embodiments, the microparticle can be of any order release kinetics, including a zero order release, first order release, second order release, delayed release, sustained release, immediate release, and a combination thereof. The microparticles can include, in addition to therapeutic agent(s), any of those materials routinely used in the art of pharmacy and medicine, including, but not limited to, erodible, nonerodible, biodegradable, or nonbiodegradable material or combinations thereof.

According to some embodiments, the microparticle is a microcapsule that contains the therapeutic agent in a solution or in a semi-solid state. According to some embodiments, the microparticle contains the therapeutic agent, in whole or in part. According to some embodiments, the microparticle can be of virtually any shape.

According to some embodiments, each microparticle is loaded with at least 40% by weight to at least 100% by weight of the therapeutic agent. According to one embodiment, each microparticle is loaded with at least 40% by weight of the therapeutic agent. According to one embodiment, each microparticle is loaded with at least 41% by weight of the therapeutic agent. According to one embodiment, each microparticle is loaded with at least 42% by weight of the therapeutic agent. According to one embodiment, each microparticle is loaded with at least 43% by weight of the therapeutic agent. According to one embodiment, each microparticle is loaded with at least 44% by weight of the therapeutic agent. According to another embodiment, each microparticle is loaded with at least 45% by weight of the therapeutic agent. According to one embodiment, each microparticle is loaded with at least 46% by weight of the therapeutic agent. According to one embodiment, each microparticle is loaded with at least 47% by weight of the therapeutic agent. According to one embodiment, each microparticle is loaded with at least 48% by weight of the therapeutic agent. According to another embodiment, each microparticle is loaded with at least 49% by weight of the therapeutic agent. According to another embodiment, each microparticle is loaded with at least 50% by weight of the therapeutic agent. According to one embodiment, each microparticle is loaded with at least 51% by weight of the therapeutic agent. According to one embodiment, each microparticle is loaded with at least 52% by weight of the therapeutic agent. According to one embodiment, each microparticle is loaded with at least 53% by weight of the therapeutic agent. According to one embodiment, each microparticle is loaded with at least 54% by weight of the therapeutic agent. According to another embodiment, each microparticle is loaded with at least 55% by weight of the therapeutic agent. According to another embodiment, each microparticle is loaded with at least 56% by weight of the therapeutic agent. According to one embodiment, each microparticle is loaded with at least 57% by weight of the therapeutic agent. According to one embodiment, each microparticle is loaded with at least 58% by weight of the therapeutic agent. According to one embodiment, each microparticle is loaded with at least 59% by weight of the therapeutic agent. According to another embodiment, each microparticle is loaded with at least 60% by weight of the therapeutic agent. According to another embodiment, each microparticle is loaded with at least 61% by weight of the therapeutic agent. According to another embodiment, each microparticle is loaded with at least 62% by weight of the therapeutic agent. According to another embodiment, each microparticle is loaded with at least 63% by weight of the therapeutic agent. According to another embodiment, each microparticle is loaded with at least 64% by weight of the therapeutic agent. According to another embodiment, each microparticle is loaded with at least 65% by weight of the therapeutic agent. According to another embodiment, each microparticle is loaded with at least 66% by weight of the therapeutic agent. According to another embodiment, each microparticle is loaded with at least 67% by weight of the therapeutic agent. According to another embodiment, each microparticle is loaded with at least 68% by weight of the therapeutic agent. According to another embodiment, each microparticle is loaded with at least 69% by weight of the therapeutic agent. According to another embodiment, each microparticle is loaded with at least 70% by weight of the therapeutic agent. According to another embodiment, each microparticle is loaded with at least 75% by weight of the therapeutic agent. According to another embodiment, each microparticle is loaded with at least 80% by weight of the therapeutic agent. According to another embodiment, each microparticle is loaded with at least 85% by weight of the therapeutic agent. According to another embodiment, each microparticle is loaded with at least 90% by weight of the therapeutic agent. According to another embodiment, each microparticle is loaded with at least 95% by weight of the therapeutic agent. According to another embodiment, each microparticle is loaded with 100% by weight of the therapeutic agent.

According to some such embodiments, the microparticles are of uniform size distribution. According to some embodiments, the uniform distribution of microparticle size is achieved by a homogenization process to form a uniform emulsion comprising microparticles. According to some such embodiments, each microparticle comprises a matrix. According to some embodiments, the matrix comprises at least one therapeutic agent.

According to another embodiment, the therapeutic agent can be provided in form of a string. The string can contain the therapeutic agent in a core surrounded by a coating, or the therapeutic agent can be dispersed throughout the string, or the therapeutic agent can be absorbed into the string. The string can be of any order release kinetics, including zero order release, first order release, second order release, delayed release, sustained release, immediate release, etc., and any combination thereof. The string can include, in addition to therapeutic agent(s), any of those materials routinely used in the art of pharmacy and medicine, including, but not limited to, erodible, nonerodible, biodegradable, or nonbiodegradable material or combinations thereof.

According to another embodiment, the therapeutic agent can be provided in form of a sheet. The sheet can contain the first therapeutic agent and additional therapeutic agent in a core surrounded by a coating, the first therapeutic agent and additional therapeutic agent can be dispersed throughout the sheet, or the first therapeutic agent can be absorbed into the sheet. The sheet can be of any order release kinetics, including zero order release, first order release, second order release, delayed release, sustained release, immediate release, etc., and any combination thereof. The sheet can include, in addition to the first therapeutic agent and additional therapeutic agent, any of those materials routinely used in the art of pharmacy and medicine, including, but not limited to, erodible, nonerodible, biodegradable, or nonbiodegradable material or combinations thereof.

According to some embodiments, the site-specific sustained release microparticulate formulation comprises a suspension of microparticles. According to one embodiment, the particulate formulation comprises a powder suspension of microparticles. According to some embodiments, the particulate formulation further comprises at least one of a suspending agent, a stabilizing agent and a dispersing agent. According to some such embodiments, the particulate formulation is presented as a dispersion. According to some such embodiments, the particulate formulation is presented as a suspension. According to some such embodiments, the particulate formulation is presented as a solution. According to some such embodiments, the particulate formulation is presented as an emulsion.

According to some embodiments, the site-specific sustained release microparticulate formulation comprises an aqueous solution of the therapeutic agent in water-soluble form. According to some embodiments, the site-specific sustained release microparticulate formulation comprises an oily suspension of the therapeutic agent. An oily suspension of the therapeutic agent can be prepared using suitable lipophilic solvents. Exemplary lipophilic solvents or vehicles include, but are not limited to, fatty oils such as sesame oil, or synthetic fatty acid esters, such as ethyl oleate or triglycerides. According to some embodiments, the site-specific sustained release microparticulate formulation comprises an aqueous suspension of the therapeutic agent. Aqueous suspensions can contain substances which increase the viscosity of the suspension, such as sodium carboxymethyl cellulose, sorbitol, hyaluronic acid, or dextran. Optionally, the suspension can also contain suitable stabilizers or agents which increase the solubility of the compounds to allow for the preparation of highly concentrated solutions. Alternatively, the therapeutic agent can be in powder form for constitution with a suitable vehicle before use. According to some embodiments, the site-specific sustained release microparticulate formulation is dispersed in a vehicle to form a dispersion, with the microparticles as the dispersed phase, and the vehicle as the dispersion medium.

The site-specific sustained release microparticulate formulation can include, for example, microencapsulated dosage forms, and if appropriate, with one or more excipients, encochleated, coated onto microscopic gold particles, contained in liposomes, pellets for implantation into the tissue, or dried onto an object to be rubbed into the tissue. As used herein, the term “microencapsulation” refers to a process in which very tiny droplets or particles are surrounded or coated with a continuous film of biocompatible, biodegradable, polymeric or non-polymeric material to produce solid structures including, but not limited to, nonpareils, pellets, crystals, agglomerates, microspheres, or nanoparticles. The site-specific sustained release microparticulate formulation can be in the form of granules, beads, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, or solubilizers are customarily used as described above. The microparticle formulations are suitable for use in a variety of drug delivery systems. For a brief review of methods for drug delivery, see Langer (1990) Science 249, 1527-1533, which is incorporated herein by reference.

According to one embodiment, the particles comprise a matrix. According to some embodiments, the therapeutic agent is impregnated in or on a naturally occurring biopolymer matrix, a synthetic polymer matrix, or a combination thereof. According to one embodiment, the particulate composition comprises a polymer matrix, wherein the therapeutic agent is impregnated in the polymer matrix. According to one embodiment, the polymer is a slow release compound. According to one embodiment, the polymer is a biodegradable polymer.

According to one embodiment, the polymer is poly (D, L-Lactide-co-glycolide) (PLGA). According to some embodiments, the lactide to glycolide ratio of PLGA is 50:50. According to some embodiments, the lactide to glycolide ratio of PLGA is 65:35. According to some embodiments, the lactide to glycolide ratio of PLGA is 75:25. According to some embodiments, the lactide to glycolide ratio of PLGA is 85:15. According to some embodiments, the PLGA is an ester. According to some embodiments, the PLGA is an acid.

According to another embodiment, the polymer is poly(orthoester). According to another embodiment, the polymer is poly(anhydride). According to another embodiment, the polymer is polylactide-polyglycolide.

Both non-biodegradable and biodegradable polymeric materials can be used in the manufacture of particles for delivering the therapeutic agents. Such polymers can be natural or synthetic polymers. The polymer is selected based on the period of time over which release is desired. Biodegradable polymers include, but are not limited to, bioerodible hydrogels as described by Sawhney et al in Macromolecules (1993) 26, 581-587, the teachings of which are incorporated herein. Exemplary bioerodible hydrogels include, but are not limited to, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). According to one embodiment, the biodegradable polymer is hyaluronic acid. According to some embodiments, the hyaluronic acid is a natural hyaluronic acid. According to some embodiments, the hyaluronic acid is a recombinant hyaluronic acid. According to some embodiments, the hyaluronic acid is characterized by a zero shear rate viscosity of 2 Poise and a molecular weight 0.500-0.750 million Da. According to some embodiments, the hyaluronic acid is characterized by a zero shear rate viscosity of 1677 Poise and a molecular weight 1.0-2.9 million Da.

According to another embodiment, the polymer enhances aqueous solubility of the particulate formulation. Exemplary polymers include but are not limited to polyethylene glycol, poly-(d-glutamic acid), poly-(1-glutamic acid), poly-(d-aspartic acid), poly-(1-aspartic acid) and copolymers thereof. Polyglutamic acids having molecular weights between about 5,000 to about 100,000, with molecular weights between about 20,000 and about 80,000, and with molecular weights between about 30,000 and about 60,000 may also be used. The polymer is conjugated via an ester linkage to one or more hydroxyls of an inventive epothilone using a protocol as essentially described by U.S. Pat. No. 5,977,163 which is incorporated herein by reference. Particular conjugation sites include the hydroxyl off carbon-21 in the case of 21-hydroxy-derivatives of the present invention. Other conjugation sites include, but are not limited, to the hydroxyl off carbon 3 and/or the hydroxyl off carbon 7.

According to some embodiments, the therapeutic agent is impregnated in or on a polyglycolide (PGA) matrix. PGA is a linear aliphatic polyester developed for use in sutures. Studies have reported PGA copolymers formed with trimethylene carbonate, polylactic acid (PLA), and polycaprolactone. Some of these copolymers may be formulated as microparticles for sustained drug release.

According to some embodiments, the therapeutic agent is impregnated in or on a polyester-polyethylene glycol matrix. Polyester-polyethylene glycol compounds can be synthesized; these are soft and may be used for drug delivery.

According to some embodiments, the therapeutic agent is impregnated in or on a poly (amino)-derived biopolymer matrix. Poly (amino)-derived biopolymers can include, but are not limited to, those containing lactic acid and lysine as the aliphatic diamine (see, for example, U.S. Pat. No. 5,399,665), and tyrosine-derived polycarbonates and polyacrylates. Modifications of polycarbonates may alter the length of the alkyl chain of the ester (ethyl to octyl), while modifications of polyarylates may further include altering the length of the alkyl chain of the diacid (for example, succinic to sebasic), which allows for a large permutation of polymers and great flexibility in polymer properties.

According to some embodiments, the therapeutic agent is impregnated in or on a polyanhydride matrix. Polyanhydrides are prepared by the dehydration of two diacid molecules by melt polymerization (see, for example, U.S. Pat. No. 4,757,128). These polymers degrade by surface erosion (as compared to polyesters that degrade by bulk erosion). The release of the drug can be controlled by the hydrophilicity of the monomers chosen.

According to some embodiments, the therapeutic agent is impregnated in or on a photopolymerizable biopolymer matrix. Photopolymerizable biopolymers include, but are not limited to, lactic acid/polyethylene glycol/acrylate copolymers.

According to some embodiments, the therapeutic agent is impregnated in or on a hydrogel matrix. The term “hydrogel” refers to a substance resulting in a solid, semisolid, pseudoplastic or plastic structure containing a necessary aqueous component to produce a gelatinous or jelly-like mass. Hydrogels generally comprise a variety of polymers, including hydrophilic polymers, acrylic acid, acrylamide and 2-hydroxyethylmethacrylate (HEMA).

According to some embodiments, the therapeutic agent is impregnated in or on a naturally-occurring biopolymer matrix. Naturally-occurring biopolymers include, but are not limited to, protein polymers, collagen, polysaccharides, and photopolymerizable compounds.

According to some embodiments, the therapeutic agent is impregnated in or on a protein polymer matrix. Protein polymers have been synthesized from self-assembling protein polymers such as, for example, silk fibroin, elastin, collagen, and combinations thereof.

According to some embodiments, the therapeutic agent is impregnated in or on a naturally-occurring polysaccharide matrix. Naturally-occurring polysaccharides include, but are not limited to, chitin and its derivatives, hyaluronic acid, dextran and cellulosics (which generally are not biodegradable without modification), and sucrose acetate isobutyrate (SAIB).

According to some embodiments, the therapeutic agent is impregnated in or on a chitin matrix. Chitin is composed predominantly of 2-acetamido-2-deoxy-D-glucose groups and is found in yeasts, fungi and marine invertebrates (shrimp, crustaceous) where it is a principal component of the exoskeleton. Chitin is not water soluble and the deacetylated chitin, chitosan, only is soluble in acidic solutions (such as, for example, acetic acid). Studies have reported chitin derivatives that are water soluble, very high molecular weight (greater than 2 million Daltons), viscoelastic, non-toxic, biocompatible and capable of crosslinking with peroxides, gluteraldehyde, glyoxal and other aldehydes and carbodiamides, to form gels.

According to some embodiments, the therapeutic agent is impregnated in or on a hyaluronic acid (HA) matrix. HA, which is composed of alternating glucuronidic and glucosaminidic bonds and is found in mammalian vitreous humor, extracellular matrix of the brain, synovial fluid, umbilical cords and rooster combs from which it is isolated and purified, also can be produced by fermentation processes.

According to some embodiments, the site-specific sustained release microparticulate pharmaceutical formulation is stable for at least 30 days at 4° C. According to some embodiments, the site-specific sustained release microparticulate pharmaceutical formulation is stable for at least 30 days at 25° C. According to some embodiments, the site-specific sustained release microparticulate pharmaceutical formulation is stable for at least 30 days at 30° C.−35° C.

Microencapsulation Process

Examples of microencapsulation processes and products; methods for the production of emulsion-based microparticles; emulsion-based microparticles and methods for the production thereof; solvent extraction microencapsulation with tunable extraction rates; microencapsulation process with solvent and salt; a continuous double emulsion process for making microparticles; drying methods for tuning microparticle properties, controlled release systems from polymer blends; polymer mixtures comprising polymers having different non-repeating units and methods for making and using the same; and an emulsion based process for preparing microparticles and workhead assembly for use with same are disclosed and described in, but not limited to U.S. Pat. No. 5,407,609 (entitled Microencapsulation Process and Products Thereof), U.S. Application Publication No. US 2007-0190154 A1 (entitled Method for the production of emulsion-based microparticles), U.S. Application Publication No. US 2007-0207211 A1 (entitled Emulsion-based microparticles and methods for the production thereof), U.S. Application Publication No. US 2010-0063179 A1 (entitled Solvent Extraction Microencapsulation With Tunable Extraction Rates), U.S. Application Publication No. US 2010-0291027 A1 (entitled Hyaluronic Acid (HA) Injection Vehicle), U.S. Application Publication No. US 2010-0069602 A1 entitled Microencapsulation Process With Solvent And Salt), U.S. Application No. US 2009-0162407 A1 (entitled Process For Preparing Microparticles Having A Low Residual Solvent Volume); U.S. Application Publication No. US 2010-0189763 A1 (entitled Controlled Release Systems From Polymer Blends); U.S. Application Publication No. US 2010-0216948 A1 (entitled Polymer Mixtures Comprising Polymers Having Different Non-Repeating Units And Methods For Making And Using Same); U.S. Application Publication No. US 2007-0092574 A1 (entitled “Controlled release compositions”); U.S. application Ser. No. 12/692,029 (entitled “Drying Methods for Tuning Microparticle Properties); U.S. Application Publication No. US 2011-0204533 A1 (entitled “Emulsion Based Process for Preparing Microparticles and Workhead for Use with Same); and U.S. Application Publication No. US 2011-0236497 A1 (entitled Composition and Methods for Improved Retention of a Pharmaceutical Composition at a Local Administration Site”). The contents of each of these patents and patent application publications are incorporated herein by reference in its entirety.

According to some embodiments, delivery of the active therapeutic agent(s) using microparticle technology involves bioresorbable, polymeric particles that encapsulate the first therapeutic agent and additional therapeutic agent.

The site-specific sustained release microparticulate pharmaceutical formulation containing a uniform distribution of microparticle size can be prepared by an emulsion based process, for example as described in U.S. Pat. No. 5,407,609, the entire content of which is incorporated herein by reference.

According to one embodiment a process for producing a bioactive agent encapsulated into particles comprises: (a) providing a substantially pure crystalline form of the bioactive agent; (b) adding the substantially pure crystalline form of the bioactive agent to a polymer solution, thereby creating a mixture of the bioactive agent and the polymer solution; (c) homogenizing the mixture to form a disperse phase; (d) mixing the disperse phase with a continuous phase comprising a continuous process medium, thereby forming an emulsion comprising the bioactive agent; (e) forming and extracting the particles comprising the substantially pure bioactive agent; and (f) drying the particles.

It is understood and herein contemplated that where a polymer solution comprises a polymer in an organic solvent forming a oil/water emulsion in the disperse phase, mixing the disperse phase with the continuous phase results in a double emulsion (i.e., a water/oil/water emulsion). Where the polymer solution comprises a polymer in an aqueous solvent such as water, only a single emulsion is formed upon mixing the dispersed phase with the continuous phase.

According to one embodiment, the continuous process medium comprises a surfactant and the bioactive agent saturated with the solvent used in the polymer solution.

According to one embodiment, the polymer solutions of the aforementioned processes comprise a polymer and a solvent. It is understood and herein contemplated that the disclosed polymers comprise in one aspect polylactide, polylactide-co-glycolide, poly(orthoester), and poly(anhydride). According to some embodiments, the polylactide co-glycolide can be in an 85:15, 75:25, 65:35, or 50:50 ratio of lactide to glycolide. In another aspect, the solvent can comprise ethyl acetate or dichloromethane.

According to another embodiment, the processes disclosed herein comprise drying the particles over a 10 to 48 hour period.

### Pharmaceutical Carrier

According to some embodiments, the site-specific sustained release microparticulate pharmaceutical formulation comprises a pharmaceutically acceptable carrier.

According to one embodiment, the pharmaceutically acceptable carrier is a solid carrier or excipient. According to another embodiment, the pharmaceutically acceptable carrier is a gel-phase carrier or excipient. Examples of carriers or excipients include, but are not limited to, calcium carbonate, calcium phosphate, various monomeric and polymeric sugars (including without limitation hyaluronic acid), starches, cellulose derivatives, gelatin, and polymers. An exemplary carrier can also include saline vehicle, e.g. hydroxyl propyl methyl cellulose (HPMC) in phosphate buffered saline (PBS). According to another embodiment, the pharmaceutically acceptable carrier is a buffer solution. Exemplary buffer solutions can include without limitation a phosphate buffered saline (PBS) solution.

Suitable injection vehicles for use in the present invention can be found in U.S. Pat. No. 6,495,164, U.S. Patent Application Publication No. 2010/0303900, U.S. Patent Application Publication No. 2010/0330184, and U.S. Patent Application Publication No. 2010/0291027, the entire disclosures of which are incorporated herein by reference. Exemplary injection vehicles suitable for use in the present invention include, but are not limited to, water, saline (sodium chloride solution, hydroxyl propyl methyl cellulose (HPMC) in phosphate buffered saline (PBS)), and hyaluronic acid and hyaluronic acid derivatives, or a combination thereof. Exemplary hyaluronic acid derivatives can include, but are not limited to, salts, esters, amides, and lactide derivatives. Exemplary hyaluronic acid derivatives suitable for use in the present invention are provided in U.S. Pat. No. 5,527,893, U.S. Pat. No. 5,017,229, and U.S. Pat. No. 4,937,270, the entire disclosures of which are incorporated herein by reference. According to one embodiment, the injection vehicle can be combined with a suitable surfactant. Exemplary surfactants can include, but are not limited to, poly(vinyl alcohol), carboxymethyl cellulose, gelatin, poly(vinyl pyrrolidone), Tween 80, Tween 20, or a combination thereof.

According to one embodiment, the pharmaceutically acceptable carrier comprises a hyaluronic acid or a hyaluronic acid derivative. According to some embodiments, the hyaluronic acid or the hyaluronic acid derivative thereof has an average molecular weight ranging between about 5 KDa to about 20,000 KDa. According to some embodiments, the hyaluronic acid or the hyaluronic acid derivative thereof has an average molecular weight of about 5 KDa, 10 KDa, 20 KDa, 30 KDa, 40 KDa, 50 KDa, 60 KDa, 70 KDa, 80 KDa, 90 KDa, 100 KDa, 200 KDa, 300 KDa, 400 KDa, 500 KDa, 600 KDa, 700 KDa, 800 KDa, 900 KDa, 1,000 KDa, 2,000 KDa, 3,000 KDa, 4,000 KDa, 5,000 KDa, 6,000 KDa, 7,000 KDa, 8,000 KDa, 9,000 KDa, 10,000 KDa, 11,000 KDa, 12,000 KDa, 13,000 KDa, 14,000 KDa, 15,000 KDa, 16,000 KDa, 17,000 KDa, 18,000 KDa, 19,000 KDa, or 20,000 KDa. According to one embodiment, the hyaluronic acid or the hyaluronic acid derivative thereof has an average molecular weight of about 500 KDa.

According to some embodiments, the pharmaceutically acceptable carrier imparts stickiness to the composition. According to one embodiment, the pharmaceutically acceptable carrier comprises hyaluronic acid. According to some embodiments, the pharmaceutically acceptable carrier comprises 0% to 5% by weight hyaluronic acid or the hyaluronic acid derivative. According to one embodiment, the pharmaceutically acceptable carrier comprises less than 0.01% by weight hyaluronic acid or the hyaluronic acid derivative. According to another embodiment, the pharmaceutically acceptable carrier comprises less than 0.05% by weight hyaluronic acid or the hyaluronic acid derivative. According to another embodiment, the pharmaceutically acceptable carrier comprises less than 0.1% by weight hyaluronic acid or the hyaluronic acid derivative. According to another embodiment, the pharmaceutically acceptable carrier comprises less than 0.2% by weight hyaluronic acid or the hyaluronic acid derivative. According to another embodiment, the pharmaceutically acceptable carrier comprises less than 0.3% by weight hyaluronic acid or the hyaluronic acid derivative. According to another embodiment, the pharmaceutically acceptable carrier comprises less than 0.4% by weight hyaluronic acid or the hyaluronic acid derivative. According to another embodiment, the pharmaceutically acceptable carrier comprises less than 0.5% by weight hyaluronic acid or the hyaluronic acid derivative. According to another embodiment, the pharmaceutically acceptable carrier comprises less than 0.6% by weight hyaluronic acid or the hyaluronic acid derivative. According to another embodiment, the pharmaceutically acceptable carrier comprises less than 0.7% by weight hyaluronic acid or the hyaluronic acid derivative. According to another embodiment, the pharmaceutically acceptable carrier comprises less than 0.8% by weight hyaluronic acid or the hyaluronic acid derivative. According to another embodiment, the pharmaceutically acceptable carrier comprises less than 0.9% by weight hyaluronic acid or the hyaluronic acid derivative. According to another embodiment, the pharmaceutically acceptable carrier comprises less than 1.0% by weight hyaluronic acid or the hyaluronic acid derivative. According to another embodiment, the pharmaceutically acceptable carrier comprises less than 1.1% by weight hyaluronic acid or the hyaluronic acid derivative. According to another embodiment, the pharmaceutically acceptable carrier comprises less than 1.2% by weight hyaluronic acid or the hyaluronic acid derivative. According to another embodiment, the pharmaceutically acceptable carrier comprises less than 1.3% by weight hyaluronic acid or the hyaluronic acid derivative. According to another embodiment, the pharmaceutically acceptable carrier comprises less than 1.4% by weight hyaluronic acid or the hyaluronic acid derivative According to another embodiment, the pharmaceutically acceptable carrier comprises less than 1.5% by weight hyaluronic acid or the hyaluronic acid derivative. According to another embodiment, the pharmaceutically acceptable carrier comprises less than 1.6% by weight hyaluronic acid or the hyaluronic acid derivative. According to another embodiment, the pharmaceutically acceptable carrier comprises less than 1.7% by weight hyaluronic acid or the hyaluronic acid derivative. According to another embodiment, the pharmaceutically acceptable carrier comprises less than 1.8% by weight hyaluronic acid or the hyaluronic acid derivative. According to another embodiment, the pharmaceutically acceptable carrier comprises less than 1.9% by weight hyaluronic acid or the hyaluronic acid derivative. According to another embodiment, the pharmaceutically acceptable carrier comprises less than 2.0% by weight hyaluronic acid or the hyaluronic acid derivative. According to another embodiment, the pharmaceutically acceptable carrier comprises less than 2.1% by weight hyaluronic acid or the hyaluronic acid derivative. According to another embodiment, the pharmaceutically acceptable carrier comprises less than 2.2% by weight hyaluronic acid or the hyaluronic acid derivative. According to another embodiment, the pharmaceutically acceptable carrier comprises less than 2.3% by weight hyaluronic acid or the hyaluronic acid derivative. According to another embodiment, the pharmaceutically acceptable carrier comprises less than 2.4% by weight hyaluronic acid or the hyaluronic acid derivative. According to another embodiment, the pharmaceutically acceptable carrier comprises less than 2.5% by weight hyaluronic acid or the hyaluronic acid derivative. According to another embodiment, the pharmaceutically acceptable carrier comprises less than 2.6% by weight hyaluronic acid or the hyaluronic acid derivative. According to another embodiment, the pharmaceutically acceptable carrier comprises less than 2.7% by weight hyaluronic acid or the hyaluronic acid derivative. According to another embodiment, the pharmaceutically acceptable carrier comprises less than 2.8% by weight hyaluronic acid or the hyaluronic acid derivative. According to another embodiment, the pharmaceutically acceptable carrier comprises less than 2.9% by weight hyaluronic acid or the hyaluronic acid derivative. According to another embodiment, the pharmaceutically acceptable carrier comprises less than 3.0% by weight hyaluronic acid or the hyaluronic acid derivative. According to another embodiment, the pharmaceutically acceptable carrier comprises less than 3.5% by weight hyaluronic acid or the hyaluronic acid derivative. According to another embodiment, the pharmaceutically acceptable carrier comprises less than 4.0% by weight hyaluronic acid or the hyaluronic acid derivative. According to another embodiment, the pharmaceutically acceptable carrier comprises less than 4.5% by weight hyaluronic acid or the hyaluronic acid derivative. According to another embodiment, the pharmaceutically acceptable carrier comprises less than 5.0% by weight hyaluronic acid or the hyaluronic acid derivative.

According to some embodiments, the pH of the hyaluronic acid or the hyaluronic acid derivative thereof is 6.8, 6.9, 7.0, 7.1, 7.2, 7.3, 7.4, 7.5, or 7.6.

According to some embodiments, the osmolarity of the hyaluronic acid or the hyaluronic acid derivative thereof is about 250 mOsm/kg, about 258 mOsm/kg, about 275 mOsm/kg, about 300 mOsm/kg, about 325 mOsm/kg, about 350 mOsm/kg, about 375 mOsm/kg, or about 381 mOsm/kg.

Exemplary hyaluronic acid derivatives can include, but are not limited to, salts, esters, amides, and lactide derivatives. Exemplary hyaluronic acid derivatives suitable for use in the present invention are provided in U.S. Pat. No. 5,527,893, U.S. Pat. No. 5,017,229, and U.S. Pat. No. 4,937,270, the entire disclosures of which are incorporated herein by reference. According to one embodiment, the injection vehicle can be combined with a suitable surfactant. Exemplary surfactants can include, but are not limited to, poly(vinyl alcohol), carboxymethyl cellulose, gelatin, poly(vinyl pyrrolidone), Tween 80, Tween 20, or a combination thereof.

Hyaluronic acid (hyaluronate sodium salt, “HA”) is a naturally occurring glycosaminoglycan found in the extracellular matrix and is an abundant component of the extracellular space of the brain. (Laurent T C et al., “The structure and function of hyaluronan: An overview,” Immunol. Cell. Biol., (1996) 74:A1-A7). It is found normally in synovial joints where it is believed to function as a lubricant, among other functions. Normal CSF also contains HA. It is composed of repeated nonsulfated disaccharide units of D-glucuronic acid and N-acetyl-D-glucosamine linked by β(1-3) and β(1-4) glycoside linkages, respectively. HA can be assembled in varying molecular weights and lyophilized or etherified to alter the rate of degradation. The HA derivatives used in most human products are synthesized by fermentation in bacteria, overcoming problems with toxicity, immunological reactions and allergies due to contaminants associated with naturally derived HA from avian sources. HA is formulated for injection into joints to treat pain from osteoarthritis (Orthovisc®, Nuflexxa®, Hyalgan® and others), for injection into the eye during ophthalmic surgery (Healon®, Viscoat®, Biolon®), as epidural injection films (Seprafilm®) and for use in otolaryngology (Merogel®). Sodium hyaluronate is listed in the FDA's inactive ingredients list for administration via several parenteral routes including intravitreal, intraarticular and intramuscular.

According to one embodiment, the site-specific sustained release microparticulate pharmaceutical formulation of the described invention can be mixed with a <2.3 w/w % bacterial-derived sodium hyaluronate solution in PBS with 0.1% polysorbate 20. According to some embodiments, the pH is about 6.8, about 6.9, about 7.0, about 7.1, about 7.2, about 7.3, about 7.4, about 7.5, or about 7.6. According to some embodiments, the osmolarity is about 250 mOsm/kg, about 258 mOsm/kg, about 275 mOsm/kg, about 300 mOsm/kg, about 325 mOsm/kg, about 350 mOsm/kg, about 375 mOsm/kg, or about 381 mOsm/kg. The average molecular weight of the sodium hyaluronate is approximately 500 kDa. The 2.3 w/w % solution is approved for use. Injection volumes of 1% HA into joints are typically 2 mL. The maximum volume of the site-specific sustained release microparticle formulation of the described invention contains a similar amount of HA.

As a polymer within a solution, hyaluronic acid shows non-Newtonian and viscoelastic behavior; non-Newtonian liquids have no fixed viscosity value because of the changes in value caused by the amount of shear applied. The most common of these changes is shear-thinning, where the viscosity decreases as the shear-rate increases. The zero shear viscosity, ηₒ, correlates with polymer solution concentration multipled by polymer molecular weight. Falcone, S J et al, “Rheological and cohesive properties of hyaluronic acid,” J. Biomed. Mat. Res. A. 76(4): 721-28 (2006). HA when applied through a small cannula is not at rest but must flow through a small orifice rapidly, i.e., it experiences high shear forces. The cohesive nature of HA polymer solutions can be increased by decreasing the solution concentration or increasing the molecular weight of the polymer in solution. Id. High molecular weight HA is more cohesive than low molecular weight HA. Id

According to some embodiments, the viscosity of the site-specific sustained release microparticulate pharmaceutical formulation at atmospheric pressure and near room temperature ranges from that of a simple liquid; to that of a warm honey; to that of a paste.

According to some embodiments, the viscosity of the site-specific sustained release microparticulate pharmaceutical formulation at atmospheric pressure and near room temperature is about 1 Poise, 2, Poise, 3 Poise, 4 Poise, 5 Poise, 6 Poise, 7 Poise, 8 Poise, 9 Poise, 10 Poise, 20 Poise, 30 Poise, 40 Poise, 50 Poise, 60 Poise, 70 Poise, 80 Poise, 90 Poise, 100 Poise, 200 Poise, 300 Poise, 400 Poise, 500 Poise, 600 Poise, 700 Poise, 800 Poise, 900 Poise, 1,000 Poise, 1,100 Poise, 1,200 Poise, 1,300 Poise, 1,400 Poise, 1,500 Poise, 1,600 Poise, 1,700 Poise, 1,800 Poise, 1,900 Poise or 2,000 Poise.

According to one embodiment, the viscosity of the site-specific sustained release microparticulate pharmaceutical formulation at atmospheric pressure and near room temperature ranges from about 1 Poise to about 2,000 Poise. According to one embodiment, the viscosity of the site-specific sustained release microparticulate pharmaceutical formulation at atmospheric pressure and near room temperature ranges from about 1 Poise to about 1,700 Poise. According to one embodiment, the viscosity of the site-specific sustained release microparticulate pharmaceutical formulation at atmospheric pressure and near room temperature ranges from about 10 Poise to about 1,000 Poise. According to one embodiment, the viscosity of the site-specific sustained release microparticulate pharmaceutical formulation at atmospheric pressure and near room temperature ranges from about 700 Poise to about 1,000 Poise. According to one embodiment, the viscosity of the site-specific sustained release microparticulate pharmaceutical formulation at atmospheric pressure and near room temperature ranges from about 1 Poise to about 10 Poise. According to one embodiment, the viscosity of the site-specific sustained release microparticulate pharmaceutical formulation at atmospheric pressure and near room temperature ranges from about 1 Poise to about 8 Poise. According to one embodiment, the viscosity of the site-specific sustained release microparticulate pharmaceutical formulation at atmospheric pressure and near room temperature ranges from about 1 Poise to about 6 Poise. According to one embodiment, the viscosity of the site-specific sustained release microparticulate pharmaceutical formulation at atmospheric pressure and near room temperature ranges from about 1 Poise to about 4 Poise. According to one embodiment, the viscosity of the site-specific sustained release microparticulate pharmaceutical formulation at atmospheric pressure and near room temperature ranges from about 1.5 Poise to about 3.5 Poise. According to one embodiment, the viscosity of the site-specific sustained release microparticulate pharmaceutical formulation at atmospheric pressure and near room temperature ranges from about 1,000 Poise to about 2,000 Poise. According to one embodiment, the viscosity of the site-specific sustained release microparticulate pharmaceutical formulation at atmospheric pressure and near room temperature ranges from about 1,500 Poise to about 1,700 Poise.

According to one embodiment, the viscosity of the site-specific sustained release microparticulate pharmaceutical formulation at atmospheric pressure and near room temperature is about 1 Poise. According to one embodiment, the viscosity of the site-specific sustained release microparticulate pharmaceutical formulation at atmospheric pressure and near room temperature is about 1.5 Poise. According to one embodiment, the viscosity of the site-specific sustained release microparticulate pharmaceutical formulation at atmospheric pressure and near room temperature is about 2 Poise. According to one embodiment, the viscosity of the site-specific sustained release microparticulate pharmaceutical formulation at atmospheric pressure and near room temperature is about 2.5 Poise. According to one embodiment, the viscosity of the site-specific sustained release microparticulate pharmaceutical formulation at atmospheric pressure and near room temperature is about 3 Poise. According to one embodiment, the viscosity of the site-specific sustained release microparticulate pharmaceutical formulation at atmospheric pressure and near room temperature is about 3.5 Poise. According to one embodiment, the viscosity of the site-specific sustained release microparticulate pharmaceutical formulation at atmospheric pressure and near room temperature is about 4 Poise. According to one embodiment, the viscosity of the site-specific sustained release microparticulate pharmaceutical formulation at atmospheric pressure and near room temperature is about 4.5 Poise. According to one embodiment, the viscosity of the site-specific sustained release microparticulate pharmaceutical formulation at atmospheric pressure and near room temperature is about 5 Poise. According to one embodiment, the viscosity of the site-specific sustained release microparticulate pharmaceutical formulation at atmospheric pressure and near room temperature is about 6 Poise. According to one embodiment, the viscosity of the site-specific sustained release microparticulate pharmaceutical formulation at atmospheric pressure and near room temperature is about 7 Poise. According to one embodiment, the viscosity of the site-specific sustained release microparticulate pharmaceutical formulation at atmospheric pressure and near room temperature is about 8 Poise. According to one embodiment, the viscosity of the site-specific sustained release microparticulate pharmaceutical formulation at atmospheric pressure and near room temperature is about 100 Poise. According to one embodiment, the viscosity of the site-specific sustained release microparticulate pharmaceutical formulation at atmospheric pressure and near room temperature is about 700 Poise. According to one embodiment, the viscosity of the site-specific sustained release microparticulate pharmaceutical formulation at atmospheric pressure and near room temperature is about 800 Poise. According to one embodiment, the viscosity of the site-specific sustained release microparticulate pharmaceutical formulation at atmospheric pressure and near room temperature is about 900 Poise. According to one embodiment, the viscosity of the site-specific sustained release microparticulate pharmaceutical formulation at atmospheric pressure and near room temperature is about 1,000 Poise. According to one embodiment, the viscosity of the site-specific sustained release microparticulate pharmaceutical formulation at atmospheric pressure and near room temperature is about 1,670 Poise.

The biocompatibility and non-immunogenicity of HA have been attributed to its relatively simple structure, which is conserved throughout all mammals, and its poor interaction with blood components. (Amarnath L P et al., “In vitro hemocompatibility testing of UV-modified hyaluronan hydrogels,” Biomaterials, 27:1416-1424 (2006)). HA is degraded in mammals by 3 types of enzymes: hyaluronase, β-D-glucuronidase, and β-N-acetyl-hexosaminidase. Generally, hyaluronase acts on the high molecular weight species to reduce the polysaccharide to oligosaccharides. β-D-glucuronidase, and β-N-acetyl-hexosaminidase in turn degrade the oligosaccharides by removing the nonreducing terminal sugars. (Chen W Y, and Abatangelo G, “Functions of hyaluronan in wound repair,” Wound Repair Regen., 7:79-89 (1999); Leach J B et al., “Development of photocrosslinkable hyaluronic acid-polyethylene glycol-peptide composite hydrogels for soft tissue engineering,” J. Biomed. Mater. Res. A, 70:74-82 (2004)).

Hyaluronate has been shown to be non-mutagenic, non-cytotoxic, and non-neurotoxic. Jansen, et al., found that HA was not cytotoxic when used as a conduit for peripheral nerve repair. (Jansen K et al., “A hyaluronan-based nerve guide: in vitro cytotoxicity, subcutaneous tissue reactions, and degradation in the rat,” Biomaterials, 25:483-489 (2004)). Product information for Orthovisc® (high molecular weight hyaluronate) shows that HA is not mutagenic in several assays including the Sister chromatid exchange assay, the chromosomal aberration assay, and the Ames Salmonella/Mammalian Microsome mutagenicity assay. (Orthovisc®, High Molecular Weight Hyaluronan, Package insert, Anika Therapeutics, Inc., Distributed by DePuy Mitek, a Johnson and Johnson Company). Chronic administration of HA did not result in reproduction toxicity in rats and rabbit at doses up to 1.43 mg/kg per treatment cycle.

Hyaluronic acid was reported to have anti-inflammatory and bacteriostatic effects. (Burns J W et al., “Preclinical evaluation of Seprafilm bioresorbable membrane,” Eur. J. Surg. Suppl., 40-48 (1997)) Injection of 0.2 mL/kg of HA (10 mg/mL, molecular weight 1100 kDa, pH 6.3-8.3) into the epidural space of 10 rabbits did not produce any clinically detectable abnormalities or neurotoxicity. (Lim Y J et al., “The neurotoxicity of epidural hyaluronic acid in rabbits: a light and electron microscopic examination,” Anesth. Analg., 97:1716-1720 (2003)).]

According to some embodiments, the pharmaceutically acceptable carrier includes, but is not limited to, a gel, slow-release solid or semisolid compound, optionally as a sustained release gel. In some such embodiments, the at least one first therapeutic agent is embedded into the pharmaceutically acceptable carrier. In some embodiments, the at least one first therapeutic agent is coated on the pharmaceutically acceptable carrier. The coating can be of any desired material, preferably a polymer or mixture of different polymers. Optionally, the polymer can be utilized during the granulation stage to form a matrix with the active ingredient so as to obtain a desired release pattern of the active ingredient. The gel, slow-release solid or semisolid compound is capable of releasing the active agent over a desired period of time. The gel, slow-release solid or semisolid compound can be implanted in a tissue within the parenchyma of human brain, including, but not limited to, in proximity to a blood vessel, such as a cerebral artery.

According to another embodiment, the pharmaceutically acceptable carrier comprises a slow-release solid compound. According to one embodiment, the therapeutic agent is embedded in the slow-release solid compound or coated on the slow-release solid compound. According to another embodiment, the pharmaceutically acceptable carrier comprises a slow-release microparticle containing at least one therapeutic agent.

According to another embodiment, the pharmaceutically acceptable carrier is a gel compound, such as a biodegradable hydrogel.

### Additional Components

According to some embodiments, the site-specific sustained release microparticulate pharmaceutical formulation further comprises a surfactant. Exemplary surfactants can include, but are not limited to, poly(vinyl alcohol), carboxymethyl cellulose, gelatin, poly(vinyl pyrrolidone), Tween 80, Tween 20, or a combination thereof.

According to some embodiments, the site-specific sustained release microparticulate pharmaceutical formulation further comprises a preservative agent. According to some such embodiments, the site-specific sustained release microparticulate pharmaceutical formulation is presented in a unit dosage form. Exemplary unit dosage forms include, but are not limited to, ampoules or multi-dose containers.

The site-specific sustained release microparticulate pharmaceutical formulation for parenteral administration include aqueous and non-aqueous sterile injection solutions that can contain anti-oxidants, buffers, bacteriostats and solutes, which render the formulation isotonic with the blood or CSF of the intended recipient; and aqueous and non-aqueous sterile suspensions, which can include suspending agents and thickening agents.

According to some embodiments, the site-specific sustained release microparticulate pharmaceutical formulation is formulated for local injection, parenteral injection, implantation, or a combination thereof. According to some such embodiments, the site-specific sustained release microparticulate pharmaceutical formulation is in the form of a pharmaceutically acceptable sterile aqueous or nonaqueous solution, dispersion, suspension, emulsion or a sterile powder for reconstitution into a sterile injectable solution or dispersion. Examples of suitable aqueous and nonaqueous carriers, diluents, solvents or vehicles include, but are not limited to, water, ethanol, dichloromethane, acetonitrile, ethyl acetate, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Suspensions can further contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

According to some embodiments, the site-specific sustained release microparticulate pharmaceutical formulation is formulated in an injectable depot form. Injectable depot forms are made by forming microencapsulated matrices of the therapeutic agent in a biodegradable polymer. Depending upon the ratio of drug to polymer and the nature of the particular polymer employed, the rate of drug release may be controlled. Such long acting formulations can be formulated with suitable polymeric or hydrophobic materials (for example as an emulsion in an acceptable oil) or ion exchange resins, or as sparingly soluble derivatives, for example, as a sparingly soluble salt. Examples of biodegradable polymers include, but are not limited to, polylactide-polyglycolide, poly(orthoesters) and poly(anhydrides). Depot injectable formulations are also prepared by entrapping the drug in liposomes or microemulsions which are compatible with body tissues.

According to some embodiments, the site-specific sustained release microparticulate pharmaceutical formulation further comprises an adjuvant. Exemplary adjuvants include, but are not limited to, preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. Isotonic agents, for example, sugars, sodium chloride and the like, can also be included. Prolonged absorption of the injectable pharmaceutical form can be brought about by the use of agents delaying absorption, for example, aluminum monostearate and gelatin.

According to some embodiments, the site-specific sustained release microparticulate pharmaceutical formulation is aseptically manufactured.

According to some embodiments, the site-specific sustained release microparticulate pharmaceutical formulation is terminally sterilized (TS). Non-limiting examples of terminal sterilization include, but are not limited to, ionizing radiation (e.g., gamma irradiation and electron beam sterilization), infrared radiation, microwaves, glass bead, vaporized hydrogen peroxide, ozone, gas plasma, formaldehyde steam, chlorine dioxide, ethylene oxide, peracetic acid, performic acid, glutaraldehyde, moist heat, dry heat, filtration through a bacterial-retaining filter, incorporation of sterilizing agents in the form of sterile solid or liquid compositions that may be dissolved or dispersed in sterile water or other sterile injectable medium just prior to use, and the like.

Injectable preparations, for example, sterile injectable aqueous or oleaginous suspensions may be formulated according to the known art using suitable dispersing or wetting agents and suspending agents. The sterile injectable preparation also may be a sterile injectable solution, suspension or emulsion in a nontoxic, parenterally acceptable diluent or solvent such as a solution in 1,3-butanediol, dichloromethane, ethyl acetate, acetonitrile, etc. Among the acceptable vehicles and solvents that may be employed are water, Ringer's solution, U.S.P. and isotonic sodium chloride solution. In addition, sterile, fixed oils conventionally are employed or as a solvent or suspending medium. For this purpose any bland fixed oil may be employed including synthetic mono- or diglycerides. In addition, fatty acids such as oleic acid are used in the preparation of injectables.

Exemplary buffering agents include: acetic acid and a salt (1-2% w/v); citric acid and a salt (1-3% w/v); boric acid and a salt (0.5-2.5% w/v); and phosphoric acid and a salt (0.8-2% w/v). Exemplary preservatives include benzalkonium chloride (0.003-0.03% w/v); chlorobutanol (0.3-0.9% w/v); parabens (0.01-0.25% w/v) and thimerosal (0.004-0.02% w/v).

According to some embodiments, the pH of the site-specific sustained release microparticulate pharmaceutical formulation is pH 6.7, 6.8, 6.9, 7.0, 7.1, 7.2, 7.3, 7.4, 7.5, or 7.6.

According to some embodiments, the site-specific sustained release microparticulate pharmaceutical formulation is capable of an initial burst of release of less than 25% of the therapeutic agent within 24 hours of administration. According to some embodiments, the site-specific sustained release microparticulate pharmaceutical formulation is capable of an initial burst of release of about 10%, about 11%, about 12%, about 13%, about 14%, about 15%, about 16%, about 17%, about 18%, about 19%, or about 20% of the therapeutic agent.

According to some embodiments, the site-specific sustained release microparticulate pharmaceutical formulation is capable of sustained release of about 50-100% of the therapeutic agent within 1 day to 30 days in plasma. According to some embodiments, the site-specific sustained release microparticulate pharmaceutical formulation is capable of sustained release of about 50-100% of the therapeutic agent within 1 day to 30 days in cerebrospinal fluid (CSF).

According to some embodiments, the site-specific sustained release microparticulate pharmaceutical formulation, upon administration, is capable of sustained release of the therapeutic agent, such that upon release, the concentration of the therapeutic agent in plasma ranges from about 0.200 ng/mL to about 200 g/mL.

According to some embodiments, the site-specific sustained release microparticulate pharmaceutical formulation, upon administration, is capable of sustained release of the therapeutic agent, such that upon release, the maximum concentration (Cₘₐₓ) of the therapeutic agent in the plasma is about 1 ng/mL to about 100 ng/mL, i.e, about 1 ng/mL, about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 70 ng/mL, about 75 ng/mL, about 80 ng/mL, about 85 ng/mL, about 90 ng/mL, about 95 ng/mL, about 96 ng/mL, about 97 ng/mL, about 98 ng/mL, about 99 ng/mL or about 100 ng/mL. According to some embodiments, the site-specific sustained release microparticulate pharmaceutical formulation, upon administration, is capable of sustained release of the therapeutic agent, such that upon release, the maximum concentration (Cₘₐₓ) of the therapeutic agent in the plasma is about 10 ng/mL to about 60 ng/mL, i. e., about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, or about 60 ng/mL. According to some embodiments, the site-specific sustained release microparticulate pharmaceutical formulation, upon administration, is capable of sustained release of the therapeutic agent, such that upon release, the maximum concentration (Cₘₐₓ) of the therapeutic agent in the plasma is about 13 ng/mL to about 55 ng/mL, i.e., about 13 ng/mL, about 16 ng/mL, about 19 ng/mL, about 22 ng/mL, about 25 ng/mL, about 28 ng/mL, about 31 ng/mL, about 34 ng/mL, about 37 ng/mL, about 40 ng/mL, about 43 ng/mL, about 46 ng/mL, about 49 ng/mL, about 43 ng/mL or about 55 ng/mL.

According to one embodiment, upon release, the area under the curve from the time of dosing to the time of the last observation (AUC₀₋ₜ) after administration is about 1000 ng*hr/mL to about 20000 ng*hr/mL, i.e., about 1000 ng*hr/mL, about 2000 ng*hr/mL, about 3000 ng*hr/mL, about 4000 ng*hr/mL, about 5000 ng*hr/mL, about 6000 ng*hr/mL, about 7000 ng*hr/mL, about 8000 ng*hr/mL, about 9000 ng*hr/mL, about 10000 ng*hr/mL, about 11000 ng*hr/mL, about 12000 ng*hr/mL, about 13000 ng*hr/mL, about 14000 ng*hr/mL, about 15000 ng*hr/mL, about 16000 ng*hr/mL, about 17000 ng*hr/mL, about 18000 ng*hr/mL, about 19000 ng*hr/mL or about 20000 ng*hr/mL.

According to one embodiment, upon release, the mean time of maximum concentration (Tₘₐₓ) is about 100 hours to about 300 hours, i.e., about 100 hours, about 110 hours, about 120 hours, about 130 hours, about 140 hours, about 150 hours, about 160 hours, about 170 hours, about 180 hours, about 190 hours, about 200 hours, about 210 hours, about 220 hours, about 230 hours, about 240 hours, about 250 hours, about 260 hours, about 270 hours, about 280 hours, about 290 hours or about 300 hours. According to another embodiment, upon release, the mean time of maximum concentration (Tₘₐₓ) is about 138 hours to about 225 hours, i.e., about 138 hours, about 141 hours, about 144 hours, about 147 hours, about 150 hours, about 153 hours, about 156 hours, about 159 hours, about 162 hours, about 165 hours, about 168 hours, about 171 hours, about 174 hours, about 177 hours, about 180 hours, about 183 hours, about 186 hours, about 189 hours, about 192 hours, about 195 hours, about 198 hours, about 201 hours, about 204 hours, about 207 hours, about 210 hours, about 213 hours, about 216 hours, about 219 hours, about 222 hours or about 225 hours.

According to some embodiments, the site-specific sustained release microparticulate pharmaceutical formulation, upon administration, is capable of sustained release of the therapeutic agent, such that upon release, the concentration of the therapeutic agent in the cerebrospinal fluid (CSF) ranges from about 5 ng/mL to about 5000 mg/mL. According to some embodiments, the site-specific sustained release microparticulate pharmaceutical formulation, upon administration, is capable of sustained release of the therapeutic agent, such that upon release, the concentration of the therapeutic agent in the cerebrospinal fluid (CSF) is about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 200 ng/mL, about 300 ng/mL, about 400 ng/mL, about 500 ng/mL, about 600 ng/mL, about 700 ng/mL, about 800 ng/mL, about 900 ng/mL, about 1 μg/mL, about 5 μg/mL, about 10 μg/mL, about 15 μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, about 100 μg/mL, about 200 μg/mL, about 300 μg/mL, about 400 μg/mL, about 500 μg/mL, about 600 μg/mL, about 700 μg/mL, about 800 μg/mL, about 900 μg/mL, about 1 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 200 mg/mL, about 300 mg/mL, about 400 mg/mL, about 500 mg/mL, about 600 mg/mL, about 700 mg/mL, about 800 mg/mL, about 900 mg/mL, about 1000 mg/mL, about 1100 mg/mL, about 1200 mg/mL, about 1300 mg/mL, about 1400 mg/mL, about 1500 mg/mL, about 1600 mg/mL, about 1700 mg/mL, about 1800 mg/mL, about 1900 mg/mL, about 2000 mg/mL, about 2100 mg/mL, about 2200 mg/mL, about 2300 mg/mL, about 2400 mg/mL, about 2500 mg/mL, about 2600 mg/mL, about 2700 mg/mL, about 2800 mg/mL, about 2900 mg/mL, about 3000 mg/mL, about 33100 mg/mL, about 200 mg/mL, about 3300 mg/mL, about 3400 mg/mL, about 3500 mg/mL, about 3600 mg/mL, about 3700 mg/mL, about 3800 mg/mL, about 3900 mg/mL, about 4000 mg/mL, about 4100 mg/mL, about 4200 mg/mL, about 4300 mg/mL, about 4400 mg/mL, about 4500 mg/mL, about 4600 mg/mL, about 4700 mg/mL, about 4800 mg/mL, about 4900 mg/mL, or about 5000 mg/mL.

### Prevention or Reduction of a Delayed Complication Caused by a Brain Injury

According to some embodiments, the therapeutic amount of the site-specific sustained release microparticulate pharmaceutical formulation, upon release of the therapeutic agent, is effective in preventing or reducing the incidence or severity of the delayed complication associated with the interruption of the cerebral artery caused by the brain injury, wherein the delayed complication is DCI comprising one or more of a cortical spreading ischemia, a delayed spreading depolarization, a plurality of microthromboemboli, or an angiographic vasospasm. According to some embodiments, the DCI comprises a cortical spreading ischemia. According to some embodiments, the DCI comprises a delayed spreading depolarization. According to some embodiments, the DCI comprises a plurality of microthromboemoli. According to some embodiments, the DCI comprises an angiographic vasospasm. According to some embodiments, the brain injury is a result of an underlying condition. Exemplary underlying conditions include, but are not limited to, aneurysm, sudden traumatic head injury, subarachnoid hemorrhage (SAH), or a combination thereof. According to one embodiment, the underlying condition is an aneurysm. According to another embodiment, the underlying condition is a traumatic head injury. According to another embodiment, the underlying condition is a subarachnoid hemorrhage (SAH). According to another embodiment, the underlying condition is a combination of an aneurysm, a sudden traumatic head injury, and a SAH.

According to another embodiment, the therapeutic amount of the site-specific sustained release microparticulate pharmaceutical formulation, upon release of the therapeutic agent, is effective in treating a DCI.

According to another embodiment, the therapeutic amount of the site-specific sustained release microparticulate pharmaceutical formulation, upon release of the therapeutic agent, is effective in reducing the risk of occurrence of delayed cerebral infarction on a Computed Tomography (CT) scan within 7 days, 15 days, or within 30 days of subarachnoid hemorrhage (SAH). According to another embodiment, the therapeutic amount of the site-specific sustained release microparticulate pharmaceutical formulation, upon release of the therapeutic agent, is effective in reducing the risk of occurrence of DCI assessable as a decrease of at least 2 points on the modified Glasgow Coma Scale (GCS) or an increase of at least 2 points on the abbreviated National Institutes of Health Stroke Scale lasting for at least 2 hours.

According to another embodiment, the therapeutic amount of the site-specific sustained release microparticulate pharmaceutical formulation, upon release of the therapeutic agent, is effective in treating a plurality of microthromboemboli. According to some embodiments, the therapeutic amount of the site-specific sustained release microparticulate pharmaceutical formulation is effective in treating microthromboemboli, such that the occurrence of at least one of the following symptoms is reduced within at least 7 days, 14 days, or within 28 days of the SAH: neurological deterioration, a seizure, or a combination thereof. Neurological deterioration can be assessed, for example, by a decrease of at least 2 points on the Glasgow Coma Scale (GCS), the National Institute of Health Stroke Scale (NIHSS), or a combination hereof.

According to another embodiment, the therapeutic amount of the site-specific sustained release microparticulate pharmaceutical formulation, upon release of the therapeutic agent, is effective in treating a cortical spreading ischemia. According to some embodiments, the therapeutic amount of the site-specific sustained release microparticulate pharmaceutical formulation is effective in treating the cortical spreading ischemia such that the occurrence of at least one of the following symptoms is reduced within at least 7 days, 14 days, or within 28 days of the subarachnoid hemorrhage (SAH): presence of blood in the cerebrospinal fluid (CSF), neurological deterioration, a seizure, or a combination thereof. Neurological deterioration can be assessed, for example, by a decrease of at least 2 points on the Glasgow Coma Scale (GCS), the National Institute of Health Stroke Scale (NIHSS), or a combination hereof.

According to one embodiment, the therapeutic amount of the site-specific sustained release microparticulate pharmaceutical formulation, upon release of the therapeutic agent, is effective in preventing or reducing the incidence or severity of an angiographic vasospasm.

According to some embodiments, the therapeutic amount of the site-specific sustained release microparticulate pharmaceutical formulation, upon release of the therapeutic agent, is effective in reducing angiographic vasospasm such that the angiographic diameter of at least one cerebral artery is increased, compared to an untreated control. According to some embodiments, the percent change in the angiographic diameter of the at least one cerebral artery is at least about 10%, at least about 20%, at least about 30%, at least about 40%, or at least about 50%, compared to an untreated control. According to some embodiments, the cerebral artery is selected from the group consisting of an anterior cerebral artery, a middle cerebral artery, an internal carotid artery, a basilar artery, a vertebral artery, or a combination thereof. According to one embodiment, the cerebral artery is an anterior cerebral artery. According to another embodiment, the cerebral artery is a middle cerebral artery. According to another embodiment, the cerebral artery is an internal carotid artery. According to another embodiment, the cerebral artery is a basilar artery. According to another embodiment, the cerebral artery is a vertebral cerebral artery.

According to one embodiment, the site-specific sustained release microparticulate pharmaceutical formulation predominantly localized pharmacologic effect is a reduction of angiographic vasospasm such that the angiographic diameter of at least one cerebral artery in the subarachnoid space at risk of interruption is increased, compared to an untreated control. According to one embodiment, the therapeutic amount of the site-specific sustained release microparticulate pharmaceutical formulation is effective to reduce angiographic vasospasm such that the angiographic diameter of at least one cerebral artery in the subarachnoid space at risk of interruption is increased, compared to an untreated control.

According to one embodiment, the therapeutic amount of the therapeutic agent produces a predominantly localized pharmacologic effect. According to some embodiments, the therapeutic agent is a lipophilic agent capable of binding to blood in the cerebrospinal fluid (CSF).

According to some embodiments, the predominantly localized pharmacologic effect is a reduction of angiographic vasospasm such that the internal diameter of the at least one cerebral artery in subarachnoid space at risk of interruption is increased, compared to an untreated control, wherein the at least one cerebral artery is at least 10 mm, at least 9.9 mm, at least 9.8 mm, at least 9.7 mm, at least 9.6 mm, at least 9.5 mm, at least 9.4 mm, at least 9.3 mm, at least 9.2 mm, at least 9.1 mm, at least 9.0 mm, at least 8.9 mm, at least 8.8 mm, at least 8.7 mm, at least 8.6 mm, at least 8.5 mm, at least 8.4 mm, at least 8.3 mm, at least 8.2 mm, at least 8.1 mm, at least 8.0 mm, at least 7.9 mm, at least 7.8 mm, at least 7.7 mm, at least 7.6 mm, at least 7.5 mm, at least 7.4 mm, at least 7.3 mm, at least 7.2 mm, at least 7.1 mm, at least 7.0 mm, at least 6.9 mm, at least 6.8 mm, at least 6.7 mm, at least 6.6 mm, at least 6.5 mm, at least 6.4 mm, at least 6.3 mm, at least 6.2 mm, at least 6.1 mm, at least 6.0 mm, at least 5.9 mm, at least 5.8 mm, at least 5.7 mm, at least 5.6 mm, at least 5.5 mm, at least 5.4 mm, at least 5.3 mm, at least 5.2 mm, at least 5.1 mm, at least 5.0 mm from the site of release in the subarachnoid space.

### Reducing Risk of Occurrence of Adverse Events

According to another embodiment, the therapeutic amount of the site-specific sustained release microparticulate pharmaceutical formulation, upon release of the therapeutic agent, is effective in reducing the risk of occurrence of adverse events, within at least 7 days, 14 days, or within 30 days of the subarachnoid hemorrhage (SAH). According to some such embodiments, the adverse event is selected from the group consisting of hypotension, occurrence of new cerebral infarcts, seizures, cerebral infarction, increased intracranial pressure, hypersensitivity reaction, paralytic ileus, elevated liver enzymes, thrombocytopenia, cardiac rhythm disturbances, angina pectoris, myocardial infarction, or a combination thereof.

According to another embodiment, the therapeutic amount of the site-specific sustained release microparticulate pharmaceutical formulation, upon release of the therapeutic agent, is effective in reducing the risk of occurrence of hypotension, defined as mean arterial pressure <70 mm Hg for 15 minutes.

According to another embodiment, the therapeutic amount of the site-specific sustained release microparticulate pharmaceutical formulation, upon release of the therapeutic agent, is effective in reducing the risk of occurrence of new cerebral infarcts within at least 7 days, 14 days, or within 30 days of the subarachnoid hemorrhage (SAH).

According to another embodiment, elevated liver enzymes can be detected by a measurement of the level of enzyme(s) in the blood serum or plasma. Exemplary liver enzymes that can be measured for occurrence of adverse events include, but are not limited to, transminase (ALT), aspartate transaminase (AST), etc.

### Reducing Risk of Occurrence of Serious Adverse Events

According to another embodiment, the therapeutic amount of the site-specific sustained release microparticulate pharmaceutical formulation, upon release of the therapeutic agent, is effective in reducing the risk of occurrence of serious adverse events up to 28 days after study drug administration.

### Restoring Cerebral Metabolism

According to another embodiment, the therapeutic amount of the site-specific sustained release microparticulate pharmaceutical formulation, upon release of the therapeutic agent, is effective in restoring cerebral metabolism (meaning the sum of the chemical and physical changes occurring in brain tissue, consisting of anabolism (those reactions that convert small molecules into large) and catabolism (those reactions that convert large molecules into small), as measured by jugular bulb oxygen saturation, intracerebral microdialysis measurements of lactate, pyruvate and glutamate, brain tissue oxygen, or a combination thereof, as compared to an untreated control. According to another embodiment, the therapeutic amount of the site-specific sustained release microparticulate pharmaceutical formulation, upon release of the therapeutic agent, is effective in restoring the integrity of the blood brain barrier.

### Reducing Need for Rescue Therapy

According to another embodiment, the therapeutic amount of the site-specific sustained release microparticulate pharmaceutical formulation, upon release of the therapeutic agent, is effective in reducing a need for rescue therapy.

According to some embodiments, the rescue therapy comprises further administering: (a) a vasopressor, (b) a vasodilator, (c) balloon angioplasty, or a combination thereof.

### Effects on Clinical Outcome

According to some embodiments, the therapeutic amount of the site-specific sustained release microparticulate pharmaceutical formulation, upon release of the therapeutic agent, is effective to improve outcome, as measured on the Glasgow outcome score (GOS), extended GOS, modified Rankin scale (mRS), or other clinical outcome measure (e.g., Montreal cognitive assessment, neurocognitive assessment) compared to the outcome expected or in patients treated with a placebo. According to one embodiment, the therapeutic amount of the site-specific sustained release microparticulate pharmaceutical formulation, upon release of the therapeutic agent, is effective to improve outcome on the GOS by a range of from 1 point to 4 points, i.e., by 1 point, by 2 points, by 3 points or by 4 points compared to the outcome in a patient treated with a placebo. According to another embodiment, the therapeutic amount of the site-specific sustained release microparticulate pharmaceutical formulation, upon release of the therapeutic agent, is effective to improve outcome on the extended GOS by a range of from 1 point to 7 points, i.e., by 1 point, by 2 points, by 3 points, by 4 points, by 5 points, by 6 points or by 7 points as compared to the outcome in a patient treated with a placebo. According to another embodiment, the therapeutic amount of the site-specific sustained release microparticulate pharmaceutical formulation, upon release of the therapeutic agent, is effective to improve outcome on the modified Rankin scale (mRS) by a range of from 1 point to 6 points, i.e., by 1 point, by 2 points, by 3 points, by 4 points, by 5 points or by 6 points as compared to the outcome in a patient treated with a negative control (e.g., a placebo composition, a preservative free saline injection, etc.).

According to another embodiment, the therapeutic amount of the site-specific sustained release microparticulate pharmaceutical formulation, upon release of the therapeutic agent, is effective to reduce the occurrence of poor outcome 90 days after subarachnoid hemorrhage (SAH). According to some embodiments, the therapeutic amount of the site-specific sustained release microparticulate pharmaceutical formulation, upon release of the therapeutic agent, is effective to reduce the occurrence of poor outcome, as measured on the Glasgow outcome score (GOS), extended GOS, modified Rankin scale (mRS), or other clinical outcome measure (e.g., Montreal cognitive assessment, neurocognitive assessment) compared to the outcome expected or in patients treated with a placebo According to one embodiment, the therapeutic amount of the site-specific sustained release microparticulate pharmaceutical formulation, upon release of the therapeutic agent, is effective to improve outcome on the Glasgow outcome score (GOS) by 1 point to 4 points, i.e., 1 point, 2 points, 3 points or 4 points compared to the outcome in a patient treated with a negative control. According to another embodiment, the therapeutic amount of the site-specific sustained release microparticulate pharmaceutical formulation, upon release of the therapeutic agent, is effective to improve outcome on the extended Glasgow outcome score by 1 point to 7 points, i.e., 1 point, 2 points, 3 points, 4 points, 5 points, 6 points or 7 points as compared to the outcome in a patient treated with a placebo. According to another embodiment, the therapeutic amount of the site-specific sustained release microparticulate pharmaceutical formulation, upon release of the therapeutic agent, is effective to improve outcome on the modified Rankin scale (mRS) by 1 to 6 points, i.e., 1 point, 2 points, 3 points, 4 points, 5 points or 6 points as compared to the outcome in a patient treated with a placebo.

### Administration

According to one embodiment, the site-specific sustained release microparticulate pharmaceutical formulation is administered via injection into the subarachnoid space in a cistern closest to the cerebral artery at risk for interruption. According to another embodiment, the site-specific sustained release microparticulate pharmaceutical formulation is administered intraventricularly so that the pharmaceutical composition is carried by CSF flow to contact the at least one artery of the subarachnoid space at risk of interruption. According to another embodiment, the site-specific sustained release microparticulate pharmaceutical formulation is administered intrathecally so that the pharmaceutical composition is carried by CSF flow to contact the at least one artery of the subarachnoid space at risk of interruption. According to another embodiment, the site-specific sustained release microparticulate pharmaceutical formulation is administered intrathecally into the spinal subarachnoid space so that the pharmaceutical composition is carried by CSF flow to contact the at least one artery of the subarachnoid space at risk of interruption.

According to some embodiments, the site-specific sustained release microparticulate pharmaceutical formulation is administered parenterally using an injection apparatus. According to some such embodiments, the injection apparatus is a needle, a cannula, a catheter, or a combination thereof.

According to some embodiments, administering is by passage through a catheter or catheterization. The term “catheterization” refers to a minimally invasive procedure by which the flowable sustained release particulate composition can access the desired areas of the brain, which can mean less risk of complications and a shorter recovery. According to some embodiments, the catheter is a silicone catheter. According to some embodiments, the catheter is a soft catheter. According to some embodiments, the catheter is a flexible catheter. According to some embodiments, the catheter is a pliable catheter.

According to some embodiments, the site of delivery in the central nervous system (CNS) is a site selected from the group consisting of an intracisternal site, an intraventricular site, an intrathecal site, or a combination thereof.

According to another embodiment, the site of delivery in the central nervous system (CNS) is an intraventricular site. According to one embodiment, the intraventricular site is into a cerebral ventricle such that the site-specific sustained release microparticulate pharmaceutical formulation comprising at least one therapeutic agent is carried by CSF circulation to the subarachnoid space so as to contact at least one cerebral artery at risk of interruption due to the brain injury. According to some embodiments, the cerebral ventricle is selected from the group consisting of a lateral ventricle, a third ventricle, a fourth ventricle, or a combination thereof. According to one embodiment, the cerebral ventricle is a lateral ventricle. According to another embodiment, the cerebral ventricle is a third ventricle. According to another embodiment, the cerebral ventricle is a fourth ventricle.

According to another embodiment, the site-specific sustained release microparticulate pharmaceutical formulation comprising the at least one therapeutic agent is administered parenterally via the injection apparatus locally into a cerebral ventricle so that the composition is carried by CSF circulation so as to contact and flow around the cerebral artery in the subarachnoid space at risk of interruption without the first therapeutic agent entering the systemic circulation in an amount to cause unwanted side effects.

The cerebral ventricles may be cannulated or catheterized by, insertion of a ventricular catheter or drain or ventriculostomy. According to some embodiments, a hole of varying size can be drilled in the skull and the outer dura mater covering the brain incised. The pia mater is incised and a catheter (a hollow tube generally made of silicone elastomer or some other biocompatible, nonabsorbable compound) is inserted through the brain into the ventricle of choice. This usually is the lateral ventricle but any ventricle could be catheterized. The catheter can be used to monitor the pressure inside the head, to drain CSF or to administer substances into the CSF.

According to one embodiment, release of the therapeutic agent from the site-specific sustained release microparticulate pharmaceutical formulation occurs in the CSF of the subarachnoid space. The circulation of CSF is often slowed after SAH due to the presence of blood clots in the subarachnoid space. Thus, the site-specific sustained release microparticulate pharmaceutical formulation can become trapped in the blood clots, thereby facilitating localized release of the pharmacological agent(s) from the particulate formulation where a pharmacological effect to the adjacent arteries and brain is achieved.

According to one embodiment, the site-specific sustained release microparticulate pharmaceutical formulation comprising at least one therapeutic agent can be delivered by inserting a catheter into the ventricle and injecting the site-specific sustained release microparticulate pharmaceutical formulation through the catheter such that the composition emanates from the end of the catheter locally into the ventricle.

According to another embodiment, the site-specific sustained release microparticulate pharmaceutical formulation is administered as a single bolus injection. According to another embodiment, the injection is repeated after a pre-determined time period. According to some such embodiments, the pre-determined time period ranges from 1 minute or more to 10 days or more. For example, a repeat injection can be given if monitoring of the patient shows that the patient still had evidence of an interruption of a cerebral artery.

According to one embodiment, the site of delivery in the central nervous system (CNS) is an intracisternal site. According to one embodiment, the intracisternal site is a cerebral cistern closest to the at least one cerebral artery at risk of interruption due to the brain injury, such that the site-specific sustained release microparticulate pharmaceutical formulation comprising the at least one therapeutic agent flows around the at least one cerebral artery at risk of interruption due to the brain injury without the first therapeutic agent entering the systemic circulation in an amount to cause unwanted side effects.

According to some embodiments, the cerebral cistern is at least one of a cisterna magna, a carotid cistern, a chiasmatic cistern, a Sylvian cistern, an interhemispheric cistern, an ambient cistern, a crural cistern, an interpeduncular cistern, a prepontine cistern, and a lateral medullary cistern. According to one embodiment, the cerebral cistern is a cisterna magna. According to another embodiment, the cerebral cistern is a carotid cistern. According to another embodiment, the cerebral cistern is a chiasmatic cistern. According to another embodiment, the cerebral cistern is a Sylvian cistern. According to another embodiment, the cerebral cistern is an interhemispheric cistern. According to another embodiment, the cerebral cistern is an ambient cistern. According to another embodiment, the cerebral cistern is a crural cistern. According to another embodiment, the cerebral cistern is an interpeduncular cistern. According to another embodiment, the cerebral cistern is a prepontine cistern. According to another embodiment, the cerebral cistern is a lateral medullary cistern.

According to another embodiment, the site-specific sustained release microparticulate pharmaceutical formulation comprising at least one therapeutic agent can be delivered by inserting a catheter into the cerebral cistern closest to the at least one cerebral artery at risk of interruption due to the brain injury.

According to another embodiment, the site of delivery in the central nervous system (CNS) is an intrathecal site. According to one embodiment, the intrathecal site is into the subarachnoid space of the spinal canal, such that the site-specific sustained release microparticulate pharmaceutical formulation comprising at least one therapeutic agent is capable of flowing from the CSF of the spinal canal to the CSF in the subarachnoid space of the brain to contact at least one cerebral artery at risk of interruption due to the brain injury without the first therapeutic agent entering the systemic circulation in an amount to cause unwanted side effects.

### Therapeutic Agent

According to some embodiments, the therapeutic agent is a calcium channel antagonist, an endothelin antagonist, a transient receptor potential (TRP) protein antagonist, or a combination thereof.

According to one embodiment, the therapeutic agent is a calcium channel antagonist. According to some embodiments, the calcium channel antagonist is selected from the group consisting of an L-type voltage dependent calcium channel inhibitor, an R-type voltage dependent calcium channel inhibitor, an N-type voltage dependent calcium channel inhibitor, a P/Q-type voltage dependent calcium channel inhibitor, a T-type voltage dependent calcium channel inhibitor, or a combination thereof. According to one embodiment, the calcium channel antagonist is an L-type voltage dependent calcium channel inhibitor. According to another embodiment, the calcium channel antagonist is an R-type voltage dependent calcium channel inhibitor. According to another embodiment, the calcium channel antagonist is an N-type voltage dependent calcium channel inhibitor. According to another embodiment, the calcium channel antagonist is a P/Q-type voltage dependent calcium channel inhibitor. According to another embodiment, the calcium channel antagonist is a T-type voltage dependent calcium channel inhibitor.

For example, L-type voltage dependent calcium channel inhibitor include, but are not limited to: dihydropyridine L-type antagonists such as nimodipine, nisoldipine, nicardipine and nifedipine, AHF (such as 4aR,9aS)-(+)-4a-Amino-1,2,3,4,4a,9a-hexahydro-4aH-fluorene, HC1), Isradipine (such as 4-(4-Benzofurazanyl)-1,-4-dihydro-2,6-dimethyl-3,5-pyridinedicarboxylic acid methyl 1-methylethyl ester), calciseptine/calciseptin (such as isolated from (Dendroaspis polylepis ploylepis), H-Arg-Ile-Cys-Tyr-Ile-His-Lys-Ala-Ser-Leu-Pro-Arg-Ala-Thr-Lys-Thr-Cys-Val-Glu-Asn-Thr-Cys-Tyr-Lys-Met-Phe-Ile-Arg-Thr-Gln-Arg-Glu-Tyr-Ile-Ser-Glu-Arg-Gly-Cys-Gly-Cys-Pro-Thr- Ala-Met-Trp-Pro-Tyr-Gln-Thr-Glu-Cys-Cys-Lys-Gly-Asp-Arg-Cys-Asn-Lys-OH (SEQ ID NO. 1), Calcicludine (such as isolated from Dendroaspis angusticeps (eastern green mamba)), (H-Trp-Gln-Pro-Pro-Trp-Tyr-Cys-Lys-Glu-Pro-Val-Arg-Ile-Gly-Ser-Cys-Lys-Lys-Gln-Phe-Ser-Ser-Phe-Tyr-Phe-Lys-Trp-Thr-Ala-Lys-Lys-Cys-Leu-Pro-Phe-Leu-Phe-Ser-Gly- Cys-Gly-Gly-Asn-Ala-Asn-Arg-Phe-Gln-Thr-Ile-Gly-Glu-Cys-Arg-Lys-Lys-Cys-Leu-Gly-Lys-OH (SEQ ID NO. 2), Cilnidipine (such as also FRP-8653, a dihydropyridine-type inhibitor), Dilantizem (such as (2S,3S)-(+)-cis-3-Acetoxy-5-(2-dimethylaminoethyl)-2,3-dihydro-2-(4-methoxyphenyl)-1,5-benzothiazepin-4(5H)-one hydrochloride), diltiazem (such as benzothiazepin-4(5H)-one, 3-(acetyloxy)-5-[2-(dimethylamino)ethyl]-2,3-dihydro-2-(4-methoxyphenyl)-, (+)-cis-, monohydrochloride), Felodipine (such as 4-(2,3-Dichlorophenyl)-1,4-dihydro-2,6-dimethyl-3,5-pyridinecarboxylic acid ethyl methyl ester), FS-2 (such as an isolate from Dendroaspis polylepis polylepis venom), FTX-3.3 (such as an isolate from Agelenopsis aperta), Neomycin sulfate (such as C₂₃H₄₆N₆O₁₃.3H₂SO₄), Nicardipine (such as 1,4-Dihydro-2,6-dimethyl-4-(3-nitrophenylmethyl-2-[methyl(phenylmethylamino]-3,5-pyridinedicarboxylic acid ethyl ester hydrochloride, also YC-93, Nifedipine (such as 1,4-Dihydro-2,6-dimethyl-4-(2-nitrophenyl)-3,5-pyridinedicarboxylic acid dimethyl ester), Nimodipine (such as 4-Dihydro-2,6-dimethyl-4-(3-nitrophenyl)-3,5-pyridinedicarboxylic acid 2-methoxyethyl 1-methylethyl ester) or (Isopropyl 2-methoxyethyl 1,4-dihydro-2,6-dimethyl-4-(m-nitrophenyl)-3,5-pyridinedicarboxylate), Nitrendipine (such as 1,4-Dihydro-2,6-dimethyl-4-(3-nitrophenyl)-3,5-pyridinedicarboxylic acid ethyl methyl ester), S-Petasin (such as (3 S,4aR,5R,6R)-[2,3,4,4a,5,6,7,8-Octahydro-3-(2-propenyl)-4a,5-dimethyl-2-o-xo-6-naphthyl]Z¬3′-methylthio-1′-propenoate), Phloretin (such as 2′,4′,6′-Trihydroxy-3-(4-hydroxyphenyl)propiophenone, also 3-(4-Hydroxyphenyl)-1-(2,4,6-trihydroxyphenyl)-1-propanone, also b-(4-Hydroxyphenyl)-2,4,6-trihydroxypropiophenone), Protopine (such as C₂₀H₁₉NO₅Cl), SKF-96365 (such as 1-[b-[3-(4-Methoxyphenyl)propoxy]-4-methoxyphenethyl]-1H-imidazole, HC1), Tetrandine (such as 6,6′,7,12-Tetramethoxy-2,2′-dimethylberbaman), (+/−)-Methoxyverapamil or (+)-Verapamil (such as 54N-(3,4-Dimethoxyphenylethyl)methylamino]-2-(3,4-dimethoxyphenyl)-2-iso-propylvaleronitrile hydrochloride), and (R)-(+)-Bay K8644 (such as R-(+)-1,4-Dihydro-2,6-dimethyl-5-nitro-442-(trifluoromethyl)phenyl]-3-pyridinecarboxylic acid methyl ester). The foregoing examples may be specific to L-type voltage-gated calcium channels or may inhibit a broader range of voltage-gated calcium channels, e.g. N, P/Q, R, and T-type.

According to some embodiments, the L-type voltage dependent calcium channel inhibitor is a dihydropyridine. Exemplary dihydropyridines include, but are not limited to, amlodipine, aranidipine, azelnidipine, bamidipine, benidipine, cinaldipine, efonidipine, felodipine, isradipine, lacidipine, lemildipine, lercanidipine, nicardipine, nifedipine, nilvadipine, nimodipine, nisoldipine, nitrendipine, manidipine, pranidipine, etc. According to one embodiment, the dihydropyridine is nimodipine.

According to some embodiments, the L-type voltage dependent calcium channel inhibitor is a phenylalkylamine. Exemplary phenylalkylamines include, but are not limited to, gallopamil, verapamil, etc. According to some embodiments, the L-type voltage dependent calcium channel inhibitor is a 1-4 benzothiazepine. According to one embodiment, the 1-4 benzothiazepine is diltiazem. According to one embodiment, the L-type voltage dependent calcium channel inhibitor is bepridil. According to another embodiment, the L-type voltage dependent calcium channel inhibitor is nimodipine.

According to another embodiment, the therapeutic agent is an endothelin antagonist. Exemplary endothelin antagonists include, but are not limited to, A-127722, ABT-627, BMS 182874, BQ-123, BQ-153, BQ-162, BQ-485, BQ-518, BQ-610, EMD-122946, FR 139317, IPI-725, L-744453, LU 127043, LU 135252, PABSA, PD 147953, PD 151242, PD 155080, PD 156707, RO 611790, SB-247083, clazosentan, atrasentan, sitaxsentan sodium, TA-0201, TBC 11251, TTA-386, WS-7338B, ZD-1611, aspirin, A-182086, CGS 27830, CP 170687, J-104132, L-751281, L-754142, LU 224332, LU 302872, PD 142893, PD 145065, PD 160672, RO-470203, bosentan, RO 462005, RO 470203, SB 209670, SB 217242, TAK-044, A-192621, A-308165, BQ-788, BQ-017, IRL 1038, IRL 2500, PD-161721, RES 701-1, RO 468443, etc.

According to another embodiment, the therapeutic agent is a transient receptor potential (TRP) protein antagonist. Exemplary transient receptor potential (TRP) protein antagonists include, but are not limited to, gadolinium chloride, lanthanum chloride, SKF 96365 (1-(beta-[3-(4-methoxy-phenyl)propoxy]-4-methoxyphenethyl)-1H-imidazole hydrochloride), and LOE 908 ((RS)-(3,4-dihydro-6,7-dimethoxyisoquinoline-1-gamma 1)-2-phenyl-N, N-di-[2-(2,3,4-trimethoxyphenyl)ethyl]acetamide).

According to some embodiments, the therapeutic agent is an isolated molecule. According to some embodiments, the therapeutic agent is substantially pure.

According to some embodiments, the therapeutic amount of the therapeutic agent ranges from about 500 mg to about 1,500 mg, i.e., about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, or about 1500 mg. According to some embodiments, the therapeutic amount of the therapeutic agent ranges from about 612 mg to about 1,200 mg, i.e., about 612 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg. According to some embodiments, the therapeutic amount of the therapeutic agent is at least about 500 mg or at least about 1,500 mg, i.e., at least about 500 mg, at least about 550 mg, at least about 600 mg, at least about 650 mg, at least about 700 mg, at least about 750 mg, at least about 800 mg, at least about 850 mg, at least about 900 mg, at least about 950 mg, at least about 1000 mg, at least about 1050 mg, at least about 1100 mg, at least about 1150 mg, at least about 1200 mg, at least about 1250 mg, at least about 1300 mg, at least about 1350 mg, at least about 1400 mg, at least about 1450 mg, or at least about 1500 mg. According to some embodiments, the therapeutic amount of the therapeutic agent is at least about 612 mg or at least about 1,200 mg, i.e., at least about 612 mg, at least about 650 mg, at least about 700 mg, at least about 750 mg, at least about 800 mg, at least about 850 mg, at least about 900 mg, at least about 950 mg, at least about 1000 mg, at least about 1050 mg, at least about 1100 mg, at least about 1150 mg, at least about 1200 mg.

According to some embodiments, the site-specific sustained release microparticulate pharmaceutical formulation is administered in at least 1 administered dose, at least 2 administered doses, at least 3 administered doses, at least 4 administered doses, at least 5 administered doses, at least 6 administered doses, at least 7 administered doses, at least 8 administered doses, at least 9 administered doses, at least 10 administered doses, at least 11 administered doses, at least 12 administered doses, at least 13 administered doses, at least 14 administered doses, at least 15 administered doses, at least 16 administered doses, at least 17 administered doses, at least 18 administered doses, at least 19 administered doses, at least 20 administered doses or more.

According to some embodiments, the administered dose contains at least about 40 mg, at least about 50 mg, at least about 60 mg, at least about 70 mg, at least about 80 mg, at least about 90 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg, at least about 540 mg, at least about 560 mg, at least about 580 mg, at least about 600 mg, at least about 620 mg, at least about 640 mg, at least about 660 mg, at least about 680 mg, at least about 700 mg, at least about 720 mg, at least about 740 mg, at least about 760 mg, at least about 780 mg, at least about 800 mg, at least about 820 mg, at least about 840 mg, at least about 860 mg, at least about 880 mg, at least about 900 mg, at least about 920 mg, at least about 940 mg, at least about 960 mg, at least about 980 mg, at least about 1,000 mg, at least about 1020 mg, at least about 1040 mg, at least about 1080 mg, at least about 1100 mg, at least about 1120 mg, at least about 1140 mg, at least about 1160 mg, at least about 1180 mg, at least about 1200 mg, at least about 1220 mg, at at least about 1240 mg, at least about 1260 mg, at least about 1280 mg, at least about 1300 mg, at least about 1320 mg, at least about 1340 mg, at least about 1360 mg, at least about 1380 mg, at least about 1400 mg, at least about 1420 mg, at least about 1440 mg, at least about 1460 mg, at least about 1480 mg, at least about 1500 mg, or more of the first therapeutic agent.

According to some embodiments, the site-specific sustained release microparticulate pharmaceutical formulation is administered at least 2 administered doses at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 3 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, site-specific sustained release microparticulate pharmaceutical formulation is administered at least 4 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 5 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 6 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 7 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 8 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 9 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 10 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 11 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 12 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 13 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 14 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 15 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 16 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 17 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 18 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 19 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

According to some embodiments, the site-specific sustained release microparticulate pharmaceutical formulation is administered at least 20 administered doses each at least 1 hour apart, at least 2 hours apart, at least 4 hours apart, at least 6 hours apart, at least 8 hours apart, at least 10 hours apart, at least 12 hours apart, at least 14 hours apart, at least 16 hours apart, at least 18 hours apart, at least 20 hours apart, at least 22 hours apart, at least 24 hours apart, at least 2 days apart, at least 3 days apart, at least 4 days apart, at least 5 days apart, at least 6 days apart, at least 7 days apart, at least 14 days apart, at least one month apart, or at least 2 months apart.

### Additional Therapeutic Agents

According to some embodiments, the site-specific sustained release microparticulate pharmaceutical formulation comprises at least one additional therapeutic agent.

According to one embodiment, the additional therapeutic agent is an anti-inflammatory agent.

According to some embodiments, the anti-inflammatory agent is a steroidal anti-inflammatory agent. The term “steroidal anti-inflammatory agent”, as used herein, refer to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflucortolone valerate, fluadrenolone, fluclorolone acetonide, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

According to another embodiment, the anti-inflammatory agent is a nonsteroidal anti-inflammatory agent. The term “non-steroidal anti-inflammatory agent” as used herein refers to a large group of agents that are aspirin-like in their action, including, but not limited to, ibuprofen (Advil®), naproxen sodium (Aleve®), and acetaminophen (Tylenol®). Additional examples of non-steroidal anti-inflammatory agents that are usable in the context of the described invention include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents also may be employed, as well as the dermatologically acceptable salts and esters of these agents. For example, etofenamate, a flufenamic acid derivative, is particularly useful for topical application.

According to another embodiment, the anti-inflammatory agent includes, without limitation, Transforming Growth Factor-beta3 (TGF-β3), an anti-Tumor Necrosis Factor-alpha (TNF-α) agent, or a combination thereof.

According to some embodiments, the additional therapeutic agent is an analgesic agent. According to some embodiments, the analgesic agent relieves pain by elevating the pain threshold without disturbing consciousness or altering other sensory modalities. According to some such embodiments, the analgesic agent is a non-opioid analgesic. “Non-opioid analgesics” are natural or synthetic substances that reduce pain but are not opioid analgesics. Examples of non-opioid analgesics include, but are not limited to, etodolac, indomethacin, sulindac, tolmetin, nabumetone, piroxicam, acetaminophen, fenoprofen, flurbiprofen, ibuprofen, ketoprofen, naproxen, naproxen sodium, oxaprozin, aspirin, choline magnesium trisalicylate, diflunisal, meclofenamic acid, mefenamic acid, and phenylbutazone. According to some other embodiments, the analgesic is an opioid analgesic. “Opioid analgesics”, “opioid”, or “narcotic analgesics” are natural or synthetic substances that bind to opioid receptors in the central nervous system, producing an agonist action. Examples of opioid analgesics include, but are not limited to, codeine, fentanyl, hydromorphone, levorphanol, meperidine, methadone, morphine, oxycodone, oxymorphone, propoxyphene, buprenorphine, butorphanol, dezocine, nalbuphine, and pentazocine.

According to another embodiment, the additional therapeutic agent is an anti-infective agent. According to another embodiment, the anti-infective agent is an antibiotic agent. The term “antibiotic agent” as used herein means any of a group of chemical substances having the capacity to inhibit the growth of, or to destroy bacteria, and other microorganisms, used chiefly in the treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefmetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

Other examples of at least one additional therapeutic agent include, but are not limited to, rose hip oil, vitamin E, 5-fluorouracil, bleomycin, onion extract, pentoxifylline, prolyl-4-hydroxylase, verapamil, tacrolimus, tamoxifen, tretinoin, colchicine, a calcium antagonist, tranilst, zinc, an antibiotic, and a combination thereof.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated by reference to disclose and describe the methods and/or materials in connection with which the publications are cited.

It must be noted that as used herein and in the appended claims, the singular forms “a”, “and”, and “the” include plural references unless the context clearly dictates otherwise. All technical and scientific terms used herein have the same meaning.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The described invention may be embodied in other specific forms without departing from the spirit or essential attributes thereof and, accordingly, reference should be made to the appended claims, rather than to the foregoing specification, as indicating the scope of the invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

### Formulation Development and Release In Vitro

Methanol, ethyl actetate, tetrahydrofuran, PBS, 4-(2-hydroxyethyl)-1-piperazine ethanesulfonic acid (HEPES), Tween 20, propylene glycol, acetonitrile, glycerol and SDS were purchased from Fisher Scientific (Pittsburgh, Pa., USA) or Sigma (St. Louis, Mo., USA). Polyvinyl alcohol (PVA) was obtained from Amresco (Solon, Ohio, USA) and Resomer Select polymers (PLGA with a 50:50 mole ratio of lactide and glycolide and acid end groups) were supplied by Evonik Corporation (Birmingham, Ala., USA). Liquid chromatography columns were purchased from Agilent (Santa Clara, Calif., USA). Citrate buffer was purchased from Ricca Chemical Company (Arlington, Tex., USA). Nimodipine was obtained from Union Quimico Farmaceutica (Barcelona, Spain). Nimodipine has an asymmetric carbon at position 4 of one of the pyridine rings. The synthesis results in a racemic mixture of the 2 enantiomers that exists in solid form as 2 polymorphs and an amorphous form. Form 1 was a yellow crystal with a melting point of 124° C. Form 2 was an almost white crystal with a melting point of 116° C. and was a conglomerate when solid. There also was an amorphous form. The polymorphs were characterized by pycnometry, solubility measurements, differential scanning calorimetry (DSC), x-ray powder diffractometry (XRPD), x-ray structure analysis and infrared, Raman and ¹H-nuclear magnetic resonance spectroscopy. Ultimately, microparticles consisting mainly of nimodipine Form 1, i.e., at least 51%, at least 55%, at least 60%, at least 65%, at least 70%, at least 75%, at least 80%, at least 85%, at least 90%, at least 95% nimodipine Form 1, relative to the total weight of nimodipine and Form 1 of nimodipine were used.

Microparticles were prepared by an oil/water emulsion microencapsulation technique using solvent extraction to precipitate the polymer, trap the drug and harden the microparticles. The dispersed phase of the emulsion comprised nimodipine and PLGA solution and the continuous phase was composed of a surfactant, e.g., PVA, dissolved in deionized water. According to one embodiment, the extraction phase was deionized water. The hardened microparticles were collected on sieves, lyophylized and stored at −20° C. The dried microparticles were sterilized using gamma irradiation (25-30 kilogray) from a cobalt 60 source (Sterix Isomedix, Libertyville, Ill., USA).

Release characteristics for 14 days in vitro were assessed for different formulations. Ten (10) mg of microparticles were weighed into a 50 mL polypropylene tube and 20 mL of SDS/PBS buffer was added. Tubes were incubated in a shaker water bath at 37° C. Buffer was sampled at 1, 3, 6 and 24 hours and then daily for 14 days.

Nimodipine concentrations in SDS/PBS buffer were measured by reverse phase high performance liquid chromatography (HPLC); in plasma and CSF they were measured by LC-MS/MS using a United States Pharmacopea method (Agilent Technologies and Perkin Elmer, Santa Clara, Calif. and Waltham, Mass., USA). Nimodipine binding to plastic from CSF samples was prevented by adding octyl β-glucopyranoside at approximately 1.5% (weight/volume). Nimodipine was extracted from samples with tetrahydrofuran for 30 minutes and then added to a solution of 25% methyl alcohol in water. A mobile phase of water:methyl alcohol:tetrahydrofuran was used (60:20:20) at 2 mL/min with detection at 235 nm.

Particle size was determined using a Beckman Coulter LS 13 320 Laser diffraction particle size analyzer, multi wavelength, micro liquid module, Pasadena, Calif., USA).

Microparticles were examined by scanning electron microscopy before and after specified days of storage at 4° C., 25° C. and 32.5° C.

Raman spectroscopy was conducted by embedding microparticles in epoxy and sectioning the epoxy on a microtome at −65° C. Full spectral images, 60×60 μm in size, 2 pixels (spectra) per μm, were taken over multiple microparticle cross-sections per lot to determine the distribution of the drug within the microparticles. After acquisition of the spectral data set, an augmented classical least squares routine (which uses the entire reference spectra from the drug, polymer, and epoxy) was implemented to deconvolute the signals of each component of multiple microparticles to determine distribution of nimodipine within the microparticles.

X-ray powder diffraction (XRPD) patterns were collected with a PANalytical X′Pert PRO MPD diffractometer or a Bruker D8 DISCOVER diffractometer and Bruker's General Area-Detector Diffraction System (GADDS, v. 4.1.20) using an incident beam of copper radiation produced using an Optix long, fine-focus source. Elliptically or parabolically graded multilayer mirrors were used to focus copper Kα X-rays through the specimen and onto the detector. Prior to the analysis, a silicon specimen (NIST SRM 640d) was analyzed to verify the silicon 111 peak position. A specimen of the sample was sandwiched between 3 μm thick films and analyzed in transmission geometry. A beam-stop, short antiscatter extension, and a helium atmosphere were used to minimize the background generated by air in some studies. Soller slits for the incident and diffracted beams were used to minimize broadening from axial divergence. Diffraction patterns were collected using a scanning position-sensitive detector (X'Celerator) 240 mm or a HIS TAR™ area detector 15 cm from the specimen and data collector software version 2.2b.

¹H nuclear magnetic resonance spectroscopy were acquired with a Varian UNITYINOVA-400 spectrometer. The samples were prepared by dissolving the nimodipine microparticles in deuterated dichloromethane containing trimethylsilylacetylene. Spectra were obtained at ambient temperature, spin rate 20 Hz and the following parameters: pulse sequence relaxation delay 2.5 seconds, pulse width 8.9 seconds (90°), acquisition time 5 seconds, spectral width 6400 Hz, 40 scans, 64000 acquired points.

Raman spectra were acquired on a FT-Raman 960 spectrometer (Thermo Nicolet, Life Technologies, Carlsbad, Calif., USA) or on a FTRaman module interfaced to a Nexus 670 FT-IR spectrophotometer (Thermo Nicolet) equipped with an indium gallium arsenide detector. Wavelength verification was performed using sulfur and cyclohexane. The sample was prepared for analysis by placing it into a glass tube and positioning the tube in a gold-coated tube holder or the sample was packed into a pellet and placed in a pellet holder. Approximately 0.4-1 W of Nd:YV04 laser power (1064 nm excitation wavelength) was used to irradiate the sample. Each spectrum represented 256 co-added scans collected at a spectral resolution of 4/cm.

Infrared spectra were acquired on a Magna-IR 560® or a Nexus 670® Fourier transform infrared spectrophotometer (Thermo-electron corporation Nicolet avatar 370DTGS, Life Technologies, Carlsbad, Calif., USA) equipped with an Ever-Glo mid/far infrared source, a potassium bromide beamsplitter and a deuterated triglycine sulfate detector. Wavelength verification was performed using NIST SRM 1921b (polystyrene). An attenuated total reflectance accessory (Thunderdome™, Thermo Spectra-Tech), with a germanium crystal was used for data acquisition. Each spectrum represented 256 co-added scans collected at a spectral resolution of 4/cm. A background data set was acquired with a clean germanium crystal. A log 1/reflectance spectrum was obtained by taking a ratio of these two data sets against each other.

Differential scanning calorimetry (DSC) was performed on a 2920 Modulated or a Q2000 DSC equipped with a refrigerated cooling system (TA Instruments, New Castle, Del., USA). Temperature calibration was performed using National Institute of Standards and Technology traceable indium metal. The sample was placed into an aluminum DSC pan and weighed. The pan was covered with a lid and the lid was crimped. A weighed, crimped aluminum pan was placed on the reference side of the cell. Data were obtained using a modulation amplitude of ±1.00° C. and a 50 second period with an underlying heating rate of 2° C./minute from −50 to 125° C. The reported glass transition temperatures were obtained from the inflection point of the step change in the reversing heat flow versus temperature curve.

TABLE 4
Characteristics of 6 Nimodipine-PLGA Formulations

| | Polymer | | | | | | | |
|---|---|---|---|---|---|---|---|---|
| | Composition | Theoretical | Actual | | | | | |
| | (Lactide: | Drug | Drug | Encap- | Main | | Residual | Residual |
| Formu- | Glycolide | Content | Content | sulation | Peak | Particle | Solvent | Water |
| lation | ratio, | (weight | (weight | Efficiency | Purity | size | (weight | (weight |
| Number | end group) | %) | %) | (%) | (%) | (μm) | %) | %) |
| 00447-098 | 65:35, | 65.0 | 62 ± 1.3 | 95.4 | 98.9 | 37.2 | 0.56 | 0.39 |
| | ester | | | | | | | |
| 00447-102 | 65:35, | 65.0 | 61 ± 0.7 | 94.5 | 99.9 | 36.9 | 0.67 | 0.31 |
| | acid | | | | | | | |
| 00447-104 | 75:25, | 65.0 | 56 ± 0.4 | 86.6 | 99.9 | 39.1 | 0.12 | 0.38 |
| | acid | | | | | | | |
| 00447-106 | 75:25, | 65.0 | 63 ± 1.5 | 96.8 | 98.8 | 38.1 | 0.64 | 0.37 |
| | acid | | | | | | | |
| 00447-108 | 50:50, | 65.0 | 64 ± 0.3 | 98 | 99.9 | 34.7 | 0.93 | 0.24 |
| | acid | | | | | | | |
| 00447-110 | 65:35, | 50.0 | 49 ± 0.3 | 97.6 | 99.9 | 38.8 | 0.37 | 0.49 |
| | ester | | | | | | | |
| 00447-116 | 65:35, | 66.5 | 61 ± 1.2 | 91.3 | 99.9 | 30.9 | 0.37 | 0.29 |
| | ester | | | | | | | |

Values are means ± standard deviations (n = 3 per measurement)

### Nimodipine Release In Vivo

All experiments using animals adhered to the National Institutes of Health Guide for the Care and Use of Laboratory Animals. Guidelines for animal research were followed [Kilkenny C, Browne W J, Cuthill I C, Emerson M, Altman D G. Improving bioscience research reporting: the ARRIVE guidelines for reporting animal research. PLoS Biol 2010; 8:e1000412.]. Endpoints were determined by investigators who were blinded to the animal groups. All animals used are reported.

Low viscosity hyaluronic acid used as a vehicle to prepare nimodipine-PLGA microparticles suspension was obtained from Fidia (Hyalgan®, Abano Terme, Italy, zero shear rate viscosity of 2 Poise, molecular weight 0.500-0.750 million Da purified natural sodium hyaluronic acid derived from rooster combs, pH 6.8-7.5, suspended in 0.9% NaCl).). In one arm of the experiments in vivo, nimodipine-PLGA microparticles injected into the cisterna magna were mixed with high-viscosity hyaluronic acid (Orthovisc®, Bedford, Mass., USA, zero shear rate viscosity of 1677 Poise, molecular weight 1.0-2.9 million Da, dissolved in 0.9% NaCl and produced through recombinant bacterial fermentation).

Formulations and pure nimodipine were implanted subcutaneously in 44 male Wistar rats instrumented with jugular vein cannulas, approximately 9 weeks of age. Neat nimodipine was prepared in 1.87% hydroxypropyl methylcellulose in PBS. The nimodipine reference solution and nimodipine-PLGA microparticle formulations 00447-098, 00447-102, 00447-104, 00447-108, 00447-110 and 00447-116 were administered via a single subcutaneous injection in the left hind limb of each animal at doses of 20 (in 0.15 mL) or 200 (0.7 mL) mg/kg. Blood samples were collected from two alternating cohorts of 2 or 3 animals/cohort in the microparticle formulation-injected groups. Collection was immediately before injection and then 1, 12, and 24 hours post dose, and on days 4, 8, 11, and 15. After the last blood sample collection interval, the surviving animals were euthanized via carbon dioxide inhalation. Animals were euthanized by cervical dislocation and the carcasses were discarded.

### Toxicity Studies

Nimodipine-PLGA microparticles were tested for toxicity in rats and beagles. The primary objectives were to assess toxicity, to determine a no observable adverse effect level (NOAEL) and to measure pharmacokinetics. Rats (CD® [Crl:CD®(SD)], Charles River, Portage, Mich., USA, 130-290 g, 190 rats) were randomly allocated with equal numbers of each sex (n=7-13 per sex per group) to undergo intraventricular injection of equal volumes of 0.9% NaCl, placebo microparticles or nimodipine microparticles at doses of 0.33 mg, 1 mg or 2 mg nimodipine on day 1 and euthanasia on day 15 or 29 (Table 5). Microparticles were suspended in low viscosity hyaluronic acid buffer prior to injection. They were anesthetized with isoflurane and placed in a stereotactic frame. The scalp was prepared sterily, a midline incision was made on the dorsum of the head and the skin was retracted. A hole was drilled in the skull above the right lateral ventricle and a 13-gauge blunt needle was lowered into the ventricle and one of the solutions was injected in a volume of 23±5 μL. The needle was removed, the drill hole was covered with bone wax and the incision was closed. Animals were observed twice daily and examined on days 1 to 7, 14, 21, and 28. Functional observational battery evaluations were conducted without knowledge on the part of the testers of the treatment groups on all animals before surgery and then on days 3, 14 and 28. Open-field evaluations were performed with each animal placed in a black box and observed for at least 3 minutes [Moser V C, McCormick J P, Creason J P, Macphail R C. Comparison of chlordimeform and carbaryl using a functional observational battery. Fundam Appl Toxicol 1988; 11:189-206; Moser V C, Becking G C, Macphail R C, Kulig B M. The IPCS collaborative study on neurobehavioral screening methods. Fundam Appl Toxicol 1997; 35:143-51.]. The observations included, but were not limited to, activity and arousal, posture, rearing, bizarre behavior, clonic and tonic movements, gait, mobility, stereotypy, righting reflex, response to stimulus (approach, click, tail pinch, and touch), palpebral closure, pupil response, piloerection, exophthalmus, lacrimation, salivation and respiration. The amount, qualitative and/or quantitative measures, of defecation and urination were also recorded. Forelimb and hindlimb grip strength and hindlimb splay were measured (Meyer O A, Tilson H A, Byrd W C, Riley M T. A method for the routine assessment of fore- and hindlimb grip strength of rats and mice. Neurobehav Toxicol 1979; 1:233-6; Edwards P M, Parker V H. A simple, sensitive, and objective method for early assessment of acrylamide neuropathy in rats. Toxicol Appl Pharmacol 1977; 40:589-91.). Pain perception was assessed by measuring the latency of response to a nociceptive (thermal) stimulus when each animal was placed on a hot plate apparatus set to 51.6 to 52.3° C. (Ankier S I. New hot plate tests to quantify antinociceptive and narcotic antagonist activities. Eur J Pharmacol 1974; 27:1-4.). Body weight and temperature were also measured. Food consumption was measured and recorded. Ophthalmoscopic examinations were conducted before injection and prior to euthanasia. Blood was collected at euthanasia for evaluation of hematology, coagulation parameters and clinical chemistry. Urine samples were collected by placing the animals in stainless steel metabolism cages for at least 12 hours.

TABLE 5
Groups for Rat Toxicity Study

| | Nimodipine | | |
|---|---|---|---|
| | Dose | Sacrifice | |
| Group | (mg) | (day) | Number of Rats (N) |
| 0.9% NaCl | 0 | 15 | 14 |
| Placebo microparticles | 0 | 15 | 14 |
| Nimodipine-PLGA microparticles | 0.33 | 15 | 14 |
| Nimodipine-PLGA microparticles | 1 | 15 | 14 |
| Nimodipine-PLGA microparticles | 2 | 15 | 14 |
| 0.9% NaCl | 0 | 29 | 14 |
| Placebo microparticles | 0 | 29 | 14 |
| Nimodipine-PLGA microparticles | 0.33 | 29 | 12 (nimodipine plasma only) |
| Nimodipine-PLGA microparticles | 1 | 29 | 14 + 12 |
| | | | (nimodipine plasma only) |
| Nimodipine-PLGA microparticles | 2 | 29 | 14 + 12 |
| | | | (nimodipine plasma only) |

Nimodipine concentration was measured in plasma 12, 24 and 36 hours after the intraventricular injection and then on days 3, 5, 8, 15 and 29, depending on the group. Samples were placed in tubes containing K₂EDTA, placed on ice and protected from light, centrifuged and plasma removed and stored at −80° C. until analysis. CSF was collected from the cisterna magna for measurement of nimodipine concentration on days 1, 3, 15, and 29. Samples were placed in glass tubes and placed on ice and protected from light prior to centrifugation at 1300 g for 10 minutes under refrigerated conditions. The supernatant CSF was removed, octyl-β-D-glucopyranoside was added (15 mg/mL), and samples stored at −80° C. until analysis using a validated LC-MS/MS assay.

Seventy-eight (78) beagles (8-12 kg, n=3 per sex per group, Marshall BioResources, North Rose, N.Y., USA) were randomly allocated to receive intraventricular or intracisternal injection of 0.9% NaCl, placebo microparticles or nimodipine microparticles (Table 6). For intraventricular injection, microparticles were suspended in low viscosity hyaluronic acid (Hyalgan®, zero shear rate viscosity of 2 Poise, molecular weight 0.500-0.750 million Da purified natural sodium hyaluronic acid derived from rooster combs, pH 6.8-7.5, suspended in 0.9% NaCl) and for intracisternal injection they were suspended in high viscosity hyaluronic acid (Orthovisc®, zero shear rate viscosity of 1677 Poise, molecular weight 1.0-2.9 million Da, dissolved in 0.9% NaCl and produced through recombinant bacterial fermentation). The injection volume was 1.15 mL and the dose of nimodipine was 17 mg, 51 mg or 103 mg.

TABLE 6
Groups for Beagle Toxicity Study (n = 6 per group)

| | Nimodipine | | Route | | |
|---|---|---|---|---|---|
| | Dose | | of | Sacrifice | Statistical |
| Group | (mg) | Vehicle | Administration | (day) | Comparison |
| 0.9% NaCl | None | None | Intraventricular | 15 | Reference |
| | | | | | group |
| Placebo | None | Low viscosity | Intraventricular | 15 | 0.9% NaCl |
| microparticles | | hyaluronic acid | | | |
| Nimodipine- | 17 | Low viscosity | Intraventricular | 15 | Placebo |
| PLGA | | hyaluronic acid | | | intraventricular |
| microparticles | | | | | |
| Nimodipine- | 51 | Low viscosity | Intraventricular | 15 | Placebo |
| PLGA | | hyaluronic acid | | | intraventricular |
| microparticles | | | | | |
| Nimodipine- | 103 | Low viscosity | Intraventricular | 15 | Placebo |
| PLGA | | hyaluronic acid | | | intraventricular |
| Microparticles | | | | | |
| Placebo | None | High viscosity | Intracisternal | 15 | Reference |
| microparticles | | hyaluronic acid | | | group |
| Nimodipine- | 103 | High viscosity | Intracisternal | 15 | Placebo |
| PLGA | | hyaluronic acid | | | intracisternal |
| microparticles | | | | | |
| 0.9% NaCl | None | None | Intraventricular | 29 | Reference |
| | | | | | group |
| Placebo | None | Low viscosity | Intraventricular | 29 | 0.9% NaCl |
| microparticles | | hyaluronic acid | | | |
| Nimodipine- | 51 | Low viscosity | Intraventricular | 29 | Placebo |
| PLGA | | hyaluronic acid | | | intraventricular |
| microparticles | | | | | |
| Nimodipine- | 103 | Low viscosity | Intraventricular | 29 | Placebo |
| PLGA | | hyaluronic acid | | | intraventricular |
| microparticles | | | | | |
| Placebo | None | High viscosity | Intracisternal | 29 | Reference |
| microparticles | | hyaluronic acid | | | group |
| Nimodipine- | 103 | High viscosity | Intracisternal | 29 | Placebo |
| PLGA | | hyaluronic acid | | | intracisternal |
| microparticles | | | | | |

Beagles were sedated with propofol (6 mg/kg intravenously) and then intubated and ventilated with 1-3% isoflurane. For intraventricular injection, they were mounted in a stereotactic frame and the scalp prepared and draped sterily. A skin incision was made 1 cm off the midline over the right coronal suture. The skin was retracted and a 5 mm hole was drilled in the skull. The dura was opened and a 14 gauge needle was advanced into the right lateral ventricle. Up to 0.50 mL of contrast was injected under fluoroscopy to confirm that the needle was in the lateral ventricle. Then 1.15 mL of the appropriate solution was administered, the needle was removed, and the skin was closed. Intracisternal injections were performed by flexing the neck of the beagle and inserting a 14 gauge needle into the cisterna magna. 0.3 mL/kg of CSF was allowed to drain spontaneously and 1.15 mL of the appropriate solution was injected. The needle was removed and animals were allowed to awaken.

All animals were observed twice a day. Clinical and behavior examinations were conducted on days 1-7, 15, 21 and 29 (Zhou C, Yamaguchi M, Colohan A R, Zhang J H. Role of p53 and apoptosis in cerebral vasospasm after experimental subarachnoid hemorrhage. J Cereb Blood Flow Metab 2005; 25:572-82.). Body weight was measured weekly and ophthalmoscopy was conducted before injections and prior to euthanasia. Awake indirect blood pressure and heart rate were measured prior to injections and on days 2 to 7, 15, 21 and 29 and ECG was obtained before injections and then prior to euthanasia. Standard ECGs were recorded at 50 mm/sec. The RR, PR and QT intervals and QRS duration were measured. Corrected QT (QTc) interval was calculated [Fridericia L S. Die systolendauer im elektrokardiogramm bei normalen menschen and bei herzkranken. Acta Med Scand 1920; 53:469-86.]. Blood was collected before euthanasia for hematology, coagulation parameters, and clinical chemistry. Urine samples were collected using steel pans placed under the cages for at least 16 hours.

Plasma for measurement of nimodipine was collected before injections and 1, 3, 6, 12, 18, 24 and 36 hours post-injection on day 1, on days 3 through 10 and on days 12, 15, 18, 21 and 29. CSF was collected from all animals from the cisterna magna or lumbar thecal sac immediately prior to the injection on day 1 and then on days 3, 15 and 29. Sample preparation was the same as for rat samples.

### Dog Efficacy Study

Forty (40) mongrel dogs (16-23 kg, n=4 per sex per group, Marshall BioResources) were randomly allocated to undergo baseline cerebral angiography (day 1) and cisternal blood injection followed by treatment with placebo microparticles, placebo microparticles plus oral nimodipine (5.2 mg/kg daily for 21 days, equivalent to a human dose of 30 mg every 4 hours for a 60 kg human based on body surface area)[Reagan-Shaw S, Nihal M, Ahmad N. Dose translation from animal to human studies revisited. FASEB J 2008; 22:659-61.], nimodipine-PLGA microparticles, 40 mg intracisternal, nimodipine-PLGA microparticles, 100 mg intracisternal or nimodipine-PLGA microparticles, 100 mg intraventricular. Dogs were euthanized on day 28 or 49. Dogs were anesthetized as described above, blood was collected and blood pressure, temperature, heart rate and oxygen saturation were monitored continuously while animals were under anesthesia. Blood gases were obtained before and after the injections. Cerebral angiography was performed through a 4F catheter inserted into the femoral artery and advanced under fluoroscopic guidance into the proximal portion of one vertebral artery using a guide wire. A single arterial phase anteroposterior angiogram was obtained by injection of up to 8 mL contrast (diatrizoate meglumine 60%, United States Pharmacopeia, Amersham Health, Princeton, N.J., USA). Images were captured digitally using identical exposure factors and magnification and an internal magnification standard was included in every angiogram.

After angiography, dogs were turned prone, tilted 30° head down and the cisterna magna was punctured percutaneously with a 14 gauge thin-walled spinal needle. 0.3 mL/kg CSF was allowed to drain spontaneously, after which approximately 0.3 to 0.5 mL/kg of fresh, autologous, arterial, non-heparinized blood was withdrawn from the femoral catheter and injected into the cisterna magna at a rate of 5 mL/minute. One half of the blood was injected, followed by injection of microparticles and then the remaining blood. The needle was withdrawn and the dogs were kept 30° head down for 15 minutes after which the femoral catheter was removed, the artery was ligated, and the incision was closed.

For intraventricular injection, the blood was injected into the cisterna magna and then the dog was placed prone in a stereotactic unit. Nimodipine microparticles were injected into the right lateral ventricle using the same procedure as described in the beagle toxicity study.

On day 3, dogs were anesthetized again and blood pressure, temperature, heart rate, and oxygen saturation were monitored continuously. Blood was injected into the cisterna magna using the same procedure as on day 1 except that microparticles were not injected. Angiography was repeated on days 8 and 15. The diameter of the basilar artery was measured at 5 equally spaced points by two blinded observers and the means averaged. Angiographic vasospasm was assessed by comparing the diameters of basilar arteries from Days 1, 8 and 15 and the time course and severity of angiographic vasospasm was determined. Dogs were weighed weekly and examined daily for 14 days, including behavior examination [Zhou C, Yamaguchi M, Colohan A R, Zhang J H. Role of p53 and apoptosis in cerebral vasospasm after experimental subarachnoid hemorrhage. J Cereb Blood Flow Metab 2005; 25:572-82.]. Behavior included appetite (0: scarcely ate, 1: left meal unfinished, 2: finished meal), activity (0: almost always lying down, 1: lying down, will stand and walk with some stimulation, 2: active, barking and standing) and neurological deficits (0: cannot walk or stand because of hemiparesis or ataxia, 1: unable to walk because of hemiparesis or ataxia, 2: no deficit). Indirect blood pressure was measured in awake dogs daily for the first 14 days and weekly thereafter.

Plasma was collected for determination of nimodipine concentration. Samples were collected before injection of microparticles and 1, 3, 6, 12, 18, 24 and 36 hours after and on days 3 to 10, 12, 14, 16, 18, 21, and 28. Samples were collected from some animals on days 38, 42, and 49. Samples were placed in tubes containing K₂EDTA, placed on ice, centrifuged and the plasma removed and stored at −80° C. until analyzed.

In addition to the CSF samples collected above on days 1 and 3, CSF was collected from all animals from the cisterna magna on days 8, 15 and 28, and from some animals on days 35, 42 and 49. They were placed on ice, centrifuged and the supernatant fluid removed. Octyl β-glucopyranoside (15 mg/mL) was added and the CSF was stored at −80° C. until analysis.

Dogs were euthanized by an intravenous overdose of sodium pentobarbital. They were perfused through the heart with PBS followed by 10% neutral buffered formalin. The brain and spinal cord were excised and axial cross sections of the midbrain, pons, medulla and cervical, thoracic and lumbar spinal cord and a coronal section of the cerebral hemispheres through the middle cerebral artery territory were taken, embedded in paraffin and stained with hematoxylin and eosin. A section from each level also were stained with fluoro-jade B, activated caspase-3 or anti-thrombin III. For fluoro-jade B, slides were deparaffinized through xylene and graded alcohols and hydrated gradually in deionized water. A 0.06% potassium permanganate solution was added, slides rinsed and then placed in 0.0002% fluoro-jade B solution in a light free environment. Slides were rinsed again in deionized water, heated in a 50° C. oven and coverslipped in non-aqueous DPX mounting media. A positive control was formalin fixed, paraffin embedded brain tissue treated with kainic acid.

For caspase 3 and antithrombin staining, formalin-fixed, paraffin-embedded sections were stained on a Ventana Discovery XT automatic stainer (Ventana Medical Systems, Tucson, Ariz., USA). Antigen retrieval was done under mild conditions using a Tris-based buffer with a slightly basic pH (CC1; Ventana, 950-1240). Slides were then incubated with the applicable primary antibody, rabbit polyclonal anti-caspase 3 primary antibody (ab4051, Abcam, Cambridge, Mass., USA) or sheep polyclonal anti-antithrombin III (Abcam, ab124259). Staining detection was performed using a ChromoMap DAB kit (Ventana; 760-159) compatible with the autostainer comprised of an inhibitor reagent to reduce endogenous peroxidase activity, 3,3′-diaminobenzidine chromogen, 3,3′-diaminobenzidine H₂O₂ substrate for peroxidase and a copper sulfate solution to enhance color. Counterstain reagents were hematoxylin and bluing reagent (Ventana; 760-2037). Finally the slides were dehydrated, cleared and cover-slipped with synthetic mounting medium.

Neuronal degeneration and apoptosis were scored by a pathologist blinded to the assigned group on a semi-quantitative scale as normal, minimal, mild, moderate or severe. Microthrombi present in three (3) 100×100 μm fields were counted.

### Data Analysis and Statistics

All data are mean±standard deviation. For the toxicity studies, we compared saline to placebo within each route of administration and time (15 or 29 day sacrifice) and placebo with nimodipine-PLGA microparticles groups within each route of administration and time (Tables 6 and 7. Leucocyte count and urinalysis results were transformed by log and rank transformation, respectively, prior to analysis. Groups were compared by ANOVA. If there was significant variance (P<0.05), pair-wise comparisons were made after adjustment for multiple comparisons by Holm-Sidak or Tukey methods [Edwards D, Berry J J. The efficiency of simulation-based multiple comparisons. Biometrics 1987; 43:913-28]. The functional observation battery was analyzed by the Cochran Mantel Haenszel test. All statistical analysis was done in Stata (version 9, College Station, Tex., USA).

### Example 1. Formulation Development

Eight (8) nimodipine—PLGA microparticle formulations were prepared. Their chemical/physical stability, sterilizability and release characteristics in vitro were determined. Manufacturing solvents and their residual levels in microparticles were acceptable for use in the brain and subarachnoid space. The microparticle size range was 20 μm to 125 μm. This range was selected because smaller microparticles (<10 μm) can be taken up by macrophages and cleared rapidly, while larger microparticles are not easy to inject through catheters of the size that are routinely used in neurosurgery. Dose estimation was based on the clinical use of 40 mg nicardipine-loaded PLGA pellets, which would equal 30 mg nimodipine based on molecular weights, not accounting for differences in drug potency. In anticipation that this dose would be at the lowest end of the dose-efficacy relationship when given intraventricularly, drug loading was maximized to minimize the injection volume. Assuming an effective human dose may be 600 mg, the initial burst of drug release was to be <25% within 24 hours since this potentially would be 150 mg nimodipine released into the systemic circulation in 24 hours. This is comparable to the oral total single day dose of 360 mg that has about 28% bioavailability. Oral nimodipine is approved for use for 21 days after SAH. However, administration for 14 days is common now, since this extends to beyond the time when DCI develops. Thus, sustained release over at least 14 days was required. The formulation also needed to be stable and preferably terminally sterilizable.

Parameters that were varied in the formulations were lactide to glycolide ratio, polymer end group chemistry, molecular weight and cosolvents. The resulting microparticles varied from 25% to 50% glycolide, with 50 to 70% theoretical drug loading and mean particle sizes of approximately 70 μm to 100 μm, all formulated with ethyl acetate solvent and >99% purity and >91% encapsulation efficiency (Table 4).

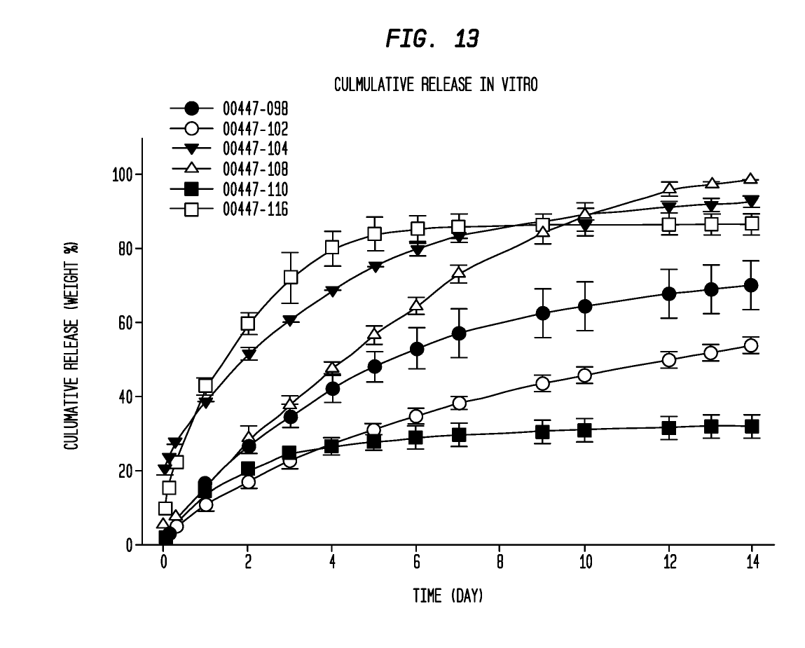

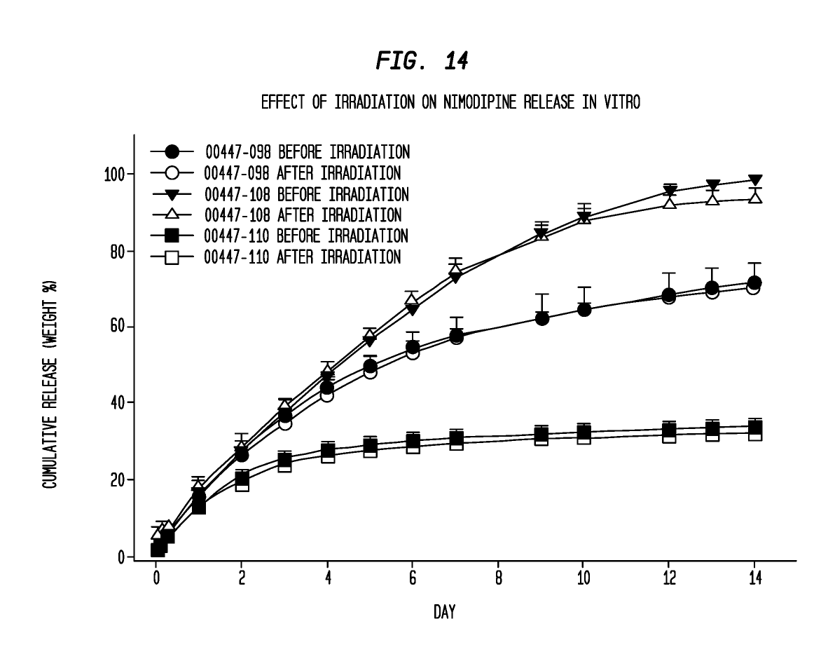

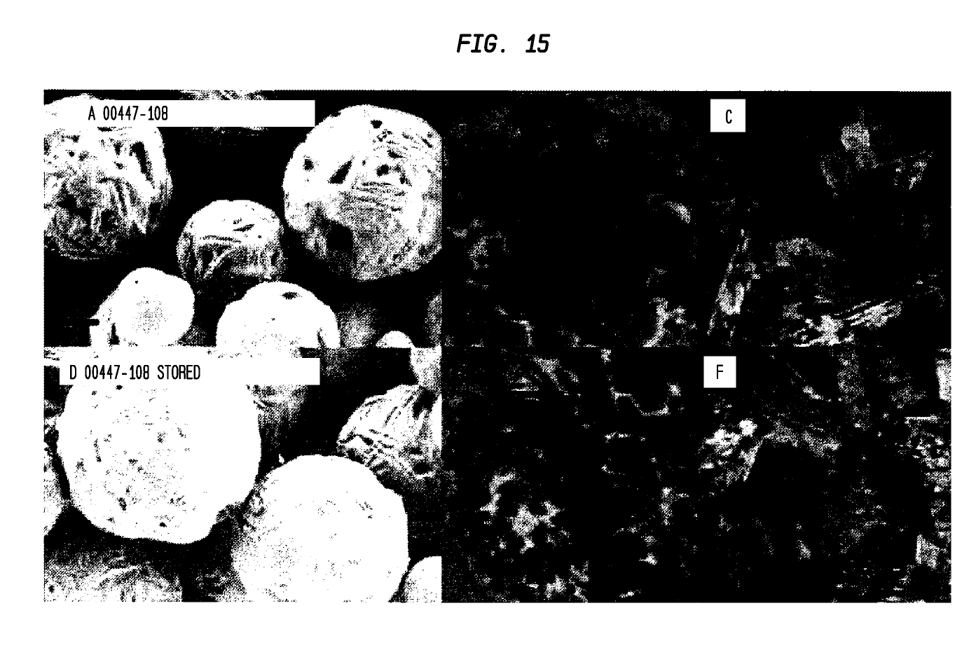

Three selected formulations did not change in drug content, main peak purity and mean particle size after sterilization with >25 kGray gamma radiation (Table 7). Nimodipine release characteristics were tested in vitro in 2% sodium dodecyl sulfate/phosphate buffered saline (SDS/PBS) under optimized sink conditions. Sterilization did not change the microparticle release characteristics in vitro (FIG. 13 and FIG. 14). This result demonstrated the ability to prepare formulations that released nimodipine with different initial bursts and cumulative release over 14 days. Selected formulations were prepared 3 times with inconsistent results for some formulations. Without being bound by theory, these results may have been due to nimodipine polymorphisms. Nimodipine used was a racemic mixture with different polymorphs found by differential scanning calorimetry and x-ray powder diffractometry to exist as a yellow crystalline form with a melting point of about 124° C. (Form 1), a less stable and almost white conglomerate with a melting point of about 116° C. (Form 2) and an amorphous form. The forms were also characterized by infrared and Raman spectroscopy (FIG. 15. Differential scanning calorimetry showed no changes in main and secondary melting temperatures of microparticles over time. Formulations containing Form 1 were the most stable and there was little to no impact when small amounts of Form 2 were present. According to analysis, pure nimodipine was mainly Form 1. Microparticles did not change significantly after storage for 1 month at 25° C. based on scanning electron microscopy and release characteristics in vitro (Table 8, FIG. 15). Raman spectroscopy and scanning electron microscopy showed nimodipine at the surface of some microparticles, which resulted in the 10-20% burst of drug release in vitro for some formulations.

TABLE 7
Effect of Gamma Irradiation (27.2 kGray) on 3 Nimodipine-
PLGA Formulations

| | | Theoretical | Actual | Encap- | Main | Par- |
|---|---|---|---|---|---|---|
| Formu- | | Drug | Drug | sulation | Peak | ticle |
| lation | | Content | Content | Efficiency | Purity | size |
| Number | Conditions | (weight %) | (weight %) | (%) | (%) | (μm) |
| 00447-098 | Before | 65 | 62 ± 1.3 | 95.4 | 98.9 | 37.2 |
| | irradiation | | | | | |
| 00447-098 | After | — | 61 ± 1.1 | 94.3 | 98.9 | 59.8 |
| | irradiation | | | | | |
| 00447-108 | Before | 65 | 60 ± 1.5 | 92.3 | 98.9 | 34.7 |
| | irradiation | | | | | |
| 00447-108 | After | — | 63 ± 2.1 | 97.5 | 98.9 | 65.2 |
| | irradiation | | | | | |
| 00447-110 | Before | 50 | 48 ± 3.1 | 95.6 | 98.9 | 38.8 |
| | irradiation | | | | | |
| 00447-110 | After | — | 49 ± 2.2 | 98.8 | 98.6 | 49.8 |
| | irradiation | | | | | |

Values are means ± standard deviations (n = 3 per measurement)

TABLE 8
Characteristics and Differential Scanning Calorimetry of 4
Nimodipine-PLGA Formulations after Storage for 30 Days

| | | Average | Encap- | | | | | |
|---|---|---|---|---|---|---|---|---|
| Formu- | Storage | Drug | sulation | Particle | Drug | | Main | Second |
| lation | Condition | Content | Efficiency | Size | Purity | T_{g} | Melt | Melt |
| Number | (° C. ) | (weight %) | (%) | (μm) | (%) | (° C. ) | (° C. ) | (° C. ) |
| 00447-098 | 4 | 61.2 | 94.2 ± 4.7 | 74.5 | 99.5 | 34.1 | 126.1 | No peak |
| 00447-098 | 25 | 63.2 | 97.2 ± 2.9 | 77.8 | 99.6 | 42.1 | 126.5 | 113.8 |
| | | | | | | | | (tiny |
| | | | | | | | | peak) |
| 00447-098 | 30-35 | 66.2 | 101.8 ± 2.1 | 95.7 | 99.8 | 49 | 125.8 | 113.5 |
| | | | | | | | | (tiny |
| | | | | | | | | peak) |
| 00447-102 | 4 | 62.8 | 96.6 ± 3.3 | 63.5 | 99.4 | 34.6 | 125 | 114.2 |
| | | | | | | | | (tiny |
| | | | | | | | | peak) |
| 00447-102 | 25 | 57.6 | 88.6 ± 8.3 | 64.9 | 99.6 | 38 | 125 | 114.6 |
| 00447-102 | 30-35 | 65 | 100.1 ± 0.5 | 70.3 | 99.8 | 48.8 | 125 | 114.6 |
| 00447-108 | 4 | 63 | 96.9 ± 9.5 | 79.3 | 99.7 | 35 | 126.5 | No peak |
| 00447-108 | 25 | 62.8 | 96.6 ± 10.4 | 76.8 | 99.8 | 39.6 | 126.1 | No peak |
| 00447-108 | 30-35 | 68 | 104.6 ± 1.6 | 79.8 | 99.8 | 49.44 | 126.5 | No peak |
| 00447-110 | 4 | 52.1 | 104.3 ± 2.5 | 59.2 | 98.1 | 19.6 | 122 | 112.3 |
| 00447-110 | 25 | 45.5 | 91.0 ± 3.0 | 127.4 | 99.7 | 33.9 | 112.7 | 122.4 |

### Example 2. Drug Release In Vivo

Drug release from sustained release formulations typically differs in vitro and in vivo. The purpose of this experiment was to characterize this difference for nimodipine-PLGA formulations.

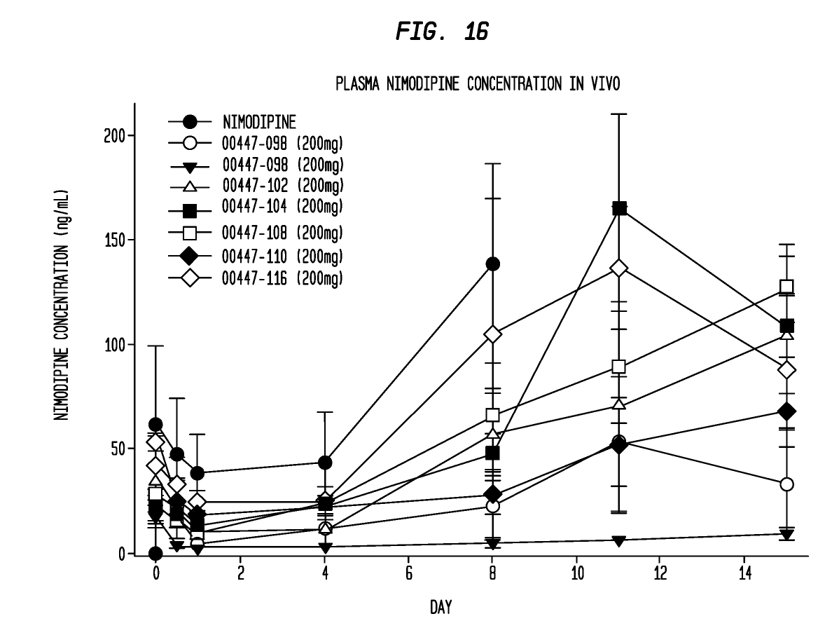

Six (6) nimodipine-PLGA formulations (n=6 per group) or pure nimodipine (n=4) were injected subcutaneously in Wistar rats at doses of 20 mg/kg or 200 mg/kg. Plasma was collected and nimodipine concentrations determined (FIG. 16). Sustained release of nimodipine over time was observed with all formulations, including pure nimodipine, which was likely due to its high lipid solubility and subsequent prolonged residence in subcutaneous fat. A poor relationship between the time course of release in vitro and in vivo was observed, although the order of release was similar. Without being bound by theory, it is hypothesized that release in vitro might be more reflective of how the formulation would release in the aqueous subarachnoid space, rather than in the fatty subcutaneous space. The rank order of release from fastest to slowest in vitro was 00447-116>00447-104>00447-108>00447-098>00447-102>00447-110. The rank order of release from fastest to slowest in vivo was 00447-116>00447-104>00447−108=00447-102>00447-110>00447-098. Thus, formulation 00447-108 was selected for preclinical and clinical development due to its stable and predetermined release characteristics in vitro. Formulation 00447-108 was manufactured in repeated 300 g batches using good manufacturing practice and shown to be stable for up to 12 months at −25° C. and 5° C.

Injectability of microparticles was tested and found to require at least an 18 gauge needle. Microparticles were injected using a 14 gauge needle that has an internal diameter of 1.6 mm, which corresponds to the internal diameter of standard clinically-used cerebral ventricular catheters. Injectability was facilitated by mixing microparticles with low viscosity hyaluronic acid (Hyalgan®, zero shear rate viscosity of 2 Poise, molecular weight 0.500-0.750 million Da purified natural sodium hyaluronic acid derived from rooster combs, pH 6.8-7.5, suspended in 0.9% NaCl). Without being bound by theory, it was hypothesized that for potential intracisternal injection, the formulation would have to remain at the site of application in the basal cisterns. If the formulation were liquid, it would potentially migrate into the subdural space during or after closure of the craniotomy. Thus, the microparticles were mixed with a high viscosity hyaluronic acid solution to make a paste-like material (Orthovisc®, zero shear rate viscosity of 1677 Poise, molecular weight 1.0-2.9 million Da, dissolved in 0.9% NaCl and produced through recombinant bacterial fermentation). Hyaluronic acid was chosen because of its use in humans for injection into the eye and synovial joints. It is an abundant glycosaminoglycan in brain extracellular matrix and is composed of repeating disaccharide units of sodium glucuronate-N-acetylglucosamine. It has been shown to be neuroprotective and it has limited toxicity when injected into the eye or brain.

### Example 3. Toxicity Studies

Toxicity studies were conducted in male and female CD® [Crl:CD®(SD)] rats and beagles.

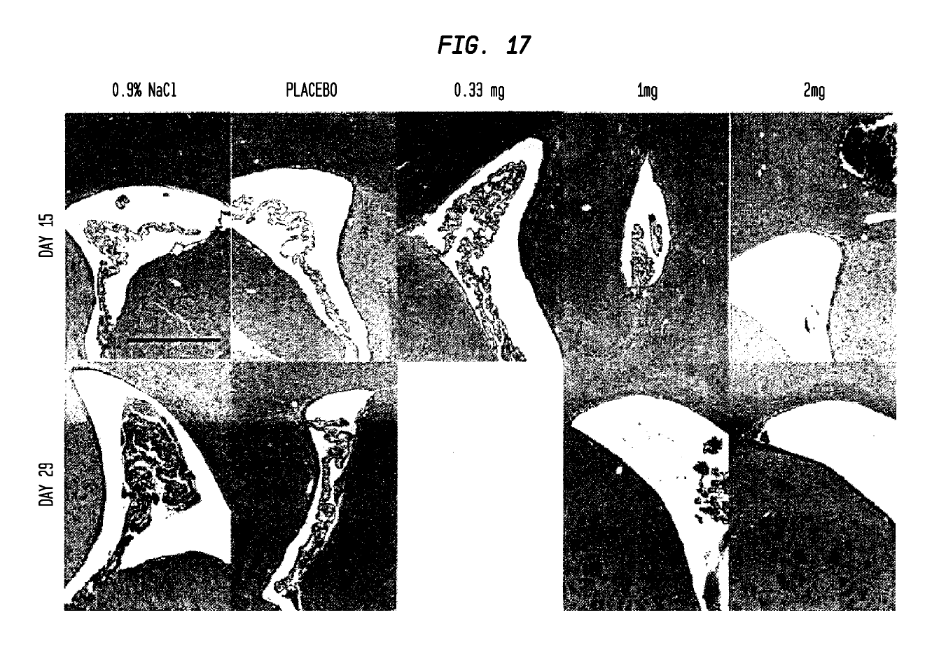

Rats (n=7 per sex per group) received a single intraventricular injection of placebo microparticles, 0.9% NaCl or nimodipine-PLGA microparticles in low-viscosity hyaluronic acid (Table 4, above). The dose of nimodipine was 0.33, 1, or 2 mg and all injections were at a volume of 23±5 μL. This corresponds to human doses of 200, 600 and 1200 mg, scaled on relative CSF volumes, assuming a 300 g rat has a CSF volume of 150 μL and a 70 kg human has a CSF volume of 150 mL, with the maximum dose being the maximum feasible dose. Additional animals received nimodipine-PLGA microparticles for toxicokinetic studies. Endpoints assessed were clinical observation, neurobehavioral evaluation, ophthalmoscopy, blood hematology and chemistry, urinalysis, plasma and CSF nimodipine and pathology at sacrifice on days 15 and 29 (day 1 was the day of injection in all studies). All animals survived to sacrifice. There were no effects of nimodipine-PLGA microparticles on clinical observations, neurobehavioral evaluations, body weight, food consumption, ophthalmoscopy, hematology, coagulation, clinical chemistry, urinalysis, organ weight, or macroscopic pathology. The only potential nimodipine-PLGA microparticle-related microscopic finding consisted of more frequent and severe intracranial hemorrhage at day 15, usually at the injection site in the brain of rats injected with 2 mg nimodipine-PLGA microparticles (FIG. 17). This finding was reversible as no differences were observed between groups by day 29.

Histopathologic changes including reactive gliosis, hydrocephalus, pigmented and foamy macrophages, focal mineralization and vacuolation at or near the injection site at both times were observed in all groups (FIG. 17). Without being limited by theory, the changes were thought to be related to the injection procedure based on the lack of dose response and the similar incidence in all groups.

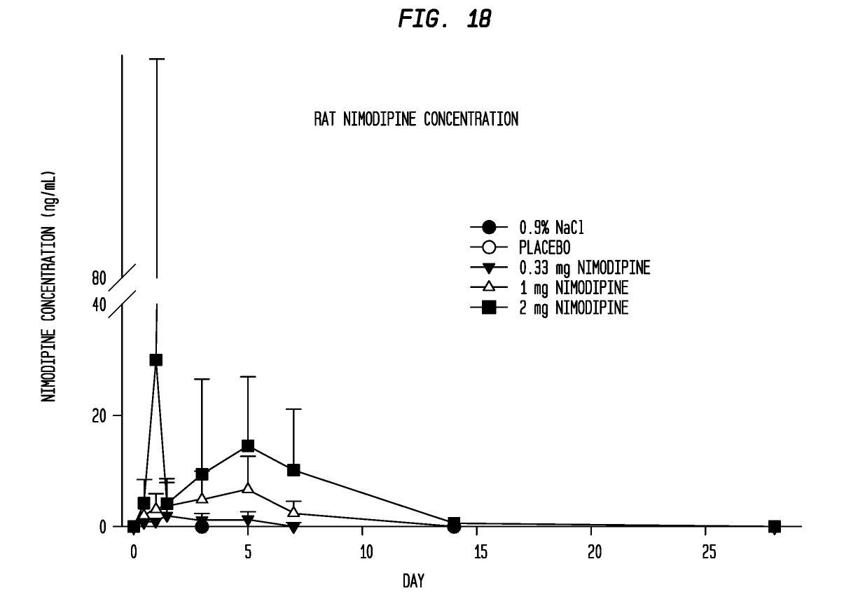

Mean plasma nimodipine concentrations that were determined with a validated liquid chromatography/mass spectrometry (LC-MS/MS) assay at 48 hours increased with increasing dose and ranged from 0.88 ng/mL to 10.6 ng/mL at 48 hours (FIG. 18). Plasma concentrations after administration of 2 mg nimodipine-PLGA microparticles were measurable on day 15 but not day 29. Mean plasma maximum observed concentration (Cₘₐₓ) and area under the curve from the time of dosing to the time of the last observation (AUC₀₋ₜ) was 40.5 ng/mL and 3902 ng*hr/mL after injection of 2 mg nimodipine-PLGA microparticles, 5.7 ng/mL and 901 ng*hr/mL after injection of 1 mg nimodipine-PLGA microparticles and 1.9 ng/mL and 177 ng*hr/mL after injection of 0.33 mg nimodipine-PLGA microparticles. Nimodipine was only measurable in 2 CSF samples obtained on day 3, secondary to difficulty sampling CSF. The no observable adverse effect level (NOAEL) was 2 mg.

A similar study was conducted in beagles in which nimodipine-PLGA microparticles were injected intraventricularly or into the cisterna magna. Thirteen (13) groups of male and female beagles (n=3 per sex per group) underwent intraventricular or intracisternal injection of 0.9% NaCl, placebo microparticles or nimodipine-PLGA microparticles (17, 51 or 103 mg nimodipine, Table 6, above). These doses corresponded to human doses of 200, 600 and 1200 mg, scaled on relative CSF volumes, assuming a CSF volume of 12.5 mL in a 12.5 kg beagle and a CSF volume of 150 mL in a 70 kg human, with the maximum dose being the maximum feasible dose (MFD). Intraventricular nimodipine-PLGA microparticles were suspended in low viscosity hyaluronic acid (Hyalgan®), and intracisternal nimodipine-PLGA microparticles were mixed with high viscosity hyaluronic acid (Orthovisc®). All injections were 1.15 mL and the animals were sacrificed 15 or 29 days after the injections. The endpoints were the same as in the rat toxicity study, except for a simpler behavior assessment (according to Zhou C, Yamaguchi M, Colohan A R, Zhang J H. Role of p53 and apoptosis in cerebral vasospasm after experimental subarachnoid hemorrhage. J Cereb Blood Flow Metab 2005; 25:572-82), that awake indirect blood pressure measurements were obtained, as well as electrocardiography (ECG).

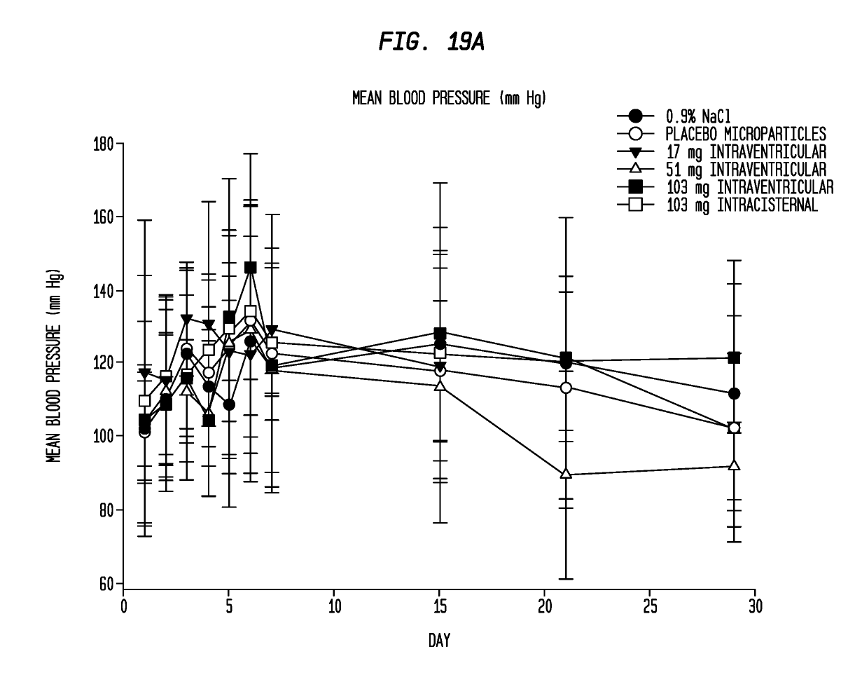

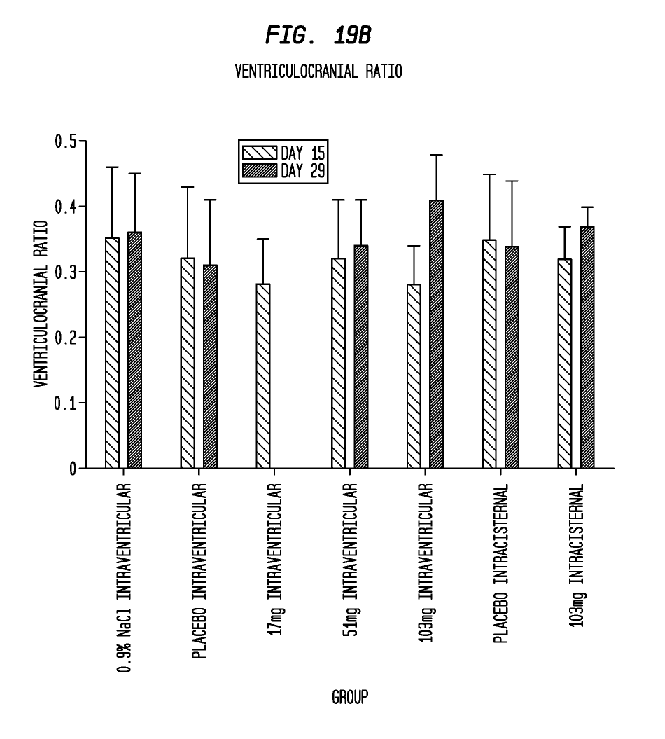

There were 2 mortalities. Otherwise, no clinical findings, changes in body weight, heart rate or blood pressure, clinical pathology, ECG or macroscopic observations were found (FIGS. 19A and 19B). Two males were euthanized in extremis prior to the completion of the study. Both received 2 mg nimodipine-PLGA microparticles intracisternally. They were euthanized on days 10 and 15. The beagle euthanized on day 10 was found on microscopy to have the fourth ventricle of the brain expanded by granulomatous inflammation admixed with subacute/chronic inflammation which infiltrated the lining of the fourth ventricle and choroid plexus. The inflammation surrounded moderate amounts of translucent, round, space occupying foreign material. No foreign material or granulomatous inflammation was observed within the brain stem, midbrain, or cerebellum. Within the subjacent brain stem was an area of mild cavitation with rarefaction and edema of the neuropil, multifocal minimal hemorrhage, and minimal axonal/myelin degeneration with presence of spheroids, suggesting that, at some point, an injection had penetrated the brainstem. In the beagle euthanized on day 15, the fourth ventricle of the brain contained minimal translucent foreign material surrounded by minimal granulomatous/subacute/chronic inflammation. No foreign material or granulomatous inflammation was observed within the brain stem, midbrain, or cerebellum.

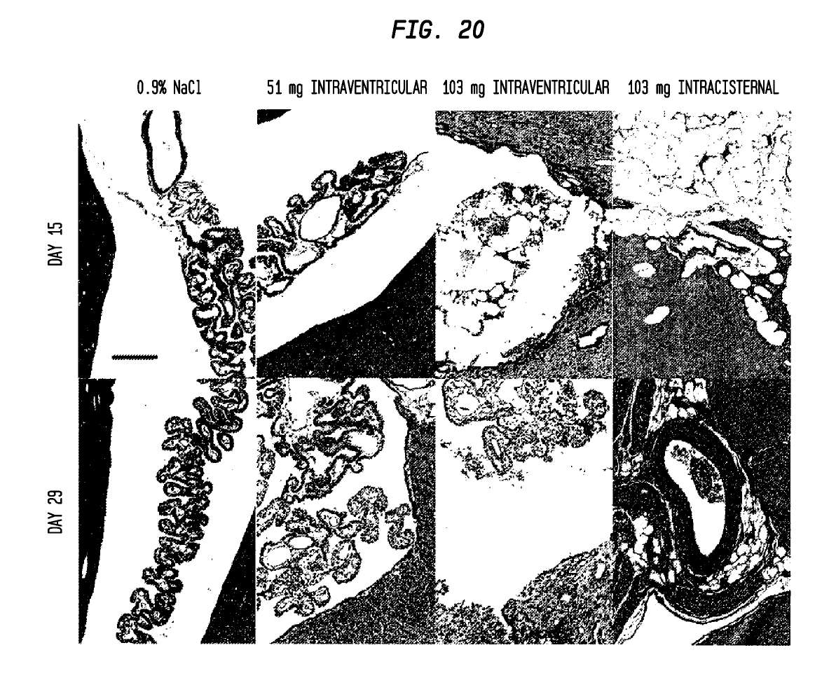

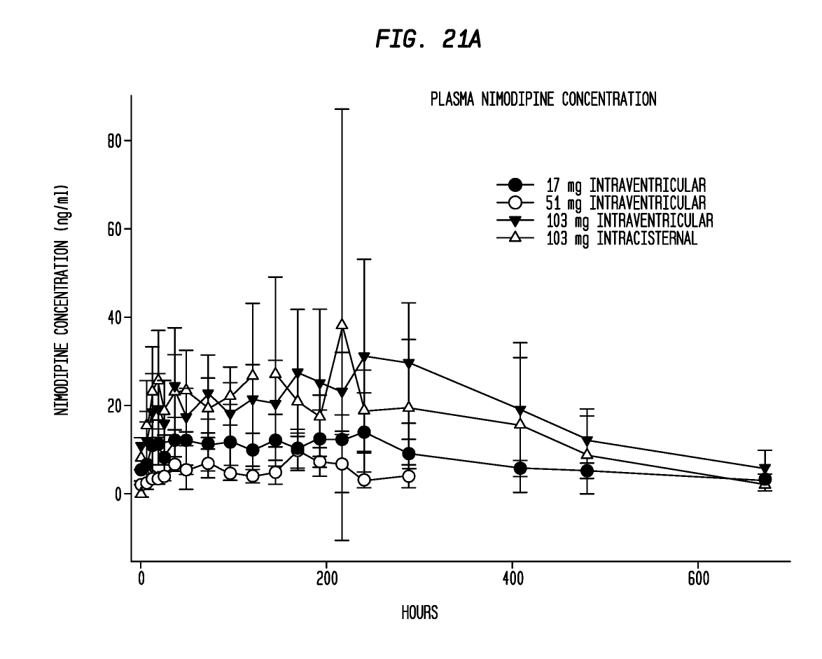

In general, microscopic findings were evident in the brain, spinal cord and heart of some beagles (FIG. 20). Intraventricular or intracisternal injection caused a granulomatous foreign body type reaction in the cerebral ventricles and subarachnoid space. This inflammation and/or the injection procedure often lead to a disruption of the ependymal lining and subsequent extension of the inflammation and/or foreign material into the adjacent periventricular neuropil. The inflammatory reaction and presence of foreign material was observed after injection of any amount of PLGA microparticles but the incidence and severity was dose-related. The periventricular extension of the inflammation was more prominent in animals receiving intraventricular injections. The foreign material and inflammatory reaction was incompletely resolved by day 29 Inflammation, fibrosis, and cardiac myofiber degeneration and necrosis of the left ventricle and interventricular septum of the heart were observed primarily in animals receiving the 103 mg nimodipine-PLGA microparticles by intracisternal injection (4 of 6 with mild changes at day 15), but was also noted in one beagle injected with 103 mg intraventricularly. There was partial resolution of the microscopic findings in the heart in the day 29 animals with mild left ventricular cardiac inflammation and fibrosis in only one beagle that received 103 mg intracisternally.

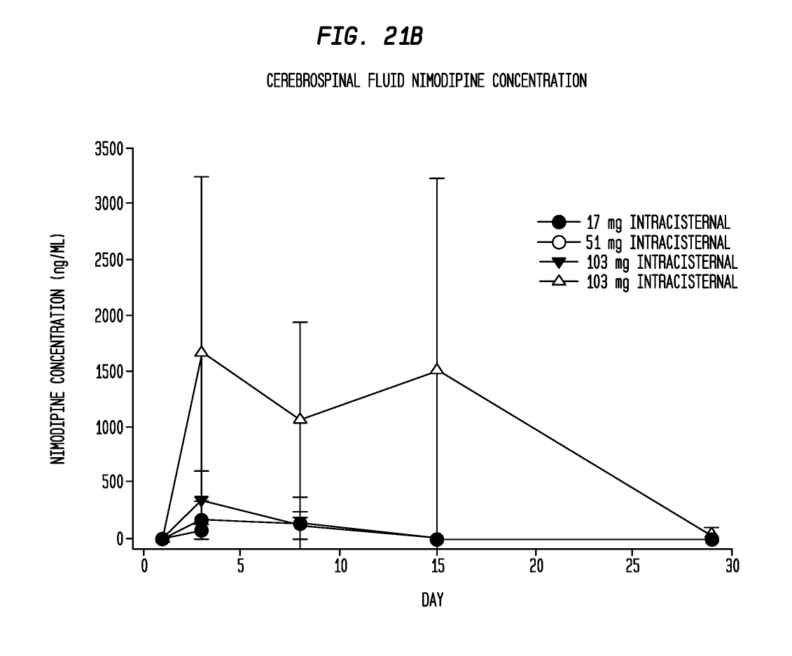

Mean plasma nimodipine concentrations following intraventricular administration of 17 mg, 51 mg or 103 mg nimodipine microparticles increased gradually during the first 48 hours and then remained generally constant until 336 hours (day 15, FIGS. 21A and 21B). Nimodipine was still detected at 672 hours in all groups receiving nimodipine-PLGA microparticles (day 29). Plasma concentrations were similar after a single intracisternal or intraventricular administration of 103 mg nimodipine-PLGA microparticles. Mean time of maximum concentration (Tₘₐₓ) ranged from 138 to 225 hours and did not vary significantly between groups when analyzed combining groups with the same dose and route of administration (analysis of variance [ANOVA], Table 9). Plasma Cₘₐₓ was significantly higher after administration of 103 mg doses compared to the 17 mg and 51 mg doses (ANOVA, P<0.005, pairwise comparisons P<0.05, Student-Newman-Keuls, Table 9). The AUC₀₋ₜ also was significantly higher for the 103 mg dose analyzed for 15 days compared to the 17 mg dose (P=0.003, ANOVA, pairwise comparisons P<0.05, Student-Newman-Keuls) and for all 3 doses/routes of administration at 29 days (P<0.001, ANOVA, pairwise comparisons P<0.05, Student-Newman-Keuls, Table 9). CSF nimodipine concentrations increased in a dose-dependent fashion and were highest at the 48 hour measurement (FIGS. 21A and 21B). Nimodipine remained detectable in CSF for 336 hours (day 15) after intraventricular administration of 51 or 103 mg or intracisternal administration of 103 mg, and at 672 hours (day 29) after intraventricular administration of 51 mg (but not intraventricular 103 mg) or intracisternal 103 with higher CSF after intracisternal administration. Based upon the results of this study, a NOAEL of 51 mg of nimodipine-PLGA microparticles administered intraventricularly in the beagle was established. These studies demonstrate sustained release of nimodipine achieving high concentrations in the subarachnoid space with systemic concentrations below those associated with systemic side effects (e.g., hypotension).

TABLE 9
Pharmacokinetics of Nimodipine-PLGA Microparticles in Beagle Dogs

| | Tₘₐₓ | Cₘₐₓ | AUC₀₋ₜ |
|---|---|---|---|
| Group | (hours, n) | (ng/mL, n) | (ng * hr/mL, n) |
| 17 mg intraventricular, day 15 | 138 ± 69 (6) | 13 ± 4 (6)* | 1783 ± 630 (6)** |
| 51 mg intraventricular, day 15 | 143 ± 110 (12) | 23 ± 9 (12)* | 4153 ± 1072 (6) |
| 51 mg intraventricular, day 29 | | | 5462 ± 1082 (6)† |
| 103 mg intraventricular, day 15 | 225 ± 113 (12) | 49 ± 16 (12) | 7250 ± 1767 (6) |
| 103 mg intraventricular, day 29 | | | 13749 ± 3134 (6)† |
| 103 mg intracisternal, day 15 | 155 ± 108 (12) | 55 ± 47 (12) | 7608 ± 4728 (6) |
| 103 mg intracisternal, day 29 | | | 9886 ± 2987 (6)† |

*Significantly different than both 103 mg groups (P = 0.005, ANOVA, pairwise comparisons P < 0.05, Student-Newman-Keuls)
**Significantly different than day 15 103 mg groups (P = 0.003, ANOVA, pairwise comparisons P < 0.05, Student-Newman-Keuls)
†Significantly different than other day 29 groups (P < 0.001, ANOVA, pairwise comparisons P < 0.05, Student-Newman-Keuls)

### Example 4. Mongrel Dog Efficacy Study

Subarachnoid hemorrhage (SAH) was induced in 40 mongrel dogs (day 1) and angiographic vasospasm, behavior, brain microthrombi and brain injury were assessed. At the time of SAH, dogs were treated with intracisternal placebo microparticles with or without oral nimodipine (5.2 mg/kg daily for 21 days), intracisternal nimodipine-PLGA microparticles (40 mg), intracisternal nimodipine-PLGA microparticles (100 mg) or intraventricular nimodipine-PLGA microparticles (100 mg). One dog injected intraventricularly with nimodipine-PLGA microparticles (100 mg) was euthanized on day 25 and one in the placebo microparticle plus oral nimodipine group was euthanized on day 35. Both animals appeared healthy in the days leading up to CSF sampling, after which they did not awaken. The cause of death was determined to be brainstem injury from aspiration of CSF from the cisterna magna.

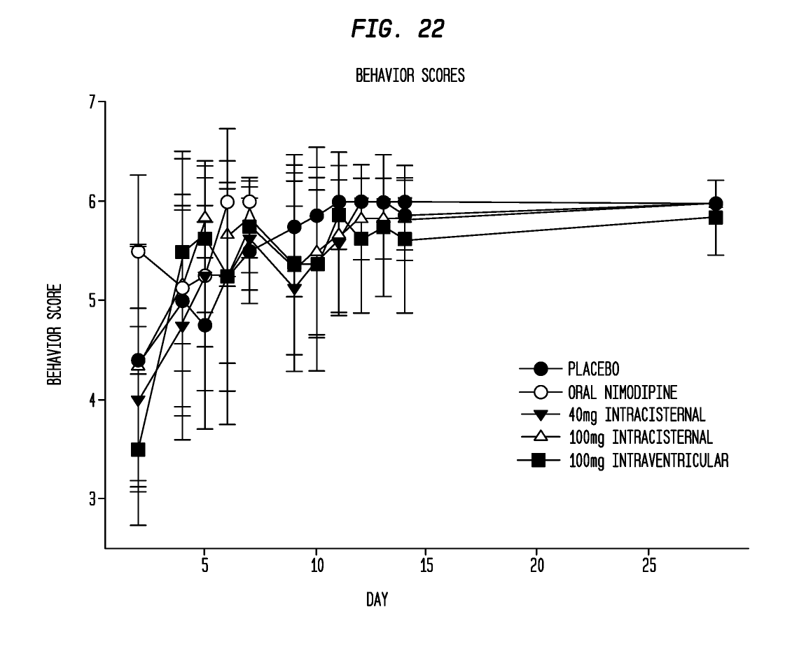

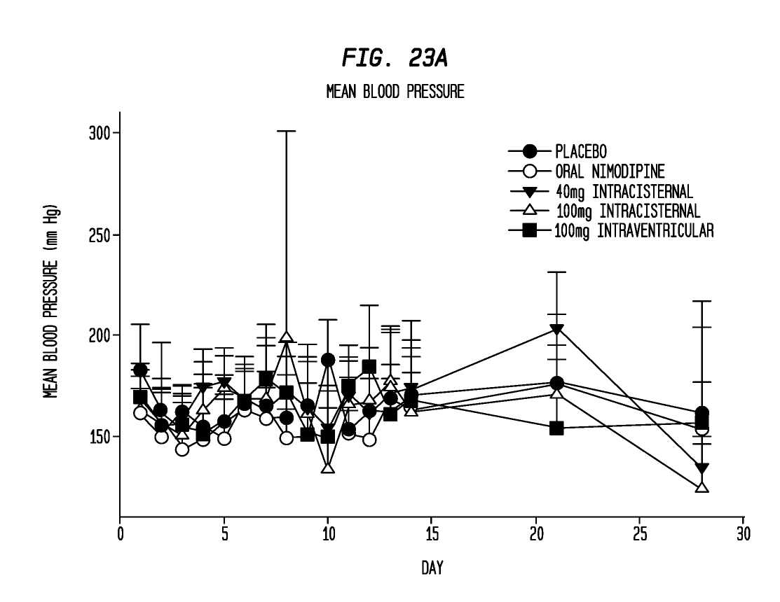

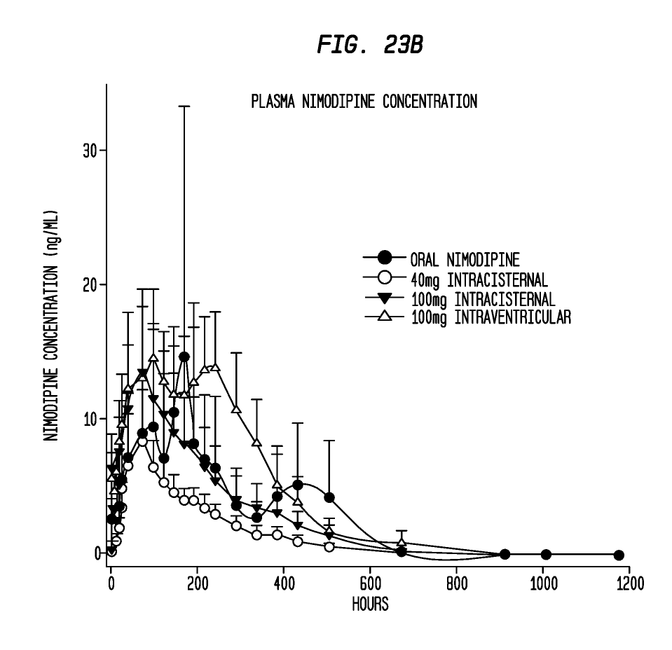

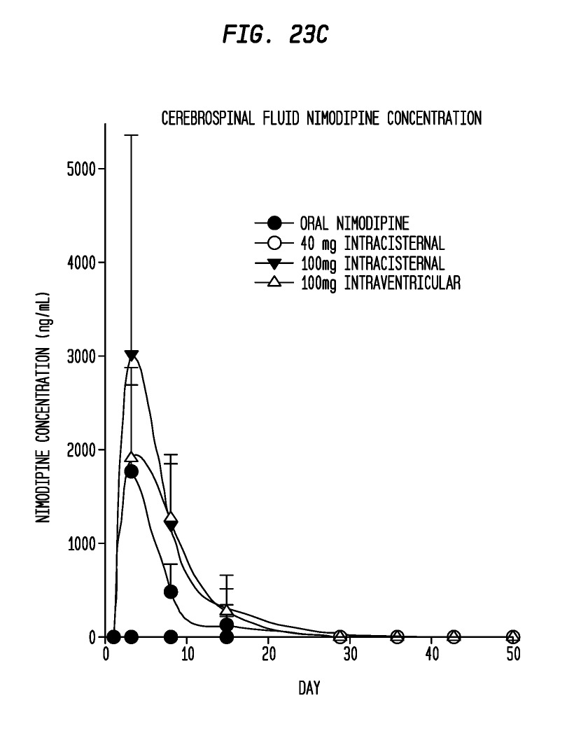

Clinical examination showed similar findings in all groups with decreased appetite and activity within the first 5 days of SAH, followed by recovery, as assessed on a 9-point scale (the between group scores at each time differed significantly only between placebo versus 40 mg intracisternal and placebo versus 100 mg intraventricular on day 2 [ANOVA P=0.005, pairwise comparisons by Holm-Sidak P<0.05], FIG. 22). There were significant differences in behavior over time within all groups, with significantly worse behavior in the placebo group on day 4 compared to days 6, 7, 11, 12, 14 and 28 (ANOVA, P<0.001, pairwise comparisons by Holm-Sidak, P<0.05), oral nimodipine day 2 significantly worse than days 9, 10, 11, 12, 13, 14 and 28 and day 5 significantly different than days 11, 13 and 14 (ANOVA P<0.001, pairwise comparisons by Holm-Sidak, P<0.05), 40 mg intracisternal day 2 significantly worse than days 7, 10, 11, 12, 13, 14 and 28 (ANOVA P=0.025, pairwise comparisons by Holm-Sidak, P<0.05), 100 mg intracisternal day 2 significantly worse than day 28 (ANOVA P<0.001, pairwise comparisons by Holm-Sidak, P<0.05) and 100 mg intraventricular day 2 significantly worse than all other days (ANOVA P<0.001, pairwise comparisons by Holm-Sidak, P<0.05). Mean blood pressures varied significantly in dogs treated with 40 mg intracisternal nimodipine-PLGA microparticles (P<0.001, ANOVA, pairwise differences between day 21 and each of days 2, 3, 7, 9, 10, 12 and 28 and between day 28 and each of days 4, 5 and 14, Holm-Sidak, FIGS. 23A, 23B and 23C). There also was significant variance in mean blood pressure in the group treated with 100 mg intracisternal nimodipine-PLGA microparticles (P<0.019, no pairwise differences). When comparing between groups over time, there was significant variance in blood pressure on day 5 but no pairwise differences between groups (ANOVA, P=0.033), on day 10 with pairwise differences between placebo and 100 mg intracisternal or intraventricular (ANOVA, P=0.004, Holm-Sidak P<0.05) and on day 21 with pairwise difference between 40 mg intracisternal and 100 mg intraventricular (ANOVA, P=0.029, Holm-Sidak pairwise P<0.05). Without being bound by theory, the lack of relationship between plasma nimodipine concentration and blood pressure suggests that intracranial administration of nimodipine-PLGA microparticles does not produce sufficient systemic exposure or fluctuations in plasma concentrations to produce substantial changes in blood pressure.

Body temperature, oxygen saturation and heart rate were monitored every 5 minutes during anesthesia on day 1 and during angiography on day 8. There were variations in heart rate that were considered to be within normal limits for mongrel dogs under anesthesia.

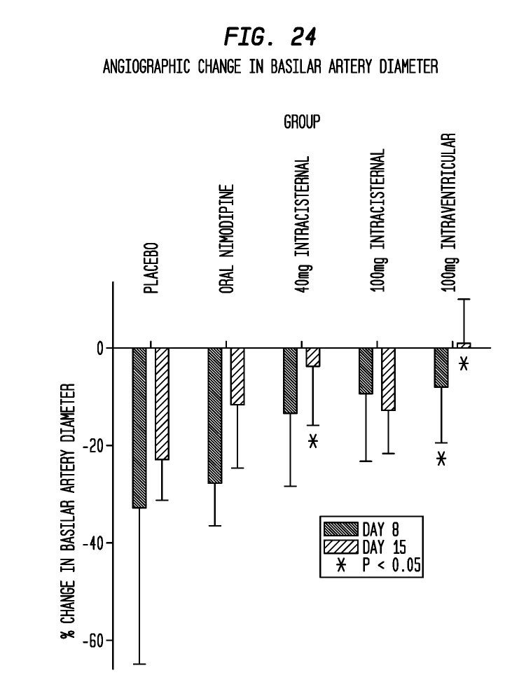

Angiography showed that there were significant reductions in basilar artery diameter 8 and 15 days after SAH in dogs treated with placebo microparticles with or without nimodipine (ANOVA, P=0.001, days 8 and 15 significantly different from day 1, P<0.05, Tukey test, FIG. 24, Table 10). When comparing the percent reduction in basilar artery diameter between groups at day 8, the group treated with 100 mg nimodipine-PLGA microparticles intraventricular had significantly less arterial narrowing than the placebo microparticle groups with or without oral nimodipine (P<0.009, ANOVA, Holm-Sidak). When comparing the percent reduction in basilar artery diameter between groups at day 15, the groups treated with 40 mg nimodipine-PLGA microparticles intracisternal or 100 mg intraventricular had significantly less arterial narrowing than the placebo microparticle groups with or without oral nimodipine (P<0.009, ANOVA, Holm-Sidak).

TABLE 10
Angiographic Diameter of Basilar Artery of Intracisternal or Intraventricular Nimodipine-PLGA
or Placebo Microparticles with or without Oral Nimodipine in Mongrel Dogs

| | | ANOVA | ANOVA | | | |
|---|---|---|---|---|---|---|
| | Diameter | between | between | | | |
| | (mm, | groups | groups | ANOVA | | |
| | ANOVA | P < 0.05, | P < 0.05, | within | ANOVA | ANOVA |
| | between | no pairwise | no pairwise | groups | between | between |
| | groups | differences | differences | raw data | groups | groups |
| | P = 0.049, | (% change | (% change | (% change | on % | on % |
| | no pairwise | compared | compared | compared | day 1 | day 1 |
| Group | differences) | to day 1) | to day 1) | to day 1) | to day 8 | to day 15 |
| | Day 1 | Day 8 | Day 15 | | P = 0.009 | P = 0.002 |
| Placebo | 1.50 ± 0.07 | 1.01 ± 0.47 | 1.15 ± 0.15 | P = 0.001, | | Significantly |
| microparticles | | (−33.0 ± 11.6%) | (−23.1 ± 3.1) | day 8 and | | different from |
| (n = 7) | | | | 15 less | | 100 mg |
| | | | | than day 1, | | nimodipine- |
| | | | | P < 0.05 | | PLGA |
| | | | | | | microparticles |
| | | | | | | intraventricular |
| | | | | | | (P = 0.002) |
| | | | | | | and 40 mg |
| | | | | | | nimodipine- |
| | | | | | | PLGA |
| | | | | | | microparticles |
| | | | | | | intracisternal |
| | | | | | | (P = 0.011) |
| Placebo | 1.51 ± 0.13 | 1.08 ± 0.12 | 1.32 ± 0.17 | P < 0.001, | Significantly | |
| microparticles + | | (−27.9 ± 3.0) | (−11.7 ± 4.5) | all groups | different from | |
| oral | | | | different | 100 mg | |
| nimodipine | | | | from each | nimodipine- | |
| (n = 8) | | | | other, | PLGA | |
| | | | | P < 0.02 | microparticles | |
| | | | | | intraventricular | |
| | | | | | (P < 0.05) | |
| 40 mg | 1.44 ± 0.17 | 1.24 ± 0.22 | 1.40 ± 0.31 | No | | Significantly |
| intracisternal | | (−13.3 ± 5.3) | (−3.6 ± 4.3) | significant | | less than oral |
| nimodipine- | | | | differences | | nimodipine |
| PLGA | | | | | | (P < 0.05) |
| microparticles | | | | | | |
| (n = 8) | | | | | | |
| 100 mg | 1.46 ± 0.06 | 1.31 ± 0.19 | 1.27 ± 0.11 | No | | |
| intracisternal | | (−9.4 ± 5.6) | (−12.7 ± 3.5) | significant | | |
| nimodipine- | | | | differences | | |
| PLGA | | | | | | |
| microparticles | | | | | | |
| (n = 6) | | | | | | |
| 100 mg | 1.31 ± 0.18 | 1.19 ± 0.09 | 1.33 ± 0.14 | No | Significantly | Significantly |
| intraventricular | | (−8.1 ± 3.9) | (1.1 ± 3.5) | significant | less than oral | less than oral |
| nimodipine- | | | | differences | nimodipine | nimodipine |
| PLGA | | | | | (P < 0.05) | (P < 0.05) |
| microparticles | | | | | | |
| (n = 8) | | | | | | |

* Values are means ± standard deviations (n = 6-8 per group). If significant difference by ANOVA, pairwise differences assessed by Holm-Sidak method.

Plasma nimodipine concentrations increased in a dose-dependent fashion to a peak between 36 hours to 4 days after administration of nimodipine-PLGA microparticles intracisternally or intraventricularly (FIGS. 23A, 23B and 23C). Nimodipine was still detected in plasma 21 days after administration of nimodipine-PLGA microparticles. The plasma concentration after administration of 100 mg nimodipine-PLGA microparticles was qualitatively similar to that achieved with oral nimodipine. Cerebrospinal fluid nimodipine concentrations were highest after administration of nimodipine-PLGA microparticles on day 3 compared to the other days it was measured, with measurable concentrations present at day 35 in all nimodipine-PLGA microparticle groups. Animals treated with oral nimodipine had very low concentrations of nimodipine in CSF that were highest on day 3 (1.56±1.17 ng/mL, n=7). Nimodipine concentrations were approximately 1000-fold higher in animals treated with nimodipine-PLGA microparticles than in these animals.

Dogs were euthanized on day 28 or 49 (n=4 per group per day). Microscopic changes in the brains consisted of granulomatous inflammation (aggregates of giant cells engulfing foreign material), subacute/chronic inflammation (mixed inflammatory infiltrate), fibroplasia, hemorrhage and pigmented macrophages. These findings were mostly located in the meninges of pons, medulla, and to a lesser extent the cervical subarachnoid space. They were more prominent at the 28 day time and had a lower incidence and severity at day 49.

Microthrombi and cerebral ischemia/infarction have been observed after SAH in humans and animals and are hypothesized to contribute to delayed cerebral ischemia. Fluoro-jade B staining conducted on coronal sections of brain from each group did not reveal any degenerative/necrotic neurons. Apoptotic neurons were not detected in any animals by immunoreactivity to activated caspase-3. Apoptosis was detected in glial cells evenly distributed throughout the brain. Microthrombi were noted in 2 dogs in the placebo microparticle, one in the placebo microparticle plus oral nimodipine, one in the 40 mg intracisternal, one in the 100 mg intracisternal and 2 in the 100 mg intraventricular nimodipine-PLGA microparticle group. Microthrombi were limited to the cerebrum with too few microthrombi observed to conduct statistical analysis.

The results of the experiments set forth above establish the sustained release and delivery, pharmacokinetics and bioactivity of a microparticulate pharmaceutical formulation comprising PLGA and a therapeutic agent such as nimodipine. Intracisternal or intraventricular injection of nimodipine-PLGA microparticles in rats and beagles demonstrated dose-dependent, sustained concentrations of nimodipine in plasma and cerebrospinal fluid for up to 29 days without toxicity in the brain or systemic tissues at doses less than 2 mg in rats and 51 mg in beagles, which would be equivalent of up to 612 to 1200 mg in humans, based on scaling relative to cerebrospinal fluid volumes. Efficacy of nimodipine-PLGA microparticles was tested in the double hemorrhage dog model of SAH. The results of this testing demonstrated that nimodipine-PLGA microparticles significantly attenuated angiographic vasospasm.

### EQUIVALENTS

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A site-specific microparticulate pharmaceutical formulation, comprising:
(a) a therapeutic amount of an L-type voltage-gated calcium channel antagonist;
(b) a poly(DL-lactide-co-glycolide) (PLGA) polymer comprising from 25% to 50% glycolide; and
(c) less than 5% hyaluronic acid,
wherein the microparticles of the formulation are characterized by:
(i) a particle size from about 20 μm to about 125 μm;
(ii) a drug load of from about 50% to about 70%;
(iii) sustained release; and
(iv) at least 99% purity.

2. The pharmaceutical formulation according to claim 1, wherein the L-type voltage-gated calcium channel antagonist is a dihydropyridine.

3. The pharmaceutical formulation according to claim 2, wherein the dihydropyridine is selected from the group consisting of nimodipine, nifedipine, nicardipine, clevidipine, and nisoldipine.

4. The pharmaceutical formulation according to claim 3, wherein the formulation comprises about 40 mg to about 1200 mg of nimodipine.

5. The pharmaceutical formulation according to claim 1, wherein the hyaluronic acid is characterized by a zero shear rate viscosity of 2 Poise and a molecular weight from about 0.500 million to about 0.750 million Da.

6. The pharmaceutical formulation according to claim 1, wherein the hyaluronic acid is characterized by a zero shear rate viscosity of 1677 Poise and a molecular weight from about 1.0 million to about 2.9 million Da.

7. The pharmaceutical formulation according to claim 4, wherein the nimodipine contains at least 51% Form 1 of nimodipine.

8. The pharmaceutical formulation according to claim 7, wherein the formulation is stable after storage for up to 12 months at −25° C. and 5° C.

9. The pharmaceutical formulation according to claim 1, wherein initial burst of release of the therapeutic agent within 24 hours of administration is <25%.

10. The pharmaceutical formulation according to claim 1, wherein the mean particle size is about 70 μm to about 100 μm.

11. The pharmaceutical formulation according to claim 1, wherein the formulation is terminally sterilized.

12. A site-specific microparticulate pharmaceutical formulation for administration into an intracisternal site of administration comprising,
(i) from 40 mg to about 1200 mg of an L-type voltage gated calcium channel antagonist;
(ii) a poly(DL-lactide-co-glycolide) (PLGA) polymer comprising 50% glycolide;
(iii) less than 5% of a hyaluronic acid characterized by a zero shear rate viscosity of 1677 Poise, molecular weight 1.0-2.9 million Da;
wherein consistency of the formulation is that of a paste,
wherein the microparticles of the formulation are characterized by:
(a) a particle size from about 20 μm to about 125 μm;
(b) a drug load of from about 50% to about 70%;
(c) sustained release; and
(d) at least 99% purity.

13. A site-specific microparticulate pharmaceutical formulation for administration into an intraventricular, intracisternal or intrathecal site of administration, comprising:
(a) from 40 mg to 1200 mg of an L-type voltage gated calcium channel antagonist;
(b) a poly(DL-lactide-co-glycolide) (PLGA) polymer comprising 50% glycolide;
(c) less than 5% of a hyaluronic acid characterized by a zero shear rate viscosity of 2 Poise, molecular weight of 0.500-0.750 million Da,
wherein viscosity of the formulation ranges from about 1.5 Poise to about 3.5 poise,
wherein the microparticles of the formulation are characterized by:
(i) a particle size from about 20 μm to about 125 μm;
(ii) a drug load of from about 50% to about 70%;
(iii) sustained release; and
(iv) at least 99% purity.

14. The site specific microparticulate pharmaceutical formulation according to any one of claims 12 and 13, wherein the mean particle size is about 70 μm to about 100 μm.

15. The site specific microparticulate pharmaceutical formulation according to any one of claims 12 and 13, wherein the formulation is terminally sterilized.

16. The site-specific microparticulate pharmaceutical formulation according to any one of claims 1, 12 and 13, prepared by a process comprising:
a) providing the at least 51% pure L-type voltage gated calcium channel antagonist;
b) adding the L-type voltage gated calcium channel antagonist to a PLGA polymer solution containing 50% glycolide and a solvent, thereby creating a mixture of the bioactive agent and the polymer solution;
c) homogenizing the mixture to form a disperse phase comprising the L-type voltage gated calcium channel antagonist and the PLGA solution;
d) mixing the disperse phase with a continuous phase comprising a surfactant dissolved in deionized water, thereby forming an emulsion comprising the L-type voltage gated calcium channel antagonist;
e) forming the particles comprising the L-type voltage gated calcium channel antagonist by precipitating the polymer and extracting the solvent;
f) collecting the microparticles on sieves, lyophilizing and storing the microparticles at −20° C.;
g) sterilizing the microparticles using gamma irradiation;
h) drying the particles; and
i) formulating the microparticles with less than 5% of a hyaluronic acid, wherein viscosity of the formulation ranges from about 1.5 Poise to about 3.5 Poise.

17. The process according to claim 16, wherein the solvent is ethyl acetate, the surfactant is PVA, and the process comprises >91% encapsulation efficiency.

18. The process according to claim 16, wherein the formulation is terminally sterilized.

19. A method for treating a delayed complication of a brain injury associated with interruption of a cerebral artery comprising a delayed cerebral ischemia, comprising administering a pharmaceutical composition containing a therapeutic amount of the formulation of any one of claim 1, 10, 12 or 13 to a subject in need thereof, wherein the therapeutic amount is effective to reduce incidence of a poor outcome, as measured on the Glasgow outcome score (GOS), extended Glasgow outcome score (GOSE), modified rankin scale (mRS), Montreal cognitive assessment, or a neurocognitive assessment compared to the outcome expected without treatment or in patients treated with preservative free saline solution without toxicity in either brain or systemic tissues.

20. The method according to claim 19, wherein the poor outcome is a score of 1, 2 or 3 on the Glasgow outcome scale (GOS).

21. The method according to claim 19, wherein the poor outcome is a score of 1, 2, 3 or 4 on the extended Glasgow outcome scale (GOSE).

22. The method according to claim 19, wherein the poor outcome is a score of 1, 2, 3, 4 or 5 on the extended Glasgow outcome scale (GOSE).

23. The method according to claim 19, wherein the formulation is terminally sterilized.


SEQUENCE LISTING (2 sequences)

SEQ ID NO: 1 - protein, 60 aa, Dendroaspis polylepis
RICYIHKASLPRATKTCVENTCYKMFIRTQREYISERGCGCPTAMWPYQTECCKGDRCNK

SEQ ID NO: 2 - protein, 60 aa, Dendroaspis angusticeps
WQPPWYCKEPVRIGSCKKQFSSFYFKWTAKKCLPFLFSGCGGNANRFQTIGECRKKCLGK